(19) United States
(12) Patent Application Publication
(10) Pub. No.: US 2014/0323581 A1
(43) Pub. Date: Oct. 30, 2014

(54) TRANSCRIPTOME WIRING ANALYSIS IN PARKINSON'S DISEASE AND USES THEREOF

(71) Applicant: THE TRUSTEES OF COLUMBIA UNIVERSITY IN THE CITY OF NEW YORK, New York, NY (US)

(72) Inventors: Asa Abeliovich, New York, NY (US); Hervé Rhinn, New York, NY (US)

(21) Appl. No.: 14/362,882

(22) PCT Filed: Dec. 5, 2012
(86) PCT No.: PCT/US12/68023

§ 371 Date: Jun. 4, 2014

Related U.S. Application Data
(60) Provisional application No. 61/566,925, filed on Dec. 5, 2011.

(51) Int. Cl. C12Q 1/68

(52) U.S. Cl. CPC C12Q 1/6883
USPC 514/654; 435/6.12; 514/657

(57) ABSTRACT

The invention is directed to methods to identify predisposition or risk to develop Parkinson's disease, methods to identify agents which have therapeutic effect on Parkinson's disease, and methods to determine the therapeutic effect of an agent in a subject suffering from Parkinson's disease, and to kits and reagents for carrying out the methods of the invention.

This application claims the priority to Application Ser. No. 61/566,925 filed Dec. 5, 2011, the content of which is hereby incorporated in its entirety.

This invention was made with government support under RO1NS064433 awarded by NIH-NINDS. The government has certain rights in the invention.

The contents of all patents, patent applications and non-patent references listed in the specification are incorporated by reference herewith.

### BACKGROUND

Parkinson's disease (PD) is a degenerative disorder of the central nervous system. It results from the death of dopamine-containing cells in the substantia nigra, a region of the midbrain; the cause of cell-death is unknown. Early in the course of the disease, the most obvious symptoms are movement-related, including shaking, rigidity, slowness of movement and difficulty with walking and gait. Later, cognitive and behavioral problems may arise, with dementia commonly occurring in the advanced stages of the disease. Other symptoms include sensory, sleep and emotional problems. PD is more common in the elderly with most cases occurring after the age of 50.

Parkinson's disease is diagnosed by a physician exam, and diagnosis is based on the medical history and a neurological examination of the patient. There is no laboratory or molecular test that will clearly identify the disease. Brain scans are sometimes used to rule out disorders that could give rise to similar symptoms. Patients may be given levodopa, or other dopamine affecting agent, and resulting relief of motor impairment tends to confirm diagnosis. The finding of Lewy bodies in the midbrain on autopsy is usually considered proof that the patient suffered from Parkinson's disease. Thus there is need for biomarkers for PD disease or treatment.

### SUMMARY

In certain aspects, the invention provides methods to determine predisposition or risk to develop Parkinson's Disease (PD) in a subject in need thereof comprising: (a) providing a biological sample from a subject in need thereof, (b) determining a ratio of SNCA long transcript to SNCA total transcript in the subject's biological sample and (c) comparing the ratio of SNCA long transcript to SNCA total transcript from the subject sample to a reference ratio of SNCA long transcript to SNCA total transcript, wherein the reference ratio is associated with a non-PD status, and wherein an increased ratio of SNCA long transcript to SNCA total transcript in the subject's sample compared to the reference ratio of SNCA long transcript to SNCA total transcript is indicative of a risk for developing Parkinson's Disease.

In certain aspects, the invention provides methods to diagnose PD in a subject in need thereof, the method comprising: (a) providing a biological sample from a subject in need thereof, (b) determining a ratio of SNCA long transcript to SNCA total transcript in the subject's sample and (c) comparing the ratio of SNCA long transcript to SNCA total transcript from the subject's sample to a ratio of SNCA long transcript to SNCA total transcript in a reference sample from healthy individuals/non-PD status, wherein an increased ratio of SNCA long transcript to SNCA total transcript in the subject's sample compared to the ratio of SNCA long transcript to SNCA total transcript in the reference non-PD status sample indicates that the subject is suffering from Parkinson's Disease.

In certain embodiments, the methods further comprise comparing the ratio of SNCA long transcript to SNCA total transcript from the subject to a reference ratio of SNCA long transcript to SNCA total transcript for a PD disease status; wherein a ratio of SNCA long transcript to SNCA total transcript in the subject's sample which is similar or comparable to the reference ratio of SNCA long transcript to SNCA total transcript for a PD status indicates that the subject is suffering from PD.

In certain aspects, the invention provides methods to diagnose PD in a subject in need thereof, comprising: (a) providing a biological sample from a subject, (b) determining a ratio of SNCA long transcript to SNCA total transcript in the sample obtained from the subject; (c) comparing the ratio of SNCA long transcript to SNCA total transcript from the subject to a reference ratio of SNCA long transcript to SNCA total transcript for a PD disease status; wherein a ratio of SNCA long transcript to SNCA total transcript in the subject's sample which is similar or comparable to the reference ratio of SNCA long transcript to SNCA total transcript for a PD status indicates that the subject is suffering from PD.

In certain embodiments, the methods further comprise comparing the ratio of SNCA long transcript to SNCA total transcript from the subject's sample to a ratio of SNCA long transcript to SNCA total transcript in a reference sample from healthy individuals/non-PD status, wherein an increased ratio of SNCA long transcript to SNCA total transcript in the subject's sample compared to the ratio of SNCA long transcript to SNCA total transcript in the reference non-PD status sample indicates that the subject is suffering from Parkinson's Disease.

In certain embodiments, the PD disease status is determined by any suitable method, including but not limited to a physical examination of the subject, a neurological examination of the subject, a brain scan, or a combination thereof. In certain embodiments, the subject is not diagnosed with PD.

In certain embodiments, the methods further comprise a physical examination of the subject, a neurological examination of the subject, a brain scan, or a combination thereof.

In certain embodiments, the methods further comprise a step of sequencing nucleic acids isolated from the subject's sample to determine the presence or absence of a PD-risk associated SNP, wherein the presence of a PD-risk associated SNP is further indicative that the subject is at risk of developing PD or is suffering from PD. In certain embodiments, the SNP is rs356168C/C risk-associated variant, rs356165 risk-associated variant, rs2736990 risk-associated variant, any other risk associated SNP, or any combination thereof, or any other suitable SNP.

In certain embodiments, the subject is suspected of having PD or is at risk of developing PD based on the presence of any one of parkinsonism symptoms, determined by any suitable method, including but not limited to a physical examination of the subject, a neurological examination of the subject, a brain scan, or a combination thereof.

In certain embodiments, the methods are carried out in the absence or presence of dopamine affecting agent administered to the subject, wherein an increased ratio of SNCA long transcript to SNCA total transcript in the presence of dopamine compared to the ratio of SNCA long transcript to SNCA total transcript in the absence of dopamine is indicative of a subject having an increased risk to develop PD.

In certain aspects, the invention provides methods to identify a candidate agent which has a therapeutic effect on PD, the method comprising: (a) providing a sample from a cortical neuron cell culture, (b) determining a ratio of SNCA long transcript to SNCA total transcript in a sample from the cortical neuron cell culture, wherein the sample is obtained in the presence and absence of a candidate agent, wherein a lowered ratio of SNCA long transcript to SNCA total transcript in the sample in the presence of the candidate agent is indicative of an agent which is a therapeutic agent for treatment of PD.

In certain aspects, the invention provides methods to identify a candidate agent which has a therapeutic effect on PD, the method comprising: (a) providing a sample from an animal model of PD; (b) determining a ratio of SNCA long transcript to SNCA total transcript in the sample from an animal model of PD, wherein the sample is obtained in the presence and absence of a candidate agent, administered to the animal model of PD, wherein a lowered ratio of SNCA long transcript to SNCA total transcript in the sample in the presence of the candidate agent is indicative of an agent which is a therapeutic agent for treatment of PD.

In certain aspects, the invention provides methods to determine a therapeutic effect of a candidate agent in a subject suffering from PD, the method comprising: (a) determining a ratio of SNCA long transcript to SNCA total transcript in a sample from a subject suffering from PD, wherein the sample is obtained in the presence and absence of a candidate agent, wherein a lowered ratio of SNCA long transcript to SNCA total transcript in the sample in the presence of the candidate agent is indicative of an agent which is a therapeutic agent for treatment of PD.

In certain embodiments of the methods, the lowered ratio of SNCA long transcript to SNCA total transcript in the sample in the presence of the candidate agent is due to a reduced level of SNCA long transcript in the presence of the candidate agent compared to level of SNCA long transcript the absence of the candidate agent.

In certain embodiments of the methods, the subject is diagnosed with PD and is not administered dopamine affecting agents (i.e. not treated for PD).

In certain embodiments of the methods, the subject is diagnosed by clinical symptoms, imaging of dopamine uptake, or combination thereof.

In certain embodiments of the methods, a ratio of SNCA long transcript to SNCA total transcript is determined by quantifying SNCA long transcript and SNCA total transcript.

In certain embodiments, the methods further comprise isolating nucleic acids from the subject's biological sample.

In certain embodiments, the methods further comprise quantifying the levels of SNCA long transcript and SNCA total transcript, wherein the levels of SNCA long transcript and SNCA total transcript are quantified by RT-qPCR, or any other suitable method.

In certain embodiments, the ratio of SNCA long transcript to SNCA total transcript is determined in a CSF sample, blood sample, plasma, or serum.

The invention provides a kit comprising PCR primers to carry out step (b) of the method of any one of the methods and instructions to carry out steps (a), (b) and (c) of these methods.

A kit comprising at least one PCR primer to selectively quantify the SNCA long transcript and SNCA total transcript in a sample from a subject according to any one of the methods, so as to determine the ratio of SNCA long transcript and SNCA total transcript, and instructions to carry out steps (a) and (b) of the method of any of the methods.

The present invention is based on the discovery that there is an increase in the SNCA long transcript to SNCA total transcript ratio in a PD patients relative to individuals unaffected by PD. The invention provides use of ratio of SNCA long transcript to SNCA total transcript in a subject's sample as a biomarker of PD disease or treatment. The invention provides use of ratio of SNCA long transcript to SNCA total transcript in a subject's sample to diagnose PD, or to confirm diagnosis of PD established by other criteria, or to determine predisposition or risk of a subject to develop PD.

Determining predisposition or risk of a subject to develop PD, or diagnosis of PD is done by comparing the ratio of SNCA long transcript to SNCA total transcript from a subject's sample to a ratio of SNCA long transcript to SNCA total transcript from a control sample, wherein an increased ratio of SNCA long transcript to SNCA total transcript in the subject's sample compared to the ratio of SNCA long transcript to SNCA total transcript in the control sample is indicative of a subject who has developed PD or of a subject who has increased risk for developing Parkinson's Disease. In certain embodiments of the methods optionally include review of medical history, conducting neurological examination, conducting brain scans to exclude PD-like symptoms, administering of dopamine affecting agents to determine if there is an improvement in the parkinsonism symptoms, for example but not limited to levodopa, or any other dopamine affecting agent.

In the instant methods, the subject's sample is a biological sample, including but not limited to a blood sample, plasma sample, serum, CSF, tissue, cell or any combination thereof. Methods to isolate nucleic acid sequences from biological samples are known in the art. Methods for quantitative determination of amount of nucleic acids in a biological sample are known in the art.

Common genetic variants in the human population may play a significant role in the pathogenesis of Parkinson's disease (PD) and other neurodegenerative disorders. As the majority of identified PD-associated variants do not alter protein coding, it is presumed that they modify gene expression, although direct evidence for this has been limited. Included herein are the results of a global transcriptome differential wiring analysis of PD patient and unaffected control brain tissues which identify a unique transcript isoform of aSynuclein (aSyn) with an extended 3′UTR, aSynL, that exhibits a dramatic correlation pattern change in diseased tissue. Strikingly, aSynL is even dyswired from other aSyn transcripts with shorter 3′UTRs, suggesting a pathogenic role for altered aSyn 3′UTR usage in disease. Consistent with this, a genome-wide association study identifies disease-associated polymorphisms within the aSyn and Parkin loci as key genetic factors in aSyn 3′UTR selection. An additional determinant of aSyn 3′UTR selection is intracellular dopamine content, suggesting a mechanism for the propensity of dopaminergic neuron cell loss in PD patient brain. Finally, we show that differential 3′UTR usage modifies the accumulation and localization of aSyn protein. Taken together, these findings identify a unifying mechanism for PD pathogenesis in the context of genetic and environmental variation.

In certain aspects the invention provides that the wiring effect on aSynL with respect to aSyn short is seen in unaffected people with disease-associated SNPs at the aSyn 3′UTR region. This effect cannot possibly be a secondary effect of the disease, as these people are unaffected.

In other aspects, the invention provides that with respect to the aSynL:total ratio, evidence for causality is that, genome wide in unaffected individuals, the top SNP that modifies the aSyn ratio is at the aSyn 3′UTR. Clearly, the SNP effect is causal, as the SNP is a genomic element.

In other aspects the invention provides the effect of the aSynL 3′UTR and SNPs on protein and localization, increase translation and mitochondrial localization.

In other aspects the invention provides that in unaffected human cortical brain samples an increase mitochondrial accumulation of aSyn protein corresponding to the PD-associated allele of the SNCA locus, thus bridging the different findings.

### BRIEF DESCRIPTION OF THE FIGURES

FIG. 1. Altered aSyn transcript co-expression networks in PD brain tissue. a-c, aSyn transcripts are globally rewired in PD brain tissue. a, the normalized DW score (y-axis) is plotted against the DE (x-axis, plotted in log2) between PD and unaffected control brain tissue cohorts. Each circle represents the DW and DE values for an Affymetrix probeset specific for an annotated transcript. The aSyn probeset GDW, 204467_s_at, highlighted in red, is most differentially wired in meta-analysis across all datasets, but is not among the most differentially expressed. b, Schematic representation of aSynL network rewiring in PD. aSyn transcripts recognized by the aSyn 204467_s_at probeset (aSynL) are shown in green. c, Correlation tables for the aSynL-specific 204467_s_at probeset in unaffected control (left) and in PD brain tissue (right) cohorts. High correlations (r=1) are denoted in red, high anti-correlation (r=−1) in blue and weak correlation in white (r=0); see methods for details. aSyn Probeset 204467_s_at is highlighted in green; a second aSyn probeset, 211546_x_at, is highlighted in blue. n=10 for unaffected, n=15 for PD. d-g, A loss of correlation in expression levels of aSyn transcript isoforms is specifically associated with PD. d, Schematic map of microarray probesets targeting aSyn mRNA CDS (blue shades) or 3′UTR (green shades). e, Correlation tables of aSyn isoform expression, as quantified by indicated probesets, in PD SN tissue samples (or unaffected controls; left panels, n=15 and 10 per group) and in an independent cohort of laser-microdissected SN dopamine neurons from PD patient tissue (or unaffected controls, as indicated; right panels; n=10 and 18 per group). High correlation (r=1) is depicted in red, weak correlation (r=0) in yellow. aSynL transcripts are relatively unwired from shorter transcripts in the PD samples. f, In contrast to PD SN patient tissue, aSyn transcript co-expression correlation is not modified in other neuropathology, such as cortical tissue from patients with sporadic FTLD-U or FTLD-U with Progranulin mutations (GSE13162, n=56). g, aSyn transcript co-expression was quantified in cortical tissue from 183 unaffected control individuals (GEO GSE15222) grouped according to their genotype for the PD-associated SNP PD risk-associated. Individuals harbor either 0 PD-risk allele (“CC”, left), 1 PD-risk allele (“CT”, middle) or 2 (“TT”, right). The homozygous disease-associated rs356168 CC genotype is associated with decreased correlation.

FIG. 2. Characterization of aSyn mRNA 3′UTR isoforms in unaffected and PD brain tissue. a, Mapping of pA-RNAseq reads from cerebral cortex brain samples of an unaffected individual (upper panel) and a PD patient (lower panel). The region shown encompasses the vicinity of the aSyn 3′UTR (chr4: 90,645,134-90,647,870 of human genome build hg19). Each blue rectangle represents an individual read at the 3′ end of a polyA transcript (middle panel). The most common aSyn 3′UTR species identified by pA-RNAseq analysis are schematized in the lower panel, grouped as short (in shades of green; 290, 480 or 560 nt), medium (in orange; 1070 nt) or long (red; 2520 nt) species. b, Relative abundance of the different aSyn 3′UTR species, as determined by pA-RNAseq analysis of 17 cortical brain samples from unaffected individuals. The frequency of each 3′UTR species color coded as in a) is expressed as the percentage of total aSyn transcript, averaged across the 17 individuals. Error bars are SEM. c, Northern blot analysis of RNA from human total brain reference or SH-SY5Y cells, as indicated. Blots were hybridized with probes targeting the aSyn CDS (Left panel; relative probe position shown below the dashed line in b, as dark blue bar) and the 3′UTR (Right panel; position shown in b as light blue bar). Nucleotide length is presented on the right; the corresponding 3′UTR size (color coded as per a) is indicated on left. d, Ratio of long 3′UTR aSyn mRNA to short 3′UTR aSyn mRNA species counts, evaluated by pA-RNAseq of cortical samples from unaffected individuals (n=17, black diamonds) and from PD patients (n=17, red triangles). Errors bars are SEM; *:p<0.05, two-tailed t-test, e, Ratio of aSynL:total transcript ratio, as quantified by RT-qPCR in cortical samples from PD (n=18), ALS (n=16) and unaffected individuals (n=8). Error bars are SEM; *:p<0.05, ANOVA followed by post-hoc Bonferroni multiple comparison test. f, aSynL:total aSyn transcript ratio in cortical tissue from 188 unaffected control individuals grouped according to their genotype for the PD-associated SNP PD risk-associated. Individuals harbor either 0 PD-risk allele (“CC”, left), 1 PD-risk allele (“CT”, middle) or 2 (“TT”, right). The statistical significance of the association between the allelic load of the variant and the ratio, as presented, was evaluated by GPLINK assoc function for quantitative traits (see Methods).

FIG. 3. Genome-wide association study for genetic determinants of aSyn transcript isoform ratio. a-b, Manhattan plot representing the SNPs associated with aSynL:total ratio. Association was evaluated for 380,157 SNPs in 364 cortical brain samples for quantitative traits association (see Methods for details). X-axis represents chromosomal location, Y-axis represents −log10 of the unadjusted p-value of association of each SNP with elevated aSyn transcript ratio. The aSyn 3′ locus SNP rs356168 (arrow) exhibited the highest association. SNPs above the p=10e-3 low-stringency threshold (blue line) were selected for further evaluation by Venn diagram analysis in (b; blue circle), and overlapping loci (within 75 kb of a given SNP) that are additionally associated with PD risk ⁹ are presented. c, Loci associated with both PD risk and aSynL:total ratio are presented (combination p-values are quantified as the geometric product of the individual p-values). d, aSynL:total ratio, quantified by RT-qPCR in whole brains of Parkin KO mice (n=4) or control mice (n=5). Error bars are SEM; **:p<0.01:two-tailed t-test.

FIG. 4. Dopaminergic and GABAergic modulation of the aSyn transcript isoform ratio. a, Rat primary cortical cultures were exposed to extracellular dopamine (0, 10, or 100 μM as indicated) and subsequently the aSynL:total ratio was quantified by RT-qPCR. High extracellular dopamine (100 μM) significantly increased the transcript ratio. b, I-Dopa treatment (20 mg/kg intraperitoneal injection daily for 5 days) of 2 month-old control (DAT-Cre/Dicer^{flox/+}) mice but not Dicer—deficient mice (DAT-Cre/Dicer^{flox/flox}); which have lost >95% of mDNs²⁴) led to a significantly increased aSynL:total ratio in midbrain tissue, as quantified by RT-qPCR. c, SHSY-5Y cells were cultured for 8 h in the presence of EU (to label newly transcribed RNA; ‘pulse’) and subsequently cultured in the absence of EU for the indicated period of time (0 h, 8 h, or 16 h; ‘chase’). EU-labeled nascent RNA, as well as total RNA, were then isolated from cell lysates and analyzed by RT-qPCR to evaluate the aSynL:total ratio. Pulse-chase analyses were conducted in the absence of dopamine (‘vehicle’; blue line in upper graph), in the presence of 100 μM dopamine during the EU labeling period only (‘dopamine pulse’; red line in graph and schematic), or during the chase exclusively (‘dopamine chase’; yellow line in graph and schematic). n=5 per group, errors bars are SEM. d, Primary cortical neuron cultures derived from aSyn PAC transgenic mice at day 4 in vitro (DIV) were treated with dopamine (100 μM), picrotoxin (100 μM), or vehicle for 24 h, and then subjected to in situ hybridization (ISH) with probes for human aSyn CDS (red) or specific for aSyn long 3′UTR (blue). Cells were co-stained with antibodies to aSyn (green) and observed by confocal microscopy. The subcellular localization of the different RNA species did not appear distinct. e, Ratio of In situ hybridization signals from probes as in d. Signal was quantified as particle count per neuron. n>10 neurons/group from 3 independent wells; error bars are SEM; ***:p<0.001, ANOVA followed by Bonferroni post hoc test versus the corresponding vehicle treatment. f, Schematic representation of the action of DAT and VMAT2 in dopaminergic neurons. DAT facilitates intracellular uptake of dopamine and thus sensitizes these cells to extracellular dopamine VMAT2 expression enables sequestration of dopamine into vesicles and away from other cytoplasmic constituents, and is thus protective. g, Rat primary cortical cultures were resistant to low extracellular dopamine (10 μM for 24 h; in the absence of DAT overexpression), whereas transfection of a vector encoding DAT sensitized these cells to extracellular dopamine (10 μM for 24 hrs), leading to an increased aSynL:total ratio as quantified by RT-qPCR.

FIG. 5. Regulation of aSyn translation through 3′UTR cis-acting elements. a, SH-SY5Y cells were treated with dopamine (100 μM), picrotoxin (100 μM) or vehicle for 48 h. Total endogenous aSyn protein levels were measured by ELISA and normalized to the total protein levels as assesses by BCA assay, n=5/group, *:p<0.05,**:p<0.01, ANOVA followed by Bonferroni post hoc test versus corresponding vehicle-treatment. b, I-Dopa treatment (20 mg/kg intraperitoneal injection daily for 5 days) of 2 month-old aSyn transgenic PAC mice led to a significantly increased aSyn protein in midbrain tissue, as quantified by ELISA, normalized to total protein level as measure by BCA. n>5 mice/group; *:p<0.05, ANOVA followed by Bonferroni post-hoc test versus corresponding vehicle-treated for each region. c, Schematic map of the aSyn 3′UTR displays the localization of known SNPs with the frequency of their minor alleles >1% in HapMap Caucasian panels. d, Human SHSY-5Y cells were transfected with a firefly luciferase-aSyn 3′UTR reporter vector (as in FIG. 4g; along with a Renilla luciferase control), or with this vector modified to encode the rs356165 (C>T) or the rs78991202 (T>G) minor alleles. Dopamine (100 μM) or picrotoxin (100 μM) were added to the culture medium for 24 hrs and luciferase activity was quantified as above and presented as the Firefly/Renilla luciferase ratio. n=6 for each group, *:p<0.05,**:p<0.01, ***:p<0.001, ANOVA followed by Bonferroni post hoc test vs. corresponding vehicle-treatment. e, Predicted local secondary structure of aSyn 3′UTR RNA near the rs356165 and rs78991202 SNPs using RNAfold ³⁰. A predicted miR-34-3p binding site is present in this region (as determined by Targetscan analysis⁶²). Insert shows the predicted global structure of the aSyn 3′UTR, with black box denoting the area of interest. f-g, Left panels: HEK293 cells were transfected with the luciferase-aSyn 3′UTR reporter vector, along with a miR-34b-mimic (f; compared to microRNA mimic control sequences) or with a miR-34b-inhibitor (g; compared to microRNA inhibitor control sequences). Luciferase activity was measured after 24 hrs. n=6 for each group, *:p<0.05,**:p<0.01, ***:p<0.001, ANOVA followed by Bonferroni post hoc test vs. corresponding vehicle-treated for each treatment. Right panels: SH-SY5Y cells were transfected with a miR-34b-mimic (I) or with a miR-34b-inhibitor (g), and total endogenous aSyn protein levels were measured by ELISA (normalized to total aSyn mRNA levels as measured by RT-qPCR). n=5 for each group, *:p<0.05, two-tailed t-test.

FIG. 6. aSyn transcript 3′UTR structure impacts aSyn protein localization. a, In SH-SY5Y cells exposed to dopamine (100 μM) or picrotoxin (100 μM) for 48 h, aSyn protein content is preferentially increased in mitochondrial preparations relative to whole cell aSyn content, as quantified by ELISA. n=5 for each group; *:p<0.05,**:p<0.01, ANOVA followed by Bonferroni post hoc test versus the corresponding vehicle treated cells. b-c, Rat primary cortical neurons cultures at 3 DIV were transfected with a vector encoding a GFP-aSyn fusion protein (green) with either a short (0.3 Kb) or a long (1.1 kb) aSyn 3′UTR and stained with Mitotracker (c, in red) followed by confocal microscopy. Increased colocalization was observed in the context of the longer 3′UTR, both within the axonal growth cone terminal fields (L1 or S1 arrows, magnified in upper inserts) as well as in axonal processes (arrows L2 or S2, magnified in lower inserts). Scale bar, 10 μm in main panel, 5 μm in insets. Colocalization between GFP-aSyn and Mitotracker was quantified by Pearson correlation coefficient in the context of each plasmid transfection, in 12 randomly chosen fields per well. Significance was assessed by Fisher transformation followed by a two-tailed t-test; Error bars are SEM; n>3 wells per condition; *,p<0.05. d, SH-SY5Y cells exposed to dopamine (100 μM) or picrotoxin (100 μM for 48 h) display a reduction in aSyn context within total cell membrane fraction, relative to whole cell protein lysate content, as determined by ELISA assay. n=5 for each group. *, p<0.05; **, p<0.01, ANOVA followed by Bonferroni post hoc test versus corresponding vehicle treatment. e, Rat primary cortical neurons cultures at were transfected with vectors encoding a GFP-aSyn fusion protein (green) with either a short (0.3 Kb) or a long (1.1 kb) aSyn 3′UTR and immunostained for synaptophsyin, followed by confocal microscopy. Colocalization between GFP-aSyn and synaptophysin staining was decreased in the context of the presence of the longer 3′UTR, as quantified by Pearson correlation coefficient between the conditions in individual cells from 12 randomly chosen fields per well. Significance was assessed by Fisher transformation followed by a two-tailed t-test; Error bars are SEM; n>3 wells per condition; *,p<0.05). f, aSyn protein content was quantified by ELISA analysis of mitochondrial protein fractions isolated from 19 human cortical brain samples from unaffected individuals; content was normalized to the total protein concentration, as evaluated by BCA assay. Samples are grouped according to their rs356165 genotype. n=3 for TT genotype, n=12 for GT genotype, and n=4 for GT genotype. Error bars are SEM. *, p<0.05, as evaluated by the gplink assoc function for quantitative traits for the effect of the rs356165 allelic load on mitochondrial aSyn protein concentration. g, Top 5 Gene Ontology categories identified by GSEA analysis to be associated with rs356168 allelic load across 186 unaffected cortical brain samples. See Methods for details. h, A model of aSyn 3′UTR regulation and its consequences. Intracellular dopamine impacts alternative polyadenylation of aSyn transcripts. Generation of the longer aSynL transcript lead to increased translation and prefential localization to mitochondria. PD risk-associated SNPs within the aSynL 3′UTR lead to increased stability of the transcript and thus potentiate protein accumulation.

FIG. 7. Altered aSyn transcript wiring in PD but not other neurological disorders. a, Altered coexpression networks of aSyn transcript isoforms in PD LMD SN neurons. Correlation heat maps of probesets as in FIG. 1c, but in samples from laser-microdissected nigral dopamine neuron instead of total nigra. Left panel represents the correlation pattern in samples from unaffected individuals, right panel represents samples from PD patients. The probesets displayed are as in FIG. 1c (those with most significantly altered wiring to the aSyn probeset 204467_s_at). High correlations (r=1) are in red, high anticorrelation (r=−1) in blue and weak correlation in white (r=0). Rows and columns corresponding to correlation with probeset 204467_s_at are bordered with a thick black line. The changes observed in the correlation pattern in the context of PD in LSM SN dopamine neurons are similar to those observed in the full SN samples (FIG. 1c). b-c, No alteration in co-expression of aSyn probesets in other neurological diseases. aSyn probeset correlations are not altered by schizophrenia or Huntington's disease in the affected tissue. Correlation tables of aSyn probesets (as in FIG. 1d), in control and Huntington's Disease caudate nucleus brain samples (b, from GEO GSE3790 (12), in control and schizophrenia brain cerebral cortex samples (c, from GEO GSE1761 (13). High correlation (r=1) are in red, weak correlation (r=0) in yellow. No significant changes in correlation between the expression levels of the different aSyn probesets were observed between the disease and control samples.

FIG. 8. aSyn transcript isoforms in PD cerebral cortical and substantia nigra tissue. a, Distribution of the different aSyn 3′UTR isoforms in cerebral cortex samples from unaffected (left) and PD (right) individuals. polyA-RNAseq read count for each isoform is presented as a percentage of total aSyn read count. N=17 individuals for each group (patient or unaffected). Error bars are SEM. b-c, aSynL:total transcript ratio evaluated in SN tissue samples from PD patients or unaffected individuals (b, GEO GSE7621 1, n=10 and 15 for unaffected and PD individuals, respectively) or SN laser microdissected dopamine neurons (c, GEO GSE20141 35, n=8 and 10 for unaffected and PD individuals, respectively). Differences did not reach statistical significance (by two-tailed t-test).

FIG. 9. Significant overlap between GWAS derived PD risk-associated loci and GWAS derived loci that are associated with an elevated aSynL:total transcript ratio. We sought to assess the statistical significance of the observed overlap between GWAS derived PD risk-associated loci (defined by genome-wide significance of p<1×10-3; a total of 384 SNPs; (8)) with GWAS derived loci that are associated with an elevated aSynL:total transcript ratio (defined by genomewide significance of p<1×10-3; a total of 316 SNPs). A total of 22 SNPs rQTL SNPs overlapped with the PD risk SNPs (overlapping loci defined as SNPs that fall within 75 kb of each other). To estimate the chance of occurrence of this many overlapping loci, we performed additional analyses of overlapping loci, but between the previously reported PD risk loci and randomly chosen sets of 316 SNPs (instead of the 316 rQTL SNPs as in FIG. 3b; we term this ‘bootstrap resampling without replacement’). We performed 5×10⁶ such control analyses of locus overlap using sets of random SNPs. Represented is the frequency distribution of the number of overlapping SNPs found between such random sets of SNPs (316 each) and the PDassociated loci (384 SNPs), over 5×106 trials. Indicated are the number of trials (red) for which a given number of SNPs (black) overlap with the PD-associated loci. With the random SNP intersections, a mean of 2.3 SNPs is observed, and the maximum observed value is 14. This distribution corresponds to the intersection expected simply by chance between 316 random SNPs and the PD associated loci. As we found that 22 rQTL SNPs overlapped with the PD-associated loci, this is significantly higher than one would expect by chance (p<10-6 by the empirical resampling analysis).

FIG. 10. Additional analyses of aSyn transcript isoform ratio regulation. a, GABA receptor but not glutamate receptor modulators alter the aSynL:total ratio. Left panel: aSynL:total ratio as measured by RT-qPCR in primary cortical neurons exposed to the GABA-A receptor antagonist picrotoxin (100 μM), the GABA-A receptor agonist muscimol (100 μM), the glutamatergic receptor agonist NMDA (100 μM), the glutamatergic receptor agonist kainic acid (50 μM), or vehicle. n=5/group, means are represented, error bars are SEM. ***, p<0.001 by two-tailed t-test. Right panel: Human SHSY-5Y neuroblastoma cells were transfected with an expression vector encoding the Firefly luciferase gene fused to the human aSyn 3′UTR (1.1 kb 3′UTR insert; along with a Renilla Luciferase control gene), then exposed for 24 h to picrotoxin (100 μM) or to muscimol (100 μM). Luciferase luminescence is presented as the Firefly/Renilla ratio. n=8/group, mean are represented, error bars are SEM. ***, p<0.001, ANOVA followed by Bonferroni multiple comparison test comparison made: treatments versus vehicle. b, Validation of specificity of in situ hybridization probes (as per FIG. 4d) detecting either the coding sequences (CDS, thus all human aSyn mRNA isoforms) or specifically long aSyn 3′UTR species. Rat primary cortical neurons cultures at 3DIV were transfected with a vector encoding a GFP-human aSyn fusion protein (green) with either a short (0.3 Kb) or a long (1.1 kb) aSyn 3′UTR and subjected to in situ hybridization with either a probe targeting human aSyn mRNA CDS (red) or a sequence of human aSyn 3′UTR specific to the long 3′UTR transcripts (blue). While no signal was observed for any probe in untransfected cells, cells transfected with the short 3′UTR construct (‘Transf. aSyn Short’, upper panels) exhibit robust red signals but no blue signal; cells transfected with the long 3′UTR Trans aSyn Long’, lower panels) exhibit both red and blue signals. c, Schematic representation of the pulse-chase procedure for nascent RNA isolation. During the pulse period (in blue), ethinyl uridine (EU) is present in the culture medium and is incorporated into the newly transcribed RNA (red). As the pulse period continues, labeled RNA progressively replaces the pre-existing unlabeled RNA (black). During the subsequent chase period, EU is not present in the media, and the newly transcribed RNA is unlabeled (black). At later stages of the chase period, unlabeled RNA (black) progressively replaces the labeled species (red). At different time points during the chase, total RNA is extracted, and from this total population, labeled RNA (red) can be specifically captured and submitted to RT-PCR analysis. d, aSynL:total ratio in nascent RNA upon dopamine treatment in SH-SY5Y cells. Cells were treated with EU for 4 hours, together with either dopamine (100 μM) or vehicle. Cells were immediately harvested and the aSynL:total ratio was evaluated in both the total RNA (left panel) and the captured EU-labeled RNA (nascent RNA) by RT-qPCR. We observe that after 4 h of dopamine treatment, the increase in aSynL:total ratio is robustly observed in the nascent population but not in total RNA. n=5/group, mean are represented, error bars are SEM. *, p<0.05, two-tailed t-test. e, Transcription inhibition suppresses the impact of dopamine treatment on the aSynL:total ratio in SH-SY5Y cells. Cells were treated with combination of dopamine (100 μM) and actinomycin D (10 μg/mL) and harvested after 12 or 24 h. The aSynL:total ratio was evaluated in total RNA by RT-qPCR. We observe that transcription inhibition prevented the dopamine-mediated increase in aSynL:total ratio otherwise observed after 24 h of treatment. n=6/group, mean are represented, error bars are SEM. **, p<0.01, two-tailed test. f, Polyadenylation site disruption mimics and occludes the dopamine-mediated potentiation of the aSynL:total ratio. Human SHSY-5Y neuroblastoma cells were transfected with an expression vector encoding a GFP-aSyn fusion protein with a 1.1 kb aSyn 3′UTR (“Wild-type 3′UTR”, left) or with such a vector that harbors a deletion of the predicted polyadenylation signal sequences utilized for generation of an aSyn transcript with a short 3′UTR (‘disrupted polyA site’, right). Cells were lysed after 48 h, and RNA was extracted and analyzed by Northern blotting using a CDS-specific probe. Upper panel:aSynL:total ratio from Northern blot quantification. n=3/group. Means are represented; error bars are SEM. *, p<0.05, onetailed t-test. Lower panel: representative Northern blot. Dopamine treatment leads to an increase in an aSynL (1070 nt 3′UTR length) transcript relative to a shorter (300 nt 3′UTR length) transcript, both encoded by the exogenous plasmid. In the context of the disrupted polyA site, relative production of aSynL is increased even in the absence of dopamine. g, Nomifensine treatment suppresses dopamine-mediated potentiation of the aSynL:total ratio. Left panel: aSynL:total ratio, evaluated by RTqPCR in rat primary cortical neurons culture. Cells were exposed to combinations of dopamine (100 μM) and nomifensine (100 μM) for 24 h, as indicated, before harvesting and RNA extraction. Mean levels are displayed; errors bars are SEM; n=6 for each group. **:, p<0.01; ***, p<0.001, ANOVA followed by Bonferroni multiple comparison test. Right panel: Human SHSY-5Y neuroblastoma cells were transfected with an expression vector encoding the Firefly luciferase gene fused to a human aSyn 3′UTR (1.1 kb insert; along with a Renilla Luciferase control gene). Cells were then exposed for 24 h to dopamine alone (100 μM) or, dopamine with nomifensine (100 μM), before luminescence measurement. Data are presented as Firefly/Renilla luminescence ratio. n=8 for each group; *, p<0.05; ***, p<0.001; ANOVA followed by Bonferroni multiple comparison test. h, Effect of dopamine agonists on aSynL:total ratio in cultured rat neurons. aSynL:total ratio, evaluated by RT-qPCR in rat primary cortical neurons cultures. Cells were exposed to dopamine (100 μM), 7-OH DPAT (100 μM), Quinpirole (20 μM) or SKF38390 (20 μM) during 24 h before cells harvest and RNA extraction. Mean levels are displayed; errors bars are SEM; n=6 for each group.

FIG. 11. Evidence for translational regulation of aSyn through distal 3′UTR sequences. a, aSynL is preferentially associated with polysomes. aSynL:total ratio evaluated by Affymetrix probesets 204467_s_at and 211546_x_at in total and polysomal-associated RNA from human MCF10A cells (using existing data from GEO GSE11011 ₂₅). The aSynL:total ratio is significantly increased is the polysome fraction, suggesting enhanced translation of the longer 3′UTR aSynL mRNAs. n=6/group; ***, p<0.001, two-tailed t-test. b, Dopamine and picrotoxin treatment do not influence the translation of short aSyn 3′UTR. Human SHSY-5Y cells were transfected with a firefly luciferase-short aSyn 3′UTR (275 nt) or firefly luciferase-long aSynL 3′UTR (1100 nt) reporter vector along with a Renilla luciferase control. Dopamine (100 μM) or picrotoxin (100 μM) were added to the culture medium for 24 hrs and luciferase activity was quantified and presented as the Firefly/Renilla luciferase ratio. n=6 for each group. c, Genomic variants in aSyn 3′UTR that do not affect its translation. Human SHSY-5Y cells were transfected with a firefly luciferase-aSynL 3′UTR reporter vector (as in FIG. 5d along with a Renilla luciferase control), or with this vector modified to encode the rs34825 (A>G), rs1701607 (C>T), rs35733299 (C>T) or rs35716318 (G>A) minor alleles (see FIG. 5c). Dopamine (100 μM) or picrotoxin (100 μM) were added to the culture medium for 24 hrs and luciferase activity was quantified as above and presented as the Firefly/Renilla luciferase ratio. n=5 for each group. d, Reduction of intracytoplasmic dopamine by VMAT2 overexpression reduces dopamine effect on aSyn 3′UTR mediated translation. SH-SY5Y cells were co-transfected with an expression vector for VMAT2 (see FIG. 4f) or vector control, along with a firefly luciferase-aSyn 3′UTR reporter vector and a Renilla luciferase control, and then exposed to dopamine (100 μM) for 48 h. Dopamine treatment potentiates aSyn 3′UTR mediated translation, but this effect is cancelled by VMAT2 overexpression. n=5-6 for each group, errors bars are SEM; *, p<0.05; **, p<0.01; ***, p<0.001. ANOVA followed by Bonferroni post-hoc test versus corresponding vehicle treatment. e, Dopamine and miR-34b affect aSyn translation independently. SH-SY5Y cells were transfected with miR-34b inhibitor or a control inhibitor and treated with dopamine (100 uM) for 24 h or vehicle only. Total endogenous aSyn protein levels were measured by ELISA and normalize to total aSyn mRNA levels measured by RT-qPCR. The respective contribution of each factor (dopamine and miR-34b) as well as their potential interaction was evaluated by fitting the following linear model for aSyn translation in function of dopamine and miR-34, allowing an interaction between both factors (aSyn=a.miR+b.Dopa+c.MirXDopa+d) using R aov function. N>5/group. Results of the fitting process for a and b were highly significant (p=7.4E-4 and 1.9E-3 respectively), confirming the significant increase of aSyn translation by both miR-34b inhibition and dopamine treatment. No interaction between those two factors was however identified (p=0.65); dopamine and Mir-34b effects are additive and thus appear independent. f, Mir-7 impacts the translation of long and short aSyn 3′UTR isoforms equivalently. Human SHSY-5Y cells were transfected with firefly luciferase reporter vectors that harbor no aSyn 3′UTR, aSyn long 3′UTR (1074 nt) or aSyn short 3′UTR (275 nt), as well as Mir-7 mimic or control miRNA mimic (and a Renilla luciferase control). Luciferase activity was quantified as above and presented as the Firefly/Renilla ratio. (n=6 for each group, mean are represented, error bars are SEM. **, p<0.01; ***, p<0.001, ANOVA followed by Bonferroni post hoc test vs. “no 3′UTR” group).

FIG. 12. aSyn transcript 3′UTR isoform impacts aSyn protein translation and protein localization. a-c, Primary cortical neuron cultures were generated from PAC transgenic mice. At day 4 in vitro (DIV), cultures were treated with picrotoxin (100 μM), dopamine (100 μM) or vehicle, for 24 h as indicated. Cultures were then stained with Mitotracker (red) as well as with an antibody specific for aSyn (green). Imaging of cultures was by confocal microscopy. a, White squares denote regions that are magnified in b; arrows in b point to mitochondria signal within a neurite process. c, Colocalization of aSyn and Mitotracker signals was quantified in digital images of 10 randomly chosen fields within each of N>3 independent wells per condition. Means are represented, error bars are SEM. Significance of the effect of drug treatments (versus vehicle) was assessed by Fisher transformation followed by a two-tailed t-test. *, p<0.05; **, p<0.01. d, Mitochondrial enrichment confirmation by Western Blot. Intact mitochondria were purified using the Qproteome mitochondria isolation kit. Total protein (left) and isolated mitochondria protein fractions (right) from two representative brain samples were probed by Western Blotting for TOM20 (upper panel), a mitochondrial protein, or synaptophysin (SYP, lower panel), a synaptic protein. e-f, Human SHSY-5Y neuroblastoma cells were transfected with an expression vector encoding a GFP-human aSyn fusion gene with a short (0.3 Kb, “aSyn-short 3′UTR”) or long (1.1 kb, “aSyn-long 3′UTR”) aSyn 3′UTR, or with GFP only (“Ctl”). e, aSyn protein level was quantified by ELISA in protein extracted from purified mitochondria. aSyn concentration is expressed relative to the total protein concentration as determined by bicinchoninic acid (BCA) assay. n=5 for each group. Means are represented. Error bars are SEM. **:p<0.01, ANOVA followed by Bonferroni post hoc test. f, aSyn protein levels were quantified by ELISA in total protein extracts. aSyn concentration is expressed relative to the total protein concentration as determined by BCA assay. n=5 for each group. Means are represented. Error bars are SEM. g, Schematic representation of the method for assessing the global functional impact of rs356165 on the transcriptome in unaffected cortical brain samples. Left panel: Unaffected individuals are characterized according to their rs356168 risk allele load: 0 for homozygous for the protective allele (AA), 1 for heterozygous (AC) and 2 for homozygous for the risk allele (CC). Using genome-wide expression profiles in cortical brain samples from unaffected individuals, for each gene, the correlation of its expression level with the risk allele load is evaluated across all samples. For instance, Gene 1 expression increases with the risk allele load, and will thus exhibit a correlation value close to 1. By contrast, Gene 3 profile leads to a negative correlation value and Gene 2 to a value close to 0. We next evaluated whether groups of genes belonging to common biological functions are overrepresented among those gene expression profiles that correlate with a one allele or the other. To this end, the correlation values for all gene expression profiles is used as a pre-ranked input for Gene Set Enrichment Analysis (GSEA). Right panel: GSEA output example, with a biological function found to be significantly associated with rs356168 allele load in control brain samples (Mitochondria Membrane Part). The majority of gene expression profiles in this category (vertical black lines) are enriched in the red zone, corresponding to strong correlation with the disease allele. The Enrichment Score is a measure of such overall correlation₃₄. Below are listed the genes (included within the Mitochondria Membrane Part annotation category) whose expression profiles correlate with rs356168 allele load, and thus account for the annotation category enrichment.

FIG. 13. aSynL:total ratio is modified by disease-associated environmental factors. a-c, aSynL:total ratio is increased by mitochondrial toxins. a, Gene expression analysis of brain regions of mice treated daily for 5 days with 30 mg/kg intraperitoneal MPTP or saline (using existing data from GEO GSE7707 ₆₂). The aSynL/total ratio was evaluated as a ratio of the Affymetrix probesets 1418493_a_at and 1436853_a_t). Comparisons were performed for MPTP-treated versus saline controls within each brain region, n=3/group, *, p <0.05, two-tailed t-test. b, Reanalysis of brain gene expression of macaques treated daily with intraperitoneal MPTP hydrochloride (0.2 mg/kg) or saline for either 6 or 12 days (“presymptomatic state”, GEO GSE4550 ₂₈). The aSynL/total ratio was evaluated with Affymetrix probesets 204466_s_at and 211546_x_at, (as the human 204467_s_at poorly detects macaque aSyn mRNA). Comparisons were done for MPTP-treated versus saline controls within each brain region, n=3-6/group; *, p<0.05, two-tailed t-test, (*):p<0.05, onetailed t-test. c, Reanalysis of transcriptome changes in data from human SK-N-MC cells treated with chronic low-dose rotenone or vehicle for one or two weeks (GEO GSE4773 63). aSynL/total ratio was evaluated as a ratio of Affymetrix probesets 204467_s_at and 211546_x_at. Comparisons were done for rotenone versus vehicle for each time point, n=3/group; *, p<0.05, two-tailed t-test; (*), p<0.05, one-tailed t-test. d, Nicotine treatment decreases the expression of an aSynL-3′UTR bearing reporter gene. Luciferase levels in human SY-5Y neuroblastoma cells, transfected with a plasmid encoding a Renilla gene and a luciferase gene fused to the human aSynL 3′UTR (1.1 kb). Combinations of dopamine (100 μM) and nicotine (100 μM) were added, as indicated, to the culture medium immediately after transfection and luciferase activity was measured after 24 h. Mean normalized luciferase Firefly/Renilla levels are displayed; errors bars are SEM; n=6 for each group. Comparisons are made between nicotine treated groups and the associated vehicle treated group.*,p<0.05; ***, p<0.001, two-tailed t-test. e, DJ-1 knockdown increases aSynL:total ratio in human neuroblastomas cells. aSynL:total ratio was analyzed in existing transcriptome data of DJ-1-silenced human SH-SY5Y neuroblastoma cells and control treated cells, measured by Affymetrix Human Genome U133 Plus 2.0 Array. The aSynL:total ratio was quantified using a ratio of expression values for Probesets 204467_s_at and 211546_x_at; GEO GSE17204 ₄₈. Mean levels are displayed; errors bars are SEM; n=4/group; ***, p<0.001, two-tailed t-test. f-g, Aging is associated with an increased aSynL:total ratio in human brain. aSynL:total ratio was quantified in existing postmortem brain sample whole transcriptome data from four different brain regions of healthy donors gathered by age, as measured by Illumina humanRef-8 v2.0 expression beadchip (f, Probes for aSynL and aSyn total are ILMN_{—}1701933 and ILMN_{—}1766165, respectively; data from GSE15745 (49), n=13-17/group) or Affymetrix Human Genome U133 Plus 2.0 Array (g, Probesets for aSynL and aSyn total are 204467_s_at and 211546_x_at, respectively; data from GSE11882 (₅₀). Mean levels are displayed; errors bars are SEM; n=17-25/group. All values are normalized to prefrontal cortex samples from youngest group. Comparisons are made between age groups within each brain region. *, p<0.05; **, p<0.01; ***, p<0.001. ANOVA followed by Bonferroni post hoc test in (f); two-tailed t-test in (g).

FIG. 14. aSynL:total ratio in human tissues. a, aSynL:total ratio in different human brain regions. aSynL:total in postmortem samples from twenty-two different brain regions of healthy donors, grouped by age, measured by Affymetrix Human Genome U133 Plus 2.0 Array (derived from the Human Body Index GEO GSE7307 dataset; see Methods). aSynL and aSyn total expression levels are determined using Probesets 204467_s_at and 211546_x_at, respectively. Mean levels are displayed; errors bars are SEM; n=7-8/group). Values are normalized relatively to the level in Substantia Nigra (in red). b, SynL:aSynT ratio in blood from PD patients and controls. aSynL:total in peripheral blood collected from 18 Parkinson's Disease patients and 12 healthy controls and measured using Affymetrix Exon 1.0 ST Array (GEO GSE18838 ₅₁). The ratio of each CDS and 3′UTR probe to the aSyn whole transcript level (estimated as the average of all probes) for PD patient group (red) is displayed relatively to the control group (black). Mean levels are displayed; errors bars are SEM; n=12-18/group). *, p<0.05, two-tailed t-test. A schematic mapping of the aSyn mRNA (green) regions detected by the probes is shown, with the 3 different 3′UTR probes represented by black boxes as indicated.

FIG. 15 shows GDW analysis with such significant threshold (exactly as in FIG. 1A) or without.

### DETAILED DESCRIPTION

SNCA and aSyn are used interchangeably. SNCA Long and aSynL are used interchangeably.

The term “about,” as used herein when referring to a measurable value such as an amount of a compound or agent of this invention, dose, time, temperature, and the like, is meant to encompass variations of +/−20%, +/−10%, +/−5%, +/−1%, +/−0.5%, or even +/−0.1% of the specified amount.

The term “Parkinson disease” (PD) as used herein is intended to encompass all types of Parkinson disease. In some embodiments, the term Parkinson disease means idiopathic Parkinson disease, or Parkinson disease of unexplained origin: that is, Parkinson disease that does not arise from acute exposure to toxic agents, traumatic head injury, or other external insult to the brain. In some embodiment, the invention is directed to detecting or screening for early or late onset Parkinson disease.

The terms dyswired, rewired, unwired and miswired are used interchangeably.

The sequence of SNCA transcripts are known in the art.

The invention is directed to methods to confirm, diagnose, determine predisposition to and/or determine risk of developing PD in a subject. The invention is based on the observation that there is an increase in the SNCA long transcript to SNCA total transcript ratio in a PD patients relative to individuals unaffected by PD.

Various embodiments of the methods of the invention are discussed. The methods can comprise, consist essentially of, or consist of the step which are discussed.

Various kits for use in the methods of the invention are discussed. The kits can comprise, consist essentially of, or consists of the various reagents discussed.

In certain embodiments, the methods include determining SNCA long transcript to SNCA total transcript ratio in a subject's sample, and comparing the subject's ratio to a reference SNCA long transcript to SNCA total transcript ratio. In certain embodiments, the reference ratio can be determined from subjects having non-PD status. In other embodiments, the reference ratio is PD status ratio, which is determined from PD subjects, for example subjects diagnosed to have PD by other means. In certain embodiments the SNCA long transcript to SNCA total transcript ratio in a subject's sample is compared to a reference ratio from subjects having non-PD status, or to a reference PD status ratio determined from PD subjects, or to both non-PD status reference ratio and PD status reference ratio, to determine whether the SNCA long transcript to SNCA total transcript ratio in the subject's sample is similar to the non-PD status reference ratio and the PD status reference ratio.

In certain embodiments, the methods comprise additional step of conducting a physical examination of the subject, or a neurological examination, or any other suitable determination to confirm, diagnose, determine predisposition to and/or determine risk of developing PD in a subject.

The present invention provides a method of identifying a subject with Parkinson disease as having an increased or decreased likelihood of responding effectively to a treatment, for example with a candidate agent to treat PD, comprising: determining SNCA long transcript to SNCA total transcript ratio in a subject's sample in the presence and absence of the candidate agent, and correlating the SNCA long transcript to SNCA total transcript ratio in a subject's sample to the ratio in a test subject effectively responding to a treatment. In certain embodiments, the treatment is a dopamine affecting agent. In certain embodiments, in the presence of the dopamine affecting agents, the SNCA long transcript to SNCA total transcript ratio in a subject's sample decreases, thereby indicating increased likelihood of effective treatment.

In further embodiments, the present invention provides a method of conducting a clinical trial on a plurality of human subjects or patients. Such methods advantageously permit the refinement of the patient population so that advantages of particular treatment regimens (typically administration of pharmaceutically active organic compound active agents) can be more accurately detected, particularly with respect to particular sub-populations of patients. Thus, the methods described herein are useful for matching particular drug or other treatments to particular patient populations for which the drug or other treatment shows any efficacy or a particular degree of efficacy and to exclude patients for whom a particular drug treatment shows a reduced degree of efficacy, a less than desirable degree of efficacy, or a detrimental effect.

treatment shows any efficacy or a particular degree of efficacy and to exclude patients for whom a particular drug treatment shows a reduced degree of efficacy, a less than desirable degree of efficacy, or a detrimental effect.

In general, such methods comprise administering a candidate agent (e.g., active drug or prodrug) or therapy to a plurality of subjects (a control or placebo therapy typically being administered to a separate but similarly characterized plurality of subjects) as a treatment for PD, determining the SNCA long transcript to SNCA total transcript ratio in the plurality of subjects and correlating the correlating with efficacy or lack of efficacy of the test agent or therapy.

In other embodiments, the invention provides methods to evaluate a treatment for PD, the method comprising determining the SNCA long transcript to SNCA total transcript ratio in a sample, wherein the sample is from a cell culture, from an animal model, or from a subject, wherein the sample is obtained in the presence or absence of the treatment for PD, wherein a lowered ratio of SNCA long transcript to SNCA total transcript ratio in the sample in the presence of the treatment compared to the absence of the treatment is indicative of a therapeutic treatment for PD.

Methods to Quantify Nucleic Acids

Methods to quantify nucleic acids from biological samples are known in the art. Any suitable method to quantify nucleic acids from biological samples are contemplated for use in the invention. In a non-limiting embodiment, RT-qPCR is done as described in reference 38. SNCA long to SNCA total ratio were quantified using ΔΔCt using primers pairs HaSynLfw (SEQ ID NO: 1 ATTGAAGTATCTGTACCTGC) HaSynLrv (SEQ ID NO: 2 AAGACCCTGCTACCATGTATTC) and HaSynTfw (SEQ ID NO: 3 AGGGTGTTCTCTATGTAGG) HaSynTrv (SEQ ID NO: 4 ACTGTCTTCTGGGCTACTGC) for human sequence, or RaSynLfw (SEQ ID NO: 5 AACTTCTTGAGAACAGCAACAA) RaSynLrv (SEQ ID NO: 6 CTCCCCTCTCACTACAG) and RaSynTfw (SEQ ID NO: 7 CAACGTGCCCAGTCA) RaSynTrv (SEQ ID NO: 25 GGATGCTGAGGGGCAGGT) for mouse and rat sequences.

Alternatively, for any of the SNCA transcript isoforms to be quantified, suitable primers specific for an isoform may be designed by known methods in the art. In other embodiments, the skilled artisan is able to modify the sequences of the above-described primers by addition and/or deletion of one or a few nucleotide(s) at the 3′ and/or 5′ end, for example but not limited to addition of nucleotides at the 5′ end of a primer.

target sequence, which is bonded to pairs of fluorophore groups or fluorophore/quenchers, such that hybridisation of the probe to its target and the successive amplification cycles cause an increase or reduction in the total fluorescence of the mixture, depending on the case, proportional to the amplification of the target sequence.

Non limiting examples of labeling systems that can be used to carry out kinetic PCR are the TaqMan™ (ABI®), the AmpliSensor™ (InGen), and the Sunrise™ (Oncor®, Appligene®) systems. The skilled artisan can chose amongst these systems or other any other labeling systems.

Apart from the primers and probe sequence, the skilled artisan can use general knowledge concerning quantitative RT-PCR in order to determine the other parameters for performing the method according to the invention, for example but not limited to, cycling parameters, quantification having regard to a housekeeping gene, etc. Examples of such parameters are well known in the art.

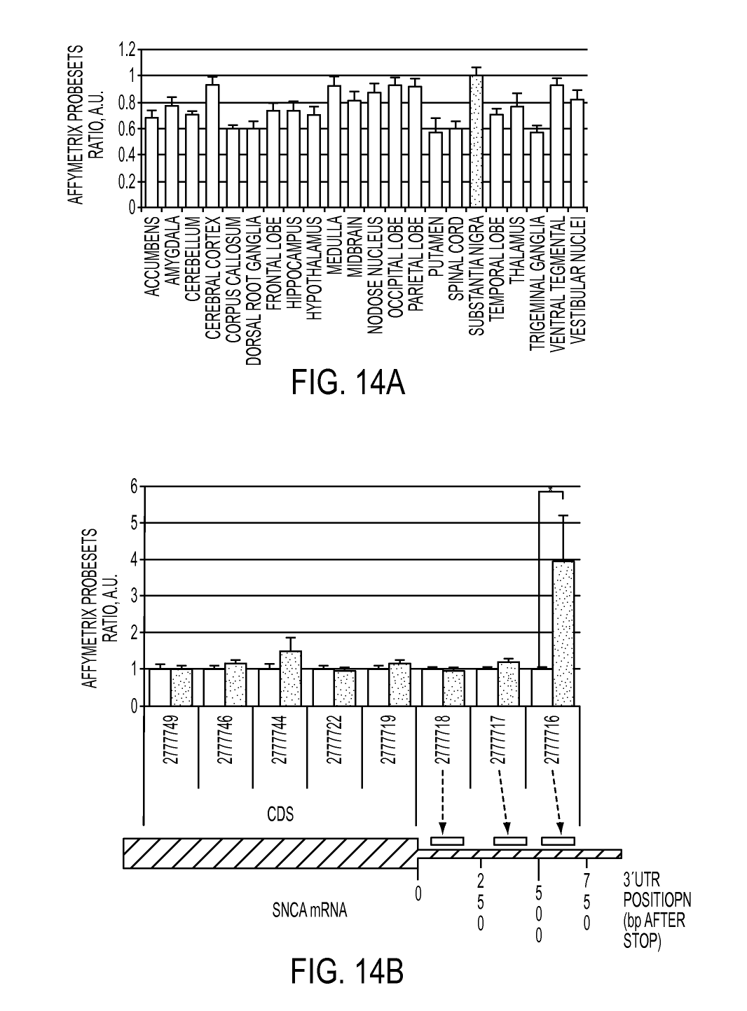

In other embodiments, SNCA long to SNCA total ratio can be quantified using nucleic acid microarrays and probes designed to detect specific transcripts. A non-limiting example of determining SNCA long to SNCA total ratio using nucleic acid microarrays is shown in FIG. 14.

Numeric values and/or ranges of fold difference in ratio for at risk subjects, PD subjects and healthy controls can readily be determined.

Any suitable biological sample can be used to determine SNCA long transcript to SNCA total transcript ratio. The biological sample can be taken from body fluid, such as urine, saliva, bone marrow, blood, and derivative blood products (sera, plasma, PBMC, circulating cells, circulating RNA). The biological sample can be taken from a human subject, from an animal, or from a cell culture. The biological sample can be obtained in vivo, in vitro or ex vivo. Non-limiting examples of biological samples include blood, serum, plasma, cerebrospinal fluid, mucus, tissue, cells, and the like, or any combination thereof. In a non-limiting embodiment the biological sample is blood. In a non-limiting embodiment the biological sample is serum. In a non-limiting embodiment the biological sample is plasma. Any suitable method to isolate nucleic acids from biological samples are contemplated for use in the invention. Biological samples for analysis are stored under suitable conditions. In non-limiting examples biological samples are kept at about 4° C. In non-limiting examples biological samples are kept at about −20° C. In non-limiting examples biological samples are kept at about −70-80° C.

Kits

In certain embodiments the invention provides kits to carry out the methods of the invention. The kits comprise reagents to carry out the steps of determining SNCA long transcript to SNCA total transcript ratio, for example but not limited to primers for RT-qPCR, and optionally other reagents for RT-PCR such as suitable polymerases, nucleotide mix, fluorescent dyes, and so forth. The kits comprise instructions to carry out the step of comparing the ratio determined in the subject's sample to a reference ratio so as to determine whether there is a difference between the ratio determined in the subject's sample and the reference ratio. For example a reference ratio is associated with a PD status, or a reference ratio is associated with a non-PD status, wherein in a non-limiting example the non-PD status ratio is based on the ratio determined from healthy controls.

Dopamine Affecting Agents

The main families of drugs useful for treating motor symptoms associated with PD are levodopa, dopamine agonists and MAO-B inhibitors. In certain embodiments, levodopa is combined with a dopa decarboxylase inhibitor or COMT inhibitor. Dopa decarboxylase inhibitors help to prevent the metabolism of L-DOPA before it reaches the dopaminergic neurons, therefore reducing side effects and increasing bioavailability. In non-limiting examples dopa decarboxylase inhibitors are given as combination preparations with levodopa. The COMT enzyme degrades dopamine Inhibitors of the COMT enzyme thereby prolonging the effects of levodopa, when administered in combination with levodopa.

Dopamine agonists that bind to dopaminergic post-synaptic receptors in the brain have similar effects to levodopa.

MAO-B inhibitors inhibit monoamine oxidase-B (MAO-B) which breaks down dopamine secreted by the dopaminergic neurons. Thus, MAO-B inhibitors, for example but not limited to selegiline and rasagiline, increase the level of dopamine in the basal ganglia by blocking its metabolism.

Animal models of PD, including but not limited to toxin-, inflammation-induced and/orgenetically manipulated models are known in the art. See Meredith G E, Sonsalla P K, Chesselet M F. “Animal models of Parkinson's disease progression.” Acta Neuropathol. 2008 April; 115(4):385-98. Epub 2008 Feb. 14.

### EXAMPLES

### Example 1

### Transcriptome Wiring Analysis Implicates α-Synuclein 3′UTR Selection in Parkinson's Disease

Common genetic variants in the human population may play a significant role in the pathogenesis of Parkinson's disease (PD) and other neurodegenerative disorders. As the majority of identified PD-associated variants do not alter protein coding, it is presumed that they modify gene expression, although direct evidence for this has been limited. Here we perform global transcriptome differential wiring analysis of PD patient and unaffected control brain tissues and identify a specific transcript isoform of aSynuclein (aSyn) with an extended 3′UTR, aSyn_{L}, that exhibits a dramatic correlation pattern change in diseased tissue. Strikingly, aSyn_{L} is even unwired from other aSyn transcripts with shorter 3′UTRs, suggesting a pathogenic role for altered aSyn 3′UTR usage in disease. Consistent with this, a genome-wide association study identifies disease-associated polymorphisms within the aSyn and Parkin loci as key genetic factors in aSyn 3′UTR selection. An additional determinant of aSyn 3′UTR selection is intracellular dopamine content, suggesting a mechanism for the propensity of dopaminergic neuron cell loss in PD patient brain. Finally, we show that differential 3′UTR usage modifies the accumulation and localization of aSyn protein. Taken together, these findings identify a unifying mechanism for PD pathogenesis in the context of genetic and environmental variation.

PD is the most common movement disorder of aging, characterized pathologically by neuronal loss that is particularly prominent among midbrain dopamine neurons (mDN). Whole transcriptome gene expression studies have afforded an unbiased screen of biological pathways that are altered with disease, and have identified specific RNA transcripts differentially expressed (DE) between PD and control brain tissues ¹⁻³. However, a pitfall inherent in such DE approaches is that the majority of alterations detected are likely to be secondary to the disease process, such as cell loss. Further limiting DE analyses, causal ‘master regulators’ may not themselves be differentially expressed during the course of the disease. In an attempt to overcome such limitations, we established a gene expression network analysis tool, termed ‘global differential wiring’ (GDW; see Methods)^{4,5}. Briefly, GDW identifies those transcripts that exhibit the greatest and most consistent change in their co-expression correlation (“rewired”) with DE transcripts when comparing panels of healthy control and patient tissue samples. Such transcripts are hypothesized to play a causal role in the disease.

A central role for aSyn in gene expression network perturbations in PD

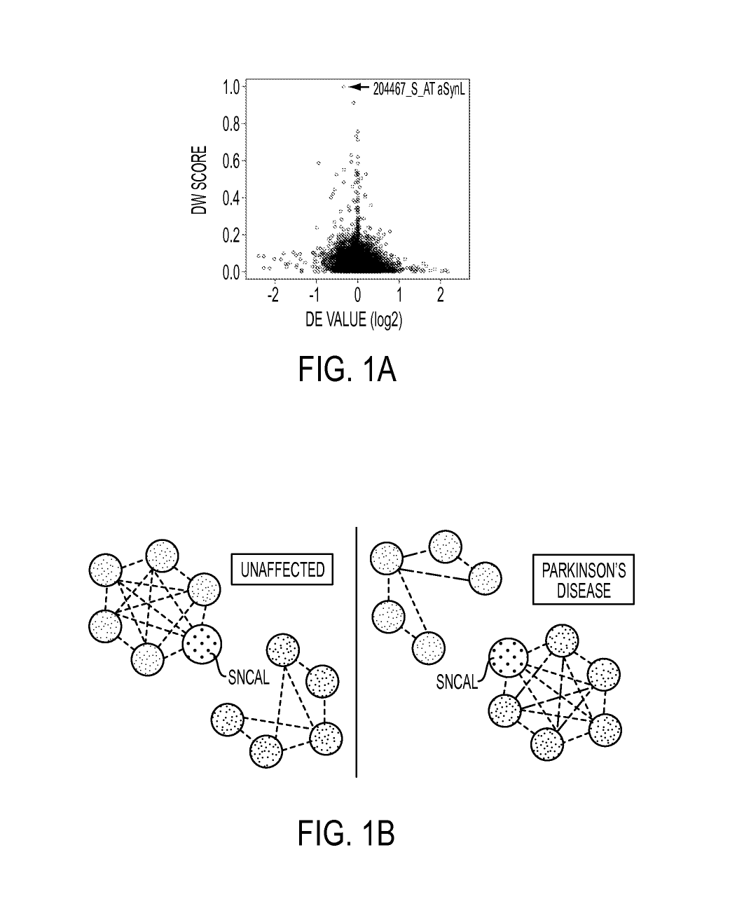

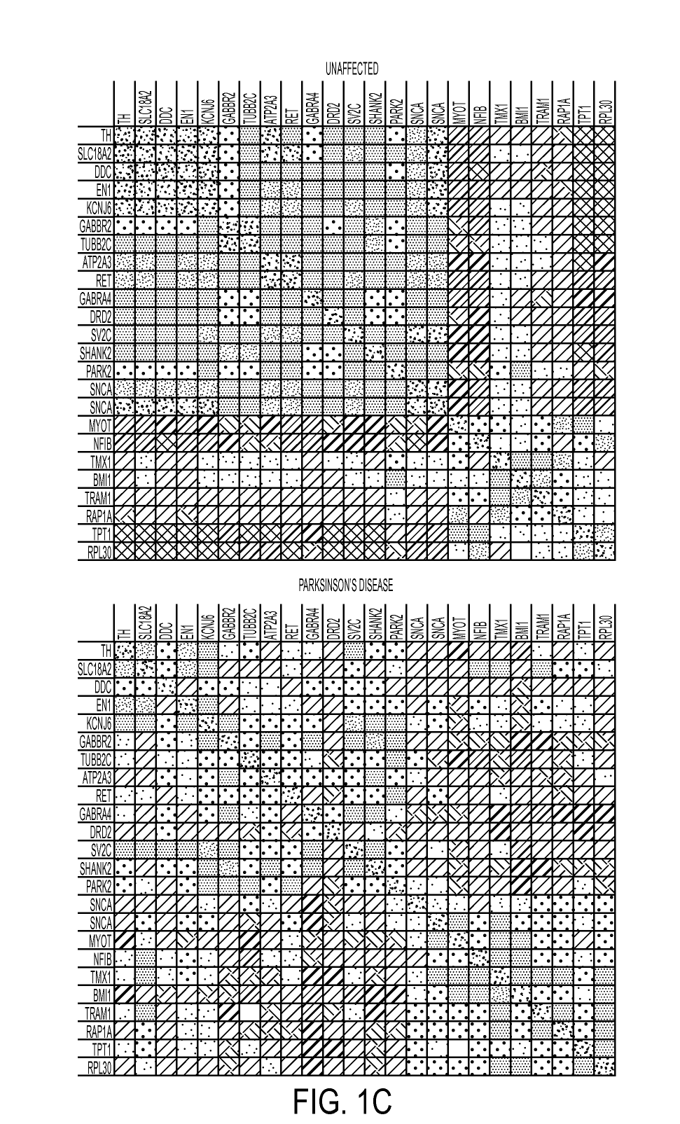

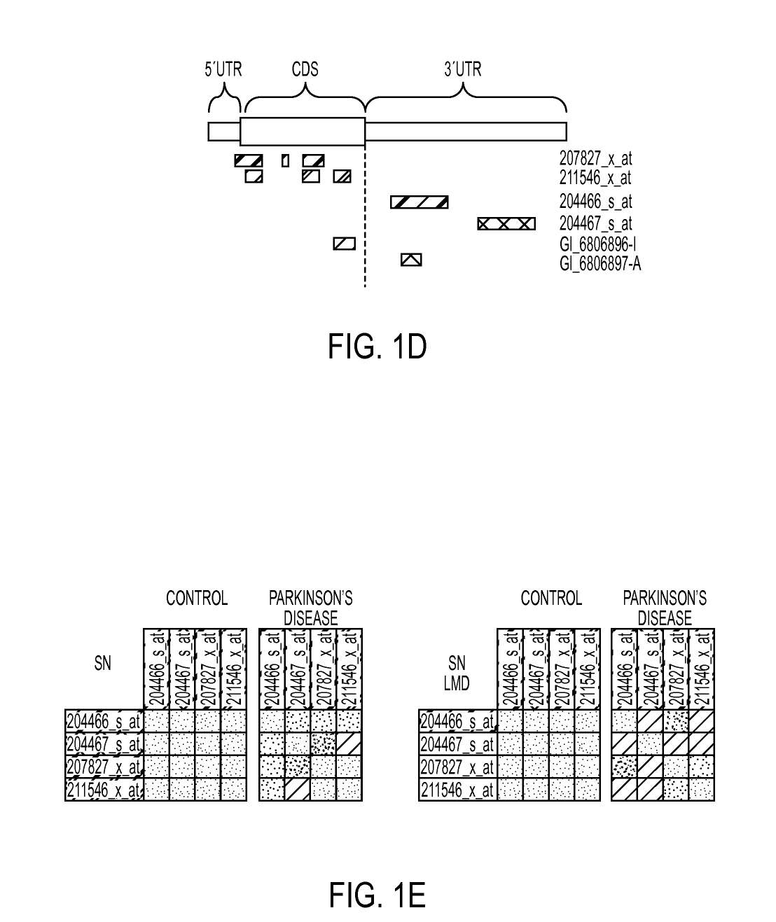

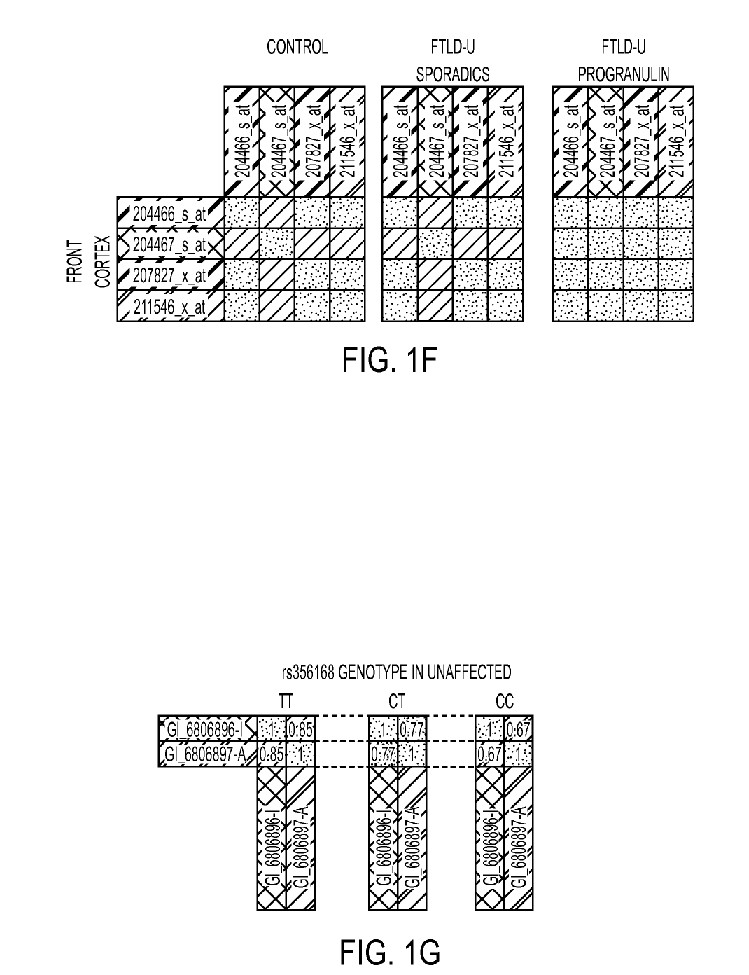

GDW analysis was performed on an existing gene expression dataset from age-matched unaffected-control and PD patient substantia nigra (SN) tissue (GEO GSE7621)¹. Strikingly, the most highly rewired probeset identified detects an aSyn isoform that harbors a longer 3′-UTR, aSynL (Supplementary Table 1). Replication of the study with independent PD and unaffected SN datasets (GEO GSE8397 ², GSE20292³, GSE20141⁶) again identified aSynL as among the most rewired transcripts, and aSynL ranked first in a combined analysis (FIG. 1a; Supplementary Table 1). Of note, despite being the most differentially wired, aSyn is not among the most differentially expressed genes between patients and controls (FIG. 1a, Supplementary Table 6). aSyn has previously been invoked in sporadic PD, as common SNPs in its locus increase PD risk⁷⁻⁹, and intraneuronal inclusions composed of aSyn protein, termed Lewy bodies, typify PD brain pathology ¹⁰. Furthermore, very rare mutations in aSyn as well as triplication of the aSyn gene locus lead to familial inherited forms of PD ^{11, 12}.

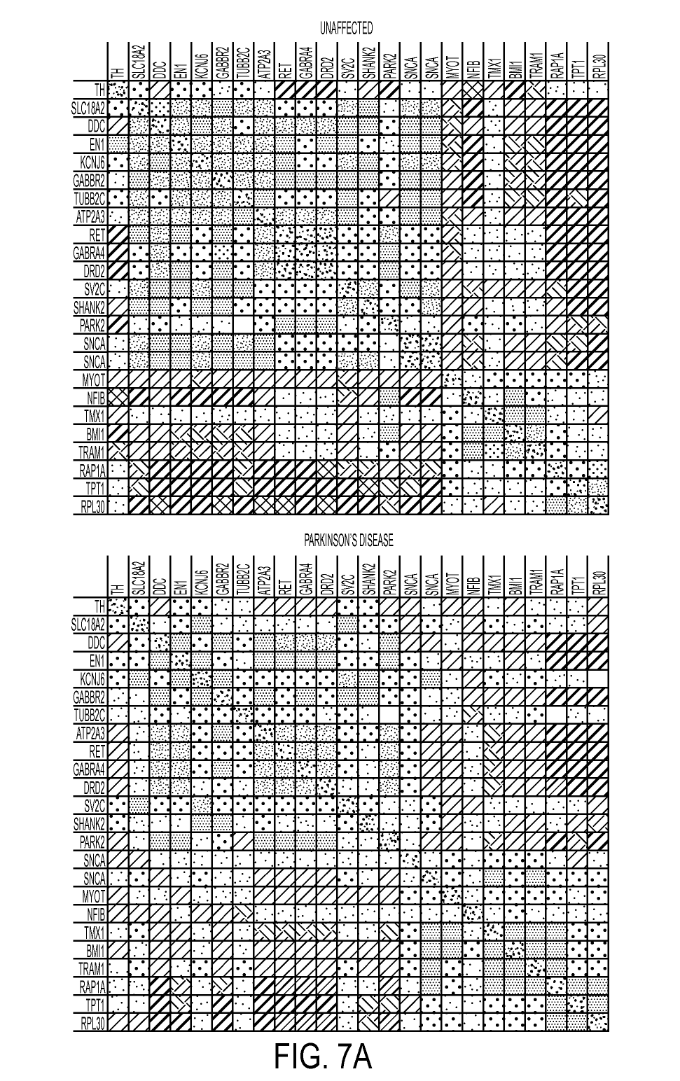

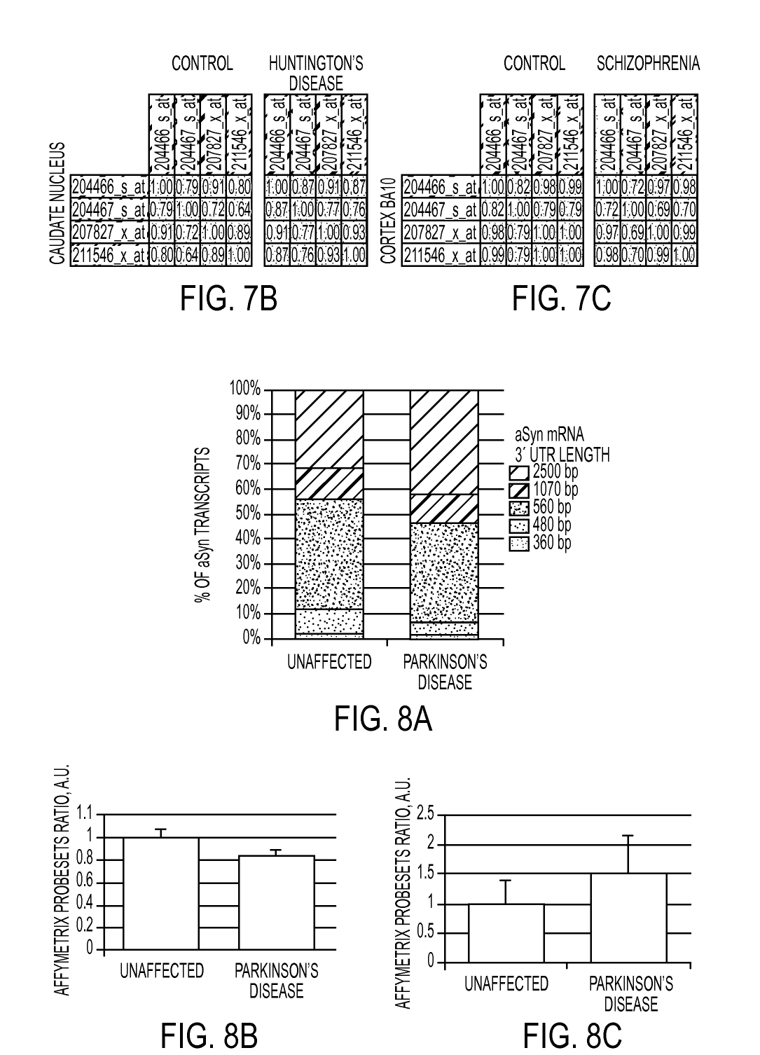

A post-hoc analysis, aimed at identifying the factors underlying the high DW score of aSynL, revealed that whereas aSynL expression is typically highly correlated with a sub-network of genes across the panel of unaffected controls, expression of aSynL becomes unwired from this sub-network in the disease sample panel, where it is instead wired to a second sub-network (FIG. 1 b-c). The first sub-network is enriched in transcripts that are associated with synaptic and vesicular transport functions and includes dopa decarboxylase (DDC) and vesicle monoamine transporter type 2 (VMAT2; SLC18A2). In contrast, the second is associated with nuclear localization and transcription regulation functions (Supplementary Table 7). The GDW of aSynL in PD midbrain is unlikely to be a trivial consequence of the loss of mDN, as similar findings were obtained with laser-dissected mDN tissue (FIG. 7a).

Surprisingly, among the transcripts that appeared rewired from aSynL in PD were other aSyn transcripts, as determined using probesets within the protein coding sequences (CDS) of aSyn (such as probeset 211546_x_at, FIG. 1b). These data suggest a role for aSyn alternative 3′UTR selection. We thus focused further on changes in correlation among aSyn probesets targeting either the 3′UTR or the CDS (FIG. 1d, Supplementary table 2). Expression of all aSyn transcripts appeared highly correlated among healthy adult brain tissue samples, as expected. In contrast, the correlation between the 3′UTR probesets and the CDS probesets decreased in the PD state in 2 independent datasets (FIG. 1e). This finding appears to be PD-specific, as we did not observe such aSyn loss of correlation in other neurological diseases including Frontotemporal Dementia (FTD), Huntington's disease (HD) or schizophrenia (FIG. 1f, FIG. 7b-c ^{13, 14}).

A PD-associated SNP is predictive of aSyn rewiring even in unaffected controls.

Transcripts that are most highly rewired in the context of disease are hypothesized to play a causal, high-impact role on global gene expression and thus represent candidate disease modifiers. In such a network model, genetic or environmental variations initially modify these ‘master regulator’ or ‘nodal’ genes, leading secondarily to global network perturbations ^{4, 5}. We thus investigated the influence of common PD-associated SNPs in the 3′ region of the aSyn locus on aSynL isoform wiring. Importantly, these analyses were performed in individuals not affected by PD, to minimize potential confounding effects of the disease pathology, using a previously reported dataset of genotyped cerebral cortex tissue samples ¹⁵. In cortical brain samples from unaffected individuals, the presence of a common SNP variant associated with increased PD risk (C at rs356168, 3 kb downstream of the aSyn 3′UTR) is correspondingly associated with significantly decreased co-expression correlation (rewiring) between aSynL and a probe detecting all aSyn transcripts (aSynT) (FIG. 1g). In the context of a global analysis comparing tissue samples homozygous for the risk-associated variant (rs356168 C/C) or homozygous for the protective variant (rs356168 T/T), aSynL expression is found to be globally unwired (in terms of co-expression correlation) from genes functionally annotated as associated with synaptic function, and to be rewired to genes associated with nuclear functions (Supplementary Table 8). In summary, even unaffected individuals harboring an aSyn PD-risk variant display both the aSyn isoform-specific and global transcriptome rewiring patterns of PD. These data argue strongly that the observed patterns are not secondary to cell loss or other aspect of the disease process.

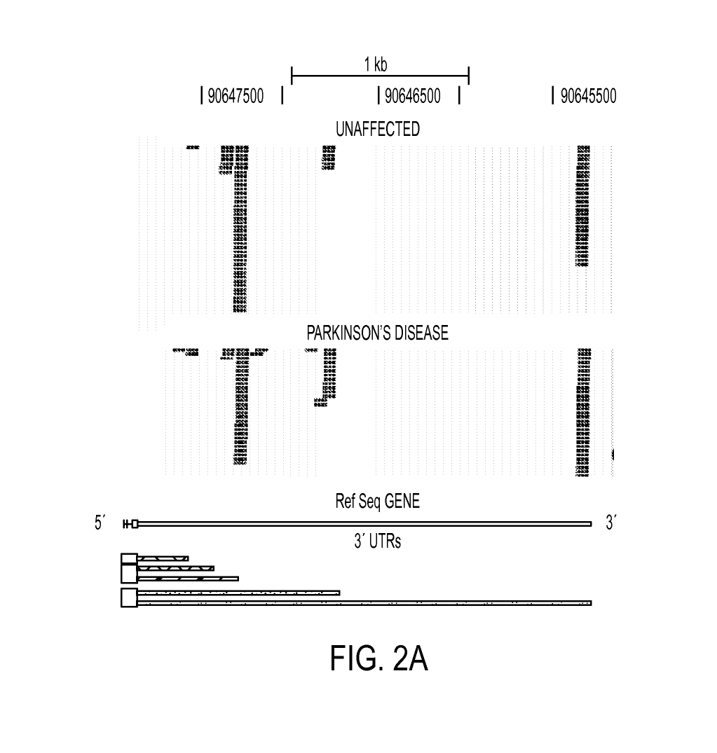

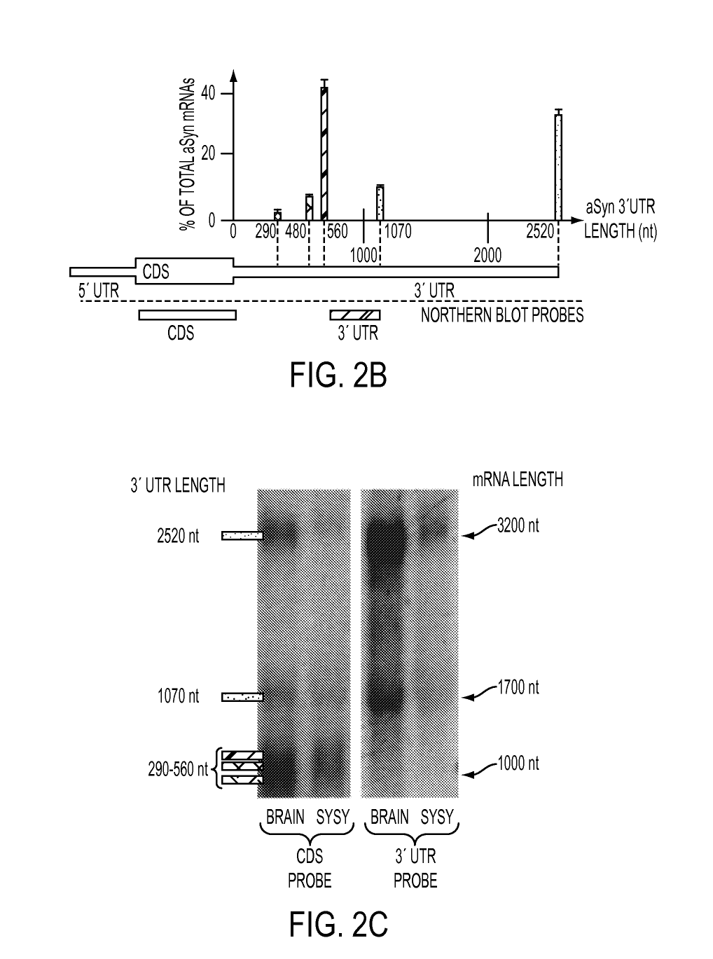

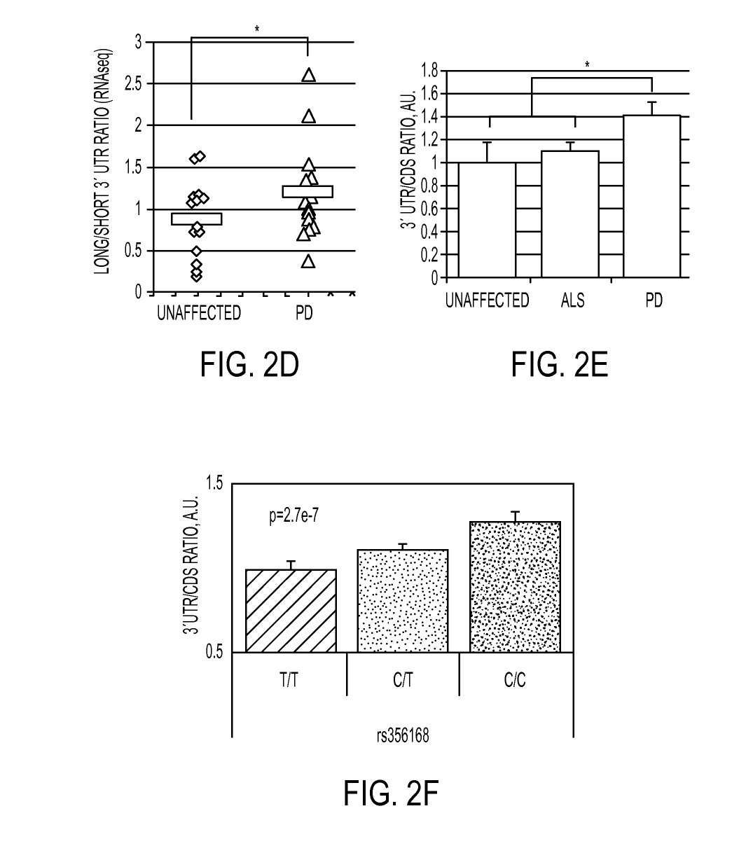

We next sought to characterize more precisely the different aSyn 3′UTR mRNA species in normal and PD human brain. For this purpose, we devised a high-throughput, whole-transcriptome method for sequencing the 3′UTR ends of polyadenylated mRNA transcripts (termed pA-RNAseq; see Methods) in a cohort of 17 unaffected and 17 PD cerebral cortical tissue samples. This revealed 5 aSyn 3′UTR isoforms, with lengths from 290 nt to 2520 nt (FIG. 2 a-b); of these, the 560 nt and 2520 nt forms were predominant. The existence and relative preponderance of these species was further confirmed by Northern Blot (FIG. 2c). We next hypothesized, based on the GDW analysis above, that aSyn 3′UTR selection might be altered in PD. Comparison of pA-RNAseq profiles from PD and unaffected cerebral cortex samples revealed an increase in the preponderance of the long 3′UTR species (>560 nt) relative to shorter species (<560 nt; FIG. 2d, FIG. 8a). Such a relative increase in aSynL was confirmed by qPCR and appears specific for PD, as this is not observed in RNA from amyotrophic lateral sclerosis patient samples (FIG. 2e). We note that the modified aSyn 3′UTR selection associated with PD patient tissue is detected in cerebral cortex tissue, which typically harbors pathological evidence of the disease process without frank cell loss; thus, this phenotype is unlikely to be a secondary consequence of neurodegeneration. Re-analysis of the aSynL:total ratio in the context of SN (FIG. 8b) or laser-microdissected SN mDNs (FIG. 8c) from PD patients or unaffected individuals did not show statistically significant change, perhaps reflecting confounding effects of the late-stage disease pathology in these samples (such as the dramatic loss of dopamine neurons).

To further circumvent potential confounding effects in disease tissue, we quantified the aSynL:total transcript ratio in unaffected brain tissue from individuals with PD risk-associated and protective SNP variants at the aSyn locus. Reanalysis of cortical tissue from unaffected individuals ¹⁶ demonstrated that the risk-associated variant (C at rs356168) was highly predictive of an elevated aSynL:total transcript ratio. This ratio quantitative trait locus (rQTL) effect was reproduced in an independent series of cerebral cortical tissue samples from AD patients ¹⁶. Combination of both datasets also led to a highly significant association (p<10⁻⁶, FIG. 2f). Taken together, these analyses implicate genetic variants at the SNCA locus as cis-acting modulators of aSyn 3′UTR selection, even in unaffected brain.

GWAS of aSynL:total ratio in cerebral cortex from unaffected controls

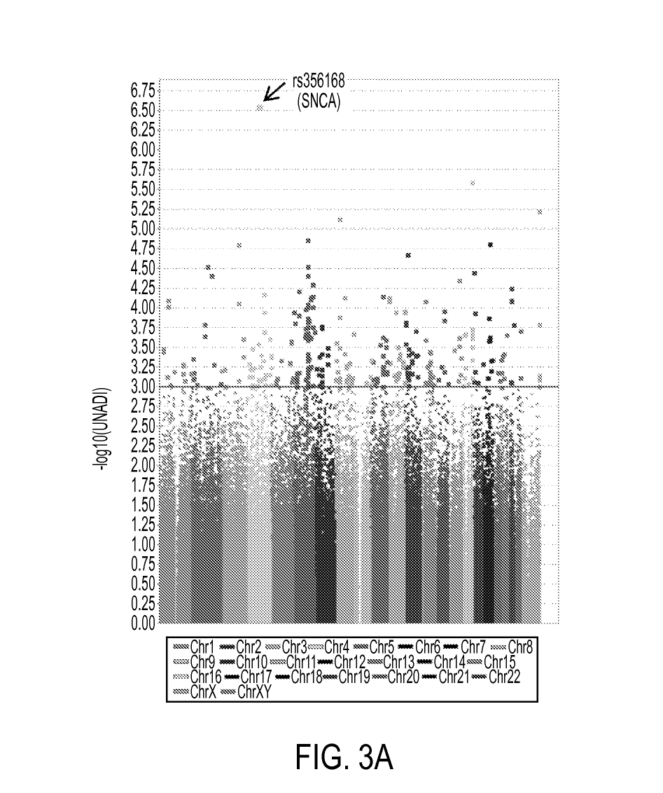

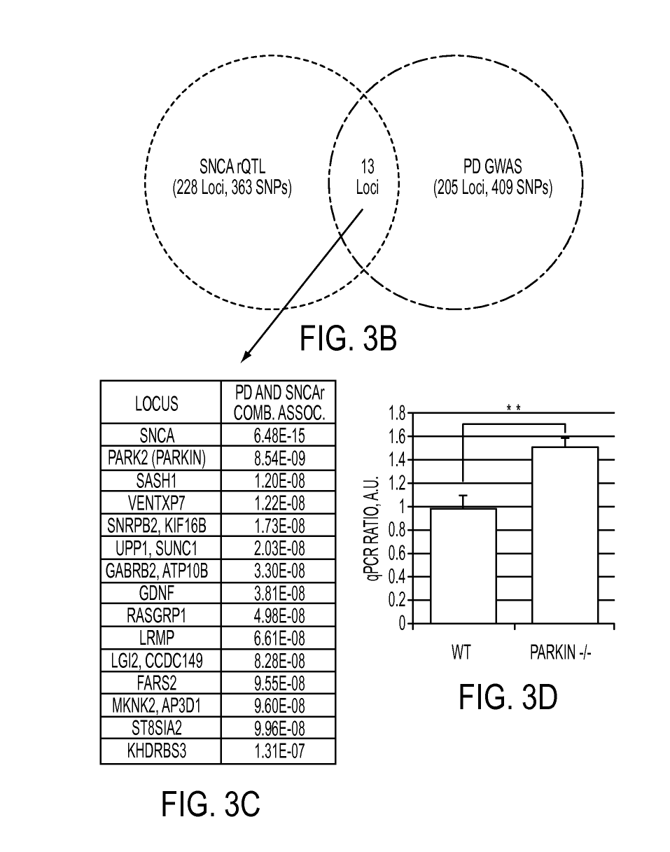

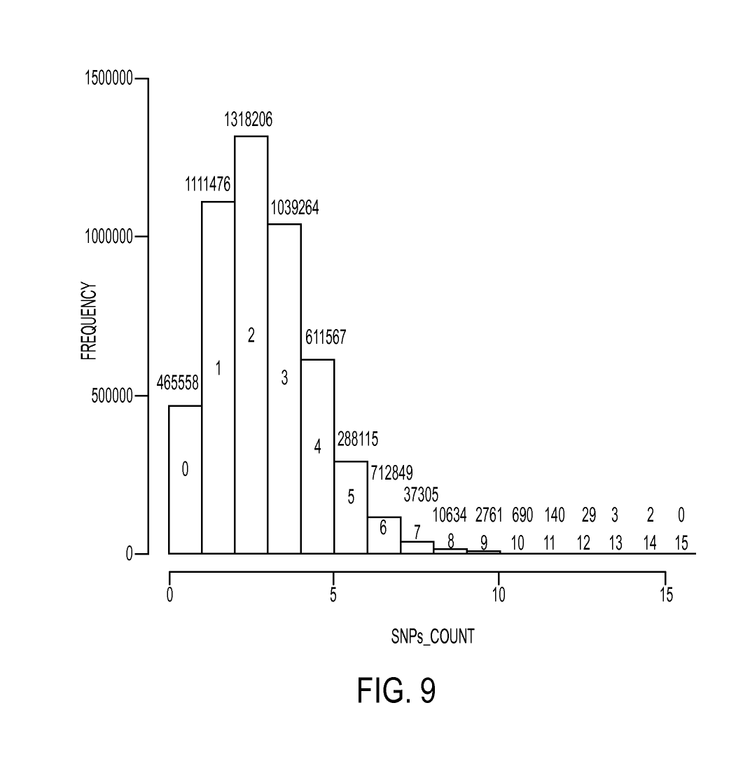

We further pursued the regulation of the aSyn rQTL using an unbiased, genome-wide approach by reanalysis of concurrent genome-wide SNP and cerebral cortical gene expression data ^{15, 16}. Strikingly, this genome-wide reanalysis identified the same PD risk-associated SNP in the 3′ region of the aSyn locus (rs356168; as in FIG. 2f) as the most highly correlated with the aSyn mRNA 3′UTR ratio (FIG. 3a). We then broadly compared genetic loci implicated by the aSyn ratio (rQTL-GWAS) genome-wide analyses with loci previously implicated by PD risk association (risk-GWAS), aiming to identify overlapping loci (other than aSyn) that would be predictive of PD risk as well as the aSyn mRNA 3′UTR ratio. 13 genetic loci were identified that harbor SNPs associated with both disease risk and ratio-QTL SNPs (p<10⁻³ for each; a lower stringency was chosen for each individual association to reduce false-negative calls¹⁷ and as such combined analyses greatly increase statistical power¹⁸; FIG. 3b-c, and Supplementary Tables 3, 9). The highly significant overlap between trans-acting loci that modify aSynL:total ratio and those associated with PD susceptibility (p<10⁻⁶ by bootstrap analysis [see Methods], with 8-fold more overlapping loci than predicted to be expected by chance; FIG. 9) further supports a role for aSyn 3′UTR selection in the disease pathology. Remarkably, aSyn and Parkin were identified as the most statistically significant loci in the overlap analysis. Furthermore, other loci identified within this list, such as GDNF and GABA-A receptor B2 subunit, have been implicated in PD pathology ¹⁹.

Rare autosomal recessive inherited mutations in Parkin lead to an early-onset form of PD ²⁰, and Parkin is thought to function in part in the regulation of mitochondrial function or integrity ²¹, which appears altered in late-stage PD pathology ²². To more directly evaluate the role of Parkin in modulating aSyn ratio, we investigated the rodent aSyn transcript 3′UTR ratio in mice that are deficient in Parkin ²³. Parkin deficient mice displayed an increased aSynL:total ratio in brain when compared to littermate controls (FIG. 3d), consistent with a role for Parkin as an upstream determinant of aSynL:total ratio. The species conservation of alternative aSyn 3′UTR regulation by Parkin supports a functional significance.

Dopamine Regulation of aSyn Polyadenylation

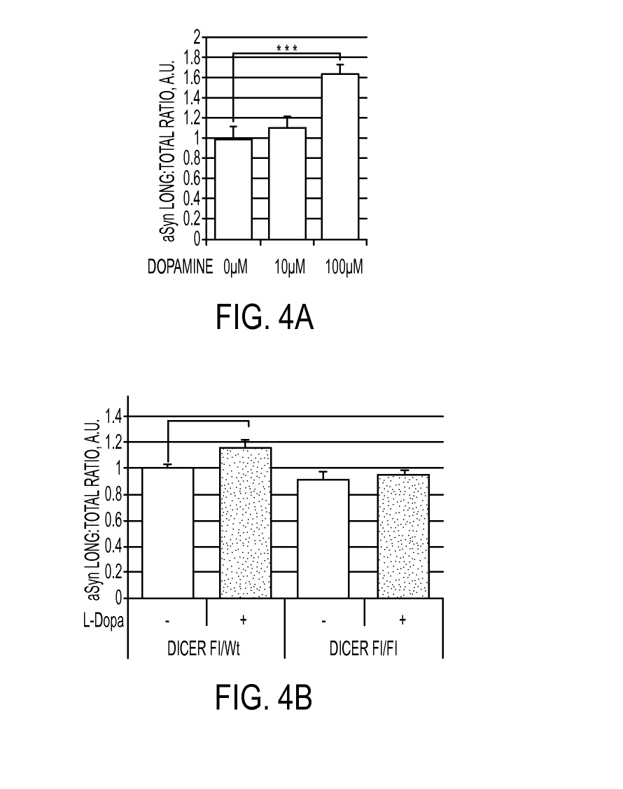

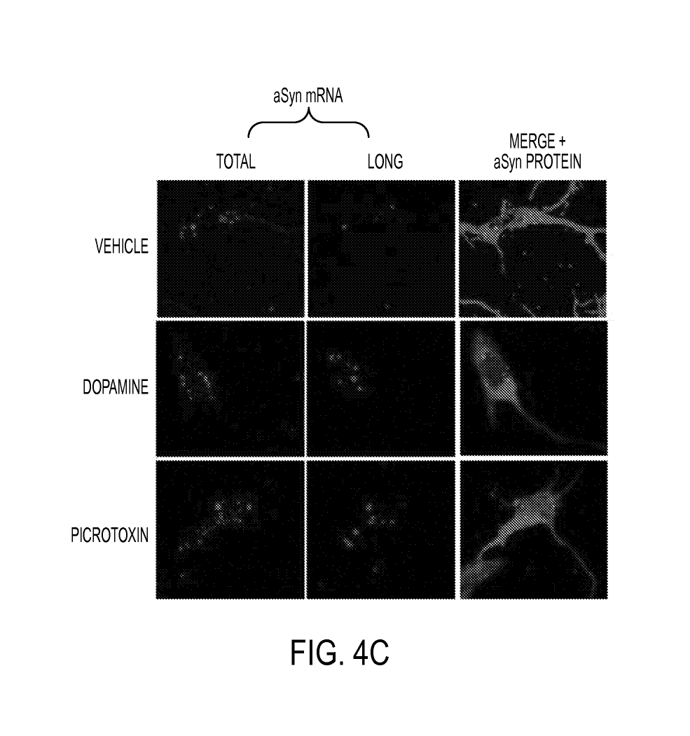

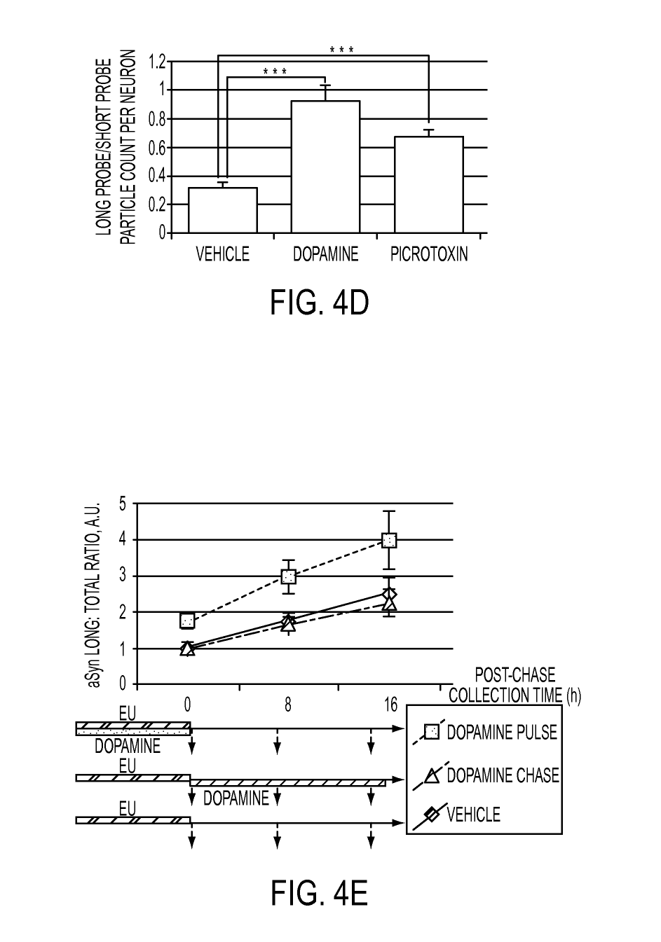

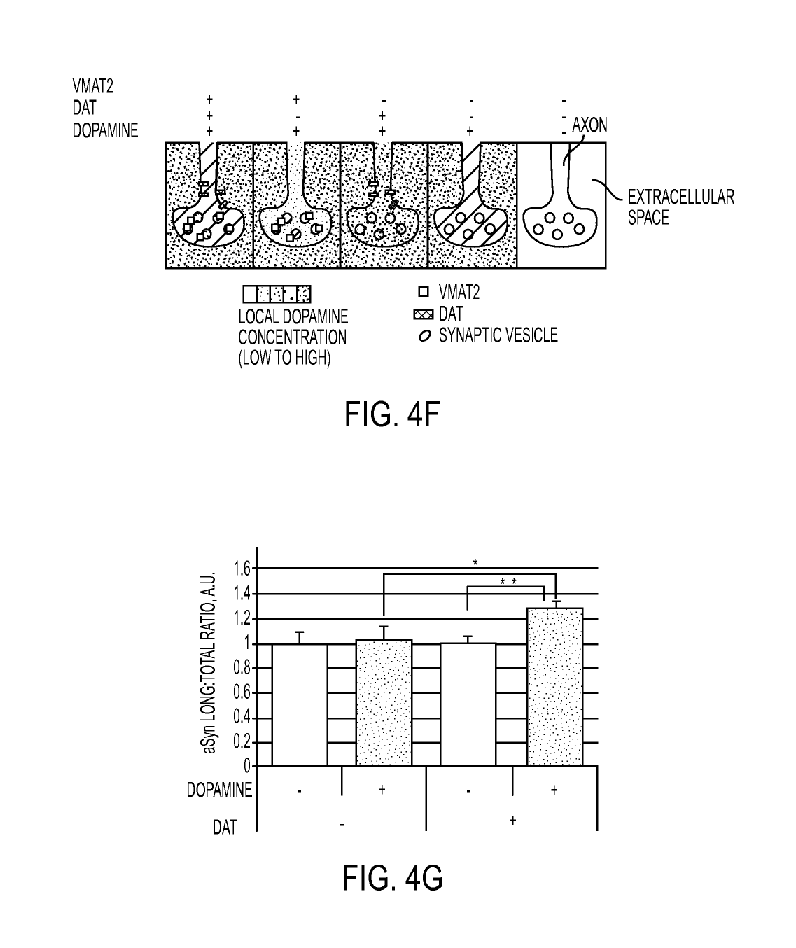

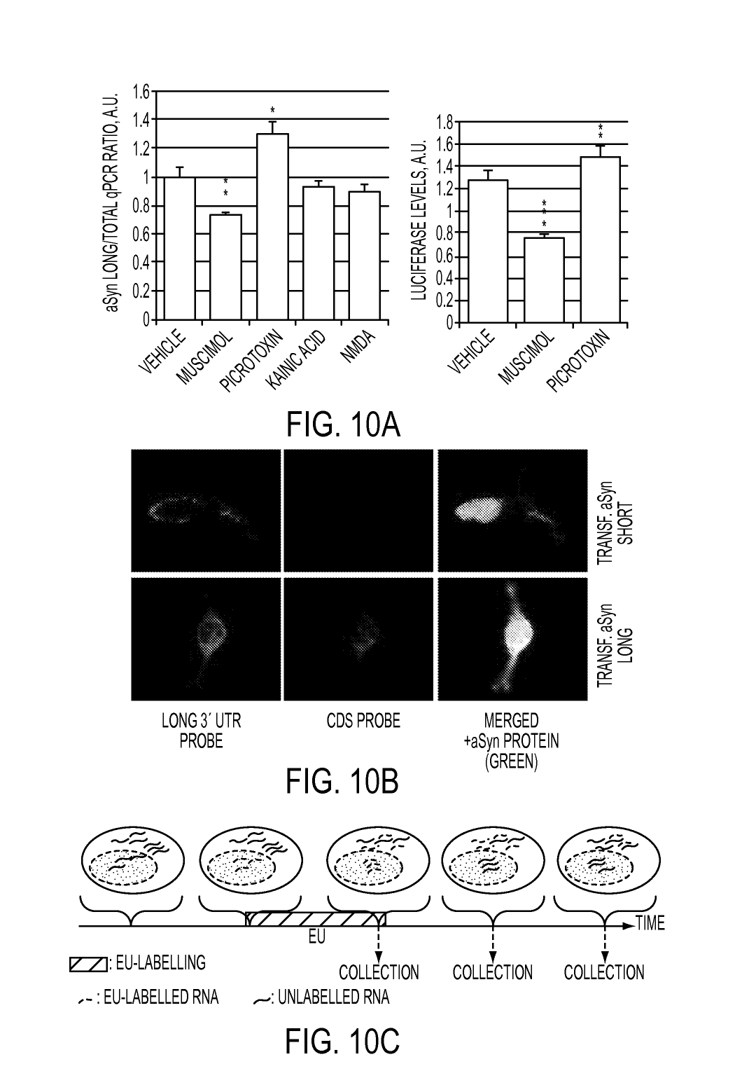

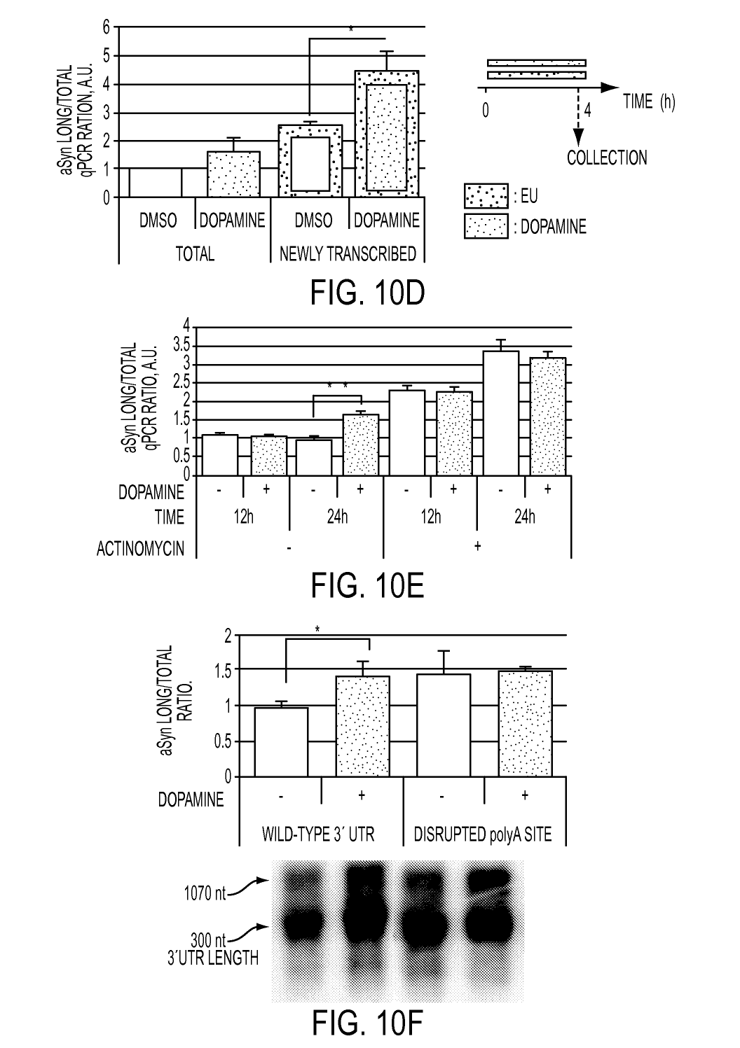

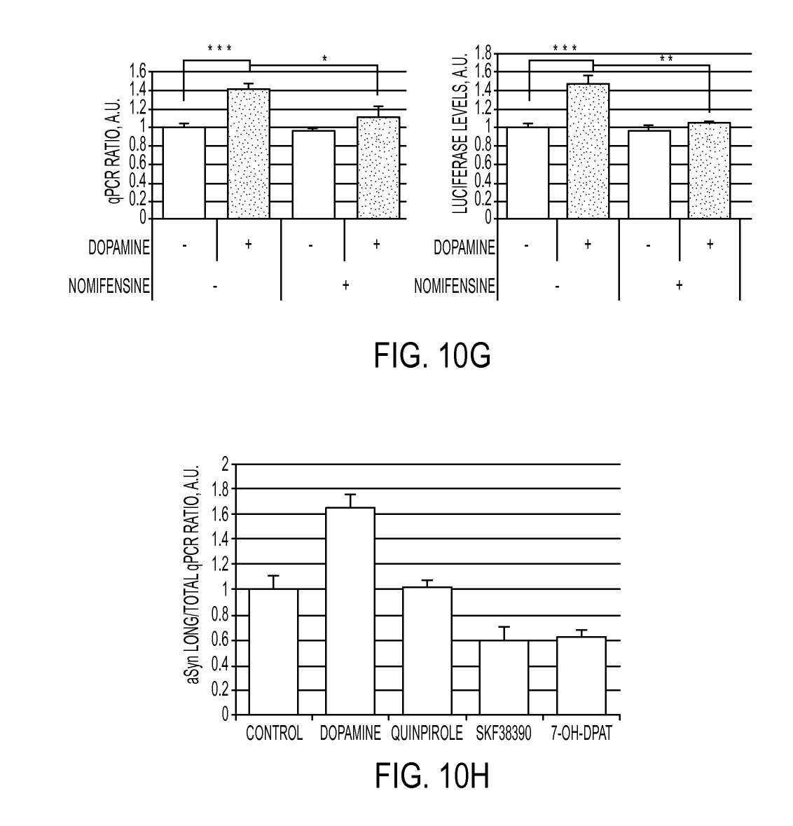

Given the pathological evidence for altered aSyn accumulation in PD mDNs, we hypothesized that dopamine could further modulate aSyn 3′ UTR usage, concomitant with genetic regulation as detailed above. We thus queried the regulation of aSyn 3′UTR selection by dopamine in a primary rat cortical neuron culture model. Treatment of these cells with high levels of dopamine (100 μM) led to an increase in the aSynL:total ratio (FIG. 4a). To examine the role of dopamine content on aSyn transcript regulation in vivo in mDNs, we compared the effect of 1-Dopa on the aSynL:total ratio in mice treated systemically with I-Dopa—which is taken up by mDNs through the dopamine transporter and leads to increased dopamine content. Whereas the aSynL:total ratio appeared significantly increased by I-Dopa treatment in 2-month old control mouse midbrain (DAT-Cre/Dicer^{flox/+}), I-Dopa treatment did not alter the ratio in midbrain tissue from littermates deficient in mDNs (DAT-Cre/Dicer^{flox/flox} mice; ²⁴, FIG. 4b). Furthermore, the effect of 1-Dopa on the ratio was not apparent in brain regions other than midbrain, such as striatal tissue. We note that this contrasts with the PD risk-associated SNP effect on the transcript ratio described above, which is readily evident in non-dopaminergic neurons, suggesting that the mechanism of dopamine action on the aSyn transcript ratio may be distinct from that of the risk SNP. Data from a publicly available Gene Expression Atlas (GEO GSE7307) further supports an elevated aSyn_{L}:total in midbrain dopamine neurons: among 22 human brain regions analyzed, SN exhibits the highest ratio (FIG. 14a). Screening of other neurotransmitter receptor signaling modulators in vitro also supported a role for GABAergic modulation in the regulation of the aSynL:total transcript ratio. Specifically, the GABA-A antagonist picrotoxin, significantly increased this ratio in cortical primary cultures (FIG. 10a). In contrast, modulation of NMDA or kainate glutamate receptors did not appear to impact the aSynL:total transcript ratio (FIG. 10a).

To confirm the modified aSyn 3′UTR usage in an independent fashion, we next performed in situ hybridization (ISH) studies on primary cortical neuron cultures from transgenic mice bearing a fragment of human chromosome 4 encompassing the whole aSyn locus including the 3′UTR ²⁵ (termed aSyn P1 Artificial Chromosome [PAC] mice). Nucleic acid probes were designed to either detect all human aSyn mRNA species or specifically the human long 3′UTR; these probes do not cross-react with endogenous rodent aSyn mRNA (FIG. 10b). As expected, treatment with either dopamine or picrotoxin led to an increase in the aSyn_{L}:total ratio (FIG. 4d-e).

To characterize the mechanism by which dopamine impacts the aSynL:total ratio, we sought to distinguish between co-transcriptional modifications (acting on nascent aSyn mRNA generation) such as alternative polyadenylation, and post-transcriptional effects on the relative stability of different isoforms. We thus proceeded to perform pulse-chase RNA labeling studies in dopamine treated or untreated human SH-SY5Y cells (FIG. 10c). Dopamine treatment exclusively during the pulse-labeling period led to a robust and durable increase in aSyn_{L}:total ratio among the labeled RNA population. By contrast, dopamine treatment exclusively post-labeling did not produce any effect on the aSyn_{L}:total ratio among the labeled RNA population (FIG. 4c). We also observed that aSyn_{L}:total ratio gradually increased as a function of time after labeling, with a rate independent of dopamine treatment (FIG. 4c). Of note, the dopamine-mediated increase of the aSyn_{L}:total ratio in the nascent RNA population was detectable after as little as 4 hrs of treatment, whereas it took much longer treatment for the dopamine effect to become detectable in the total RNA population (at least 16 hrs; FIG. 10d). Taken together, these data suggest that dopamine acts co-transcriptionally to modify alternative 3′UTR polyadenylation, rather than acting post-transcriptionally on the stability of the mRNA isoforms. Consistent with such a mechanism, treatment with the transcriptional inhibitor, actinomycin D, along with dopamine, prevented the increase in aSyn_{L}:total ratio (FIG. 10e). Furthermore, disruption of a polyadenylation site corresponding to the short 3′UTR within an aSyn mini-gene plasmid occluded the dopamine-mediated increase in the aSyn_{L}:total ratio in the mini-gene context (FIG. 10f).

The regulation of aSyn 3′UTR selection by dopamine may either be a consequence of accumulation of intracellular dopamine, or due to receptor-mediated dopaminergic signaling. We sought to distinguish these mechanisms. As intracellular accumulation of dopamine is greatly facilitated by the dopamine transporter (DAT) in SN neurons but is absent from cortical neurons, we overexpressed DAT in cortical neuron cultures; this significantly increased the sensitivity of primary cortical neurons to dopamine (at 10 μM) with respect to aSynL:total ratio modification (FIG. 4f-g). In contrast, the monoamine reuptake inhibitor nomifensine suppressed sensitivity to high-dose dopamine (100 μM), supporting a role for intracellular dopamine accumulation through monoamine transporters (Supplementary FIG. 4g). We cannot exclude additional extracellular roles for dopamine through receptor signaling, but analysis of receptor agonists was inconclusive (FIG. 10h).

aSynL 3′UTR is associated with increased aSyn translation

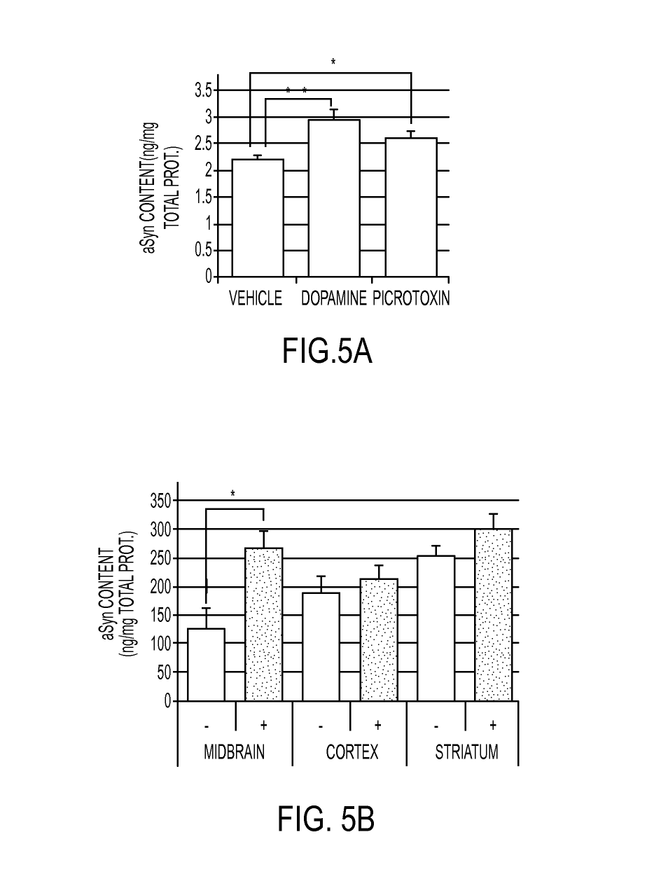

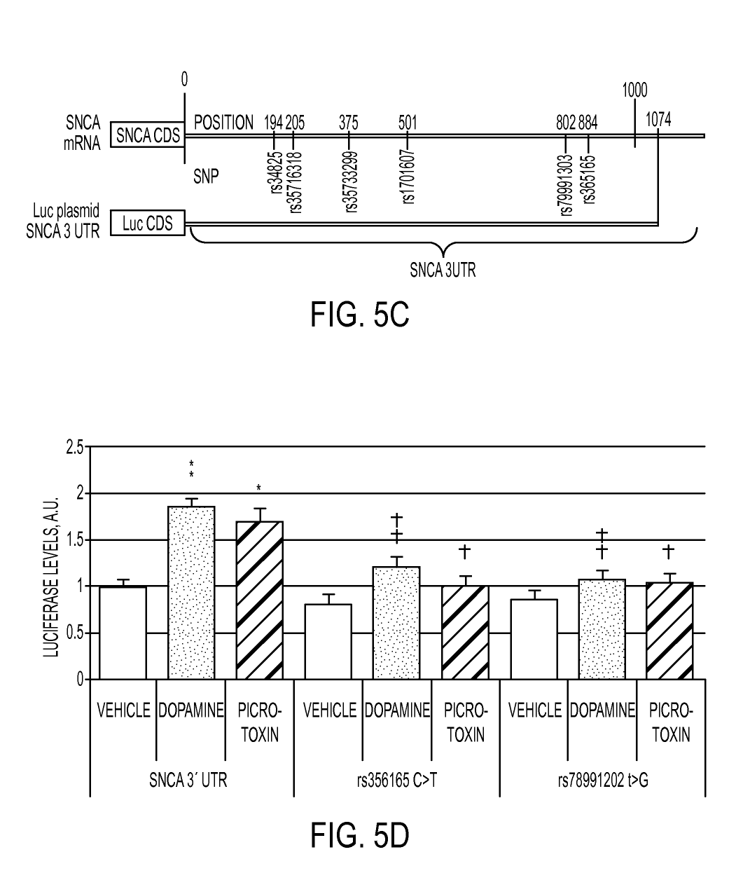

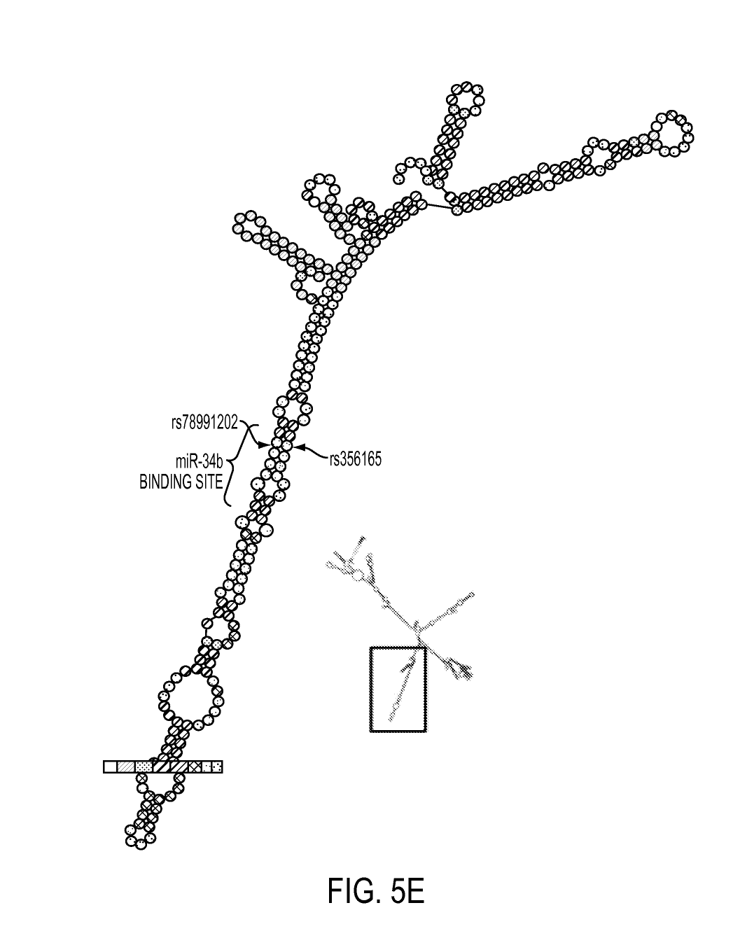

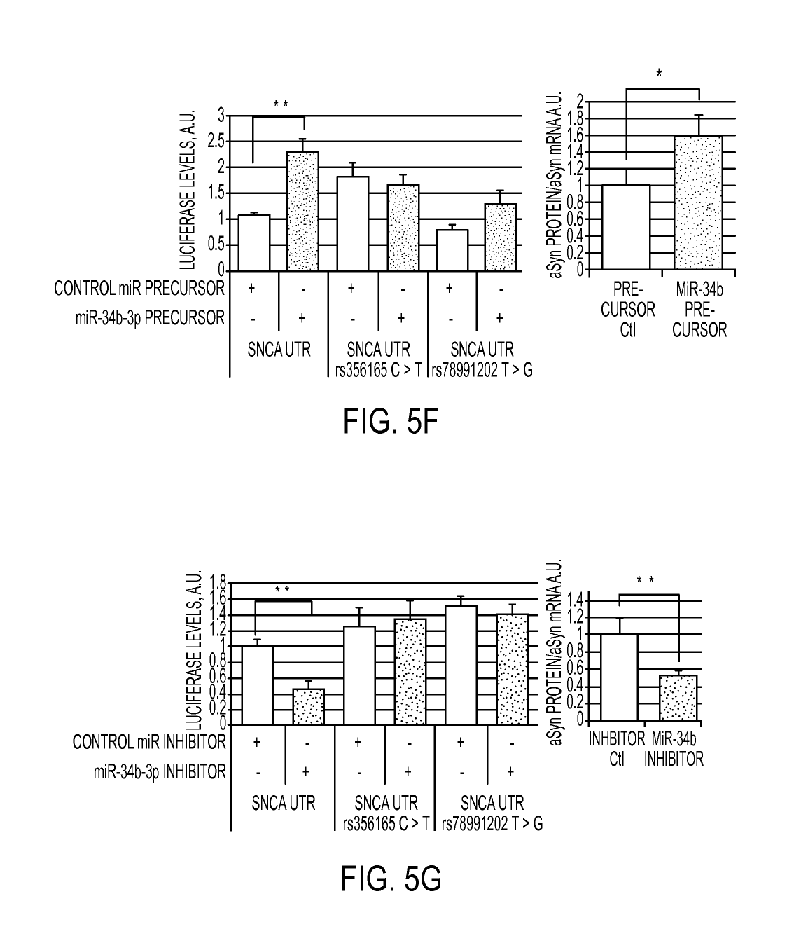

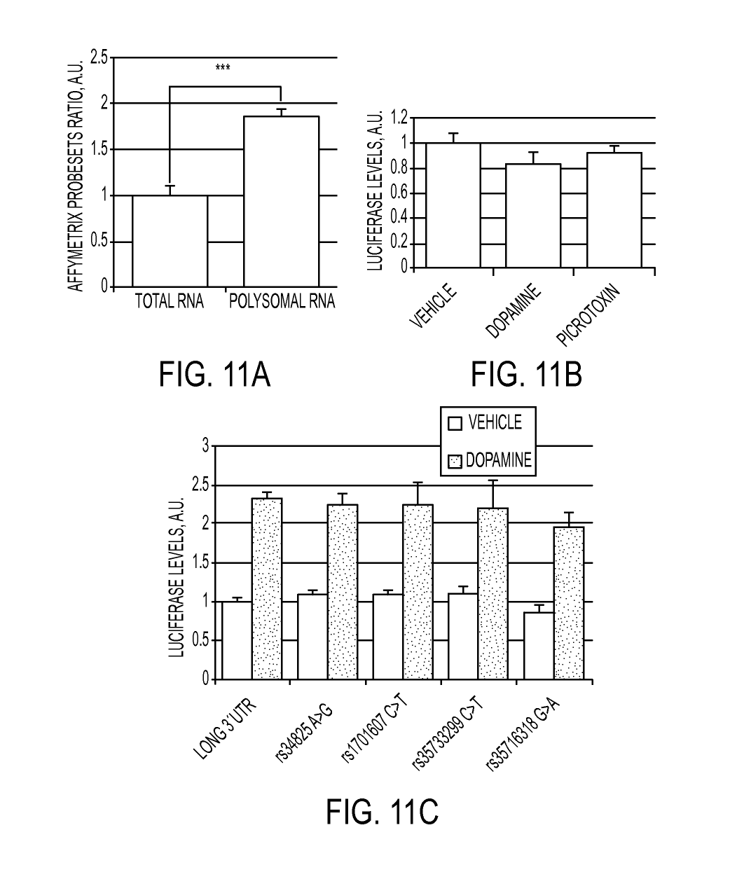

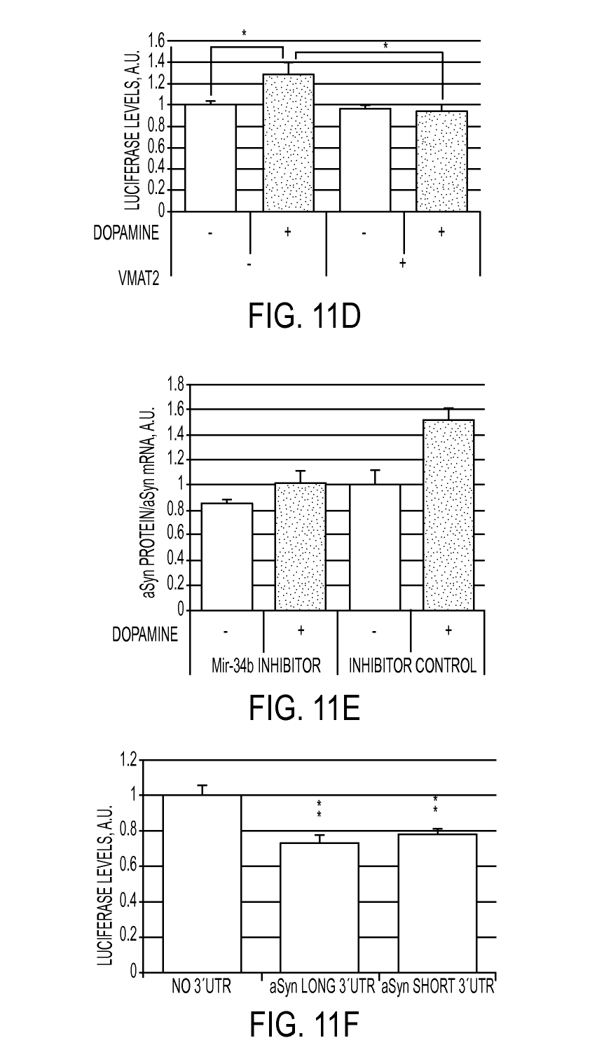

3′UTR sequence elements can lead to both positive and negative effects on mRNA accumulation, translation, or stability. Reanalysis of global RNA studies in cultured cells indicated that aSyn_{L} is enriched in the polysomal fraction relatively to total aSyn, consistent with a positive effect of the aSyn 3′UTR on mRNA translation (FIG. 11a ²⁶). Consistent with this, either dopamine or picrotoxin treatment, previously shown to increase the aSynL:total ratio also significantly increased endogenous aSyn protein levels in SH-SY5Y cells, as quantified by ELISA (FIG. 5a). Similarly, I-Dopa treatment of 10-mo old human aSyn PAC transgenic mice (as above in FIG. 4b) significantly increased the accumulation of human aSyn protein in midbrain but not in other brain regions such as striatum or cortex (FIG. 5b). To further parse the role of the aSyn 3′UTR element, we transfected a luciferase assay vector that harbors a 1.1 kb human aSyn 3′UTR element into SH-SY5Y human neuroblastoma cells (FIG. 5c). Treatment of vector-transfected SH-SY5Y cells with dopamine led to increased luciferase expression (FIG. 5d) that was by contrast not observed for a vector harboring only the first 560 bp of human aSyn 3′UTR (FIG. 11b). Additionally, the GABA-A receptor agonist muscimol decreased luciferase accumulation, whereas the GABA-A antagonist picrotoxin increased this (FIG. 10a). Taken together, these findings directly implicate the distal part of aSyn 3′UTR that is specific to aSynL transcript as a cis-acting element leading to increased mRNA translation. The dopamine effect is mediated by preferential generation of the aSynL due to alternative polyadenylation, leading secondarily to increased protein translation.

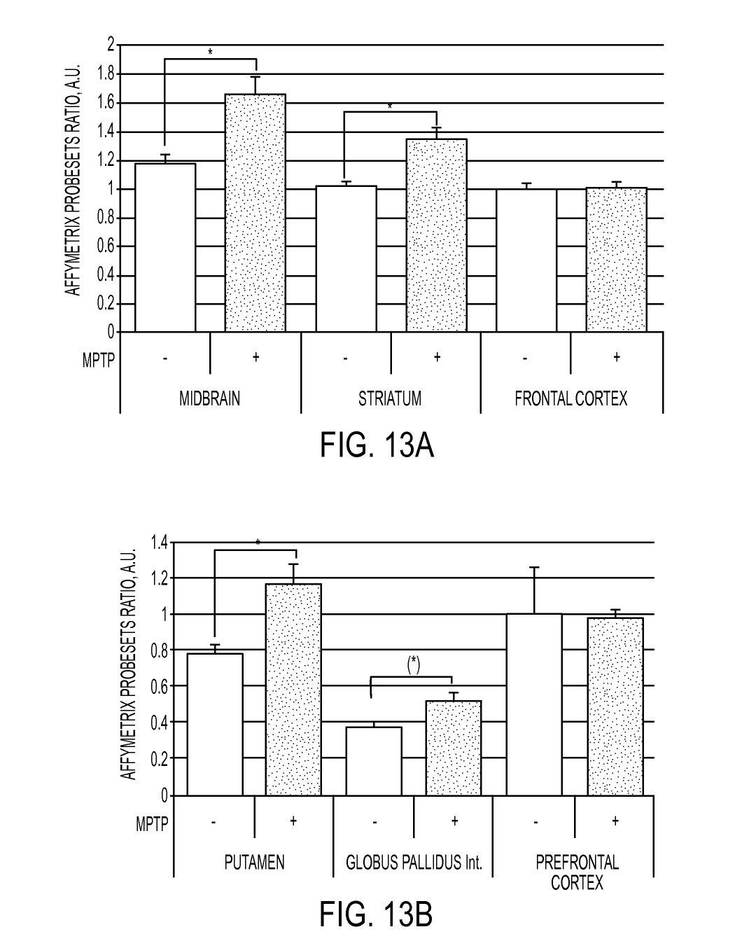

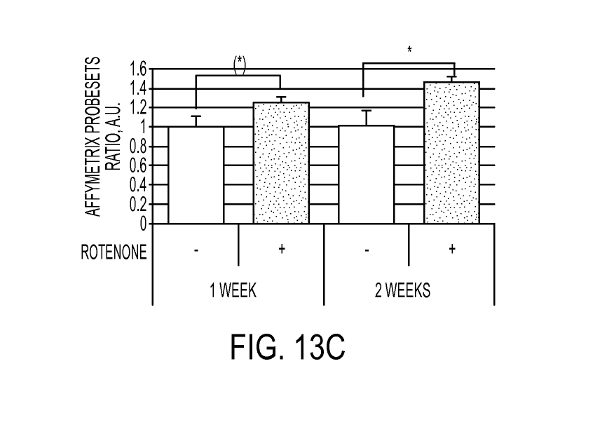

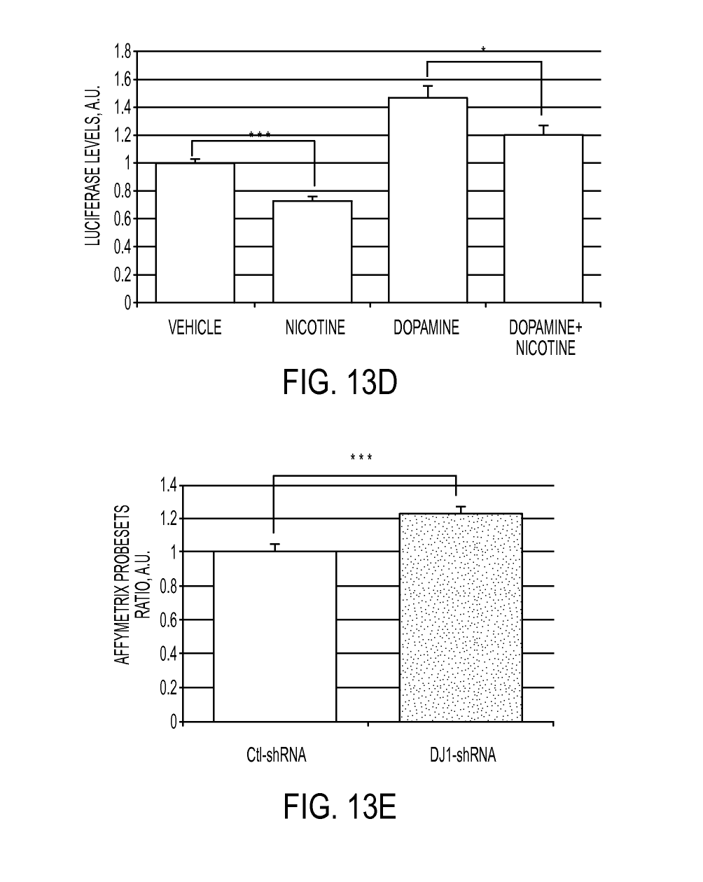

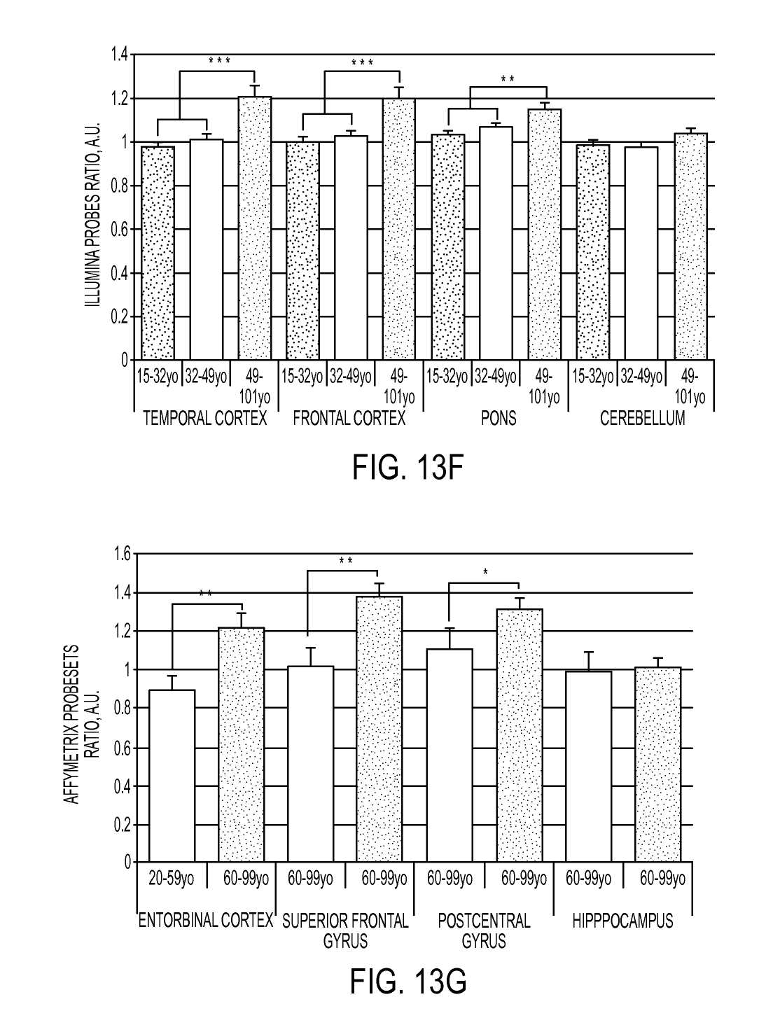

We next used this luciferase assay to study the specific role of intracellular dopamine on aSyn translation activation through the aSyn 3′UTR. As expected—given the impact of intracellular dopamine of the aSyn transcript ratio (FIG. 4a, b)—the dopamine reuptake inhibitor nomifensine suppressed the increased luciferase accumulation in SH-SY5Y cells transfected with the luciferase-aSyn 3′UTR vector and treated with dopamine as above (FIG. 10g). Similarly, overexpression of VMAT2-which sequesters dopamine in vesicles and away from other cytoplasmic constituents (FIG. 4f) and is thus protective ²⁷—also suppressed the dopamine-mediated increase in luciferase accumulation (FIG. 11d). These data support a specific role for cytoplasmic dopamine in regulation of the aSynL 3′UTR ratio and translation, perhaps as a consequence of mitochondrial disruption as previously described with cytoplasmic dopamine accumulation²⁸. Consistent with this model, in vivo treatment of mice with 1-methyl-4-phenyl-1,2,3,6-tetrahydropyridine (MPTP)—a mitochondrial toxin that accumulates in dopamine neuron cytoplasm because of selective uptake of its MPP+metabolite through DAT—led to an increased aSynL:total ratio in vivo (FIG. 13a, b ²⁹).

Common aSyn 3′UTR SNPs are cis-acting modifiers of aSynL translation

We hypothesized that SNPs present within the aSyn transcript 3′UTR and that are associated with PD risk (within the linkage disequilibrium (LD) haplotype block associated with increased PD risk) may play a direct role in aSynL translation regulation by modifying key 3′UTR cis-acting elements ³⁰. We identified 6 candidate SNPs that are present in aSyn 3′UTR and show variability in the population (minor allele frequency >1%; FIG. 5c). Luciferase assay vector analysis in SH-SY5Y cells revealed that only 2 out of these 6 SNPs—rs356165 and rs78991202-modify dopamine responsiveness of the aSyn 3′UTR (FIG. 5d, FIG. 11c). Strikingly, both of these are specific to the aSynL 3′UTR. Furthermore, whereas these SNPs are separated by approximately 80 nt within the primary 3′UTR sequence, RNA secondary structure analysis predicts that both are located within complementary strands of a single stem-loop structural element (FIG. 5e). We further note that rs356165 is tightly linked (in LD) with the SNP identified in the rQTL analysis above as regulating the aSynL:total ratio (rs356168; Supplementary Table 4b) and is strongly associated with PD risk (Supplementary Table 4a; linkage for rs78991202 is undetermined). Importantly, the protective allele of rs356165 was found to reduce aSyn 3′UTR-mediated translation (FIG. 5d). Taken together, these data implicate rs356165 as a candidate causative variant within the aSyn 3′UTR.

Scanning for potential cis-acting regulatory modules within the aSyn 3′UTR that could be altered by the SNP variants, we identified a miR-34b binding site that overlaps with the rs356165 and rs78991202 sites ³² (using TargetScan analysis; FIG. 5e). Co-transfection of HEK-293 cells with a miR-34b-3p precursor, along with a luciferase vector bearing the aSyn 3′UTR, significantly induced the level of the luciferase reporter (FIG. 5f; relative to a control miRNA precursor). In contrast, transfection of a miR-34b-3p-specific inhibitor significantly decreased luciferase expression (FIG. 5g; relative to a control miRNA inhibitor). Strikingly, those effects were abolished in the context of constructs harboring either of the 2 SNP variants as above (rs3561165 C>T, rs78991202 T>G). The effects of the miR-34b-3p mimic and inhibitor on aSyn translation were further confirmed in the context of endogenous human aSyn in human neuroblastoma SH-SY5Y cells (Right panels, FIG. 5f, g). Although miRNAs typically inhibit the stability of targeted mRNAs, other examples of microRNA-mediated translational induction have been described ³⁴. The miR-34b effect appears independent of the dopamine effect, as the effects appear simply additive in SH-SY5Y cells (FIG. 11b). We note that unlike the miR-34b target site (which is present in aSynL but not short aSyn 3′UTR transcript isoforms), the predicted target sites for other miRNAs previously implicated in the regulation of aSyn, such as miR-7 ³³, are present within the proximal region of the aSyn 3′UTR and thus impact expression of both long and short isoforms equivalently (FIG. 11c).

aSyn 3′UTR selection modifies the subcellular localization of aSyn protein

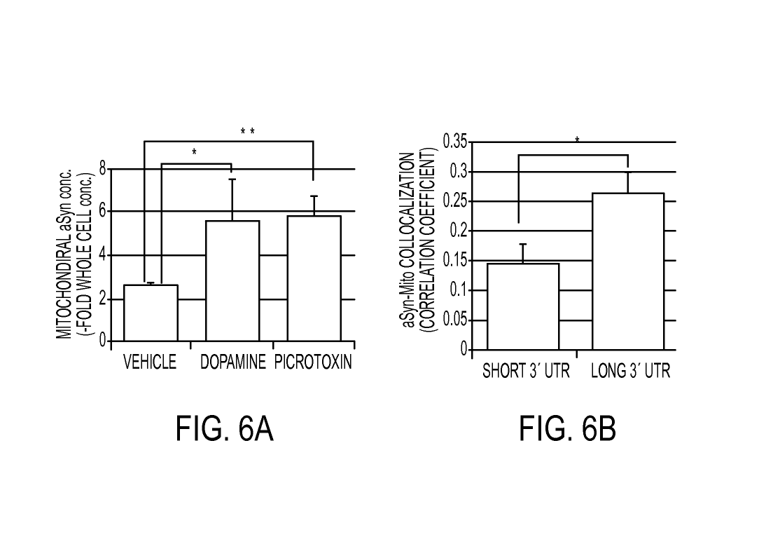

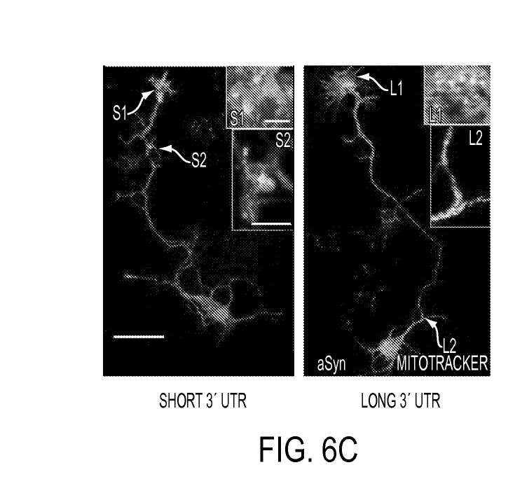

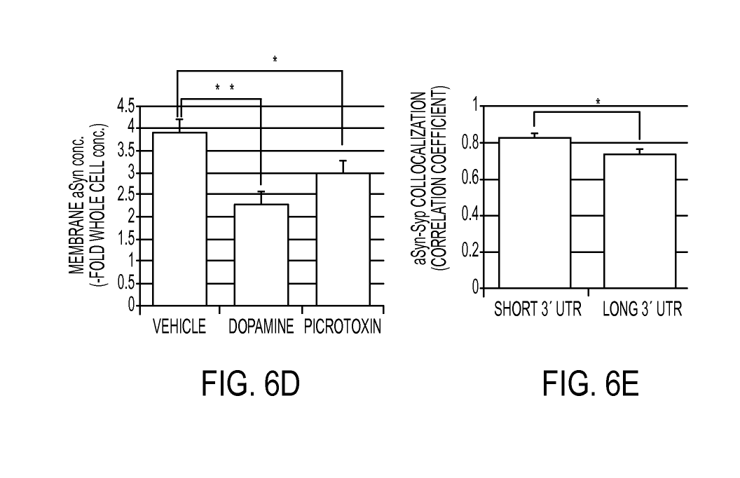

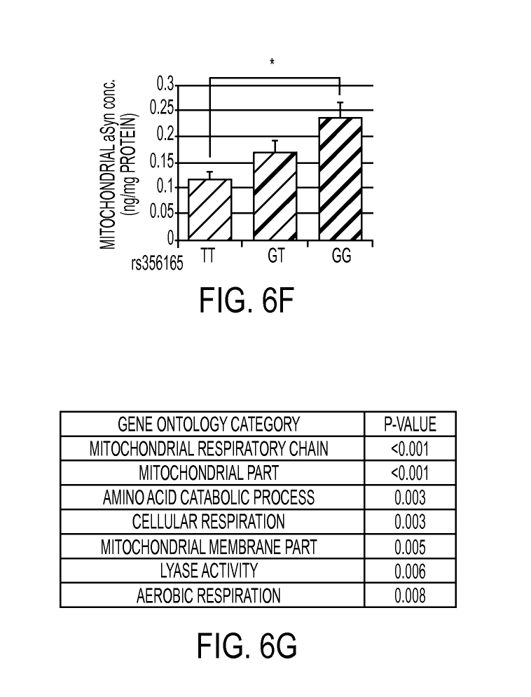

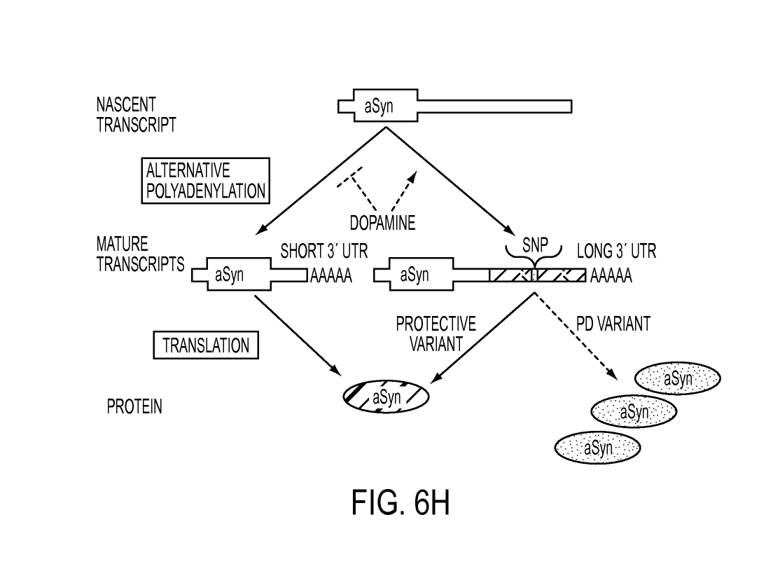

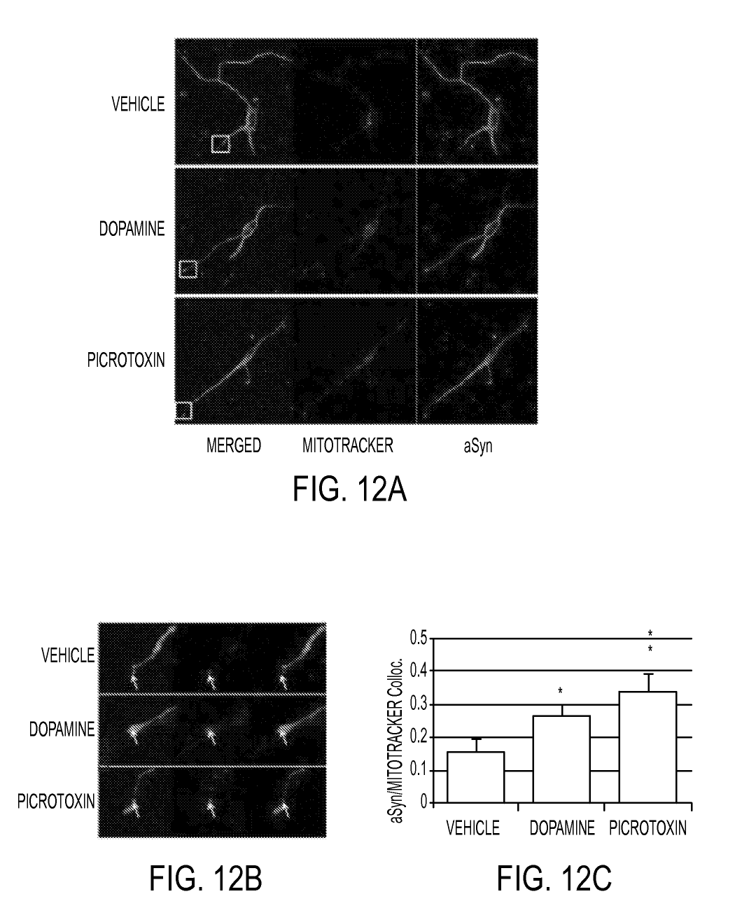

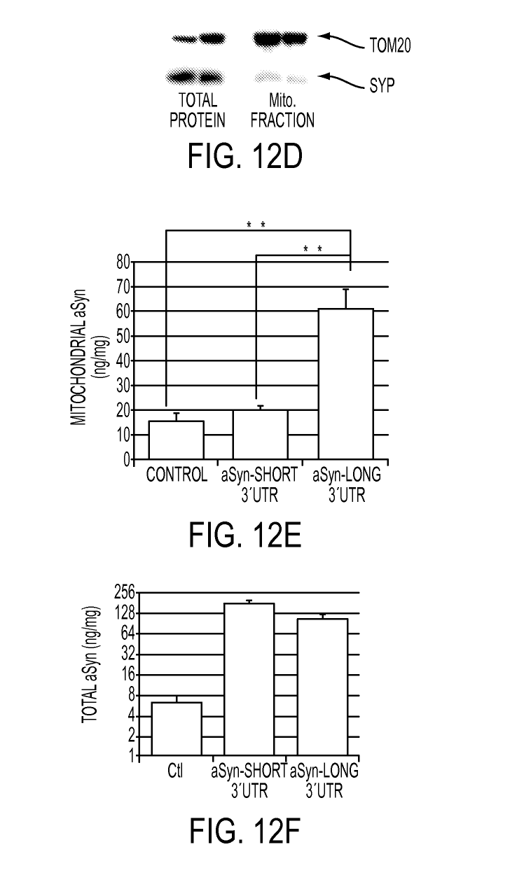

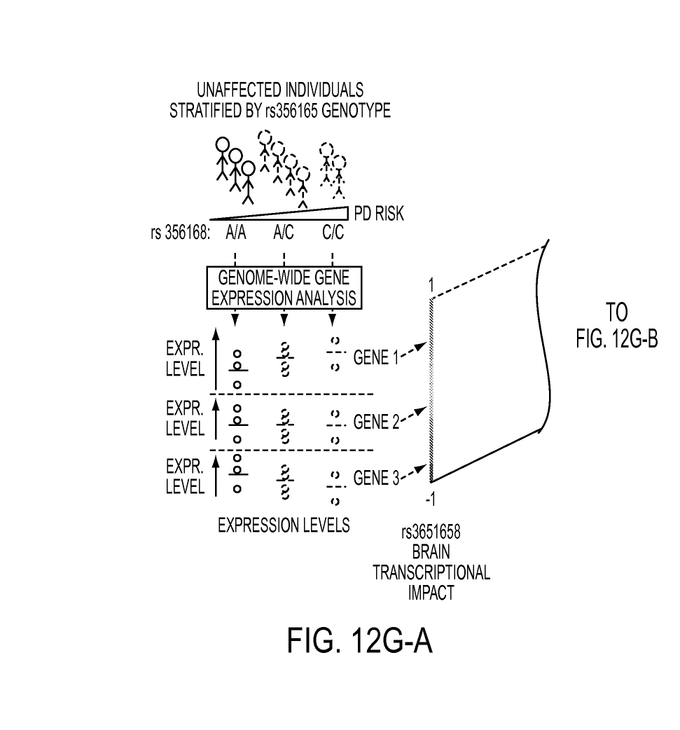

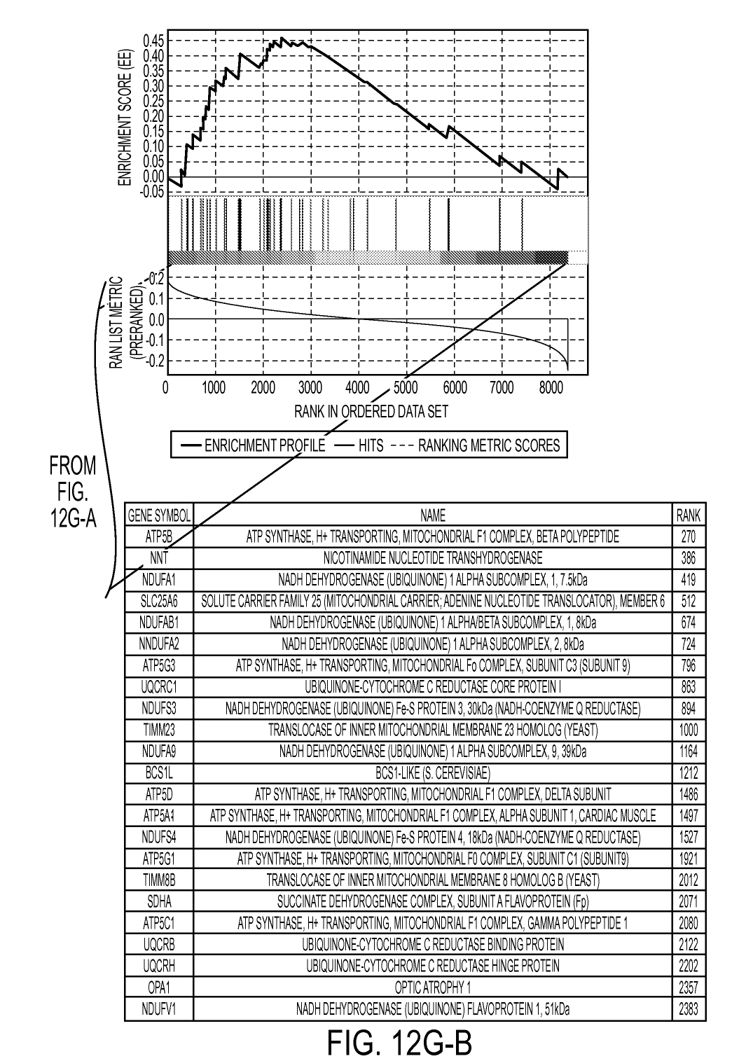

We next probed the potential impact of aSyn 3′UTR regulation on aSyn protein accumulation and subcellular localization in primary neurons. Treatment of primary mouse cortical neurons with dopamine or picrotoxin, shown above to increase the proportion of aSynL transcripts, also significantly increased the fraction of aSyn protein that colocalized with mitochondria (FIG. 12a-c). This was confirmed by biochemical analysis in human SH-SY5Y cells, as dopamine or picrotoxin treatment was associated with an increased proportion of endogenous aSyn protein within mitochondrial fractions (FIG. 6a). To relate these findings more directly to the aSyn 3′UTR, we transfected vectors encoding a GFP-aSyn fusion gene, bearing either a short or long aSyn 3′UTR (300 and 1100 nt, respectively), into primary rat cortical neuron cultures. Consistent with a regulatory role for the aSyn 3′UTR, expression of transcripts that harbor the 1.1 kb aSyn 3′UTR led to increased aSyn protein co-localization with mitochondria, relative to expression of transcripts that harbor a short 3′UTR (FIG. 6b-c). Similar transfection experiments in SH-SY5Y cells, followed by biochemical purification of mitochondrial protein fractions, confirmed the preferential mitochondrial accumulation of aSyn protein in the context of long aSyn 3′UTR transcript expression FIG. 12d-f) Concomitant with mitochondrial relocalization in the context of dopamine or picrotoxin treatment of SH-SY5Y cells, endogenous aSyn protein concentration in the total membrane protein fraction was decreased (FIG. 6d). Similarly, in primary rat cortical neuron cultures transfected with a GFP-aSyn fusion gene, colocalization with the presynaptic marker synaptophysin was reduced in the context of the longer aSyn 3′UTR (FIG. 6e). Taken together, these results suggest a preferential mitochondrial localization of aSyn protein produced from the aSynL transcript.

If indeed aSynL leads to the preferential localization of aSyn protein at mitochondria, one prediction would be that such localization would be apparent in human brain tissue from unaffected individuals that harbor the PD risk-associated P allele (as such tissue displays an increased aSynL/Total ratio; FIG. 2f). Strikingly, aSyn protein concentration in mitochondrial fractions was significantly increased in the context of the aSyn locus SNP risk allele in an allele dose-dependent manner (FIG. 6f), whereas such an increase was not apparent for total aSyn concentration. A final prediction is that aSyn relocalization to mitochondria might lead to evidence of mitochondrial dysfunction ³⁵, even in brain tissue from unaffected individuals that harbor the PD risk allele. To this end, we identified those transcriptome-wide gene expression changes that are most highly dependent on the allelic load of the PD risk-associated SNP variant across a panel of 183 cortical brain samples from unaffected individuals (see Methods). The set of modified transcripts was then functionally annotated by Gene Set Enrichment analysis (GSEA ³⁶; FIG. 12f). Among the 7 Gene Ontology categories most impacted by the risk SNP (p<0.01, FDR<25%) the majority relate to mitochondrial function (FIG. 6g). This is consistent with prior studies of patient-derived PD substantia nigra autopsy tissue using differential expression GSEA analysis ⁶.

Discussion

The identification of disease-associated common genetic variants in GWAS has led to excitement as well as significant controversy over their relevance ³⁷. A particular challenge is to bridge the gap between the observed associations and biological mechanisms. Indeed, such disease associations may be a synthetic consequence of nearby rare mutations, or other variants in LD ^{38 30}. Our analysis combines GDW and complex QTL analysis to gain sufficient insight and provide a plausible biological mechanism for the role of such variants in sporadic PD.

Our experimental data point to a model of alternative aSyn 3′UTR usage in PD (FIG. 6h). In this model, dopamine regulates the selection of the polyadenylation site during aSyn transcript maturation and favors the production of a transcript bearing a long 3′UTR. Long aSyn 3′UTR is associated with increased translation and mitochondrial localization of aSyn protein. We provide evidence that risk-associated SNP variants within the long 3′UTR directly modify protein translation; these variants appear to function by interfering with the action of trans-acting regulators such as miR-34b. An additional candidate trans factor is ELAVL4, a gene linked to sporadic PD ³⁹ and that encodes HuD, an RNA binding protein known to alter 3′UTR usage and that appears to bind to aSyn mRNA ⁴⁰. The mechanism by which the long aSyn 3′UTR confers mitochondrial localization of aSyn protein is less clear. We note that 3′UTR-dependant recruitment of mRNAs to the vicinity of mitochondria has been described for other transcripts ⁴¹⁻⁴³.

It is striking that the longer 3′UTR appears to reduce synaptic and increase mitochondrial protein accumulation, a pattern that is reminiscent of the disease state. Furthermore, this shift in protein localization parallels the shift in wiring correlation observed for aSynL expression within either brain tissue of PD patients or unaffected brain from individuals homozygous for a PD-associated variant. An interpretation of these data is that aSyn, normally at the axonal terminal, serves an upstream regulatory or signaling role in determining the expression level of other synaptic function-associated genes. In response to pathological genetic or environmental variation, relocalized aSyn no longer functions in this capacity, but instead impacts the expression of other genes.

We also identified common SNP variants in the aSyn and Parkin loci as regulators of the aSyn transcript ratio. It is of high interest how Parkin may effect this change, and whether this relates to the function of Parkin in mitochondria^{21 44}. Elevated intracytoplasmic dopamine as well the MPTP, which increase the aSynL:total ratio, are indeed similarly considered to disrupt mitochondria ⁴⁵. This supports a role for aSyn in mitochondrial toxicity, consistent with the apparent protective effect of aSyn deficiency in the context of mitochondrial toxins ^{46, 47}. We hypothesize that, in addition to Parkin, other genes associated with familial forms of PD may be relevant in aSyn transcript 3′UTR selection. DJ-1 is an RNA-binding protein that is mutated in familial autosomal recessive PD ⁴⁸, and re-analysis of gene expression in SH-SY5Y cells deficient in DJ-1 reveals a significant increase in aSyn ratio (FIG. 13e ⁴⁹).

A major challenge throughout human molecular genetics currently is how to mechanistically pursue SNP associations, particularly from GWAS studies 3. Our goal here is to understand the mechanism and impact of these disease-associated SNPs, and we present a novel approach to do so.

There are two points that relate directly to the choice of SNPs in this Example. The first one concerns the rQTL analysis (FIG. 2) and the second the molecular assays (FIG. 5).

(1) The rQTL analysis is an association study and as such, SNPs are simply markers for specific local haplotypes. The identification of SNPs in GWAS point to an linkage disequilibrium (LD) block (as can be determined based on the HapMap project data) rather than to a single SNP. In other words, looking at one SNP or another in LD would lead to very similar results, as shown in a recent Perspective published in Nature Genetics. See Freedman, M. L. et al. Principles for the post-GWAS functional characterization of cancer risk loci. Nat Genet. 43, 513-518 (2011).

Specifically with the analysis and the choice of SNPs: rs356168 was used as a proxy for the SNCA locus 3′ LD region, as identified in the recent GWAS from Simon-Sanchez et al. (2009). The use of such a proxy was justified by the perfect LD (r2=1, d=1) observed between rs356168 and rs2736990—the SNP exhibiting the lowest p-value in the GWAS. The reason why we chose to consider the European GWAS from Simon-Sanchez et al. (2009) to evaluate our proxy was that all the brains used for our rQTL analysis are from Caucasian origin 5. We thus assumed that the results from Simon-Sanchez et al., generated in a population closer to ours than those from Satake et al. (2009) were the more appropriate in our case and mentioned only those for the sake of concision. It could be noted however that rs356168 could also be a very good proxy for the two best SNCA locus SNPs found to be associated with PD in Satake et al (2009), as rs356168 exhibit a strong LD with them in the Japanese panel of HapMap (r2=0.818 and D′=1 for both rs3857059 and rs11931074). These LD considerations are now presented in supplementary table 4.

(2). When querying the potential direct biological role of SNP variants at a molecular level, each SNP needs to be considered independently. We thus tested all previously annotated SNP variants, from the HapMap website at NIH) and 1000 genomes studies with data available at the 1000 genomes project website, that fall within the long 3′UTR candidate region that are also in LD with the PD associated SNPs identified by the GWAS. A total of 2 SNPs met these criteria—rs356165 and rs78991202—and thus we studied both.

We include evidence in vitro and in vivo, as well as in human brain, that the SNPs and the long 3′UTR lead to increased accumulation of mitochondrial aSyn. We do not go on to show that this is pathological, but there are numerous manuscripts to that effect, which we now explicitly cite. Furthermore, simple triplication of the locus can lead to disease, consistent with pathological role for more protein. Finally, we add data showing that, even in unaffected individuals with the SNP that increases disease risk and increases the aSynL:total ratio, there is a specific alteration in the expression of mitochondrial genes. A similar differential expression pattern has been described in end-stage PD.

In addition to the evidence for a genetic link detailed above, non-genetic risk factors associated with PD—such as aging or rotenone exposure (associated with increased PD risk) or nicotine exposure (associated with decreased risk)—predictably modify the aSynL:total ratio (FIG. 13cdfg ^{37,50,51}). Our data imply that modifiers of the aSynL:total ratio such as the GABA-A receptor agonist muscimol may be of potential therapeutic value (although additional symptomatic effects would limit the utility of GABA-A receptor modulators in late-stage PD). Finally, we note that the aSynL:total ratio is also elevated in gene expression analysis of patient blood samples relative to unaffected controls (FIG. 14b ⁵²), suggesting utility as a biomarker for disease or treatment.

Methods

Primary Neurons Cultures.

Cultures of rodent neurons were prepared as described in ⁵³. Cells were maintained in vitro for 3-5 days before drug treatments or transfection using Lipofectamine 2000 (Iinvitrogen) following manufacturer's instructions.

Methods to make neurons from human fibroblasts are also known in the art. See for example WO 12/100,083, including but not limited to paragraphs [0215] to [0255], the entire contents of which are hereby incorporated by reference. See also Vierbuchen T, “Direct conversion of fibroblasts to functional neurons by defined factors.” Nature. 2010 Feb. 25; 463(7284):1035-41. Epub 2010 Jan. 27; Ambasudhan et al. “Direct Reprogramming of Adult Human Fibroblasts to Functional Neurons under Defined Conditions” Cell Stem Cell, Volume 9, Issue 2, 113-118, 28 Jul. 2011, the entire contents of which publications are hereby incorporated by reference in their entirety.

Western Blotting.

Western blot analyses were performed as described previously ⁵⁴ with alpha-synuclein antibody (C20, Santa Cruz), Tom20 (Abnova), synaptophysin (Millipore) and β-actin (Abcam, 1:400).

Northern Blotting.

Northern Blots were performed using the NorthernMax kit (Ambion) following manufacturer's instructions. 10 μg of total RNA was purified using miRNeasy kit (Qiagen) and loaded per lane. Probes for Northern blots were generated from a human brain cDNA template by PCR amplification using primers HNBaSynTfw (AGCCATGGATGTATTCATGAAAGGA) SEQ ID NO: 8 and HNBaSynTrv (TTAGGCTTCAGGTTCGTAGTC) SEQ ID NO: 9 for the human aSyn CDS probe, and HNBaSynLfw (GATGTGTTTTATTCACTTGTG) SEQ ID NO: 10 and HNBaSynLrv (AAAAGGCTCAATTAAAAATGTATAAC) SEQ ID NO: 11 for the 3′UTR-specific probe.

aSyn Protein Quantification.

Mitochondria were purified using Qproteome Mitochondria Isolation Kit (Qiagen) and membrane fractions were isolated using Subcellular Protein Fractionation Kit (Pierce) following manufacturers' instructions. Human aSyn protein levels were determined using the aSyn Human ELISA kit (Invitrogen). Absorbance was read on a VersaMax ELISA Microplate Reader (Molecular Devices, Inc) at 450 nm. The amount of human aSyn was normalized to total cellular protein as determined with the DC Protein Assay Reagent kit (Bio-Rad). Mitochondrial preparations were validated by Western blot analysis for Tom20 and synaptophysin (see FIG. 12d).

In Situ Hybridization.

In situ hybridization were performed using QuantiGene® ViewRNA ISH Cell Assay (Panomics) following manufacturer's instructions, with QG ViewRNA TYPE 8 Probe Sets (Panomics) designed to target either human aSyn CDS sequences (bases 264-634 from NM_{—}000345.3; Panomics) or to target human aSynL 3′UTR sequences (bases 1180-1760 from NM_{—}000345.3).

Nascent RNA Capture.

Total RNA was isolated using a miRNeasy kit (Qiagen) and nascent RNA was purified using the Click-iT® Nascent RNA Capture Kit (Invitrogen) following manufacturer's instructions; total and nascent RNA were then subjected to RT-qPCR analysis as below.

polyA-RNAseq.

RNAseq libraries were constructed essentially as previously described for the NNSR method ^{55 56} with the following modifications. First, the tagged first strand NNSR primer for the reverse transcription reaction was replaced with a tagged, barcoded polyA oligonucleotide mix (TCCGATCTCTNXXXXXXTTTTTTTTTTTTTTTTTTVN (SEQ ID NO: 12; with V=A,C,G mix, N=A,C,T,G mix, and XXXXXX denoting a barcode to allow for subsequent multiplexing of different samples in a single sequencing lane). 100 bp single-end reads were obtained by sequencing of the libraries on an Illumina HiSeq 2000 platform to generate more than 300 million reads for the 34 samples. Data was analyzed using Galaxy ⁵². Reads were mapped using Burrows-Wheeler Alignment tools ⁵⁸.

Immunocytochemistry.

Immunostainings were done as previously described ⁵⁹ with Rabbit anti-aSyn (Santa Cruz, 1:200) or Mouse anti-Synaptophysin (Millipore, 1:100) as primary antibodies, and Alexa 555 goat anti-rabbit IgG or Alexa 633 goat anti-mouse IgG (Invitrogen) secondary antibodies. MitoTracker-Orange (Invitrogen, 1:10000) was added into media for 15-20 min in living cell culture. Collocalization analyses were done in R using EBImage package; using Pearson's correlation coefficient.

In Vivo 1-Dopa Treatment.

2-month old DAT-Cre/Dicer^{flox/flox} and DAT-Cre/Dicer^{flox/+24} or forty-weeks old male PAC-Tg (SNCA)+/−;Snca+/− (a gift from Dr. Robert L. Nussbaum, University of California San Francisco, ²⁵) received 20 mg/kg I-Dopa with 12 mg/kg benserazide (or PBS vehicle only) by intraperitoneal injection daily for 5 days. Benserazide, a DOPA decarboxylase inhibitor that does not cross the blood-brain barrier, was used in combination with I-Dopa as it is with PD patients, to prevent I-Dopa decarboxylation outside of the brain. 1 hour after the last dose, mice were anesthetized by inhaled isoflurane and the brains rapidly removed. The prefrontal cortex, striatum and midbrain were dissected out and stored at −80° C.

Quantitative Real-Time RT-PCR.

RT-qPCR analyses were performed as described ⁶⁰. The human aSynL:Total mRNA ratio was quantified in terms of AACt ⁶⁰ using primer pair Lh for the human long form (HaSynLfw [CTGACACAAAGGACAAA] SEQ ID NO: 13, and HaSynLry [TTCCGAGTGTAGGGTTAATGTT]) SEQ ID NO: 14 and primer pair Th for human total (HaSynTfw [AGGGTGTTCTCTATGTAGG] SEQ ID NO: 15 and HaSynTrv [ACTGTCTTCTGGGCTACTGC] SEQ ID NO: 16). For analysis of either mouse or rat, primer pairs mrL (RaSynLfw [AACTTCTTGAGAACAGCAACAA] SEQ ID NO: 17 and RaSynLrv [CTCCCCTCTCACTACAG] SEQ ID NO: 18) and mrT (RaSynTfw [CAACGTGCCCAGTCA] SEQ ID NO: 19, RaSynTry [GGATGCTGAGGGGCAGGT] SEQ ID NO: 20) were used.

Luciferase Assays.

The human SH-SY5Y neuroblastoma cell line (ATCC) was cultured following the distributor's instructions. Cells were plated at a density of 4×10⁵ cells per well (in 48-well plates) in wells coated with 0.1% gelatin (Specialty Media, Millipore) 24 hours prior to transfection. Transfections were performed with Lipofectamine 2000 reagent (Invitrogen) following the manufacturer's instructions. The human HEK-293T cell line (ATCC) was cultured in DMEM (Invitrogen) supplemented with 10% FBS (Invitrogen). Cells were plated at a density of 8×10³ cells per well (96-well plates). Mir-34b-3p precursor, specific Anti-miR Mir-34b-3p inhibitor, Anti-miR Negative Control #1, and Pre-miR Negative Control #1 were purchased from Ambion. Cells were co-transfected with luciferase reporter plasmids and a small RNA or inhibitor (as indicated) using siPort NeoFx reagent (Ambion) following manufacturer's protocol. Luciferase and Renilla activities were measured 24 h or 48 h after transfection using Dual-Glo luciferase assay system (Promega).

Plasmids

Dual hRen/hLuc pEZX-MT01 plasmid with the first 1074 bp (“Long”, HmiT017582-MT01) or 560 bp (“Short”, HmiT017583-MT01) of human aSyn 3′UTR downstream of luciferase or control vector (CmiT000001-MT01) were purchased from Genecopoeia. Point mutant corresponding to rs356165 C>T and rs78991202 A>C were generated from HmiT017582-MT01 by site-directed mutagenesis (Genewiz). Plasmids expressing a GFP-aSyn fusion with either a long (1074 bp) or a short (560 bp) 3′UTR were generated by insertion in a pEGFP-C1 vector (Clontech) between its XhoI and HindIII restriction sites of HindIII/XhoI digested PCR products obtained from human brain cDNA using the forward primer XhoI-Start (ATCTCGAGCCATGGATGTATTCATGAAAGGA SEQ ID NO: 21) with either HindIII-275 (CAAAGCTTAGGTGTTTTTAATTTGTTTTAACATCGT SEQ ID NO: 22) or HindIII-1074 (CAAAGCTTCATGGTCGAATATTATTTATTGTCAGAA SEQ ID NO: 23) as a reverse primer. PolyA-disrupted vector was generated from pEGFP-aSyn-Long 3′UTR; the putative polyadenylation signal at position 542 to 552 of the aSyn 3′UTR “AATTAAAATAA” SEQ ID NO: 24 was deleted by site directed mutagenesis (Genewiz).

Human Autopsied Brain Samples.

Age-matched samples from Parkinson's disease patients (5 female, 12 male, average age 78.29±5.95), unaffected individuals (5 female, 12 male, average age 74.05±13.04) or ALS patients (7 female, 9 male, average age 70.02±11.01) BA9 area brain samples were obtained from the New York Brain Bank ⁶¹. Samples were provided devoid of any personal information.

Statistical Analysis.

Results are given as mean±S.E.M. Where appropriate, statistical analysis were performed with analysis of variance (ANOVA) test followed by Bonferroni corrected tests. Otherwise, comparisons between groups were conducted using Student's t test.

QTL Analysis.

Data from cerebral cortex transcriptome-wide gene expression analyses, as well as genome-wide SNP analyses for the same 188 individuals, were previously described ¹⁶. These data were obtained from the Laboratory of Functional Neurogenomic at the University of Miami School of Medicine, and reanalyzed. The rQTL for each sample was determined as a ratio of the value for probes GI_{—}6806896-I and GI_{—}6806897-A. Subsequently, the rQTL value was provided as a continuous numeric trait variable in the gplink ⁵⁵ assoc function, filtering for minor allele frequency below 0.05, genotype missingness above 0.1 and Hardy-Weinberg equilibrium threshold of 0.001. Haploview was used to generate a Manhattan plot of the output data. For loci intersection analysis as in FIG. 3b, the output of the rQTL analysis was queried at SNPs previously reported to be associated with PD risk (p-value<10⁻³) in GWAS analysis of individuals from a European ancestry ⁹. This GWAS data for PD risk were taken directly from the results presented in the supplementary data of Simon-Sanchez et al. We considered the European GWAS from Simon-Sanchez et al. for the intersection as the brains used for our rQTL analysis are from Caucasian origin ¹⁶. Resampling analysis were done in R: To assess the statistical significance of the intersection, a resampling without replacement procedure was done using R by selecting 316 SNPs out of the one used in the rQTL study. The number of SNPs whose 75 kb radius locus overlap with the PD-associated loci is evaluated. This process is repeated 5 million times and the results obtained from the actual data are compared to the random distribution generated.

Differential Wiring Analysis.

Datasets were downloaded from the Gene Expression Omnibus website of the National Center for Biotechnology Information at the NIH; specific dataset identification numbers are provided in supplementary table 5. All subsequent data manipulations and analyses were done using R Bioconductor package. Correlations between gene expression levels were assessed using cosine similarity on log-transformed levels; briefly, two genes whose expression levels are simultaneously high or low across many samples are in phase and will have a correlation coefficient close to 1. On the contrary, if one gene shows high expression levels when another one shows low across many samples, those two genes are in anti-phase and will have a correlation coefficient close to −1. The absence of linear relationship between the expression levels of both genes will result in a correlation coefficient close to 0. Comparisons between correlations obtained in two independent groups were done using a Fischer's Z transformation followed by a statistical test using pnorm R function.

The principle underlying DW algorithms ^{4, 5} is that for a given candidate ‘master regulator’ node gene X, the global DW score—when comparing two experimental conditions 1 and 2—is the sum of DW subscores between gene X and each of the other genes Gi queried. The subscore between the gene of interest X (for which the DW score is calculated) and a gene Gi is proportional to:

(i) the extent of the shift in correlation between the expression levels of Gi and X when comparing conditions 2 and 1 (thus genes exhibiting a high number of strong shifts in correlation with many other genes are assumed to be relevant nodes in the differential gene expression network between conditions 1 and 2);

(ii) the extent of differential expression of Gi between conditions 1 and 2 (averaged across the panel of samples for each condition; thus, the more a gene is on average differentially expressed between 2 conditions, the more it is predicted to have a phenotypic impact);

(iii) the level of expression Gi (a more highly expressed gene is thought to have a higher phenotypic impact; this is to compensate for the fact that lowly expressed genes are more likely to exhibit strong shifts in expression between the two conditions).

The two main modifications we introduce to the previously described wiring algorithms ^{4, 5} are: (i) We broadened the analysis of possible ‘master regulator’ genes from only annotated transcription factors to all genes, (ii) We introduced significance threshold tests for the interactor genes: as we included all the genes as candidate ‘master disease regulators’, instead of only all the annotated TF we wanted to avoid artificial results when working at a genome-wide scale than with hundreds of selected genes. Low-selective threshold (p-value=0.05) were however chosen to keep a high sensitivity.

The differential wiring score for a gene X between two experimental groups (1 and 2 with respectively n1 and n2 elements) was thus calculated as the sum over all the genes Gi of the absolute value of the product of:

(i) the conditional Z-distance evaluating the difference observed between the two groups for the correlation between the expression levels of genes X and Gi (<Δ_{Gi 1vs2}>ₚ in the formal DW formula below). Thus, for a given threshold p-value (0.05 here), it has a null value if the correlation shift is not significant. The amplitude of the Z-distance is proportional to the shift in correlation between the two experimental conditions. Fischer's Z-transformation corrects for the non-normal distribution of the correlation value (between −1 and 1). As a consequence, a shift in correlation form 0.7 to 0.9 will lead a Z-distance value higher than a shift from −0.1 to 0.1.

(ii) the conditional log-scaled amplitude of the differential expression of gene Gi (<δ(X, Gᵢ)₁ᵥₛ₂>ₚ in the DW formula below). For a given threshold p-value (0.05 here), it has a null value if the gene is found to not be differentially expressed between the two conditions. If the gene is differentially expressed for the chosen p-value, the value will be the log of the ratio between the averaged gene expression levels in each group.

(iii) the averaged expression level of gene Gi among all samples ((E_{Gi})₁ᵤ₂ in the formula below).

As a consequence of the use of significance threshold tests, only those genes which are differentially expressed between the two experimental conditions, and that see their correlation with gene X significantly changed between the two experimental conditions, will participate in the DW score.

Formally, the DW score was thus calculated as:

Differential wiring score for gene X:

DW(X)₁ᵥₛ₂=Σ_{G}_{ᵢ}|(Δ_{G}_{ᵢ}₁ᵥₛ₂<δ(X,Gᵢ)₁ᵥₛ₂>ₚ| (̅E̅_̅{̅G̅}̅_̅{̅ᵢ̅}̅)̅_̅{̅1̅∪̅2̅}̅

### With:

$${〈 {δ}_{1 vs 2} 〉}_{p} = \{ \begin{matrix}{δ}_{1 vs 2} & if pnorm ( {δ}_{1 vs 2} ) < p \\ 0 & if pnorm ( {δ}_{1 vs 2} ) \geq p\end{matrix} Conditional Z \text{-} distance for a p \text{-} value p \\ {δ ( X , G )}_{1 vs 2} = \frac{{F}_{z} ( {r ( X , G )}_{1} ) - {F}_{z} ( {r ( X , G )}_{2} )}{\sqrt{\frac{1}{{n}_{1} - 3} + \frac{1}{{n}_{2} - 3}}} Z \text{-} distance between {r ( X , G )}_{1} and {r ( X , G )}_{2}$$

r(X, G)₁, r(X, G)₂ correlation coefficient between the expression levels of genes X and G, evaluated in experimental groups 1 (n1 elements) and 2 (n₂ elements).

$${F}_{z} ( r ) = \frac{1}{2} log ( \frac{1 + r}{1 - r} ) {Fischer}^{′} s z transformation for a correlation coefficient r$$

$${〈 {Δ}_{G 1 vs 2} 〉}_{p} = \{ \begin{matrix}log ( \frac{\overline{{( {E}_{G} )}_{1}}}{\overline{{( {E}_{G} )}_{2}}} ) if p . value ( t . test ( {( {E}_{G} )}_{1} , {( {E}_{G} )}_{2} ) ) < p \\ if p . value ( t . test ( {( {E}_{G} )}_{1} , {( {E}_{G} )}_{2} ) ) \geq p\end{matrix} conditional DE amplitude of Gi$$

(E_{G})ᵢ: collection of the expression level values for gene G among the experimental group 1 All calculations were performed using the R statistical environment.

Two conceptual aspects differentiate the wiring approach used in this study from previous wiring analyses. Hudson, N.J., Reverter, A. & Dalrymple, B. P. A differential wiring analysis of expression data correctly identifies the gene containing the causal mutation. PLoS Comput Biol 5, e1000382 (2009); Reverter, A., Hudson, N.J., Nagaraj, S. H., Perez-Enciso, M. & Dalrymple, B. P. Regulatory impact factors: unraveling the transcriptional regulation of complex traits from expression data. Bioinformatics 26, 896-904 (2010). We expanded the scope of the potential network nodes (“master regulators”) to all genes, rather than only transcription factors (TF). Our strategy was motivated first by the knowledge that PD and other neurodegenerative disorders are not likely to be primarily due to modification of transcription factors. The identification of aSynL as the top result would not have been possible if we limited to annotated TFs. But more generally: among the (50) most highly ranked GDW genes in our analysis, fewer than 10% are annotated TFs. Thus our finding with aSynL is not an exceptional case. We believe that the network properties underlying the DW approach are not limited to TFs. 2) We hypothesized that a heterogeneous, sporadic human disease would be amenable to this technique.

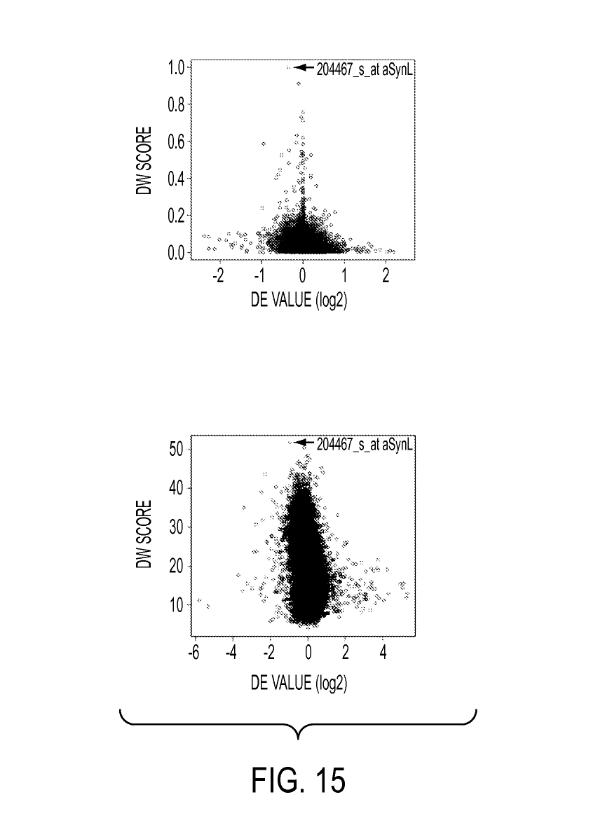

A previous study did use a wiring network approach to correctly identify the extreme rewiring of a ‘master regulator’ gene transcript in the context of an inherited coding mutation in that gene in cattlel (leading to dysfunction of the TF). But ‘sporadic’ PD is not thought to be a consequence of such a unique coding mutation. We nonetheless reasoned that global wiring analysis would be sensitive enough to detect extreme alterations in the wiring of ‘master regulator’ transcripts that are functionally altered in other ways—even in the absence of an inherited coding mutation, and even in a heterogeneous disease and tissue. We further surmised that such dysfunction/rewiring, in the absence of coding mutations, may be due to altered regulation at the transcription or posttranscription level. For instance, altered gene expression may be imparted by synonymous (non-coding) PD risk-associated SNPs; whereas, dysfunction in the context of post-transcriptional modifications (such as misfolding) may be due to environmental insults such as implicated in PD, including toxins. A more technical aspect of our repurposing of the wiring network approach is also relevant. Given the inherent variability in post-mortem human brain tissue analysis, and the scale of any whole-transcriptome network approach, we decided to include statistical thresholds in terms of whether or not to consider any individual transcript-to-transcript correlation as signal or noise; very weak connections were then discarded (because the sum of many such weak erroneous connections would potentially incorrectly bias the analysis; see Methods for details). To illustrate this last point more directly we reproduced the GDW analysis with such significant threshold (exactly as in FIG. 1A) or without. The use of thresholds greatly sharpens the contrast between the top results and others; however in this analysis aSyn is still on top, which is a reassuring sign that our ultimate finding is not strictly due to the threshold testing. FIG. 15.

### REFERENCES

1. Lesnick, T. G. et al. A genomic pathway approach to a complex disease: axon guidance and Parkinson disease. PLoS Genet. 3, e98 (2007).
2. Moran, L. B. et al. Whole genome expression profiling of the medial and lateral substantia nigra in Parkinson's disease. Neurogenetics 7, 1-11 (2006).
3. Zhang, Y., James, M., Middleton, F. A. & Davis, R. L. Transcriptional analysis of multiple brain regions in Parkinson's disease supports the involvement of specific protein processing, energy metabolism, and signaling pathways, and suggests novel disease mechanisms. Am J Med Genet B Neuropsychiatr Genet. 137B, 5-16 (2005).
4. Reverter, A., Hudson, N.J., Nagaraj, S. H., Perez-Enciso, M. & Dalrymple, B. P. Regulatory impact factors: unraveling the transcriptional regulation of complex traits from expression data. Bioinformatics 26, 896-904 (2010).
5. Hudson, N.J., Reverter, A. & Dalrymple, B. P. A differential wiring analysis of expression data correctly identifies the gene containing the causal mutation. PLoS Comput Biol 5, e1000382 (2009).
6. Zheng, B. et al. PGC-1alpha, a potential therapeutic target for early intervention in Parkinson's disease. Sci Transl Med 2, 52ra73 (2010).
7. Edwards, T. L. et al. Genome-wide association study confirms SNPs in SNCA and the MAPT region as common risk factors for Parkinson disease. Ann Hum Genet. 74, 97-109 (2010).
8. Satake, W. et al. Genome-wide association study identifies common variants at four loci as genetic risk factors for Parkinson's disease. Nat Genet. 41, 1303-1307 (2009).
9. Simon-Sanchez, J. et al. Genome-wide association study reveals genetic risk underlying Parkinson's disease. Nat Genet. 41, 1308-1312 (2009).
10. Spillantini, M. G. et al. Alpha-synuclein in Lewy bodies. Nature 388, 839-840 (1997).
11. Singleton, A. B. et al. alpha-Synuclein locus triplication causes Parkinson's disease. Science 302, 841 (2003).
12. Polymeropoulos, M. H. et al. Mutation in the alpha-synuclein gene identified in families with Parkinson's disease. Science 276, 2045-2047 (1997).
13. Hodges, A. et al. Regional and cellular gene expression changes in human Huntington's disease brain. Hum Mol Genet. 15, 965-977 (2006).
14. Maycox, P. R. et al. Analysis of gene expression in two large schizophrenia cohorts identifies multiple changes associated with nerve terminal function. Mol Psychiatry 14, 1083-1094 (2009).
15. Myers, A. J. et al. A survey of genetic human cortical gene expression. Nat Genet. 39, 1494-1499 (2007).
16. Webster, J. A. et al. Genetic control of human brain transcript expression in Alzheimer disease. Am J Hum Genet. 84, 445-458 (2009).
17. Williams, S. M. & Haines, J. L. Correcting away the hidden heritability. Ann Hum Genet. 75, 348-350 (2011).
18. Nalls, M. A. et al. Imputation of sequence variants for identification of genetic risks for Parkinson's disease: a meta-analysis of genome-wide association studies. Lancet 377, 641-649 (2011).
19. Kordower, J. H. et al. Neurodegeneration prevented by lentiviral vector delivery of GDNF in primate models of Parkinson's disease. Science 290, 767-773 (2000).
20. Kitada, T. et al. Mutations in the parkin gene cause autosomal recessive juvenile parkinsonism. Nature 392, 605-608 (1998).
21. Palacino, J. J. et al. Mitochondrial dysfunction and oxidative damage in parkin-deficient mice. J Biol Chem 279, 18614-18622 (2004).
22. Schapira, A. H. et al. Mitochondria in the etiology and pathogenesis of Parkinson's disease. Ann Neurol 44, S89-98 (1998).
23. Goldberg, M. S. et al. Parkin-deficient mice exhibit nigrostriatal deficits but not loss of dopaminergic neurons. J Biol Chem 278, 43628-43635 (2003).
24. Kim, J. et al. A MicroRNA feedback circuit in midbrain dopamine neurons. Science 317, 1220-1224 (2007).
25. Kuo, Y. M. et al. Extensive enteric nervous system abnormalities in mice transgenic for artificial chromosomes containing Parkinson disease-associated alpha-synuclein gene mutations precede central nervous system changes. Hum Mol Genet. 19, 1633-1650 (2010).
26. Ramirez-Valle, F., Braunstein, S., Zavadil, J., Formenti, S. C. & Schneider, R. J. eIF4GI links nutrient sensing by mTOR to cell proliferation and inhibition of autophagy. J Cell Biol 181, 293-307 (2008).
27. Guillot, T. S. & Miller, G. W. Protective actions of the vesicular monoamine transporter 2 (VMAT2) in monoaminergic neurons. Mol Neurobiol 39, 149-170 (2009).
28. Mosharov, E. V. et al. Interplay between cytosolic dopamine, calcium, and alpha-synuclein causes selective death of substantia nigra neurons. Neuron 62, 218-229 (2009).
29. Storvik, M. et al. Genes regulated in MPTP-treated macaques and human Parkinson's disease suggest a common signature in prefrontal cortex. Neurobiol Dis 38, 386-394 (2010).
30. Freedman, M. L. et al. Principles for the post-GWAS functional characterization of cancer risk loci. Nat Genet. 43, 513-518 (2011).
31. Lill CM, R. J., McQueen M B, Bagade S, Kavvoura F, Schjeide B M M, Allen N C, Tanzi R, Khoury M J, Ioannidis J P A, Bertram L. The PDGene Database. Alzheimer Research Forum. (Accessed 2010/09/01).
32. Lewis, B. P., Burge, C. B. & Bartel, D. P. Conserved seed pairing, often flanked by adenosines, indicates that thousands of human genes are microRNA targets. Cell 120, 15-20 (2005).
33. Junn, E. et al. Repression of alpha-synuclein expression and toxicity by microRNA-7. Proc Natl Acad Sci USA 106, 13052-13057 (2009).
34. Vasudevan, S., Tong, Y. & Steitz, J. A. Switching from repression to activation: microRNAs can up-regulate translation. Science 318, 1931-1934 (2007).
35. Kamp, F. et al. Inhibition of mitochondrial fusion by alpha-synuclein is rescued by PINK1, Parkin and DJ-1. EMBO J 29, 3571-3589 (2010).
36. Subramanian, A. et al. Gene set enrichment analysis: a knowledge-based approach for interpreting genome-wide expression profiles. Proc Natl Acad Sci USA 102, 15545-15550 (2005).
37. McClellan, J. & King, M. C. Genetic heterogeneity in human disease. Cell 141, 210-217 (2010).
38. Dickson, S. P., Wang, K., Krantz, I., Hakonarson, H. & Goldstein, D. B. Rare variants create synthetic genome-wide associations. PLoS Biol 8, e1000294 (2010).
39. DeStefano, A. L. et al. Replication of association between ELAVL4 and Parkinson disease: the GenePD study. Hum Genet. 124, 95-99 (2008).
40. Bolognani, F., Contente-Cuomo, T. & Perrone-Bizzozero, N. I. Novel recognition motifs and biological functions of the RNA-binding protein HuD revealed by genome-wide identification of its targets. Nucleic Acids Res 38, 117-130 (2009).
41. Sylvestre, J., Margeot, A., Jacq, C., Dujardin, G. & Corral-Debrinski, M. The role of the 3′ untranslated region in mRNA sorting to the vicinity of mitochondria is conserved from yeast to human cells. Mol Biol Cell 14, 3848-3856 (2003).
42. Corral-Debrinski, M., Blugeon, C. & Jacq, C. In yeast, the 3′ untranslated region or the presequence of ATM1 is required for the exclusive localization of its mRNA to the vicinity of mitochondria. Mol Cell Biol 20, 7881-7892 (2000).
43. Russo, A. et al. cis-acting sequences and trans-acting factors in the localization of mRNA for mitochondrial ribosomal proteins. Biochim Biophys Acta 1779, 820-829 (2008).
44. Vives-Bauza, C. et al. PINK1-dependent recruitment of Parkin to mitochondria in mitophagy. Proc Natl Acad Sci USA 107, 378-383 (2009).
45. Ramsay, R. R., Salach, J. I. & Singer, T. P. Uptake of the neurotoxin 1-methyl-4-phenylpyridine (MPP+) by mitochondria and its relation to the inhibition of the mitochondrial oxidation of NAD+-linked substrates by MPP+. Biochem Biophys Res Commun 134, 743-748 (1986).
46. Klivenyi, P. et al. Mice lacking alpha-synuclein are resistant to mitochondrial toxins. Neurobiol Dis 21, 541-548 (2006).
47. Dauer, W. et al. Resistance of alpha-synuclein null mice to the parkinsonian neurotoxin MPTP. Proc Natl Acad Sci USA 99, 14524-14529 (2002).
48. Bonifati, V. et al. Mutations in the DJ-1 gene associated with autosomal recessive early-onset parkinsonism. Science 299, 256-259 (2003).
49. Foti, R. et al. Parkinson disease-associated DJ-1 is required for the expression of the glial cell line-derived neurotrophic factor receptor RET in human neuroblastoma cells. J Biol Chem 285, 18565-18574 (2010).
50. Gibbs, J. R. et al. Abundant quantitative trait loci exist for DNA methylation and gene expression in human brain. PLoS Genet. 6, e1000952 (2010).
51. Berchtold, N. C. et al. Gene expression changes in the course of normal brain aging are sexually dimorphic. Proc Natl Acad Sci USA 105, 15605-15610 (2008).
52. Shehadeh, L. A. et al. SRRM2, a potential blood biomarker revealing high alternative splicing in Parkinson's disease. PLoS One 5, e9104 (2010).
53. Qiang, L., Yu, W., Andreadis, A., Luo, M. & Baas, P. W. Tau protects microtubules in the axon from severing by katanin. J Neurosci 26, 3120-3129 (2006).
54. Staropoli, J. F. et al. Parkin is a component of an SCF-like ubiquitin ligase complex and protects postmitotic neurons from kainate excitotoxicity. Neuron 37, 735-749 (2003).
55. Armour, C. D. et al. Digital transcriptome profiling using selective hexamer priming for cDNA synthesis. Nat Methods 6, 647-649 (2009).
56. Levin, J. Z. et al. Comprehensive comparative analysis of strand-specific RNA sequencing methods. Nat Methods 7, 709-715 (2010).
57. Goecks, J., Nekrutenko, A. & Taylor, J. Galaxy: a comprehensive approach for supporting accessible, reproducible, and transparent computational research in the life sciences. Genome Biol 11, R86 (2010).
58. Langmead, B., Trapnell, C., Pop, M. & Salzberg, S. L. Ultrafast and memory-efficient alignment of short DNA sequences to the human genome. Genome Biol 10, R25 (2009).
59. Martinat, C. et al. Cooperative transcription activation by Nurr1 and Pitx3 induces embryonic stem cell maturation to the midbrain dopamine neuron phenotype. Proc Natl Acad Sci USA 103, 2874-2879 (2006).
60. Rhinn, H. et al. Housekeeping while brain's storming Validation of normalizing factors for gene expression studies in a murine model of traumatic brain injury. BMC Mol Biol 9, 62 (2008).
61. Vonsattel, J. P., Del Amaya, M. P. & Keller, C. E. Twenty-first century brain banking. Processing brains for research: the Columbia University methods. Acta Neuropathol 115, 509-532 (2008).
62. Minones-Moyano, E. et al. MicroRNA profiling of Parkinson's disease brains identifies early downregulation of miR-34b/c which modulate mitochondrial function. Hum Mol Genet. 20, 3067-3078 (2011).
63. Chen-Plotkin, A. S. et al. Variations in the progranulin gene affect global gene expression in frontotemporal lobar degeneration. Hum Mol Genet. 17, 1349-1362 (2008).
64. Chin, M. H. et al. Mitochondrial dysfunction, oxidative stress, and apoptosis revealed by proteomic and transcriptomic analyses of the striata in two mouse models of Parkinson's disease. J Proteome Res 7, 666-677 (2008).
65. Greene, J. G., Greenamyre, J. T. & Dingledine, R. Sequential and concerted gene expression changes in a chronic in vitro model of parkinsonism. Neuroscience 152, 198-207 (2008).

AppendixA: Supplementary Tables 1-9

### APPENDIX A

SUPPLEMENTARY TABLE 1
Global differential wiring results

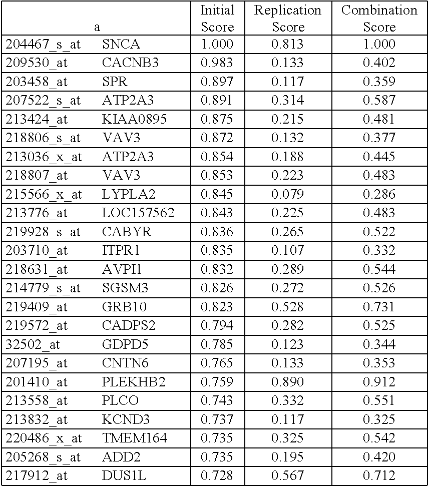

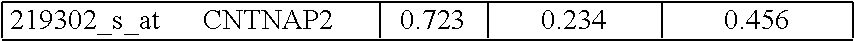

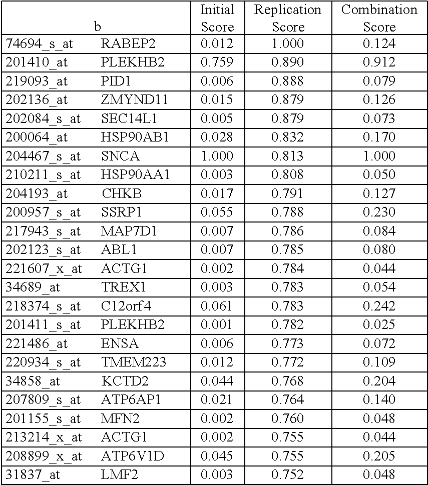

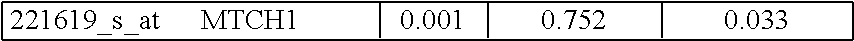

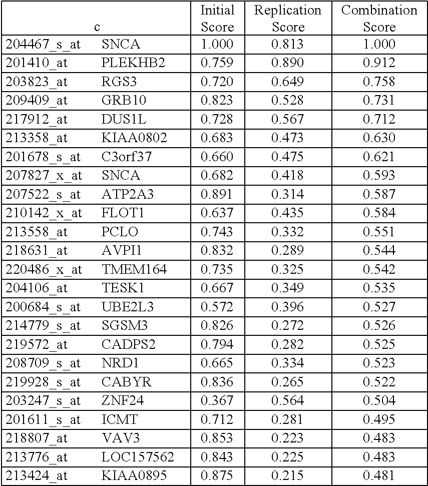

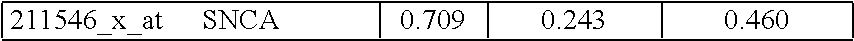

Top 25 probesets identified by the initial GDW analysis of PD vs Ctl in SN samples (a) of PD vs Ctl SN and SN laser-microdissected neurons samples (b) and by the combination of both analysis. For each analysis, the maximum score was set to one. For each table, the score for the analysis of interest is in black, and the score of the same probeset in the two other analysis in indicated in grey as an information. Highlighted in red and orange (Plekhb2) are the two genes (aSyn and Plekhb2, respectively) that appear as top-ranking for each of the analysis.

SUPPLEMENTARY TABLE 2
Genomic coordinates (hg18) of the
Affymetrix and Illumina aSyn probes

| Probe | Chr. | Strand | start | stop |
|---|---|---|---|---|
| 207827_x_at | 4 | — | 90962438 | 90975855 |
| 211546_x_at | 4 | — | 90869367 | 90975803 |
| 204466_s_at | 4 | — | 90866502 | 90866801 |
| 204467_s_at | 4 | — | 90865759 | 90866067 |
| GI_6806896-I | 4 | — | 90866611 | 90866660 |
| GI_6806897-A | 4 | — | 90869395 | 90869444 |

SUPPLEMENTARY TABLE 3
Loci associated to PD and aSyn ratio

| | | Combined |
|---|---|---|
| Position | Gene(s) | p-value |
| chr4: 90818453-91028750 | SNCA | 6.48E−15 |
| chr6: 162558416-162618882 | PARK2 | 8.54E−09 |
| chr6: 148905000-149036593 | SASH1 | 1.20E−08 |
| chr20: 16612675-16681299 | SNRPB2 | 1.73E−08 |
| chr7: 47984125-48127326 | SUNC1/HUS1/UPP1 | 2.03E−08 |
| chr5: 160563131-160587671 | GABRB2 | 3.30E−08 |
| chr5: 37874300-37877141 | GDNF | 3.81E−08 |
| chr15: 36900712-36989378 | RASGRP1 | 4.98E−08 |
| chr12: 25041441-25152643 | LRMP | 6.61E−08 |
| chr4: 24491750-24554434 | CCDC149/LGI2/SOD3 | 8.28E−08 |
| chr19: 1925313-1955168 | CSNK1G2/BTBD2/MKNK2 | 9.60E−08 |
| chr15: 90636309-90775680 | ST8SIA2 | 9.96E−08 |
| chr8: 136969530-137050409 | KHDRBS3 | 1.31E−07 |

SUPPLEMENTARY TABLE 4
PD-associated SNPs linkage disequilibrium and frequencies.
a

| SNP | OR | 95% C.I. |
|---|---|---|
| rs2736990 | 1.25 | [1.20, 1.30] |
| rs356165 | 1.33 | [1.21, 1.46] |
| rs356168 | 1.21 | [1.06, 1.38] |

b

| | rs2736990 | rs356165 | rs356168 |
|---|---|---|---|
| rs2736990 | x | 0.817 | 1 |
| rs356165 | 31655 | x | 0.817 |
| rs356168 | 4110 | 27545 | x |

a: Allelic variants associated to PD by PDGene meta-analysis ²². rs2736990 is the SNP found to be the most-associated to PD risk in two GWAS ^{6, 8}. rs356168 was found to be the SNP most-associated to aSynL:total ratio in human brain cortex in our analysis (FIG. 3). Rs356165 is located in aSyn 3′UTR and was found to regulate its translation in response to dopamine (FIG. 5).
b: Linkage disequilibrium between the 3 aSyn locus SNPs of interest, evaluated using SNAP ⁵⁰ in the HapMap CEU panel (Upper Right, Red); genomic distance in bp (Lower Left, Blue)

SUPPLEMENTARY TABLE 5
GEO datasets used for the study

| GEO dataset(s) | Description | Use | Figure(s) | Reference |
|---|---|---|---|---|
| GSE7621 | PD and unaffected SN tissue | PD vs Ctl GDW analysis | 1ace | 1 |
| | | aSynL: total ratio in PD vs Ctl SN | 52b | |
| GSE8397 | PD and unaffected SN tissue | PD vs Ctl GDW analysis | 1a | 2 |
| GSE20292 | PD and unaffected SN tissue | PD vs Ctl GDW analysis | 1a | 3 |
| GSE20141 | PD and unaffected LMD mDN | PD vs Ctl GDW analysis | 1ae S1a | 6 |
| | | aSynL: total ratio in PD vs Ctl LMD SN mDN | 52c | |
| GSE13152 | FTD and unaffected brain cortex | aSyn probesets coexpression in FTD | 1f | 63 |
| GSE3790 | HD and unaffected caudate nucleus | aSyn probesets coexpression in HD | 51b | 13 |
| GSE17612 | Schizophrenia and unaffected brain cortex | aSyn probesets coexpression schizophrenia | 51a | 14 |
| GSE15222 | Genotyped unaffected (and AD) brain cortex | aSyn probesets coexpression and ratio in function | 1g, 2f | 16 |
| | | of rs356168 | | |
| | | aSynL: total rQTL analysis | 3abc | |
| | | rs356168 global impact in unaffected individuals | 6g | |
| GSE11011 | polysome associated and non-associated | aSyn probesets differential translation | 55a | 26 |
| | RNA from MCF10 cells | | | |
| GSE7707 | MPTP-treated mice brain tissue | Mitochondrial toxin increases aSynL: total ratio | 57a | 64 |
| GSE4550 | MPTP-treated macaque brain tissue | Mitochondrial toxin increases aSynL: total ratio | 57b | 29 |
| GSE4773 | Rotenone-treated SX-N-MC cells | Mitochondrial toxin increases aSynL: total ratio | 57c | 65 |
| GSE17204 | Dj-1 knock-down in SH-SYSY cells | DJ-1 knock-down increases aSynL: total ratio | 57e | 49 |
| GSE15745 | unaffected brain samples | aging increases aSynL: total ratio in brain samples | 57f | 50 |
| GSE11882 | unaffected brain samples | aging increases aSynL: total ratio in brain samples | 57g | 51 |
| GSE7307 | unaffected brain samples | aSynL: total ratio in different brain regions | 58a | x |
| GSE18838 | Blood samples from PD and unaffected | aSynL: total ratio is increased in PD vs unaffected | 58b | 52 |
| | individuals | blood | | |

SUPPLEMENTARY TABLE 6
This file contains the list of probesets differentially expressed in
PD vs unaffected SN samples (GEO GSE7621) that show an fold change
superior to 1 (absolute value on a log scal) and a pvalue <5.10e−2.

| | | | Diff_Expr | |
|---|---|---|---|---|
| | | | PD vs Ctl | Relative |
| ProbeSet | Gene | Locus | (log2) | Expre level |
| 204338_s_at | RGS4 | chr1q23.3 | −4.14 | 1.89E−01 |
| 213920_at | CUX2 | chr12q24.11-q24.12 | −2.45 | 9.74E−03 |
| 205857_at | SLC18A2 | chr10q25 | −2.40 | 4.07E−02 |
| 205311_at | DDC | chr7p12.2 | −2.19 | 1.17E−02 |
| 210454_s_at | KCNJ6 | chr21q22.1\|21q22.13-q22.2 | −2.06 | 1.49E−02 |
| 206836_at | SLC6A3 | chr5p15.3 | −2.04 | 5.20E−02 |
| 205825_at | PCSK1 | chr5q15-q21 | −2.04 | 7.15E−03 |
| 216086_at | SV2C | chr5q13.3 | −1.99 | 6.01E−02 |
| 212224_at | ALDH1A1 | chr9q21.13 | −1.89 | 5.38E−02 |
| 211421_s_at | RET | chr10q11.2 | −1.87 | 1.34E−02 |
| 206935_at | PCDH8 | chr13q14.3-q21.1 | −1.86 | 5.98E−03 |
| 208427_s_at | ELAVL2 | chr9p21 | −1.85 | 4.10E−03 |
| 205110_s_at | FGF13 | chrXq26.3 | −1.83 | 2.50E−02 |
| 208319_s_at | RBM3 | chrXp11.2 | −1.77 | 8.04E−03 |
| 204337_at | RGS4 | chr1q23.3 | −1.65 | 3.05E−02 |
| 215342_s_at | RABGAP1L | chr1q24 | −1.64 | 6.64E−03 |
| 203282_at | GBE1 | chr3p12.3 | −1.60 | 2.25E−02 |
| 214230_at | CDC42 | chr1p36.1 | −1.60 | 1.37E−02 |
| 203476_at | TPBG | chr6q14-q15 | −1.58 | 1.17E−02 |
| 212092_at | PEG10 | chr7q21 | −1.57 | 1.24E−02 |
| 208175_s_at | DMP1 | chr4q21 | −1.55 | 6.12E−04 |
| 204667_at | FOXA1 | chr14q12-q13 | −1.51 | 4.72E−03 |
| 219572_at | CADPS2 | chr7q31.3 | −1.51 | 2.83E−02 |
| 205551_at | SV2B | chr15q26.1 | −1.50 | 2.60E−02 |
| 219895_at | FAM70A | chrXq24 | −1.50 | 2.51E−02 |
| 204364_s_at | REEP1 | chr2p11.2 | −1.46 | 4.15E−02 |
| 220030_at | STYK1 | chr12p13.2 | −1.41 | 6.49E−04 |
| 34726_at | CACNB3 | chr12q13 | −1.40 | 1.08E−02 |
| 204621_s_at | NR4A2 | chr2q22-q23 | −1.36 | 4.86E−03 |
| 204365_s_at | REEP1 | chr2p11.2 | −1.34 | 2.65E−02 |
| 215303_at | DCLK1 | chr13q13 | −1.33 | 7.33E−03 |
| 220559_at | EN1 | chr2q13-q21 | −1.32 | 7.64E−03 |
| 213938_at | ERC2 | chr3p14.3 | −1.31 | 1.89E−02 |
| 214841_at | CNIH3 | chr1q42.12 | −1.31 | 2.70E−03 |
| 213913_s_at | TBC1D30 | chr12q14.3 | −1.31 | 5.45E−03 |
| 203999_at | SYT1 | chr12cen-q21 | −1.30 | 1.02E−01 |
| 213967_at | RALYL | chr8q21.2 | −1.30 | 1.82E−02 |
| 219073_s_at | OSBPL10 | chr3p22.3 | −1.27 | 7.59E−03 |
| 206343_s_at | NRG1 | chr8p12 | −1.27 | 2.18E−03 |
| 203797_at | VSNL1 | chr2p24.3 | −1.26 | 2.03E−01 |
| 205968_at | KCNS3 | chr2p24 | −1.25 | 5.80E−03 |
| 207958_at | UGT2A1 /// | chr4q13 /// | −1.25 | 1.74E−03 |
| | UGT2A2 | chr4q13.3 | | |
| 215014_at | KCND3 | chr1p13.3 | −1.24 | 1.06E−02 |
| 209254_at | KLHDC10 | chr7q32.2 | −1.23 | 4.65E−03 |
| 206091_at | MATN3 | chr2p24-p23 | −1.23 | 3.41E−03 |
| 212979_s_at | FAM115A | chr7q35 | −1.22 | 2.32E−02 |
| 205691_at | SYNGR3 | chr16p13 | −1.21 | 5.29E−02 |
| 205389_s_at | ANK1 | chr8p11.1 | −1.20 | 2.31E−03 |
| 216248_s_at | NR4A2 | chr2q22-q23 | −1.19 | 8.20E−03 |
| 214811_at | RIMBP2 | chr12q24.33 | −1.19 | 6.49E−03 |
| 205391_x_at | ANK1 | chr8p11.1 | −1.19 | 1.11E−02 |
| 206382_s_at | BDNF | chr11p13 | −1.19 | 4.00E−03 |
| 214589_at | FGF12 | chr3q28 | −1.18 | 2.59E−03 |
| 208352_x_at | ANK1 | chr8p11.1 | −1.18 | 1.20E−02 |
| 214848_at | — | — | −1.18 | 6.23E−03 |
| 205257_s_at | AMPH | chr7p14-p13 | −1.17 | 1.48E−02 |
| 209560_s_at | DLK1 | chr14q32 | −1.16 | 6.54E−03 |
| 206984_s_at | RIT2 | chr18q12.3 | −1.16 | 1.36E−02 |
| 214930_at | SLITRK5 | chr13q31.2 | −1.16 | 5.12E−03 |
| 205632_s_at | P1P5K1B | chr9q13 | −1.15 | 1.56E−02 |
| 266_s_at | CD24 | chr6q21 | −1.14 | 6.92E−03 |
| 200810_s_at | CIRBP | chr19p13.3 | −1.13 | 7.13E−02 |
| 205348_s_at | DYNC1I1 | chr7q21.3-q22.1 | −1.13 | 4.31E−02 |
| 201340_s_at | ENC1 | chr5q12-q13.3 | −1.13 | 3.43E−03 |
| 210123_s_at | [symbol] 7A /// | chr15q13.1 /// | −1.13 | 2.73E−03 |
| | CHRNA7 /// | chr15q14 | | |
| | LO [symbol] | | | |
| 204675_at | SRD5A1 | chr5p15 | −1.13 | 5.56E−03 |
| 204260_at | CHGB | chr20pter-p12 | −1.12 | 2.05E−02 |
| 204723 at | SCN3B | chr11q23.3 | −1.11 | 1.00E−02 |
| 206502_s_at | INSM1 | chr20p11.2 | −1.10 | 3.88E−03 |
| 219736_at | TRIM36 | chr5q22.3 | −1.10 | 1.22E−02 |
| 205413_at | MPPED2 | chr11p13 | −1.10 | 7.42E−03 |
| 219603_s_at | ZNF226 | chr19q13.2 | −1.10 | 9.72E−03 |
| 218807_at | VAV3 | chr1p13.3 | −1.10 | 1.51E−02 |
| 208353_x_at | ANK1 | chr8p11.1 | −1.09 | 1.19E−02 |
| 204471_at | GAP43 | chr3q13.1-q13.2 | −1.07 | 4.56E−02 |
| 203680_at | PRKAR2B | chr7q22 | −1.07 | 1.55E−02 |
| 214156_at | MYRIP | chr3p22.1 | −1.07 | 1.12E−02 |
| 207869_s_at | CACNA1G | chr17q22 | −1.07 | 2.59E−03 |
| 213832_at | KCND3 | chr1p13.3 | −1.06 | 4.34E−02 |
| 219855_at | NUDT11 | chrXp11.22 | −1.06 | 5.35E−03 |
| 206014_at | ACTL6B | chr7q22 | −1.06 | 1.12E−03 |
| 203290_at | HLA-DQA1 | chr6p21.3 | −1.06 | 3.15E−03 |
| 204604_at | PFTK1 | chr7q21-q22 | −1.06 | 3.02E−02 |
| 204339_s_at | RGS4 | chr1q23.3 | −1.05 | 7.21E−03 |
| 204622_x_at | NR4A2 | chr2q22-q23 | −1.05 | 9.28E−03 |
| 209755_at | NMNAT2 | chr1q25 | −1.05 | 1.17E−02 |
| 207087_x_at | ANK1 | chr8p11.1 | −1.05 | 1.10E−02 |
| 213484_at | — | — | −1.05 | 2.09E−02 |
| 205377_s_at | ACHE | chr7q22 | −1.05 | 4.74E−03 |
| 221727_at | SUB1 | chr5p13.3 | −1.04 | 1.76E−02 |
| 203498_at | RCAN2 | chr6p12.3 | −1.04 | 8.98E−02 |
| 212094_at | PEG10 | chr7q21 | −1.04 | 4.34E−02 |
| 204419_x_at | HBG1 /// | chr11p15.5 | −1.03 | 1.34E−02 |
| | HBG2 | | | |
| 204035_at | SCG2 | chr2q35-q36 | −1.02 | 4.82E−02 |
| 212604_at | MRPS31 | chr13q14.11 | −1.02 | 9.38E−03 |
| 216073_at | ANKRD34C | chr15q25.1 | −1.02 | 5.36E−03 |
| 205399_at | DCLK1 | chr13q13 | −1.02 | 3.87E−02 |
| 208002_s_at | ACOT7 | chr1p36.31-p36.11 | −1.02 | 3.83E−02 |
| 221509_at | DENR | chr12q24.31 | −1.01 | 2.80E−02 |
| 212992_at | AHNAK2 | chr14q32.33 | −1.01 | 7.82E−03 |
| 204424_s_at | LMO3 | chr12p12.3 | −1.01 | 5.72E−02 |
| 209569_x_at | D4S234E | chr4p16.3 | −1.01 | 1.71E−02 |
| 205795_at | NRXN3 | chr14q31 | −1.00 | 1.74E−02 |
| 204814_at | CADPS | chr3p14.2 | −1.00 | 1.61E−02 |
| 209728_at | HLA-DRB4 | chr6p21.3 | 1.04 | 7.99E−03 |
| 221123_x_at | ZNF395 | chr8p21.1 | 1.04 | 4.57E−03 |
| 213089_at | LOC100272216 | — | 1.06 | 1.58E−02 |
| 219728_at | MYOT | chr5q31 | 1.06 | 1.37E−02 |
| 207907_at | TNFSF14 | chr19p13.3 | 1.07 | 2.96E−04 |
| 214799_at | NFASC | chr1q32.1 | 1.08 | 1.13E−02 |
| 221755_at | EHBP1L1 | chr11q13.1 | 1.08 | 3.64E−04 |
| 212177_at | SFRS18 | chr6q16.3 | 1.09 | 1.24E−02 |
| 222299_x_at | — | — | 1.10 | 4.73E−04 |
| 213164_at | SLC5A3 | chr21q22.12 | 1.11 | 2.56E−02 |
| 207142_at | KCNJ3 | chr2q24.1 | 1.12 | 3.12E−04 |
| 215071_s_at | HIST1H2AC | chr6p21.3 | 1.13 | 2.01E−02 |
| 209230_s_at | NUPR1 | chr16p11.2 | 1.14 | 5.37E−02 |
| 215555_at | — | — | 1.14 | 8.23E−04 |
| 218566_s_at | CHORDC1 | chr11q14.3 | 1.17 | 2.78E−02 |
| 213716_s_at | SECTM1 | chr17q25 | 1.18 | 9.84E−04 |
| 220924_s_at | SLC38A2 | chr12q | 1.19 | 6.37E−02 |
| 209309_at | AZGP1 | chr7q22.1 | 1.22 | 1.64E−02 |
| 201841_s_at | HSPB1 | chr7q11.23 | 1.22 | 8.98E−02 |
| 214682_at | LOC399491 | chr16p13.1 | 1.24 | 2.10E−03 |
| 218041_x_at | SLC38A2 | chr12q | 1.24 | 7.07E−02 |
| 208513_at | FOXB1 | chr15q21-q26 | 1.36 | 3.74E−04 |
| 209339_at | SIAH2 | chr3q25 | 1.40 | 1.67E−03 |
| 215352_at | GIMAP5 | chr7q36.1 | 1.46 | 9.74E−05 |
| 209015_s_at | DNAJB6 | chr7q36.3 | 1.51 | 1.36E−02 |
| 200800_s_at | HSPA1A /// | chr6p21.3 | 1.56 | 1.29E−01 |
| | HSPA1B | | | |
| 213479_at | NPTX2 | chr7q21.3-q22.1 | 1.69 | 2.41E−02 |
| 208088_s_at | CFHR5 | chr1q22-q23 | 1.94 | 1.93E−06 |

[symbol]

indicates data missing or illegible when filed

SUPPLEMENTARY TABLE 7
This file contains the lists of genes differentially correlated with aSynL in PD vs Unaffected SN.

| Affymetrix | Gene | | Correlation in | Correlation | Diff. |
|---|---|---|---|---|---|
| Probeset | Symbol | Cytoband | Control SN | in PD SN | Score |
| 205311_at | DDC | chr7p12.2 | 0.998 | 0.333 | −6.208 |
| 205110_s_at | FGF13 | chrXq26.3 | 0.990 | −0.087 | −5.587 |
| 207859_s_at | CHRNB3 | chr8p11.2 | 0.989 | −0.047 | −5.360 |
| 205445_at | PRL | chr6p22.2-p21.3 | 0.960 | −0.521 | −5.126 |
| 203997_at | PTPN3 | chr9q31 | 0.955 | −0.478 | −4.884 |
| 210454_s_at | KCNJ6 | chr21q22.1\|21q22.13-q22.2 | 0.989 | 0.267 | −4.736 |
| 216047_x_at | SEZ6L | chr22q12.1 | 0.978 | −0.078 | −4.736 |
| 209324_s_at | RGS16 | chr1q25-q31 | 0.961 | −0.289 | −4.582 |
| 218807_at | VAV3 | chr1p13.3 | 0.972 | −0.083 | −4.467 |
| 211894_x_at | SEZ6L | chr22q12.1 | 0.966 | −0.088 | −4.291 |
| 208092_s_at | FAM49A | chr2p24.3 | 0.877 | −0.613 | −4.209 |
| 207094_at | IL8RA | chr2q35 | 0.895 | −0.527 | −4.122 |
| 207522_s_at | ATP2A3 | chr17p13.3 | 0.961 | −0.070 | −4.113 |
| 209325_s_at | RGS16 | chr1q25-q31 | 0.928 | −0.368 | −4.109 |
| 207033_at | GIF | chr11q13 | 0.943 | −0.255 | −4.104 |
| 211562_s_at | LMOD1 | chr1q32 | 0.896 | −0.519 | −4.102 |
| 210547_x_at | ICA1 | chr7p22 | 0.818 | −0.690 | −4.051 |
| 218806_s_at | VAV3 | chr1p13.3 | 0.951 | −0.158 | −4.048 |
| 32502_at | GDPD5 | chr11q13.4-q13.5 | 0.892 | −0.509 | −4.040 |
| 206935_at | PCDH8 | chr13q14.3-q21.1 | 0.976 | 0.205 | −4.031 |
| 207873_x_at | SEZ6L | chr22q12.1 | 0.946 | −0.168 | −3.971 |
| 201287_s_at | SDC1 | chr2p24.1 | 0.941 | −0.202 | −3.952 |
| 207195_at | CNTN6 | chr3p26-p25 | 0.877 | −0.525 | −3.948 |
| 221576_at | GDF15 | chr19p13.11 | 0.793 | −0.696 | −3.930 |
| 205696_s_at | GFRA1 | chr10q26.11 | 0.929 | −0.264 | −3.896 |
| 208291_s_at | TH | chr11p15.5 | 0.960 | 0.025 | −3.891 |
| 219895_at | FAM70A | chrXq24 | 0.981 | 0.417 | −3.832 |
| 212801_at | CIT | chr12q24 | 0.902 | −0.384 | −3.825 |
| 219772_s_at | SMPX | chrXp22.1 | 0.942 | −0.125 | −3.818 |
| 216086_at | SV2C | chr5q13.3 | 0.961 | 0.085 | −3.808 |
| 205944_s_at | CLTCL1 | chr22q11.2\|22q11.21 | 0.868 | −0.498 | −3.792 |
| 214811_at | RIMBP2 | chr12q24.33 | 0.940 | −0.128 | −3.788 |
| 220256_s_at | OXCT2 | chr1p34 | 0.945 | −0.083 | −3.784 |
| 213832_at | KCND3 | chr1p13.3 | 0.972 | 0.268 | −3.750 |
| 220539_at | C10orf92 | chr10q26.3 | 0.882 | −0.424 | −3.721 |
| 203282_at | GBE1 | chr3p12.3 | 0.979 | 0.408 | −3.709 |
| 219073_s_at | OSBPL10 | chr3p22.3 | 0.962 | 0.143 | −3.705 |
| 219093_at | PID1 | chr2q36.3 | 0.941 | −0.079 | −3.697 |
| 218208_at | [symbol] OC100131178 /// | chr18q23 | 0.890 | −0.379 | −3.689 |
| | PQL [symbol] | | | | |
| 205857_at | SLC18A2 | chr10q25 | 0.980 | 0.439 | −3.687 |
| 218631_at | AVPI1 | chr10q24.2 | 0.903 | −0.319 | −3.684 |
| 212316_at | NUP210 | chr3p25.1 | 0.923 | −0.204 | −3.677 |
| 213424_at | KIAA0895 | chr7p14.2 | 0.967 | 0.230 | −3.673 |
| 205825_at | PCSK1 | chr5q15-q21 | 0.890 | −0.362 | −3.648 |
| 209530_at | CACNB3 | chr12q13 | 0.962 | 0.177 | −3.642 |
| 215566_x_at | LYPLA2 | chr1p36.12-p35.1 | 0.962 | 0.181 | −3.637 |
| 203680_at | PRKAR2B | chr7q22 | 0.969 | 0.288 | −3.619 |
| 201410_at | PLEKHB2 | chr2q21.1 | 0.964 | 0.213 | −3.617 |
| 204337_at | RGS4 | chr1q23.3 | 0.956 | 0.109 | −3.616 |
| 221957_at | PDK3 | chrXp22.11 | 0.901 | −0.288 | −3.596 |
| 209981_at | CSDC2 | chr22q13.2-q13.31 | 0.865 | −0.426 | −3.586 |
| 215217_at | — | — | 0.884 | −0.353 | −3.575 |
| 215153_at | NOS1AP | chr1q23.3 | 0.812 | −0.557 | −3.572 |
| 220559_at | EN1 | chr2q13-q21 | 0.969 | 0.300 | −3.565 |
| 204556_s_at | DZIP1 | chr13q32.1 | 0.964 | 0.234 | −3.562 |
| 215771_x_at | RET | chr10q11.2 | 0.972 | 0.366 | −3.515 |
| 216548_x_at | HMGB3L1 | chr20q11.22 | 0.887 | −0.315 | −3.511 |
| 204269_at | PIM2 | chrXp11.23 | 0.941 | 0.019 | −3.493 |
| 220762_s_at | GNB1L | chr22q11.2 | 0.839 | −0.463 | −3.479 |
| 211546_x_at | SNCA | chr4q21 | 0.951 | 0.139 | −3.457 |
| 214156_at | MYRIP | chr3p22.1 | 0.961 | 0.257 | −3.445 |
| 221344_at | OR12D2 | chr6p22.2-p21.31 | 0.888 | −0.279 | −3.439 |
| 214347_s_at | DDC | chr7p12.2 | 0.909 | −0.168 | −3.427 |
| 201286_at | SDC1 | chr2p24.1 | 0.940 | 0.050 | −3.421 |
| 202681_at | USP4 | chr3p21.3 | 0.957 | 0.224 | −3.404 |
| 222113_s_at | EPS15L1 | chr19p13.11 | 0.816 | −0.488 | −3.398 |
| 211173_at | CCKAR | chr4p15.1-p15.2 | 0.913 | −0.126 | −3.383 |
| 204261_s_at | PSEN2 | chr1q31-q42 | 0.931 | −0.004 | −3.383 |
| 205195_at | AP1S1 | chr7q22.1 | 0.852 | −0.383 | −3.381 |
| 210448_s_at | P2RX5 | chr17p13.3 | 0.914 | −0.107 | −3.366 |
| 213150_at | HOXA10 | chr7p15-p14 | 0.868 | −0.312 | −3.336 |
| 204105_s_at | NRCAM | chr7q31.1-q31.2 | 0.868 | −0.300 | −3.313 |
| 207167_at | IGSF2 | chr1p13 | 0.680 | −0.667 | −3.312 |
| 203823_at | RGS3 | chr9q32 | 0.822 | −0.436 | −3.301 |
| 202141_s_at | COPS8 | chr2q37.3 | 0.883 | −0.233 | −3.296 |
| 213938_at | ERC2 | chr3p14.3 | 0.957 | 0.286 | −3.279 |
| 203999_at | SYT1 | chr12cen-q21 | 0.932 | 0.060 | −3.272 |
| 217442_at | LOC100131825 | chr1q23.2 | 0.771 | −0.529 | −3.265 |
| 210286_s_at | SLC4A7 | chr3p22 | 0.652 | −0.682 | −3.263 |
| 211323_s_at | ITPR1 | chr3p26-p25 | 0.942 | 0.160 | −3.238 |
| 221273_s_at | RNF208 | chr9q34.3 | 0.937 | 0.119 | −3.233 |
| 218404_at | SNX10 | chr7p15.2 | 0.907 | −0.080 | −3.229 |
| 204604_at | PFTK1 | chr7q21-q22 | 0.939 | 0.137 | −3.222 |
| 204059_s_at | ME1 | chr6q12 | 0.912 | −0.048 | −3.212 |
| 219203_at | FAM158A | chr14q11.2 | 0.888 | −0.161 | −3.191 |
| 212446_s_at | LASS6 | chr2q24.3 | 0.954 | 0.293 | −3.185 |
| 204339_s_at | RGS4 | chr1q23.3 | 0.921 | 0.024 | −3.181 |
| 203413_at | NELL2 | chr12q13.11-q13.12 | 0.896 | −0.115 | −3.174 |
| 210989_at | LAMA4 | chr6q21 | 0.700 | −0.602 | −3.166 |
| 216376_x_at | — | — | 0.862 | −0.254 | −3.163 |
| 209947_at | UBAP2L | chr1q21.3 | 0.836 | −0.334 | −3.149 |
| 216944_s_at | ITPR1 | chr3p26-p25 | 0.943 | 0.208 | −3.145 |
| 220904_at | C6orf208 | chr6q27 | 0.620 | −0.679 | −3.144 |
| 206456_at | GABRA5 | chr15q11.2-q12 | 0.867 | −0.226 | −3.142 |
| 209560_s_at | DLK1 | chr14q32 | 0.936 | 0.152 | −3.140 |
| 203710_at | ITPR1 | chr3p26-p25 | 0.944 | 0.222 | −3.134 |
| 211624_s_at | DRD2 | chr11q23 | 0.841 | −0.310 | −3.131 |
| 213476_x_at | TUBB3 | chr16q24.3 | 0.846 | −0.293 | −3.128 |
| 204224_s_at | GCH1 | chr14q22.1-q22.2 | 0.965 | 0.444 | −3.123 |
| 210331_at | HECW1 | chr7p14.1-p13 | 0.821 | −0.363 | −3.120 |
| 207937_x_at | FGFR1 | chr8p11.2-p11.1 | 0.895 | −0.092 | −3.119 |
| 209783_at | DBP | chr19q13.3 | 0.839 | −0.312 | −3.118 |
| 211421_s_at | RET | chr10q11.2 | 0.966 | 0.454 | −3.114 |
| 203439_s_at | STC2 | chr5q35.2 | 0.813 | −0.379 | −3.109 |
| 220721_at | ZNF614 | chr19q13.33 | 0.760 | −0.492 | −3.108 |
| 213726_x_at | TUBB2C | chr9q34 | 0.836 | −0.316 | −3.107 |
| 206950_at | SCN9A | chr2q24 | 0.823 | −0.352 | −3.106 |
| 205318_at | KIF5A | chr12q13.13 | 0.906 | −0.026 | −3.099 |
| 208463_at | GABRA4 | chr4p12 | 0.809 | −0.384 | −3.098 |
| 202606_s_at | TLK1 | chr2q31.1 | 0.865 | −0.214 | −3.096 |
| 209985_s_at | ASCL1 | chr12q23.2 | 0.601 | −0.680 | −3.086 |
| 204491_at | PDE4D | chr5q12 | 0.890 | −0.098 | −3.085 |
| 55065_at | MARK4 | chr19q13.3 | 0.879 | −0.143 | −3.073 |
| 208016_s_at | AGTR1 | chr3q21-q25 | 0.759 | −0.477 | −3.067 |
| 213609_s_at | SEZ6L | chr22q12.1 | 0.905 | −0.010 | −3.062 |
| 221844_x_at | SPCS3 | chr4q34.2 | 0.950 | 0.308 | −3.058 |
| 216391_s_at | KLHL1 | chr13q21 | 0.820 | −0.335 | −3.052 |
| 219282_s_at | TRPV2 | chr17p11.2 | 0.915 | 0.053 | −3.046 |
| 212992_at | AHNAK2 | chr14q32.33 | 0.876 | −0.143 | −3.046 |
| 200064_at | HSP90AB1 | chr6p12 | 0.880 | −0.127 | −3.045 |
| 203354_s_at | PSD3 | chr8pter-p23.3 | 0.911 | 0.030 | −3.044 |
| 216256_at | GRM8 | chr7q31.3-q32.1 | 0.803 | −0.376 | −3.043 |
| 211909_x_at | PTGER3 | chr1p31.2 | 0.860 | −0.201 | −3.033 |
| 212653_s_at | EHBP1 | chr2p15 | 0.938 | 0.225 | −3.022 |
| 211252_x_at | PTCRA | chr6p21.3 | 0.818 | −0.328 | −3.021 |
| 205835_s_at | YTHDC2 | chr5q22.2 | 0.902 | −0.009 | −3.020 |
| 210380_s_at | CACNA1G | chr17q22 | 0.874 | −0.139 | −3.017 |
| 214434_at | HSPA12A | chr10q26.12 | 0.910 | 0.039 | −3.017 |
| 202501_at | MAPRE2 | chr18q12.1 | 0.787 | −0.399 | −3.009 |
| 209683_at | FAM49A | chr2p24.3 | 0.761 | −0.452 | −3.009 |
| 213558_at | PCLO | chr7q11.23-q21.3 | 0.903 | 0.006 | −3.008 |
| 211593_s_at | MAST2 | chr1p34.1 | 0.943 | 0.275 | −3.006 |
| 212590_at | RRAS2 | chr11p15.2 | 0.923 | 0.125 | −3.001 |
| 213920_at | CUX2 | chr12q24.11-q24.12 | 0.784 | −0.401 | −3.000 |
| 218018_at | PDXK | chr21q22.3 | 0.867 | −0.153 | −2.988 |
| 204175_at | ZNF593 | chr1p36.11 | 0.905 | 0.031 | −2.978 |
| 205632_s_at | PIP5K1B | chr9q13 | 0.962 | 0.470 | −2.975 |
| 200650_s_at | LDHA | chr11p15.4 | 0.901 | 0.007 | −2.974 |
| 203458_at | SPR | chr2p14-p12 | 0.957 | 0.416 | −2.966 |
| 200870_at | STRAP | chr12p12.3 | 0.900 | 0.014 | −2.960 |
| 214121_x_at | PDLIM7 | chr5q35.3 | 0.881 | −0.078 | −2.954 |
| 213902_at | ASAH1 | chr8p22-p21.3 | 0.872 | −0.114 | −2.953 |
| 205390_s_at | ANK1 | chr8p11.1 | 0.947 | 0.330 | −2.953 |
| 206941_x_at | SEMA3E | chr7q21.11 | 0.788 | −0.369 | −2.948 |
| 216215_s_at | RBM9 | chr22q13.1 | 0.924 | 0.159 | −2.948 |
| 221406_s_at | C6orf26 /// | chr6p21.3 /// | 0.736 | −0.471 | −2.946 |
| | MSH5 | chr6p21.33 | | | |
| 210008_s_at | MRPS12 | chr19q13.1-q13.2 | 0.819 | −0.291 | −2.946 |
| 212224_at | ALDH1A1 | chr9q21.13 | 0.937 | 0.253 | −2.944 |
| 217128_s_at | CAMK1G | chr1q32-q41 | 0.822 | −0.281 | −2.942 |
| 202832_at | GCC2 | chr2q12.3 | 0.875 | −0.096 | −2.940 |
| 216253_s_at | PARVB | chr22q13.2-q13.33 | 0.792 | −0.355 | −2.936 |
| 206592_s_at | AP3D1 | chr19p13.3 | 0.909 | 0.076 | −2.930 |
| 214217_at | GRM5 | chr11q14.2-q14.3 | 0.813 | −0.299 | −2.929 |
| 205551_at | SV2B | chr15q26.1 | 0.918 | 0.129 | −2.928 |
| 209444_at | RAP1GDS1 | chr4q23-q25 | 0.856 | −0.165 | −2.927 |
| 208034_s_at | PROZ | chr13q34 | 0.801 | −0.327 | −2.919 |
| 212851_at | DCUN1D4 | chr4q12 | 0.777 | −0.380 | −2.914 |
| 215728_s_at | ACOT7 | chr1p36.31-p36.11 | 0.890 | −0.011 | −2.907 |
| 200622_x_at | CALM3 | chr19q13.2-q13.3 | 0.838 | −0.212 | −2.899 |
| 213601_at | SLIT1 | chr10q23.3-q24 | 0.941 | 0.308 | −2.896 |
| 209237_s_at | SLC23A2 | chr20p13 | 0.721 | −0.478 | −2.895 |
| 212960_at | TBC1D9 | chr4q31.21 | 0.929 | 0.224 | −2.883 |
| 202684_s_at | RNMT | chr18p11.22-p11.23 | 0.701 | −0.501 | −2.878 |
| 207501_s_at | FGF12 | chr3q28 | 0.896 | 0.031 | −2.877 |
| 34408_at | RTN2 | chr19q13.32 | 0.910 | 0.108 | −2.876 |
| 37950_at | PREP | chr6q22 | 0.894 | 0.026 | −2.867 |
| 202042_at | HARS | chr5q31.3 | 0.885 | −0.015 | −2.867 |
| 205566_at | ABHD2 | chr15q26.1 | 0.828 | −0.229 | −2.867 |
| 206046_at | ADAM23 | chr2q33 | 0.866 | −0.097 | −2.864 |
| 204991_s_at | NF2 | chr22q12.2 | 0.794 | −0.318 | −2.862 |
| 209635_at | AP1S1 | chr7q22.1 | 0.689 | −0.511 | −2.858 |
| 220131_at | FXYD7 | chr19q13.12 | 0.794 | −0.316 | −2.853 |
| 211577_s_at | IGF1 | chr12q22-q23 | 0.876 | −0.048 | −2.849 |
| 205196_s_at | AP1S1 | chr7q22.1 | 0.570 | −0.639 | −2.846 |
| 206502_s_at | INSM1 | chr20p11.2 | 0.921 | 0.186 | −2.844 |
| 211383_s_at | WDR37 | chr10p15.3 | 0.887 | 0.005 | −2.843 |
| 202154_x_at | TUBB3 | chr16q24.3 | 0.831 | −0.207 | −2.838 |
| 206527_at | ABAT | chr16p13.2 | 0.790 | −0.318 | −2.836 |
| 208845_at | VDAC3 | chr8p11.2 | 0.905 | 0.100 | −2.834 |
| 205348_s_at | DYNC1I1 | chr7q21.3-q22.1 | 0.919 | 0.184 | −2.833 |
| 202722_s_at | GFPT1 | chr2p13 | 0.864 | −0.086 | −2.829 |
| 219365_s_at | CAMKV | chr3p21.31 | 0.796 | −0.299 | −2.829 |
| 207422_at | ADAM20 | chr14q24.1 | 0.706 | −0.475 | −2.827 |
| 201714_at | TUBG1 | chr17q21 | 0.803 | −0.279 | −2.825 |
| 216092_s_at | SLC7A8 | chr14q11.2 | 0.871 | −0.056 | −2.824 |
| 202759_s_at | [symbol] /// | chr9q31-q33 | 0.825 | −0.218 | −2.823 |
| | PALM2 /// | | | | |
| | PALM2- [symbol] | | | | |
| 207582_at | PIN1L | chr1p31 | 0.888 | 0.020 | −2.821 |
| 206732_at | SLITRK3 | chr3q26.1 | 0.794 | −0.301 | −2.821 |
| 214306_at | OPA1 | chr3q28-q29\|3q28-q29 | 0.823 | −0.221 | −2.817 |
| 204977_at | DDX10 | chr11q22-q23 | 0.914 | 0.158 | −2.816 |
| 121_at | PAX8 | chr2q12-q14 | 0.759 | −0.377 | −2.816 |
| 205747_at | CBLN1 | chr16q12.1 | 0.936 | 0.305 | −2.811 |
| 37965_at | PARVB | chr22q13.2-q13.33 | 0.712 | −0.458 | −2.807 |
| 206746_at | BFSP1 | chr20p11.23-p12.1 | 0.778 | −0.332 | −2.804 |
| 212956_at | TBC1D9 | chr4q31.21 | 0.920 | 0.201 | −2.804 |
| 202913_at | ARHGEF11 | chr1q21 | 0.924 | 0.226 | −2.801 |
| 213273_at | ODZ4 | chr11q14.1 | 0.632 | −0.563 | −2.798 |
| 218613_at | PSD3 | chr8pter-p23.3 | 0.887 | 0.027 | −2.798 |
| 212946_at | KIAA0564 | chr13q14.11 | 0.865 | −0.066 | −2.797 |
| 220449_at | MGC5566 | chr20q13.12 | 0.805 | −0.261 | −2.795 |
| 201870_at | TOMM34 | — | 0.854 | −0.106 | −2.793 |
| 218189_s_at | NANS | chr9p24.1-p23 | 0.908 | 0.141 | −2.788 |
| 218856_at | TNFRSF21 | chr6p21.1-p12.2 | 0.515 | −0.667 | −2.787 |
| 214848_at | — | — | 0.855 | −0.099 | −2.786 |
| 206421_s_at | SERPINB7 | chr18q21.33 | 0.791 | −0.292 | −2.784 |
| 207591_s_at | ARID1A | chr1p35.3 | 0.691 | −0.480 | −2.782 |
| 206065_s_at | DPYS | chr8q22 | 0.837 | −0.161 | −2.782 |
| 212442_s_at | LASS6 | chr2q24.3 | 0.933 | 0.299 | −2.779 |
| 215150_at | YOD1 | chr1q32.1 | 0.680 | −0.494 | −2.777 |
| 40284_at | FOXA2 | chr20p11 | 0.856 | −0.090 | −2.775 |
| 203890_s_at | DAPK3 | chr19p13.3 | 0.756 | −0.364 | −2.772 |
| 212255_s_at | ATP2C1 | chr3q22.1 | 0.787 | −0.296 | −2.771 |
| 204338_s_at | RGS4 | chr1q23.3 | 0.859 | −0.078 | −2.771 |
| 203456_at | PRAF2 | chrXp11.23 | 0.860 | −0.073 | −2.771 |
| 219302_s_at | CNTNAP2 | chr7q35-q36 | 0.899 | 0.101 | −2.770 |
| 204035_at | SCG2 | chr2q35-q36 | 0.847 | −0.120 | −2.770 |
| 219916_s_at | RNF39 | chr6p21.3 | 0.688 | −0.480 | −2.768 |
| 210652_s_at | TTC39A | chr1p32.3 | 0.904 | 0.124 | −2.768 |
| 212728_at | DLG3 | chrXq13.1 | 0.846 | −0.123 | −2.767 |
| 215124_at | ZNF550 | chr19q13.43 | 0.627 | −0.557 | −2.766 |
| 205357_s_at | AGTR1 | chr3q21-q25 | 0.939 | 0.347 | −2.763 |
| 207349_s_at | UCP3 | chr11q13 | 0.833 | −0.165 | −2.763 |
| 213808_at | — | — | 0.864 | −0.053 | −2.761 |
| 209992_at | PFKFB2 | chr1q31 | 0.795 | −0.267 | −2.754 |
| 213059_at | CREB3L1 | chr11p11.2 | 0.606 | −0.576 | −2.754 |
| 216475_at | — | — | 0.854 | −0.089 | −2.754 |
| 209453_at | SLC9A1 | chr1p36.1-p35 | 0.818 | −0.205 | −2.751 |
| 200078_s_at | ATP6V0B | chr1p32.3 | 0.880 | 0.018 | −2.750 |
| 207827_x_at | SNCA | chr4q21 | 0.936 | 0.332 | −2.749 |
| 203389_at | KIF3C | chr2p23 | 0.933 | 0.315 | −2.745 |
| 212475_at | AVL9 | chr7p14.3 | 0.895 | 0.093 | −2.743 |
| 219032_x_at | OPN3 | chr1q43 | 0.816 | −0.207 | −2.742 |
| 221560_at | MARK4 | chr19q13.3 | 0.753 | −0.357 | −2.740 |
| 212157_at | SDC2 | chr8q22-q23 | 0.947 | 0.425 | −2.739 |
| 206679_at | APBA1 | chr9q13-q21.1 | 0.637 | −0.535 | −2.738 |
| 217162_at | — | — | 0.709 | −0.433 | −2.731 |
| 204365_s_at | REEP1 | chr2p11.2 | 0.923 | 0.253 | −2.730 |
| 213330_s_at | STIP1 | chr11q13 | 0.875 | 0.005 | −2.730 |
| 207804_s_at | FCN2 | chr9q34.3 | 0.790 | −0.267 | −2.726 |
| 215014_at | KCND3 | chr1p13.3 | 0.927 | 0.282 | −2.725 |
| 204586_at | BSN | chr3p21.31 | 0.893 | 0.094 | −2.724 |
| 215097_at | CAPZB | chr1p36.1 | 0.898 | 0.115 | −2.724 |
| 209935_at | ATP2C1 | chr3q22.1 | 0.753 | −0.350 | −2.724 |
| 213901_x_at | RBM9 | chr22q13.1 | 0.888 | 0.068 | −2.722 |
| 221509_at | DENR | chr12q24.31 | 0.926 | 0.278 | −2.721 |
| 213870_at | COL11A2 | chr6p21.3 | 0.770 | −0.310 | −2.717 |
| 217979_at | TSPAN13 | chr7p21.1 | 0.894 | 0.103 | −2.715 |
| 206815_at | SPAG8 | chr9p13.3 | 0.746 | −0.359 | −2.714 |
| 206869_at | CHAD | chr17q21.33 | 0.513 | −0.648 | −2.712 |
| 207096_at | SAA4 | chr11p15.1-p14 | 0.738 | −0.373 | −2.709 |
| 219270_at | CHAC1 | chr15q15.1 | 0.842 | −0.108 | −2.706 |
| 204590_x_at | VPS33A | chr12q24.31 | 0.810 | −0.204 | −2.705 |
| 205011_at | VWA5A | chr11q23 | 0.920 | 0.248 | −2.703 |
| 207521_s_at | ATP2A3 | chr17p13.3 | 0.812 | −0.197 | −2.702 |
| 34726_at | CACNB3 | chr12q13 | 0.930 | 0.311 | −2.701 |
| 219736_at | TRIM36 | chr5q22.3 | 0.805 | −0.217 | −2.700 |
| 221269_s_at | SH3BGRL3 | chr1p35-p34.3 | 0.877 | 0.030 | −2.699 |
| 206104_at | ISL1 | chr5q11.2 | 0.720 | −0.401 | −2.699 |
| 204730_at | RIMS3 | chr1pter-p22.2 | 0.786 | −0.263 | −2.696 |
| 208977_x_at | TUBB2C | chr9q34 | 0.789 | −0.257 | −2.695 |
| 216805_at | — | — | 0.616 | −0.545 | −2.694 |
| 204574_s_at | MMP19 | chr12q14 | 0.757 | −0.327 | −2.693 |
| 222255_at | PRX | chr19q13.13-q13.2 | 0.698 | −0.434 | −2.691 |
| 201439_at | GBF1 | chr10q24 | 0.911 | 0.201 | −2.689 |
| 216963_s_at | GAP43 | chr3q13.1-q13.2 | 0.885 | 0.076 | −2.681 |
| 213198_at | ACVR1B | chr12q13 | 0.870 | 0.010 | −2.681 |
| 212383_at | ATP6V0A1 | chr17q21 | 0.825 | −0.149 | −2.679 |
| 216967_at | GAP43 | chr3q13.1-q13.2 | 0.862 | −0.020 | −2.678 |
| 205879_x_at | RET | chr10q11.2 | 0.931 | 0.331 | −2.676 |
| 205241_at | SCO2 | chr22q13.33 | 0.945 | 0.431 | −2.676 |
| 204813_at | MAPK10 | chr4q22.1-q23 | 0.886 | 0.084 | −2.675 |
| 218956_s_at | PTCD1 | chr7q22.1 | 0.903 | 0.166 | −2.674 |
| 220482_s_at | SERGEF | chr11p14.3 | 0.915 | 0.235 | −2.673 |
| 216533_at | PCCA | chr13q32 | 0.856 | −0.040 | −2.672 |
| 212055_at | C18orf10 | chr18q12.2 | 0.899 | 0.151 | −2.668 |
| 212820_at | DMXL2 | chr15q21.2 | 0.876 | 0.043 | −2.668 |
| 212695_at | CRY2 | chr11p11.2 | 0.845 | −0.078 | −2.665 |
| 217120_s_at | MED14 | chrXp11.4-p11.2 | 0.661 | −0.477 | −2.661 |
| 204816_s_at | DHX34 | chr19q13.3 | 0.836 | −0.106 | −2.661 |
| 202539_s_at | HMGCR | chr5q13.3-q14 | 0.874 | 0.037 | −2.657 |
| 214874_at | PKP4 | chr2q23-q31 | 0.750 | −0.326 | −2.656 |
| 220173_at | C14orf45 | chr14q24.3 | 0.871 | 0.027 | −2.655 |
| 214359_s_at | HSP90AB1 | chr6p12 | 0.791 | −0.231 | −2.652 |
| 203476_at | TPBG | chr6q14-q15 | 0.928 | 0.326 | −2.651 |
| 212048_s_at | YARS | chr1p35.1 | 0.820 | −0.149 | −2.649 |
| 34221_at | HMGXB3 | chr5q33.1 | 0.793 | −0.223 | −2.648 |
| 214452_at | BCAT1 | chr12p12.1 | 0.778 | −0.260 | −2.648 |
| 217512_at | KNG1 | chr3q27 | 0.715 | −0.388 | −2.647 |
| 205095_s_at | ATP6V0A1 | chr17q21 | 0.752 | −0.317 | −2.646 |
| 219338_s_at | LRRC49 | chr15q23 | 0.857 | −0.023 | −2.645 |
| 210153_s_at | ME2 | chr6p25-p24\|18q21 | 0.826 | −0.129 | −2.645 |
| 202263_at | CYB5R1 | chr1p36.13-q41 | 0.910 | 0.220 | −2.644 |
| 207135_at | HTR2A | chr13q14-q21 | 0.810 | −0.176 | −2.644 |
| 221633_at | NCAPH2 | chr22q13.33 | 0.592 | −0.554 | −2.643 |
| 200734_s_at | ARF3 | chr12q13 | 0.900 | 0.168 | −2.643 |
| 221533_at | FAM162A | chr3q21.1 | 0.740 | −0.339 | −2.638 |
| 202801_at | PRKACA | chr19p13.1 | 0.829 | −0.117 | −2.637 |
| 206481_s_at | LDB2 | chr4p16 | 0.781 | −0.248 | −2.636 |
| 203266_s_at | MAP2K4 | chr17p11.2 | 0.912 | 0.236 | −2.636 |
| 216360_x_at | RRP12 | chr10q24.1 | 0.755 | −0.305 | −2.636 |
| 208693_s_at | GARS | chr7p15 | 0.812 | −0.166 | −2.635 |
| 206233_at | B4GALT6 | chr18q11 | 0.884 | 0.095 | −2.632 |
| 210650_s_at | PCLO | chr7q11.23-q21.3 | 0.834 | −0.096 | −2.632 |
| 222234_s_at | DBNDD1 | chr16q24.3 | 0.839 | −0.080 | −2.631 |
| 209236_at | SLC23A2 | chr20p13 | 0.819 | −0.142 | −2.630 |
| 220201_at | RC3H2 | chr9q34 | 0.782 | −0.243 | −2.630 |
| 202142_at | COPS8 | chr2q37.3 | 0.805 | −0.182 | −2.628 |
| 204744_s_at | IARS | chr9q21 | 0.858 | −0.011 | −2.627 |
| 52837_at | KIAA1644 | — | 0.735 | −0.342 | −2.625 |
| 204814_at | CADPS | chr3p14.2 | 0.905 | 0.201 | −2.624 |
| 212213_x_at | OPA1 | chr3q28-q29\|3q28-q29 | 0.838 | −0.080 | −2.621 |
| 204217_s_at | RTN2 | chr19q13.32 | 0.872 | 0.049 | −2.619 |
| 218704_at | RNF43 | chr17q22 | 0.892 | 0.142 | −2.615 |
| 214581_x_at | TNFRSF21 | chr6p21.1-p12.2 | 0.584 | −0.553 | −2.614 |
| 212104_s_at | RBM9 | chr22q13.1 | 0.902 | 0.191 | −2.614 |
| 209599_s_at | PRUNE | chr1q21 | 0.645 | −0.480 | −2.613 |
| 204722_at | SCN3B | chr11q23.3 | 0.863 | 0.017 | −2.612 |
| 216400_at | GBA /// GBAP | chr1q21 | 0.743 | −0.318 | −2.608 |
| 40255_at | DDX28 | chr16q22.1 | 0.734 | −0.337 | −2.607 |
| 206440_at | LIN7A | chr12q21 | 0.824 | −0.116 | −2.606 |
| 221750_at | HMGCS1 | chr5p14-p13 | 0.838 | −0.069 | −2.604 |
| 203017_s_at | SSX2IP | chr1p22.3 | 0.873 | 0.063 | −2.599 |
| 217930_s_at | TOLLIP | chr11p15.5 | 0.787 | −0.214 | −2.597 |
| 202752_x_at | SLC7A8 | chr14q11.2 | 0.839 | −0.063 | −2.592 |
| 201500_s_at | PPP1R11 | chr6p21.3 | 0.848 | −0.029 | −2.592 |
| 211892_s_at | PTGIS | chr20q13.13 | 0.501 | −0.622 | −2.591 |
| 202872_at | ATP6V1C1 | chr8q22.3 | 0.853 | −0.008 | −2.586 |
| 216932_at | — | — | 0.799 | −0.176 | −2.582 |
| 209923_s_at | BRAP | chr12q24 | 0.824 | −0.103 | −2.580 |
| 216444_at | — | — | 0.579 | −0.546 | −2.580 |
| 205406_s_at | SPA17 | chr11q24.2 | 0.812 | −0.136 | −2.576 |
| 222206_s_at | NCLN | chr19p13.3 | 0.774 | −0.236 | −2.575 |
| 212242_at | TUBA4A | chr2q35 | 0.839 | −0.052 | −2.573 |
| 217319_x_at | CYP4A22 | chr1p33 | 0.788 | −0.202 | −2.573 |
| 202648_at | — | — | 0.754 | −0.279 | −2.572 |
| 208824_x_at | PCTK1 | chrXp11.3-p11.23 | 0.749 | −0.289 | −2.571 |
| 220627_at | CST8 | chr20p11.21 | 0.791 | −0.191 | −2.570 |
| 214673_s_at | HUWE1 | chrXp11.22 | 0.891 | 0.158 | −2.567 |
| 203826_s_at | PITPNM1 | chr11q13 | 0.841 | −0.042 | −2.563 |
| 212101_at | KPNA6 | chr1p35.1-p34.3 | 0.811 | −0.134 | −2.563 |
| 201050_at | PLD3 | chr19q13.2 | 0.840 | −0.039 | −2.555 |
| 203067_at | PDHX | chr11p13 | 0.892 | 0.169 | −2.555 |
| 218662_s_at | NCAPG | chr4p15.33 | 0.973 | 0.706 | −2.554 |
| 215169_at | SLC35E2 | chr1p36.33 | 0.726 | −0.328 | −2.554 |
| 219117_s_at | FKBP11 | chr12q13.12 | 0.928 | 0.367 | −2.554 |
| 215492_x_at | PTCRA | chr6p21.3 | 0.837 | −0.051 | −2.554 |
| 202651_at | LPGAT1 | chr1q32 | 0.780 | −0.211 | −2.552 |
| 206573_at | KCNQ3 | chr8q24 | 0.589 | −0.525 | −2.552 |
| 208899_x_at | ATP6V1D | chr14q23-q24.2 | 0.828 | −0.076 | −2.552 |
| 202874_s_at | ATP6V1C1 | chr8q22.3 | 0.855 | 0.015 | −2.551 |
| 220136_s_at | CRYBA2 | chr2q34-q36 | 0.837 | −0.047 | −2.549 |
| 204948_s_at | FST | chr5q11.2 | 0.543 | −0.571 | −2.546 |
| 214436_at | FBXL2 | chr3p22.3 | 0.869 | 0.071 | −2.545 |
| 206078_at | KALRN | chr3q21.1-q21.2 | 0.629 | −0.475 | −2.544 |
| 217908_s_at | IQWD1 | chr1q24.2 | 0.891 | 0.170 | −2.544 |
| 212607_at | AKT3 | chr1q43-q44 | 0.798 | −0.161 | −2.544 |
| 208017_s_at | MCF2 | chrXq27 | 0.808 | −0.134 | −2.544 |
| 203030_s_at | PTPRN2 | chr7q36 | 0.851 | 0.008 | −2.540 |
| 204974_at | RAB3A | chr19p13.2 | 0.868 | 0.071 | −2.538 |
| 215894_at | PTGDR | chr14q22.1 | 0.721 | −0.330 | −2.537 |
| 213308_at | SHANK2 | chr11q13.3 | 0.842 | −0.024 | −2.536 |
| 214096_s_at | SHMT2 | chr12q12-q14 | 0.890 | 0.169 | −2.536 |
| 211714_x_at | TUBB | chr6p21.33 | 0.767 | −0.235 | −2.536 |
| 207658_s_at | FOXG1 | chr14q13 | 0.612 | −0.492 | −2.535 |
| 222230_s_at | ACTR10 | chr14q23.1 | 0.861 | 0.046 | −2.534 |
| 222005_s_at | GNG3 | chr11p11 | 0.873 | 0.095 | −2.534 |
| 213319_s_at | CSDA | chr12p13.1 | 0.675 | −0.405 | −2.532 |
| 208872_s_at | REEP5 | chr5q22-q23 | 0.808 | −0.127 | −2.530 |
| 200945_s_at | SEC31A | chr4q21.22 | 0.854 | 0.022 | −2.529 |
| 201660_at | ACSL3 | chr2q34-q35 | 0.804 | −0.134 | −2.521 |
| 214969_at | MAP3K9 | chr14q24.3-q31 | 0.862 | 0.058 | −2.518 |
| 209926_at | LOC729991 | chr19p13.11 | 0.685 | −0.383 | −2.517 |
| 213469_at | PGAP1 | chr2q33.1 | 0.778 | −0.198 | −2.516 |
| 206721_at | C1orf114 | chr1q24 | 0.907 | 0.264 | −2.515 |
| 218817_at | SPCS3 | chr4q34.2 | 0.881 | 0.136 | −2.515 |
| 219414_at | CLSTN2 | chr3q23-q24 | 0.761 | −0.238 | −2.515 |
| 215764_x_at | AP2A2 | chr11p15.5 | 0.813 | −0.105 | −2.512 |
| 209877_at | SNCG | chr10q23.2-q23.3 | 0.874 | 0.110 | −2.510 |
| 216073_at | ANKRD34C | chr15q25.1 | 0.872 | 0.103 | −2.509 |
| 209186_at | ATP2A2 | chr12q23-q24.1 | 0.722 | −0.315 | −2.506 |
| 214445_at | ELL2 | chr5q15 | 0.595 | −0.501 | −2.503 |
| 209011_at | TRIO | chr5p15.2 | 0.882 | 0.148 | −2.502 |
| 205268_s_at | ADD2 | chr2p14-p13 | 0.879 | 0.137 | −2.501 |
| 211712_s_at | ANXA9 | chr1q21 | 0.770 | −0.210 | −2.501 |
| 216076_at | L3MBTL | chr20q13.12 | 0.781 | −0.185 | −2.501 |
| 204165_at | WASF1 | chr6q21-q22 | 0.807 | −0.116 | −2.499 |
| 204795_at | PRR3 | chr6p21.33 | 0.664 | −0.407 | −2.496 |
| 219939_s_at | CSDE1 | chr1p22 | 0.922 | 0.354 | −2.496 |
| 209026_x_at | TUBB | chr6p21.33 | 0.762 | −0.226 | −2.496 |
| 220486_x_at | [symbol] C100130886 /// | chrXq22.3 | 0.921 | 0.350 | −2.496 |
| | TMEM [symbol] | | | | |
| 41047_at | C9orf16 | chr9q34.1 | 0.881 | 0.147 | −2.496 |
| 218633_x_at | ABHD10 | chr3q13.2 | 0.830 | −0.044 | −2.495 |
| 1487_at | ESRRA | chr11q13 | 0.837 | −0.021 | −2.495 |
| 203841_x_at | MAPRE3 | chr2p23.3-p23.1 | 0.863 | 0.076 | −2.494 |
| 201433_s_at | PTDSS1 | chr8q22 | 0.921 | 0.349 | −2.494 |
| 201527_at | ATP6V1F | chr7q32 | 0.857 | 0.051 | −2.493 |
| 202921_s_at | ANK2 | chr4q25-q27 | 0.819 | −0.075 | −2.493 |
| 209857_s_at | SPHK2 | chr19q13.2 | 0.824 | −0.059 | −2.492 |
| 206537_at | XIAP | chrXq25 | 0.806 | −0.109 | −2.485 |
| 201269_s_at | NUDCD3 | chr7p13-p12 | 0.861 | 0.074 | −2.480 |
| 221069_s_at | CCDC44 | chr17q23.3 | 0.828 | −0.042 | −2.479 |
| 204743_at | TAGLN3 | chr3q13.2 | 0.886 | 0.176 | −2.478 |
| 204869_at | PCSK2 | chr20p11.2 | 0.835 | −0.017 | −2.478 |
| 209556_at | NCDN | chr1p34.3 | 0.791 | −0.146 | −2.477 |
| 222064_s_at | AARSD1 | chr17q21.31 | 0.875 | 0.129 | −2.476 |
| 219557_s_at | NRIP3 | chr11p15.3 | 0.842 | 0.009 | −2.473 |
| 208316_s_at | OCRL | chrXq25-q26.1 | 0.843 | 0.011 | −2.473 |
| 212853_at | DCUN1D4 | chr4q12 | 0.691 | −0.353 | −2.470 |
| 205636_at | SH3GL3 | chr15q24 | 0.647 | −0.420 | −2.469 |
| 204837_at | MTMR9 | chr8p23-p22 | 0.759 | −0.220 | −2.466 |
| 205549_at | PCP4 | chr21q22.2 | 0.746 | −0.246 | −2.464 |
| 210191_s_at | PHTF1 | chr1p13 | 0.857 | 0.066 | −2.463 |
| 217098_s_at | ZSCAN12 | chr6p22.2-p21.3 | 0.699 | −0.335 | −2.461 |
| 218884_s_at | GUF1 | chr4p13 | 0.724 | −0.291 | −2.461 |
| 220117_at | ZNF385D | chr3p24.3 | 0.843 | 0.019 | −2.457 |
| 207869_s_at | CACNA1G | chr17q22 | 0.782 | −0.158 | −2.452 |
| 205376_at | INPP4B | chr4q31.21 | 0.805 | −0.098 | −2.450 |
| 205047_s_at | ASNS | chr7q21.3 | 0.792 | −0.132 | −2.450 |
| 220942_x_at | FAM162A | chr3q21.1 | 0.758 | −0.214 | −2.449 |
| 212214_at | OPA1 | chr3q28- | 0.805 | −0.097 | −2.449 |
| | | q29\|3q28-q29 | | | |
| 219928_s_at | CABYR | chr18q11.2 | 0.862 | 0.091 | −2.447 |
| 202043_s_at | SMS | chrXp22.1 | 0.866 | 0.108 | −2.446 |
| 221901_at | KIAA1644 | — | 0.775 | −0.169 | −2.439 |
| 212987_at | FBXO9 | chr6p12.3-p11.2 | 0.867 | 0.118 | −2.438 |
| 201609_x_at | ICMT | chr1p36.21 | 0.856 | 0.074 | −2.437 |
| 219572_at | CADPS2 | chr7q31.3 | 0.963 | 0.651 | −2.437 |
| 208002_s_at | ACOT7 | chr1p36.31-p36.11 | 0.872 | 0.139 | −2.437 |
| 206435_at | B4GALNT1 | chr12q13.3 | 0.838 | 0.013 | −2.437 |
| 217393_x_at | UBE2NL | chrXq27.3 | 0.879 | 0.168 | −2.436 |
| 219637_at | ARMC9 | chr2q37.1 | 0.799 | −0.105 | −2.435 |
| 208372_s_at | LIMK1 | chr7q11.23 | 0.676 | −0.361 | −2.431 |
| 209853_s_at | PSME3 | chr17q21 | 0.919 | 0.365 | −2.431 |
| 221566_s_at | NOL3 | chr16q21-q23 | 0.783 | −0.144 | −2.430 |
| 204505_s_at | EPB49 | chr8p21.1 | 0.872 | 0.142 | −2.429 |
| 214293_at | 40432 | chr4q21.1 | 0.761 | −0.197 | −2.428 |
| 218260_at | DDA1 | chr19p13.11 | 0.797 | −0.107 | −2.428 |
| 221211_s_at | C21orf7 | chr21q22.3 | 0.847 | 0.048 | −2.425 |
| 200873_s_at | CCT8 | chr21q22.11 | 0.896 | 0.251 | −2.424 |
| 208888_s_at | NCOR2 | chr12q24 | 0.740 | −0.238 | −2.419 |
| 205282_at | LRP8 | chr1p34 | 0.841 | 0.032 | −2.419 |
| 211428_at | SERPINA1 | chr14q32.1 | 0.662 | −0.377 | −2.419 |
| 206339_at | CARTPT | chr5q13.2 | 0.823 | −0.028 | −2.417 |
| 221471_at | SERINC3 | chr20q13.1-q13.3 | 0.841 | 0.032 | −2.416 |
| 216097_at | — | — | 0.717 | −0.282 | −2.416 |
| 200720_s_at | ACTR1A | chr10q24.32 | 0.816 | −0.047 | −2.416 |
| 210527_x_at | TUBA3C | chr13q11 | 0.725 | −0.268 | −2.416 |
| 221792_at | RAB6B | chr3q22.1 | 0.855 | 0.083 | −2.414 |
| 209397_at | ME2 | chr6p25-p24\|18q21 | 0.811 | −0.059 | −2.412 |
| 206137_at | RIMS2 | chr8q22.3 | 0.803 | −0.083 | −2.410 |
| 202554_s_at | GSTM3 | chr1p13.3 | 0.833 | 0.009 | −2.409 |
| 213036_x_at | ATP2A3 | chr17p13.3 | 0.830 | −0.002 | −2.409 |
| 204720_s_at | DNAJC6 | chr1pter-q31.3 | 0.875 | 0.166 | −2.407 |
| 217231_s_at | MAST1 | chr19p13.2 | 0.802 | −0.082 | −2.407 |
| 205265_s_at | SPEG | chr2q35 | 0.807 | −0.069 | −2.407 |
| 209096_at | UBE2V2 | chr8q11.21 | 0.848 | 0.060 | −2.406 |
| 210244_at | CAMP | chr3p21.3 | 0.606 | −0.450 | −2.406 |
| 213307_at | SHANK2 | chr11q13.3 | 0.832 | 0.007 | −2.406 |
| 221727_at | SUB1 | chr5p13.3 | 0.870 | 0.143 | −2.406 |
| 209410_s_at | GRB10 | chr7p12-p11.2 | 0.640 | −0.404 | −2.405 |
| 210065_s_at | UPK1B | chr3q13.3-q21 | 0.676 | −0.350 | −2.405 |
| 204870_s_at | PCSK2 | chr20p11.2 | 0.816 | −0.042 | −2.404 |
| 214844_s_at | DOK5 | chr20q13.2 | 0.855 | 0.087 | −2.403 |
| 202572_s_at | DLGAP4 | chr20q11.23 | 0.807 | −0.068 | −2.402 |
| 206993_at | ATP5S | chr14q22.1 | 0.836 | 0.022 | −2.402 |
| 218725_at | SLC25A22 | chr11p15.5 | 0.874 | 0.163 | −2.402 |
| 219685_at | TMEM35 | chrXq22.1 | 0.887 | 0.220 | −2.402 |
| 211971_s_at | LRPPRC | chr2p21 | 0.837 | 0.025 | −2.401 |
| 214137_at | PTPRJ | chr11p11.2 | 0.670 | −0.358 | −2.400 |
| 205217_at | TIMM8A | chrXq22.1 | 0.774 | −0.152 | −2.399 |
| 206144_at | MAGI1 | chr3p14.1 | 0.899 | 0.275 | −2.398 |
| 218359_at | NRSN2 | chr20p13 | 0.750 | −0.206 | −2.397 |
| 211566_x_at | BRE | chr2p23.2 | 0.756 | −0.194 | −2.397 |
| 200807_s_at | HSPD1 | chr2q33.1 | 0.807 | −0.063 | −2.396 |
| 214792_x_at | VAMP2 | chr17p13.1 | 0.793 | −0.101 | −2.396 |
| 200894_s_at | FKBP4 | chr12p13.33 | 0.838 | 0.032 | −2.394 |
| 209345_s_at | PI4K2A | chr10q24 | 0.876 | 0.177 | −2.394 |
| 219043_s_at | LOC285359 /// | chr2q11.2 /// | 0.910 | 0.333 | −2.392 |
| | PDCL3 | chr3q12.3 | | | |
| 215976_at | — | — | 0.713 | −0.279 | −2.391 |
| 219856_at | C1orf116 | chr1q32.1 | 0.706 | −0.292 | −2.391 |
| 201822_at | TIMM17A | chr1q32.1 | 0.727 | −0.251 | −2.389 |
| 221066_at | RXFP3 | chr5p15.1-p14 | 0.867 | 0.141 | −2.388 |
| 210868_s_at | ELOVL6 | chr4q25 | 0.720 | −0.262 | −2.384 |
| 200691_s_at | HSPA9 | chr5q31.1 | 0.818 | −0.026 | −2.383 |
| 207087_x_at | ANK1 | chr8p11.1 | 0.938 | 0.500 | −2.380 |
| 215568_x_at | [symbol] 03956 /// | [symbol] 13.2 /// | 0.882 | 0.206 | −2.380 |
| | LYPLA2 /// | chr1p36.12-p35.1 /// | | | |
| | L [symbol] | chr6 [symbol] | | | |
| 211123_at | SLC5A5 | chr19p13.2-p12 | 0.610 | −0.435 | −2.380 |
| 205937_at | CGREF1 | chr2p23.3 | 0.827 | 0.004 | −2.379 |
| 221657_s_at | ASB6 | | 0.866 | 0.144 | −2.375 |
| 210963_s_at | GYG2 | chrXp22.3 | 0.814 | −0.032 | −2.374 |
| 213132_s_at | MCAT | chr22q13.31 | 0.856 | 0.106 | −2.374 |
| 211779_x_at | AP2A2 | chr11p15.5 | 0.835 | 0.032 | −2.373 |
| 212361_s_at | ATP2A2 | chr12q23-q24.1 | 0.793 | −0.092 | −2.373 |
| 203527_s_at | APC | chr5q21-q22 | 0.882 | 0.213 | −2.372 |
| 205810_s_at | WASL | chr7q31.3 | 0.637 | −0.394 | −2.370 |
| 207438_s_at | SNUPN | chr15q24.2 | 0.886 | 0.230 | −2.369 |
| 222216_s_at | MRPL17 | chr11p15.5-p15.4 | 0.837 | 0.042 | −2.367 |
| 208915_s_at | GGA2 | chr16p12 | 0.819 | −0.013 | −2.366 |
| 214772_at | C11orf41 | chr11p13 | 0.825 | 0.007 | −2.363 |
| 211811_s_at | PCDHA6 | chr5q31 | 0.716 | −0.260 | −2.361 |
| 221959_at | FAM110B | chr8q12.1 | 0.823 | 0.001 | −2.360 |
| 32541_at | PPP3CC | chr8p21.3 | 0.784 | −0.107 | −2.358 |
| 201174_s_at | TERF2IP | chr16q23.1 | 0.889 | 0.250 | −2.358 |
| 40273_at | SPHK2 | chr19q13.2 | 0.809 | −0.038 | −2.357 |
| 217847_s_at | THRAP3 | chr1p34.3 | 0.762 | −0.161 | −2.357 |
| 201002_s_at | [symbol] M189-UBE2V1 /// | chr20q13.2 | 0.837 | 0.049 | −2.356 |
| | UB [symbol] | | | | |
| 210924_at | OLFM1 | chr9q34.3 | 0.756 | −0.173 | −2.355 |
| 221324_at | TAS2R1 | chr5p15 | 0.703 | −0.282 | −2.355 |
| 206355_at | GNAL | chr18p11.22-p11.21 | 0.820 | −0.005 | −2.353 |
| 203998_s_at | SYT1 | chr12cen-q21 | 0.820 | −0.005 | −2.352 |
| 200825_s_at | HYOU1 | chr11q23.1-q23.3 | 0.753 | −0.178 | −2.352 |
| 207053_at | SLC8A1 | chr2p23-p22 | 0.756 | −0.172 | −2.352 |
| 204675_at | SRD5A1 | chr5p15 | 0.844 | 0.075 | −2.352 |
| 212729_at | DLG3 | chrXq13.1 | 0.749 | −0.188 | −2.350 |
| 216277_at | BUB1 | chr2q14 | 0.545 | −0.499 | −2.350 |
| 208308_s_at | GPI /// | chr19q13.1 | 0.824 | 0.010 | −2.349 |
| | LOC100133951 | | | | |
| 212971_at | CARS | chr11p15.5 | 0.831 | 0.031 | −2.349 |
| 202504_at | TRIM29 | chr11q22-q23 | 0.594 | −0.443 | −2.349 |
| 220323_at | CNTD2 | chr19q13.2 | 0.650 | −0.366 | −2.348 |
| 202540_s_at | HMGCR | chr5q13.3-q14 | 0.856 | 0.119 | −2.345 |
| 213222_at | PLCB1 | chr20p12 | 0.678 | −0.321 | −2.344 |
| 201760_s_at | WSB2 | chr12q24.23 | 0.897 | 0.293 | −2.344 |
| 204540_at | EEF1A2 | chr20q13.3 | 0.845 | 0.083 | −2.340 |
| 210525_x_at | C14orf143 | chr14q32.11 | 0.634 | −0.385 | −2.339 |
| 206610_s_at | F11 | chr4q35 | 0.761 | −0.153 | −2.336 |
| 213324_at | SRC | chr20q12-q13 | 0.850 | 0.103 | −2.336 |
| 59437_at | C9orf116 | chr9q34.3 | 0.833 | 0.045 | −2.333 |
| 206859_s_at | PAEP | chr9q34 | 0.750 | −0.176 | −2.331 |
| 212877_at | KLC1 | chr14q32.3 | 0.855 | 0.124 | −2.331 |
| 202349_at | TOR1A | chr9q34 | 0.829 | 0.036 | −2.328 |
| 214819_at | IQSEC2 | chrXp11.22 | 0.524 | −0.513 | −2.327 |
| 55093_at | CSGLCA-T | chr7q36.1 | 0.813 | −0.012 | −2.326 |
| 211249_at | GPR68 | chr14q31 | 0.687 | −0.297 | −2.324 |
| 205271_s_at | CCRK | chr9q22.1 | 0.804 | −0.037 | −2.324 |
| 204565_at | ACOT13 | chr6p22.2 | 0.839 | 0.071 | −2.324 |
| 209973_at | NFKBIL1 | chr6p21.3 | 0.776 | −0.111 | −2.324 |
| 200815_s_at | PAFAH1B1 | chr17p13.3 | 0.747 | −0.178 | −2.322 |
| 33767_at | NEFH | chr22q12.2 | 0.849 | 0.107 | −2.321 |
| 204256_at | ELOVL6 | chr4q25 | 0.698 | −0.273 | −2.319 |
| 211253_x_at | PYY | chr17q21.1 | 0.589 | −0.437 | −2.319 |
| 220878_at | — | — | 0.678 | −0.309 | −2.317 |
| 219222_at | RBKS | chr2p23.3 | 0.885 | 0.251 | −2.317 |
| 220260_at | TBC1D19 | chr4p15.2 | 0.828 | 0.037 | −2.316 |
| 219486_at | DUS2L | chr16q22.1 | 0.746 | −0.176 | −2.314 |
| 209990_s_at | GABBR2 | chr9q22.1-q22.3 | 0.818 | 0.009 | −2.314 |
| 213967_at | RALYL | chr8q21.2 | 0.907 | 0.352 | −2.313 |
| 211433_x_at | KIAA1539 | chr9p13.3 | 0.635 | −0.373 | −2.312 |
| 212148_at | PBX1 | chr1q23 | 0.921 | 0.424 | −2.312 |
| 201848_s_at | BNIP3 | chr10q26.3 | 0.775 | −0.106 | −2.311 |
| 202777_at | SHOC2 | chr10q25 | 0.899 | 0.317 | −2.308 |
| 204466_s_at | SNCA | chr4q21 | 0.948 | 0.586 | −2.308 |
| 205543_at | HSPA4L | chr4q28 | 0.840 | 0.084 | −2.306 |
| 215458_s_at | SMURF1 | chr7q22.1 | 0.811 | −0.007 | −2.306 |
| 206836_at | SLC6A3 | chr5p15.3 | 0.914 | 0.389 | −2.306 |
| 207239_s_at | PCTK1 | chrXp11.3-p11.23 | 0.671 | −0.314 | −2.306 |
| 210135_s_at | SHOX2 | chr3q25-q26.1 | 0.535 | −0.493 | −2.305 |
| 205839_s_at | BZRAP1 | chr17q22-q23 | 0.838 | 0.078 | −2.302 |
| 220091_at | SLC2A6 | chr9q34 | 0.878 | 0.227 | −2.302 |
| 202260_s_at | STXBP1 | chr9q34.1 | 0.845 | 0.100 | −2.302 |
| 207000_s_at | PPP3CC | chr8p21.3 | 0.830 | 0.053 | −2.301 |
| 217887_s_at | EPS15 | chr1p32 | 0.714 | −0.236 | −2.300 |
| 211622_s_at | ARF3 | chr12q13 | 0.880 | 0.237 | −2.300 |
| 213997_at | KIAA0574 | chr15q12 | 0.790 | −0.064 | −2.299 |
| 211203_s_at | CNTN1 | chr12q11-q12 | 0.835 | 0.071 | −2.298 |
| 218623_at | HMP19 | chr5q35.2 | 0.855 | 0.138 | −2.297 |
| 203231_s_at | ATXN1 | chr6p23 | 0.752 | −0.155 | −2.296 |
| 208850_s_at | THY1 | chr11q22.3-q23 | 0.827 | 0.047 | −2.295 |
| 214762_at | ATP6V1G2 | chr6p21.3 | 0.830 | 0.055 | −2.295 |
| 212151_at | PBX1 | chr1q23 | 0.935 | 0.512 | −2.295 |
| 214270_s_at | MAPRE3 | chr2p23.3-p23.1 | 0.845 | 0.105 | −2.295 |
| 201411_s_at | PLEKHB2 | chr2q21.1 | 0.767 | −0.119 | −2.295 |
| 200640_at | YWHAZ | chr8q23.1 | 0.790 | −0.060 | −2.294 |
| 205822_s_at | HMGCS1 | chr5p14-p13 | 0.817 | 0.016 | −2.293 |
| 217711_at | TEK | chr9p21 | 0.587 | −0.428 | −2.292 |
| 214077_x_at | MEIS3P1 | chr17p12 | 0.885 | 0.261 | −2.292 |
| 209627_s_at | OSBPL3 | chr7p15 | 0.873 | 0.209 | −2.292 |
| 204650_s_at | APBB3 | chr5q31 | 0.755 | −0.145 | −2.291 |
| 211167_s_at | GCK | chr7p15.3-p15.1 | 0.782 | −0.079 | −2.290 |
| 202912_at | ADM | chr11p15.4 | 0.760 | −0.134 | −2.290 |
| 207452_s_at | CNTN5 | chr11q21-q22.2 | 0.708 | −0.243 | −2.290 |
| 213550_s_at | TMCO6 | chr5q31.3 | 0.825 | 0.043 | −2.289 |
| 208851_s_at | THY1 | chr11q22.3-q23 | 0.860 | 0.165 | −2.286 |
| 201089_at | ATP6V1B2 | chr8p22-p21 | 0.842 | 0.099 | −2.286 |
| 221371_at | TNFSF18 | chr1q23 | 0.809 | −0.004 | −2.286 |
| 219400_at | CNTNAP1 | chr17q21 | 0.861 | 0.169 | −2.285 |
| 214272_at | CYLD | chr16q12.1 | 0.712 | −0.231 | −2.282 |
| 217574_at | CDH8 | chr16q22.1 | 0.763 | −0.123 | −2.282 |
| 201889_at | FAM3C | chr7q31 | 0.825 | 0.046 | −2.282 |
| 217457_s_at | RAP1GDS1 | chr4q23-q25 | 0.769 | −0.109 | −2.282 |
| 208353_x_at | ANK1 | chr8p11.1 | 0.928 | 0.477 | −2.282 |
| 65521_at | UBE2D4 | chr7p13 | 0.735 | −0.185 | −2.281 |
| 205691_at | SYNGR3 | chr16p13 | 0.861 | 0.169 | −2.281 |
| 222132_s_at | AGK | chr7q34 | 0.844 | 0.110 | −2.280 |
| 215407_s_at | ASTN2 | chr9q33.1 | 0.797 | −0.036 | −2.280 |
| 204723_at | SCN3B | chr11q23.3 | 0.847 | 0.123 | −2.275 |
| 205257_s_at | AMPH | chr7p14-p13 | 0.865 | 0.188 | −2.275 |
| 213374_x_at | HIBCH | chr2q32.2 | 0.865 | 0.188 | −2.275 |
| 202517_at | CRMP1 | chr4p16.1-p15 | 0.775 | −0.090 | −2.274 |
| 209372_x_at | TUBB2A /// | chr6p25 | 0.673 | −0.296 | −2.273 |
| | TUBB2B | | | | |
| 202854_at | HPRT1 | chrXq26.1 | 0.857 | 0.160 | −2.273 |
| 206751_s_at | PCYT1B | chrXp22.11 | 0.748 | −0.151 | −2.272 |
| 200695_at | PPP2R1A | chr19q13.33 | 0.744 | −0.159 | −2.270 |
| 209029_at | COPS7A | chr12p13.31 | 0.782 | −0.069 | −2.270 |
| 202158_s_at | CUGBP2 | chr10p13 | 0.739 | −0.170 | −2.269 |
| 204527_at | MYO5A | chr15q21 | 0.811 | 0.010 | −2.268 |
| 220105_at | RTDR1 | chr22q11.2 | 0.606 | −0.394 | −2.266 |
| 205638_at | BAI3 | chr6q12 | 0.839 | 0.100 | −2.265 |
| 202582_s_at | RANBP9 | chr6p23 | 0.909 | 0.384 | −2.265 |
| 209014_at | MAGED1 | chrXp11.23 | 0.836 | 0.090 | −2.264 |
| 201266_at | TXNRD1 | chr12q23-q24.1 | 0.726 | −0.194 | −2.264 |
| 205827_at | CCK | chr3p22-p21.3 | 0.747 | −0.150 | −2.264 |
| 201972_at | ATP6V1A | chr3q13.2-q13.31 | 0.852 | 0.148 | −2.262 |
| 212358_at | CLIP3 | chr19q13.12 | 0.792 | −0.039 | −2.261 |
| 205735_s_at | AFF3 | chr2q11.2-q12 | 0.736 | −0.173 | −2.261 |
| 218434_s_at | AACS | chr12q24.31 | 0.834 | 0.086 | −2.256 |
| 212094_at | PEG10 | chr7q21 | 0.937 | 0.536 | −2.256 |
| 209249_s_at | GHITM | chr10q23.1 | 0.830 | 0.075 | −2.256 |
| 210628_x_at | LTBP4 | chr19q13.1-q13.2 | 0.584 | −0.416 | −2.254 |
| 218955_at | BRF2 | chr8p12 | 0.866 | 0.200 | −2.254 |
| 206876_at | SIM1 | chr6q16.3-q21 | 0.661 | −0.307 | −2.254 |
| 221262_s_at | SLC2A11 | chr22q11.2 | 0.702 | −0.236 | −2.253 |
| 220479_at | LOC29034 | chr2q34 | 0.691 | −0.256 | −2.253 |
| 214079_at | DHRS2 | chr14q11.2 | 0.648 | −0.327 | −2.253 |
| 213617_s_at | C18orf10 | chr18q12.2 | 0.818 | 0.039 | −2.252 |
| 205004_at | NKRF | chrXq24 | 0.789 | −0.042 | −2.251 |
| 208737_at | ATP6V1G1 | chr9q32 | 0.830 | 0.077 | −2.249 |
| 206528_at | TRPC6 | chr11q21-q22 | 0.625 | −0.359 | −2.248 |
| 220841_s_at | AHI1 | chr6q23.3 | 0.748 | −0.140 | −2.248 |
| 204486_at | — | — | 0.666 | −0.296 | −2.247 |
| 201453_x_at | RHEB | chr7q36 | 0.846 | 0.133 | −2.246 |
| 218560_s_at | JMJD4 | chr1q42.13 | 0.735 | −0.167 | −2.246 |
| 212137_at | LARP1 | chr5q33.2 | 0.898 | 0.339 | −2.246 |
| 201678_s_at | C3orf37 | chr3q21.3 | 0.887 | 0.291 | −2.245 |
| 211609_x_at | PSMD4 | chr1q21.2 | 0.825 | 0.063 | −2.244 |
| 202965_s_at | CAPN6 | chrXq23 | 0.618 | −0.369 | −2.244 |
| 211156_at | CDKN2A | chr9p21 | 0.764 | −0.101 | −2.243 |
| 212880_at | WDR7 | chr18q21.1-q22 | 0.825 | 0.067 | −2.242 |
| 200982_s_at | ANXA6 | chr5q32-q34 | 0.788 | −0.039 | −2.241 |
| 205864_at | SLC7A4 | chr22q11.21 | 0.786 | −0.045 | −2.240 |
| 210966_x_at | LARP1 | chr5q33.2 | 0.855 | 0.169 | −2.240 |
| 214164_x_at | CA12 | chr15q22 | 0.551 | −0.450 | −2.239 |
| 215167_at | MED14 | chrXp11.4-p11.2 | 0.646 | −0.323 | −2.236 |
| 211153_s_at | TNFSF11 | chr13q14 | 0.683 | −0.263 | −2.236 |
| 201849_at | BNIP3 | chr10q26.3 | 0.691 | −0.248 | −2.235 |
| 201557_at | VAMP2 | chr17p13.1 | 0.848 | 0.145 | −2.234 |
| 213869_x_at | THY1 | chr11q22.3-q23 | 0.847 | 0.142 | −2.234 |
| 212353_at | SULF1 | chr8q13.2-q13.3 | 0.652 | −0.312 | −2.231 |
| 221903_s_at | CYLD | chr16q12.1 | 0.757 | −0.111 | −2.230 |
| 216236_s_at | SLC2A14 /// | chr12p13.3 /// | 0.807 | 0.018 | −2.230 |
| | SLC2A3 | chr12p13.31 | | | |
| 207059_at | PAX9 | chr14q12-q13 | 0.732 | −0.166 | −2.230 |
| 213439_x_at | RUNDC3A | chr17q21.31 | 0.890 | 0.309 | −2.229 |
| 212159_x_at | AP2A2 | chr11p15.5 | 0.847 | 0.147 | −2.226 |
| 204117_at | PREP | chr6q22 | 0.768 | −0.084 | −2.226 |
| 209587_at | PITX1 | chr5q31 | 0.645 | −0.320 | −2.225 |
| 200863_s_at | RAB11A | chr15q21.3-q22.31 | 0.844 | 0.137 | −2.225 |
| 214976_at | RPL13 | chr16q24.3\|17p11.2 | 0.622 | −0.354 | −2.225 |
| 201313_at | ENO2 | chr12p13 | 0.809 | 0.027 | −2.224 |
| 200740_s_at | SUMO3 | chr21q22.3 | 0.865 | 0.212 | −2.224 |
| 203179_at | GALT | chr9p13 | 0.858 | 0.185 | −2.223 |
| 204554_at | PPP1R3D | chr20q13.3 | 0.907 | 0.393 | −2.223 |
| 210517_s_at | AKAP12 | chr6q24-q25 | 0.867 | 0.220 | −2.222 |
| 206356_s_at | GNAL | chr18p11.22-p11.21 | 0.844 | 0.137 | −2.222 |
| 218137_s_at | SMAP1 | chr6q13 | 0.833 | 0.101 | −2.221 |
| 56829_at | TRAPPC9 | chr8q24.3 | 0.730 | −0.165 | −2.221 |
| 215634_at | — | — | 0.828 | 0.085 | −2.220 |
| 204146_at | RAD51AP1 | chr12p13.2-p13.1 | 0.809 | 0.029 | −2.219 |
| 205359_at | AKAP6 | chr14q13.1 | 0.818 | 0.056 | −2.219 |
| 217655_at | LOC100127972 | chr19q13.12 | 0.544 | −0.450 | −2.219 |
| 204412_s_at | NEFH | chr22q12.2 | 0.834 | 0.107 | −2.216 |
| 209537_at | EXTL2 | chr1p21 | 0.853 | 0.170 | −2.216 |
| 222021_x_at | SDHALP1 | chr3q29 | 0.768 | −0.078 | −2.215 |
| 221063_x_at | RNF123 | chr3p24.3 | 0.743 | −0.136 | −2.215 |
| 217785_s_at | YKT6 | chr7p15.1 | 0.735 | −0.152 | −2.214 |
| 207789_s_at | DPP6 | chr7q36.2 | 0.845 | 0.146 | −2.214 |
| 203817_at | GUCY1B3 | chr4q31.3-q33 | 0.825 | 0.079 | −2.214 |
| 205030_at | FABP7 | chr6q22-q23 | 0.918 | 0.447 | −2.214 |
| 219350_s_at | DIABLO | chr12q24.31 | 0.800 | 0.008 | −2.213 |
| 209720_s_at | SERPINB3 | chr18q21.3 | 0.665 | −0.283 | −2.212 |
| 216316_x_at | GK /// | chr4q32.1 /// | 0.619 | −0.352 | −2.212 |
| | GK3P | chrXp21.3 | | | |
| 49077_at | PPME1 | chr11q13.4 | 0.836 | 0.116 | −2.211 |
| 210510_s_at | NRP1 | chr10p12 | 0.549 | −0.441 | −2.208 |
| 217186_at | ZNF259P | chr6q21 | 0.776 | −0.053 | −2.206 |
| 217093_at | RNASE1 | chr14q11.2 | 0.821 | 0.072 | −2.206 |
| 202196_s_at | DKK3 | chr11p15.2 | 0.827 | 0.090 | −2.206 |
| 216938_x_at | DRD2 | chr11q23 | 0.798 | 0.005 | −2.204 |
| 217606_at | — | — | 0.817 | 0.060 | −2.204 |
| 205773_at | CPEB3 | chr10q23.32 | 0.946 | 0.610 | −2.203 |
| 204970_s_at | MAFG | chr17q25.3 | 0.738 | −0.139 | −2.203 |
| 39582_at | CYLD | chr16q12.1 | 0.779 | −0.043 | −2.201 |
| 203931_s_at | MRPL12 | chr17q25 | 0.797 | 0.005 | −2.201 |
| 209772_s_at | CD24 | chr6q21 | 0.536 | −0.452 | −2.201 |
| 221029_s_at | WNT5B | chr12p13.3 | 0.605 | −0.368 | −2.200 |
| 222125_s_at | P4HTM | chr3p21.31 | 0.734 | −0.147 | −2.200 |
| 209206_at | SEC22B | chr1q21.1 | 0.703 | −0.210 | −2.200 |
| 205673_s_at | ASB9 | — | 0.516 | −0.474 | −2.200 |
| 209211_at | KLF5 | chr13q22.1 | 0.842 | 0.142 | −2.199 |
| 219060_at | WDYHV1 | chr8q24.13 | 0.744 | −0.125 | −2.199 |
| 206347_at | PDK3 | chrXp22.11 | 0.764 | −0.079 | −2.198 |
| 216477_at | — | — | 0.772 | −0.060 | −2.198 |
| 204471_at | GAP43 | chr3q13.1-q13.2 | 0.828 | 0.098 | −2.196 |
| 210465_s_at | SNAPC3 | chr9p22.3 | 0.736 | −0.141 | −2.195 |
| 214230_at | CDC42 | chr1p36.1 | 0.824 | 0.085 | −2.194 |
| 203820_s_at | IGF2BP3 | chr7p11 | 0.722 | −0.169 | −2.194 |
| 218111_s_at | CMAS | chr12p12.1 | 0.867 | 0.234 | −2.194 |
| 220538_at | ADM2 | chr22q13.33 | 0.640 | −0.313 | −2.193 |
| 206290_s_at | RGS7 | chr1q43\|1q23.1 | 0.840 | 0.140 | −2.190 |
| 218412_s_at | GTF2IRD1 | chr7q11.23 | 0.708 | −0.194 | −2.188 |
| 204513_s_at | ELMO1 | chr7p14.2 | 0.812 | 0.053 | −2.188 |
| 206976_s_at | HSPH1 | chr13q12.3 | 0.724 | −0.163 | −2.187 |
| 200820_at | PSMD8 | chr19q13.2 | 0.802 | 0.025 | −2.187 |
| 207594_s_at | SYNJ1 | chr21q22.2 | 0.788 | −0.011 | −2.185 |
| 204364_s_at | REEP1 | chr2p11.2 | 0.860 | 0.211 | −2.184 |
| 207210_at | GABRA3 | chrXq28 | 0.753 | −0.096 | −2.183 |
| 204629_at | PARVB | chr22q13.2-q13.33 | 0.769 | −0.060 | −2.183 |
| 200822_x_at | TPI1 | chr12p13 | 0.749 | −0.107 | −2.182 |
| 205389_s_at | ANK1 | chr8p11.1 | 0.921 | 0.474 | −2.182 |
| 221458_at | HTR1F | chr3p12 | 0.621 | −0.336 | −2.182 |
| 220896_at | FBXL18 | chr7p22.2 | 0.807 | 0.043 | −2.182 |
| 204352_at | TRAF5 | chr1q32 | 0.669 | −0.261 | −2.180 |
| 218568_at | AGK | chr7q34 | 0.801 | 0.025 | −2.179 |
| 205795_at | NRXN3 | chr14q31 | 0.849 | 0.175 | −2.177 |
| 203538_at | CAMLG | chr5q23 | 0.899 | 0.374 | −2.176 |
| 213486_at | COPG2IT1 | chr7q32 | 0.851 | 0.185 | −2.176 |
| 201543_s_at | SAR1A | chr10q22.1 | 0.652 | −0.287 | −2.175 |
| 218482_at | ENY2 | chr8q23.1 | 0.833 | 0.125 | −2.174 |
| 203895_at | PLCB4 | chr20p12 | 0.845 | 0.164 | −2.173 |
| 208524_at | GPR15 | chr3q11.2-q13.1 | 0.551 | −0.424 | −2.173 |
| 215108_x_at | TOX3 | chr16q12.1 | 0.775 | −0.040 | −2.172 |
| 217004_s_at | MCF2 | chrXq27 | 0.758 | −0.081 | −2.172 |
| 205567_at | CHST1 | chr11p11.2-p11.1 | 0.767 | −0.060 | −2.172 |
| 207387_s_at | GK | chrXp21.3 | 0.697 | −0.206 | −2.171 |
| 201658_at | ARL1 | chr12q23.2 | 0.726 | −0.151 | −2.171 |
| 213011_s_at | TPI1 | chr12p13 | 0.737 | −0.127 | −2.171 |
| 202849_x_at | GRK6 | chr5q35 | 0.541 | −0.435 | −2.171 |
| 200641_s_at | YWHAZ | chr8q23.1 | 0.742 | −0.116 | −2.170 |
| 213433_at | ARL3 | chr10q23.3 | 0.765 | −0.062 | −2.169 |
| 212471_at | AVL9 | chr7p14.3 | 0.709 | −0.183 | −2.167 |
| 200639_s_at | YWHAZ | chr8q23.1 | 0.695 | −0.209 | −2.166 |
| 201415_at | GSS | chr20q11.2 | 0.805 | 0.044 | −2.166 |
| 219100_at | OBFC1 | chr10q24.33 | 0.699 | −0.202 | −2.166 |
| 212372_at | MYH10 | chr17p13 | 0.812 | 0.064 | −2.165 |
| 218456_at | CAPRIN2 | chr12p11 | 0.879 | 0.293 | −2.164 |
| 203797_at | VSNL1 | chr2p24.3 | 0.815 | 0.075 | −2.162 |
| 217305_s_at | ADCY10 | chr1q24 | 0.603 | −0.353 | −2.162 |
| 222261_at | KIAA1609 | chr16q24.1 | 0.519 | −0.456 | −2.161 |
| 205852_at | CDK5R2 | chr2q35 | 0.824 | 0.103 | −2.161 |
| 218100_s_at | IFT57 | chr3q13.12 | 0.741 | −0.113 | −2.161 |
| 209384_at | PROSC | chr8p11.2 | 0.890 | 0.342 | −2.160 |
| 208352_x_at | ANK1 | chr8p11.1 | 0.930 | 0.532 | −2.158 |
| 220416_at | ATP8B4 | chr15q21.2 | 0.614 | −0.336 | −2.158 |
| 203159_at | GLS | chr2q32-q34 | 0.853 | 0.201 | −2.157 |
| 214098_at | KIAA1107 | chr1p22.1 | 0.776 | −0.029 | −2.157 |
| 209755_at | NMNAT2 | chr1q25 | 0.864 | 0.241 | −2.154 |
| 218737_at | SBNO1 | chr12q24.31 | 0.853 | 0.201 | −2.154 |
| 203446_s_at | OCRL | chrXq25-q26.1 | 0.852 | 0.199 | −2.153 |
| 215101_s_at | CXCL5 | chr4q12-q13 | 0.699 | −0.195 | −2.152 |
| 216623_x_at | TOX3 | chr16q12.1 | 0.777 | −0.024 | −2.150 |
| 219508_at | GCNT3 | chr15q21.3 | 0.679 | −0.230 | −2.150 |
| 213066_at | RUSC2 | chr9p13.3 | 0.749 | −0.089 | −2.150 |
| 208276_at | — | — | 0.729 | −0.134 | −2.149 |
| 206812_at | ADRB3 | chr8p12-p11.2 | 0.752 | −0.083 | −2.148 |
| 205850_s_at | GABRB3 | chr15q11.2-q12 | 0.779 | −0.018 | −2.148 |
| 212497_at | MAPK1IP1L | chr14q22.3 | 0.545 | −0.420 | −2.148 |
| 218106_s_at | MRPS10 | chr6p21.1 | 0.826 | 0.116 | −2.147 |
| 200638_s_at | YWHAZ | chr8q23.1 | 0.794 | 0.023 | −2.147 |
| 208702_x_at | APLP2 | chr11q23-q25\|11q24 | 0.807 | 0.060 | −2.147 |
| 214745_at | PLCH1 | chr3q25.31 | 0.907 | 0.423 | −2.147 |
| 212554_at | CAP2 | chr6p22.3 | 0.811 | 0.072 | −2.146 |
| 211679_x_at | GABBR2 | chr9q22.1-q22.3 | 0.788 | 0.006 | −2.145 |
| 214757_at | PMS2L2 | chr7q11-q22 | 0.750 | −0.085 | −2.144 |
| 201122_x_at | EIF5A | chr17p13-p12 | 0.635 | −0.298 | −2.144 |
| 35666_at | SEMA3F | chr3p21.3 | 0.630 | −0.307 | −2.144 |
| 211320_s_at | PTPRU | chr1p35.3-p35.1 | 0.728 | −0.133 | −2.144 |
| 206044_s_at | BRAF | chr7q34 | 0.799 | 0.039 | −2.143 |
| 203410_at | AP3M2 | chr8p11.2 | 0.754 | −0.076 | −2.143 |
| 203368_at | CRELD1 | chr3p25.3 | 0.655 | −0.266 | −2.143 |
| 207184_at | SLC6A13 | chr12p13.3 | 0.531 | −0.434 | −2.142 |
| 221567_at | NOL3 | chr16q21-q23 | 0.835 | 0.144 | −2.142 |
| 204375_at | CLSTN3 | chr12p13.31 | 0.849 | 0.194 | −2.142 |
| 209859_at | TRIM9 | chr14q22.1 | 0.736 | −0.115 | −2.141 |
| 210616_s_at | SEC31A | chr4q21.22 | 0.692 | −0.202 | −2.139 |
| 204044_at | QPRT | chr16p11.2 | 0.829 | 0.130 | −2.139 |
| 203172_at | FXR2 | chr17p13.1 | 0.848 | 0.192 | −2.138 |
| 220615_s_at | FAR2 | chr12p11.22 | 0.776 | −0.020 | −2.138 |
| 221859_at | SYT13 | chr11p12-p11 | 0.755 | −0.071 | −2.137 |
| 212798_s_at | ANKMY2 | chr7p21 | 0.835 | 0.148 | −2.137 |
| 212009_s_at | STIP1 | chr11q13 | 0.707 | −0.171 | −2.137 |
| 219591_at | CEND1 | chr11p15.5 | 0.818 | 0.097 | −2.136 |
| 211207_s_at | ACSL6 | chr5q31 | 0.768 | −0.038 | −2.136 |
| 214803_at | — | — | 0.725 | −0.134 | −2.135 |
| 218393_s_at | SMU1 | chr9p12 | 0.776 | −0.018 | −2.133 |
| 214999_s_at | RAB11FIP3 | chr16p13.3 | 0.790 | 0.020 | −2.133 |
| 205792_at | WISP2 | chr20q12-q13.1 | 0.694 | −0.194 | −2.130 |
| 221393_at | TAAR3 | chr6q23-q24 | 0.780 | −0.005 | −2.130 |
| 205539_at | AVIL | chr12q14.1 | 0.691 | −0.199 | −2.129 |
| 201403_s_at | MGST3 | chr1q23 | 0.792 | 0.027 | −2.129 |
| 210050_at | TPI1 | chr12p13 | 0.833 | 0.147 | −2.128 |
| 212990_at | SYNJ1 | chr21q22.2 | 0.857 | 0.228 | −2.128 |
| 216190_x_at | ITGB1 | chr10p11.2 | 0.707 | −0.167 | −2.127 |
| 206217_at | EDA | chrXq12-q13.1 | 0.656 | −0.257 | −2.127 |
| 211586_s_at | [symbol] P2B2 /// | chr3p25.3 | 0.730 | −0.118 | −2.124 |
| | LOC1001342 [symbol] | | | | |
| 206031_s_at | USP5 | chr12p13 | 0.688 | −0.201 | −2.123 |
| 209991_x_at | GABBR2 | chr9q22.1-q22.3 | 0.768 | −0.033 | −2.122 |
| 213912_at | FLJ41278 /// | chr12q14.3 | 0.828 | 0.134 | −2.121 |
| | TBC1D30 | | | | |
| 203157_s_at | GLS | chr2q32-q34 | 0.837 | 0.162 | −2.121 |
| 204106_at | TESK1 | chr9p13 | 0.791 | 0.027 | −2.120 |
| 211761_s_at | CACYBP | chr1q24-q25 | 0.864 | 0.256 | −2.120 |
| 221048_x_at | C17orf80 | chr17q25.1 | 0.809 | 0.078 | −2.119 |
| 209767_s_at | GP1BB /// | [symbol] 11.21 /// | 0.759 | −0.052 | −2.117 |
| | SEPT5 | chr22q11.21- | | | |
| | | q11.23\|22 [symbol] | | | |
| 222175_s_at | MED15 | chr22q11.2 | 0.659 | −0.248 | −2.117 |
| 212565_at | STK38L | chr12p11.23 | 0.860 | 0.245 | −2.116 |
| 221345_at | FFAR2 | chr19q13.1 | 0.603 | −0.333 | −2.115 |
| 201523_x_at | UBE2N | chr12q22 | 0.787 | 0.020 | −2.115 |
| 214607_at | PAK3 | chrXq22.3 | 0.783 | 0.009 | −2.115 |
| 210305_at | PDE4DIP | chr1q12 | 0.742 | −0.088 | −2.112 |
| 204611_s_at | PPP2R5B | chr11q12-q13 | 0.804 | 0.067 | −2.112 |
| 203773_x_at | BLVRA | chr7p14-cen | 0.864 | 0.261 | −2.112 |
| 201193_at | IDH1 | chr2q33.3 | 0.823 | 0.123 | −2.112 |
| 217339_x_at | CTAG1A /// | chrXq28 | 0.557 | −0.391 | −2.111 |
| | CTAG1B | | | | |
| 220830_at | IMPG2 | chr3q12.2-q12.3 | 0.726 | −0.121 | −2.110 |
| 216948_at | — | — | 0.682 | −0.204 | −2.109 |
| 209658_at | CDC16 | chr13q34 | 0.752 | −0.062 | −2.108 |
| 220405_at | [symbol] C100127998 /// | chr8q11-q12 /// | 0.813 | 0.097 | −2.106 |
| | SNT [symbol] | chr8q11.22 | | | |
| 213531_s_at | RAB3GAP1 | chr2q21.3 | 0.872 | 0.293 | −2.106 |
| 205204_at | NMB | chr15q22-qter | 0.810 | 0.088 | −2.105 |
| 215910_s_at | FNDC3A | chr13q14.2 | 0.523 | −0.429 | −2.105 |
| 218680_x_at | HYPK | chr15q15.3 | 0.843 | 0.189 | −2.105 |
| 203158_s_at | GLS | chr2q32-q34 | 0.796 | 0.049 | −2.105 |
| 212149_at | EFR3A | chr8q24.22 | 0.837 | 0.172 | −2.104 |
| 39705_at | SIN3B | chr19p13.11 | 0.854 | 0.229 | −2.104 |
| 215670_s_at | SCAND2 | chr15q25-q26 | 0.682 | −0.203 | −2.104 |
| 211985_s_at | CALM1 | chr14q24-q31 | 0.749 | −0.067 | −2.102 |
| 202836_s_at | TXNL4A | chr18q23 | 0.786 | 0.024 | −2.102 |
| 200987_x_at | PSME3 | chr17q21 | 0.878 | 0.319 | −2.101 |
| 219688_at | BBS7 | chr4q27 | 0.757 | −0.047 | −2.099 |
| 215785_s_at | CYFIP2 | chr5q33.3 | 0.754 | −0.053 | −2.099 |
| 213927_at | MAP3K9 | chr14q24.3-q31 | 0.833 | 0.160 | −2.099 |
| 210143_at | ANXA10 | chr4q33 | 0.569 | −0.371 | −2.098 |
| 212333_at | FAM98A | chr2p22.3 | 0.765 | −0.027 | −2.098 |
| 204076_at | ENTPD4 | chr8p21.3 | 0.839 | 0.182 | −2.098 |
| 208075_s_at | CCL7 | chr17q11.2-q12 | 0.653 | −0.249 | −2.097 |
| 212362_at | ATP2A2 | chr12q23-q24.1 | 0.600 | −0.329 | −2.095 |
| 214376_at | — | — | 0.834 | 0.167 | −2.094 |
| 207816_at | LALBA | chr12q13 | 0.537 | −0.408 | −2.093 |
| 205391_x_at | ANK1 | chr8p11.1 | 0.924 | 0.525 | −2.092 |
| 221597_s_at | TMEM208 | chr16q22.1 | 0.827 | 0.144 | −2.092 |
| 213875_x_at | C6orf62 | chr6p22.2 | 0.672 | −0.215 | −2.092 |
| 211195_s_at | TP63 | chr3q28 | 0.772 | −0.006 | −2.092 |
| 216913_s_at | RRP12 | chr10q24.1 | 0.712 | −0.141 | −2.092 |
| 202394_s_at | ABCF3 | chr3q27.1 | 0.814 | 0.106 | −2.091 |
| 208709_s_at | NRD1 | chr1p32.2-p32.1 | 0.851 | 0.222 | −2.091 |
| 201994_at | MORF4L2 | chrXq22 | 0.726 | −0.110 | −2.090 |
| 204480_s_at | C9orf16 | chr9q34.1 | 0.804 | 0.079 | −2.089 |
| 203589_s_at | TFDP2 | chr3q23 | 0.806 | 0.084 | −2.089 |
| 210976_s_at | PFKM | chr12q13.3 | 0.821 | 0.128 | −2.087 |
| 214447_at | ETS1 | chr11q23.3 | 0.649 | −0.251 | −2.087 |
| 222088_s_at | SLC2A14 /// | chr12p13.3 /// | 0.727 | −0.106 | −2.086 |
| | SLC2A3 | chr12p13.31 | | | |
| 216330_s_at | POU6F1 | chr12q13.13 | 0.869 | 0.292 | −2.085 |
| 205556_at | MSX2 | chr5q34-q35 | 0.687 | −0.185 | −2.085 |
| 221181_at | — | — | 0.631 | −0.278 | −2.084 |
| 216489_at | TRPM3 | chr9q21.11-q21.12 | 0.693 | −0.173 | −2.084 |
| 219275_at | PDCD5 | chr19q12-q13.1 | 0.820 | 0.129 | −2.084 |
| 214268_s_at | MTMR4 | chr17q22-q23 | 0.829 | 0.155 | −2.082 |
| 219901_at | FGD6 | chr12q22 | 0.680 | −0.195 | −2.081 |
| 221214_s_at | NELF | chr9q34.3 | 0.610 | −0.308 | −2.081 |
| 220910_at | FRAS1 | chr4q21.21 | 0.520 | −0.422 | −2.080 |
| 213406_at | WSB1 | chr17q11.1 | 0.647 | −0.251 | −2.080 |
| 202033_s_at | RB1CC1 | chr8q11 | 0.809 | 0.097 | −2.079 |
| 202372_at | RAB3GAP2 | chr1q41 | 0.831 | 0.163 | −2.079 |
| 206181_at | SLAMF1 | chr1q22-q23 | 0.541 | −0.396 | −2.078 |
| 214774_x_at | TOX3 | chr16q12.1 | 0.735 | −0.087 | −2.078 |
| 201381_x_at | CACYBP | chr1q24-q25 | 0.840 | 0.193 | −2.077 |
| 213295_at | CYLD | chr16q12.1 | 0.797 | 0.066 | −2.076 |
| 203029_s_at | PTPRN2 | chr7q36 | 0.796 | 0.062 | −2.075 |
| 214975_s_at | MTMR1 | chrXq28 | 0.822 | 0.137 | −2.075 |
| 207088_s_at | SLC25A11 | chr17p13.3 | 0.717 | −0.121 | −2.075 |
| 211925_s_at | PLCB1 | chr20p12 | 0.702 | −0.151 | −2.074 |
| 217755_at | HN1 | chr17q25.1 | 0.871 | 0.306 | −2.073 |
| 202422_s_at | ACSL4 | chrXq22.3-q23 | 0.859 | 0.260 | −2.072 |
| 221693_s_at | MRPS18A | chr6p21.3 | 0.668 | −0.213 | −2.072 |
| 214342_at | ATXN7L1 | chr7q22.2 | 0.568 | −0.361 | −2.071 |
| 214383_x_at | KLHDC3 | chr6p21.1 | 0.803 | 0.085 | −2.070 |
| 201709_s_at | NIPSNAP1 | chr22q12.2 | 0.825 | 0.151 | −2.069 |
| 201434_at | TTC1 | chr5q33.3 | 0.851 | 0.236 | −2.068 |
| 202513_s_at | PPP2R5D | chr6p21.1 | 0.780 | 0.024 | −2.068 |
| 214277_at | COX11 | chr17q22 | 0.785 | 0.040 | −2.066 |
| 218550_s_at | LRRC20 | chr10q22.1 | 0.744 | −0.060 | −2.066 |
| 217129_at | — | — | 0.606 | −0.307 | −2.066 |
| 203814_s_at | NQO2 | chr6pter-q12 | 0.783 | 0.034 | −2.066 |
| 217310_s_at | FOXJ3 | chr1pter-q31.3 | 0.657 | −0.228 | −2.066 |
| 204945_at | PTPRN | chr2q35-q36.1 | 0.791 | 0.057 | −2.064 |
| 219335_at | ARMCX5 | chrXq22.1-q22.3 | 0.733 | −0.083 | −2.063 |
| 221316_at | C19orf15 | chr19q13.1 | 0.651 | −0.237 | −2.062 |
| 203333_at | KIFAP3 | chr1q24.2 | 0.825 | 0.153 | −2.062 |
| 212842_x_at | [symbol] // | chr2q12.3 /// | 0.717 | −0.116 | −2.062 |
| | RGPD5 /// | chr2q13 | | | |
| | RGPD6 // [symbol] | | | | |
| 222286_at | SNAPC3 | chr9p22.3 | 0.886 | 0.370 | −2.061 |
| 221912_s_at | CCDC28B | chr1p35.1 | 0.799 | 0.079 | −2.059 |
| 200978_at | MDH1 | chr2p13.3 | 0.784 | 0.040 | −2.059 |
| 200895_s_at | FKBP4 | chr12p13.33 | 0.754 | −0.034 | −2.058 |
| 210959_s_at | SRD5A1 | chr5p15 | 0.714 | −0.120 | −2.057 |
| 217356_s_at | PGK1 | chrXq13 | 0.755 | −0.029 | −2.055 |
| 207022_s_at | LDHC | chr11p15.5-p15.3 | 0.870 | 0.309 | −2.055 |
| 203438_at | STC2 | chr5q35.2 | 0.670 | −0.200 | −2.055 |
| 201556_s_at | VAMP2 | chr17p13.1 | 0.782 | 0.037 | −2.054 |
| 214095_at | SHMT2 | chr12q12-q14 | 0.708 | −0.130 | −2.053 |
| 204564_at | PCGF3 | chr4p16.3 | 0.880 | 0.348 | −2.050 |
| 211347_at | CDC14B | chr9q22.33 | 0.729 | −0.086 | −2.049 |
| 215608_at | — | — | 0.743 | −0.053 | −2.048 |
| 213683_at | ACSL6 | chr5q31 | 0.802 | 0.095 | −2.047 |
| 217841_s_at | PPME1 | chr11q13.4 | 0.836 | 0.194 | −2.047 |
| 203224_at | RFK | chr9q21.13 | 0.853 | 0.253 | −2.047 |
| 209003_at | SLC25A11 | chr17p13.3 | 0.726 | −0.089 | −2.046 |
| 205399_at | DCLK1 | chr13q13 | 0.848 | 0.234 | −2.046 |
| 205029_s_at | FABP7 | chr6q22-q23 | 0.906 | 0.460 | −2.046 |
| 211016_x_at | HSPA4 | chr5q31.1-q31.2 | 0.715 | −0.110 | −2.044 |
| 211547_s_at | PAFAH1B1 | chr17p13.3 | 0.716 | −0.106 | −2.040 |
| 203727_at | SKIV2L | chr6p21 | 0.772 | 0.020 | −2.040 |
| 206232_s_at | B4GALT6 | chr18q11 | 0.767 | 0.008 | −2.037 |
| 214512_s_at | SUB1 | chr5p13.3 | 0.798 | 0.089 | −2.036 |
| 217840_at | DDX41 | chr5q35.3 | 0.822 | 0.157 | −2.036 |
| 202941_at | NDUFV2 | chr18p11.31-p11.2 | 0.862 | 0.289 | −2.035 |
| 202671_s_at | PDXK | chr21q22.3 | 0.701 | −0.134 | −2.032 |
| 206436_at | MPPED1 | chr22q13.31 | 0.806 | 0.112 | −2.030 |
| 203618_at | FAIM2 | chr12q13 | 0.614 | −0.279 | −2.029 |
| 217448_s_at | LOC285412 /// | chr14q11.2 /// | 0.742 | −0.045 | −2.029 |
| | TOX4 | chr4q25 | | | |
| 210103_s_at | FOXA2 | chr20p11 | 0.720 | −0.094 | −2.029 |
| 216462_at | — | — | 0.658 | −0.209 | −2.029 |
| 208536_s_at | BCL2L11 | chr2q13 | 0.561 | −0.351 | −2.028 |
| 208122_x_at | KIR2DS3 | chr19q13.4 | 0.583 | −0.322 | −2.027 |
| 208733_at | RAB2A | chr8q12.1 | 0.748 | −0.032 | −2.027 |
| 207971_s_at | CEP68 | chr2p14 | 0.614 | −0.278 | −2.027 |
| 218163_at | MCTS1 | chrXq22-q24 | 0.785 | 0.058 | −2.026 |
| 211210_x_at | SH2D1A | chrXq25-q26 | 0.505 | −0.417 | −2.026 |
| 214444_s_at | PVR | chr19q13.2 | 0.546 | −0.369 | −2.026 |
| 211680_at | PDLIM5 | chr4q22 | 0.535 | −0.382 | −2.026 |
| 201248_s_at | SREBF2 | chr22q13 | 0.793 | 0.079 | −2.025 |
| 201772_at | AZIN1 | chr8q22.3 | 0.729 | −0.071 | −2.023 |
| 221115_s_at | LENEP | chr1q22 | 0.649 | −0.221 | −2.022 |
| 209825_s_at | UCK2 | chr1q23 | 0.760 | −0.001 | −2.022 |
| 213404_s_at | RHEB | chr7q36 | 0.804 | 0.112 | −2.020 |
| 211685_s_at | NCALD | chr8q22.2 | 0.604 | −0.289 | −2.020 |
| 211630_s_at | GSS | chr20q11.2 | 0.749 | −0.026 | −2.020 |
| 203843_at | RPS6KA3 | chrXp22.2-p22.1 | 0.878 | 0.356 | −2.020 |
| 202178_at | PRKCZ | chr1p36.33-p36.2 | 0.745 | −0.036 | −2.019 |
| 216105_x_at | PPP2R4 | chr9q34 | 0.632 | −0.246 | −2.019 |
| 212431_at | HMGXB3 | chr5q33.1 | 0.677 | −0.171 | −2.019 |
| 208833_s_at | ATXN10 | chr22q13.31 | 0.833 | 0.199 | −2.019 |
| 202780_at | OXCT1 | chr5p13.1 | 0.795 | 0.090 | −2.018 |
| 202264_s_at | TOMM40 | chr19q13 | 0.747 | −0.030 | −2.017 |
| 206196_s_at | RUNDC3A | chr17q21.31 | 0.815 | 0.147 | −2.016 |
| 211426_x_at | GNAQ | chr9q21 | 0.621 | −0.262 | −2.016 |
| 210689_at | CLDN14 | chr21q22.3 | 0.730 | −0.066 | −2.015 |
| 215406_at | — | — | 0.603 | −0.287 | −2.014 |
| 202228_s_at | NPTN | chr15q22 | 0.825 | 0.175 | −2.014 |
| 202499_s_at | SLC2A3 | chr12p13.3 | 0.792 | 0.084 | −2.013 |
| 208898_at | ATP6V1D | chr14q23-q24.2 | 0.834 | 0.206 | −2.013 |
| 214681_at | GK | chrXp21.3 | 0.691 | −0.142 | −2.013 |
| 214348_at | TACR2 | chr10q11-q21 | 0.528 | −0.384 | −2.010 |
| 203114_at | SSSCA1 | chr11q13.1 | 0.775 | 0.041 | −2.010 |
| 204048_s_at | PHACTR2 | chr6q24.2 | 0.560 | −0.344 | −2.010 |
| 217077_s_at | GABBR2 | chr9q22.1-q22.3 | 0.756 | −0.005 | −2.010 |
| 213909_at | LRRC15 | chr3q29 | 0.619 | −0.261 | −2.009 |
| 216298_at | TRGV5 | chr7p14 | 0.655 | −0.205 | −2.009 |
| 211017_s_at | NF2 | chr22q12.2 | 0.711 | −0.103 | −2.009 |
| 209445_x_at | C7orf44 | chr7p13 | 0.741 | −0.038 | −2.008 |
| 213262_at | SACS | chr13q12 | 0.789 | 0.079 | −2.008 |
| 206690_at | ACCN1 | chr17q12 | 0.867 | 0.318 | −2.008 |
| 221504_s_at | ATP6V1H | chr8q11.2 | 0.823 | 0.173 | −2.007 |
| 219054_at | C5orf23 | chr5p13.3 | 0.608 | −0.277 | −2.007 |
| 205820_s_at | APOC3 | chr11q23.1-q23.2 | 0.650 | −0.212 | −2.006 |
| 202143_s_at | COPS8 | chr2q37.3 | 0.662 | −0.191 | −2.006 |
| 204216_s_at | ZC3H14 | chr14q31.3 | 0.710 | −0.102 | −2.005 |
| 219767_s_at | CRYZL1 | chr21q21.3 | 0.811 | 0.139 | −2.005 |
| 208462_s_at | ABCC9 | chr12p12.1 | 0.687 | −0.145 | −2.005 |
| 214260_at | COPS8 | chr2q37.3 | 0.815 | 0.151 | −2.004 |
| 204521_at | C12orf24 | chr12q24.11 | 0.841 | 0.232 | −2.003 |
| 210720_s_at | NECAB3 | chr20q11.22 | 0.731 | −0.058 | −2.003 |
| 213200_at | SYP | chrXp11.23-p11.22 | 0.811 | 0.140 | −2.003 |
| 208778_s_at | TCP1 | chr6q25.3-q26 | 0.797 | 0.101 | −2.002 |
| 219600_s_at | TMEM50B | chr21q22.11 | 0.738 | −0.043 | −2.002 |
| 210123_s_at | [symbol] 7A /// | chr15q13.1 /// | 0.755 | −0.003 | −2.002 |
| | CHRNA7 /// | chr15q14 | | | |
| | LO [symbol] | | | | |
| 209934_s_at | ATP2C1 | chr3q22.1 | 0.560 | −0.341 | −2.002 |
| 218918_at | MAN1C1 | chr1p35 | 0.826 | 0.185 | −2.000 |
| 207026_s_at | ATP2B3 | chrXq28 | 0.668 | −0.178 | −2.000 |
| 214160_at | — | — | 0.653 | −0.204 | −2.000 |
| 36994_at | ATP6V0C | chr16p13.3 | 0.773 | 0.040 | −1.998 |
| 204049_s_at | PHACTR2 | chr6q24.2 | 0.634 | −0.234 | −1.998 |
| 220928_s_at | PRDM16 | chr1p36.23-p33 | 0.799 | 0.109 | −1.997 |
| 203237_s_at | NOTCH3 | chr19p13.2-p13.1 | 0.560 | −0.339 | −1.997 |
| 203303_at | DYNLT3 | chrXp21 | 0.840 | 0.231 | −1.997 |
| 208398_s_at | TBPL1 | chr6q22.1-q22.3 | 0.823 | 0.178 | −1.996 |
| 206197_at | NME5 | chr5q31 | 0.765 | 0.022 | −1.996 |
| 216786_at | LOC159110 | chrYq11.221 | 0.651 | −0.205 | −1.996 |
| 204812_at | ZW10 | chr11q23.2 | 0.827 | 0.192 | −1.996 |
| 216643_at | — | — | 0.652 | −0.203 | −1.995 |
| 218547_at | DHDDS | chr1p36.11 | 0.895 | 0.433 | −1.994 |
| 215227_x_at | ACP1 | chr2p25 | 0.718 | −0.080 | −1.994 |
| 219033_at | PARP8 | chr5q11.1 | 0.739 | −0.036 | −1.994 |
| 217881_s_at | CDC27 | chr17q12-q23.2 | 0.768 | 0.031 | −1.993 |
| 201431_s_at | DPYSL3 | chr5q32 | 0.842 | 0.239 | −1.993 |
| 221127_s_at | DKK3 | chr11p15.2 | 0.821 | 0.175 | −1.993 |
| 221251_x_at | INO80B | chr2p13.1 | 0.873 | 0.346 | −1.992 |
| 214665_s_at | CHP | chr15q13.3 | 0.819 | 0.170 | −1.992 |
| 221839_s_at | UBAP2 | chr9p13.3 | 0.830 | 0.204 | −1.991 |
| 206452_x_at | PPP2R4 | chr9q34 | 0.648 | −0.207 | −1.990 |
| 206051_at | ELAVL4 | chr1p34 | 0.860 | 0.301 | −1.990 |
| 216686_at | FLJ40330 | chr2p11.2 | 0.705 | −0.104 | −1.989 |
| 202166_s_at | PPP1R2 | chr3q29 | 0.755 | 0.003 | −1.988 |
| 210460_s_at | PSMD4 | chr1q21.2 | 0.851 | 0.272 | −1.988 |
| 210450_at | LOC90925 | chr14q32.33 | 0.649 | −0.204 | −1.987 |
| 211883_x_at | CEACAM1 | chr19q13.2 | 0.789 | 0.088 | −1.987 |
| 205233_s_at | PAFAH2 | chr1p36 | 0.709 | −0.095 | −1.986 |
| 214074_s_at | CTTN | chr11q13 | 0.740 | −0.029 | −1.986 |
| 214728_x_at | SMARCA4 | chr19p13.2 | 0.769 | 0.037 | −1.986 |
| 212862_at | CDS2 | chr20p13 | 0.809 | 0.143 | −1.985 |
| 220570_at | RETN | chr19p13.2 | 0.695 | −0.121 | −1.984 |
| 204953_at | SNAP91 | chr6q14.2 | 0.827 | 0.195 | −1.984 |
| 203810_at | DNAJB4 | chr1p31.1 | 0.637 | −0.223 | −1.984 |
| 211662_s_at | VDAC2 | chr10q22 | 0.804 | 0.129 | −1.984 |
| 208093_s_at | NDEL1 | chr17p13.1 | 0.802 | 0.124 | −1.984 |
| 215093_at | NSDHL | chrXq28 | 0.760 | 0.018 | −1.984 |
| 204622_x_at | NR4A2 | chr2q22-q23 | 0.930 | 0.592 | −1.983 |
| 89948_at | PCIF1 | chr20q13.12 | 0.689 | −0.132 | −1.983 |
| 204169_at | IMPDH1 | chr7q31.3-q32 | 0.875 | 0.359 | −1.982 |
| 207928_s_at | GLRA3 | chr4q33-q34 | 0.685 | −0.139 | −1.982 |
| 219005_at | TMEM59L | chr19p12 | 0.755 | 0.007 | −1.982 |
| 203894_at | TUBG2 | chr17q21 | 0.723 | −0.063 | −1.981 |
| 210098_s_at | — | — | 0.683 | −0.142 | −1.981 |
| 212855_at | DCUN1D4 | chr4q12 | 0.819 | 0.174 | −1.981 |
| 214231_s_at | KIAA0564 | chr13q14.11 | 0.504 | −0.400 | −1.980 |
| 208121_s_at | PTPRO | chr12p13.3- | 0.853 | 0.280 | −1.980 |
| | | p13.2\|12p13-p12 | | | |
| 207853_s_at | SNCB | chr5q35 | 0.785 | 0.081 | −1.980 |
| 218503_at | KIAA1797 | chr9p21 | 0.826 | 0.194 | −1.980 |
| 214641_at | COL4A3 | chr2q36-q37 | 0.662 | −0.178 | −1.979 |
| 208864_s_at | TXN | chr9q31 | 0.806 | 0.138 | −1.979 |
| 208699_x_at | TKT | chr3p14.3 | 0.632 | −0.228 | −1.979 |
| 212271_at | MAPK1 | chr22q11.2\|22q11.21 | 0.775 | 0.055 | −1.978 |
| 220069_at | TUBA8 | chr22q11.1 | 0.551 | −0.342 | −1.977 |
| 218609_s_at | NUDT2 | chr9p13 | 0.588 | −0.292 | −1.976 |
| 212600_s_at | UQCRC2 | chr16p12 | 0.772 | 0.050 | −1.976 |
| 221721_s_at | LZTS1 | chr8p22 | 0.691 | −0.125 | −1.976 |
| 212764_at | ZEB1 | chr10p11.2 | 0.787 | 0.088 | −1.975 |
| 216286_at | — | — | 0.793 | 0.103 | −1.975 |
| 50314_i_at | C20orf27 | chr20p13 | 0.849 | 0.270 | −1.975 |
| 216323_x_at | TUBA3D | chr2q21.1 | 0.606 | −0.265 | −1.975 |
| 210676_x_at | [symbol] PD5 /// | chr2q13 | 0.682 | −0.141 | −1.974 |
| | RGPD6 /// | | | | |
| | RG [symbol] | | | | |
| 211729_x_at | BLVRA | chr7p14-cen | 0.819 | 0.178 | −1.973 |
| 218681_s_at | SDF2L1 | chr22q11.21 | 0.632 | −0.225 | −1.973 |
| 203001_s_at | STMN2 | chr8q21.13 | 0.846 | 0.261 | −1.973 |
| 212518_at | PIP5K1C | chr19p13.3 | 0.851 | 0.280 | −1.972 |
| 218421_at | CERK | chr22q13.31 | 0.879 | 0.380 | −1.972 |
| 202791_s_at | SAPS2 | chr22q13.33 | 0.572 | −0.312 | −1.972 |
| 202460_s_at | LPIN2 | chr18p11.31 | 0.886 | 0.406 | −1.972 |
| 216266_s_at | ARFGEF1 | chr8q13 | 0.684 | −0.135 | −1.971 |
| 204525_at | PHF14 | chr7p21.3 | 0.644 | −0.205 | −1.970 |
| 210416_s_at | CHEK2 | chr22q11\|22q12.1 | 0.643 | −0.205 | −1.970 |
| 202967_at | GSTA4 | chr6p12.1 | 0.750 | 0.002 | −1.969 |
| 212984_at | ATF2 | chr2q32 | 0.857 | 0.301 | −1.969 |
| 202281_at | GAK | chr4p16 | 0.773 | 0.057 | −1.965 |
| 203945_at | ARG2 | chr14q24.1-q24.3 | 0.696 | −0.109 | −1.965 |
| 202472_at | MPI | chr15q22-qter | 0.723 | −0.056 | −1.965 |
| 205737_at | KCNQ2 | chr20q13.3 | 0.800 | 0.128 | −1.965 |
| 217187_at | MUC5AC | chr11p15.5 | 0.538 | −0.353 | −1.964 |
| 210335_at | RASSF9 | chr12q21.31 | 0.644 | −0.202 | −1.963 |
| 213103_at | STARD13 | chr13q12-q13 | −0.846 | 0.817 | 4.840 |
| 219728_at | MYOT | chr5q31 | −0.695 | 0.725 | 3.598 |
| 208214_at | ADRB1 | chr10q24-q26 | −0.658 | 0.733 | 3.493 |
| 208371_s_at | RING1 | chr6p21.3 | −0.682 | 0.711 | 3.490 |
| 213185_at | KIAA0556 | chr16p12.1-p11.2 | −0.811 | 0.530 | 3.485 |
| 203648_at | TATDN2 | chr3p25.3 | −0.631 | 0.706 | 3.290 |
| 209289_at | NFIB | chr9p24.1 | −0.734 | 0.584 | 3.255 |
| 220739_s_at | CNNM3 | chr2p12-p11.2 | −0.752 | 0.547 | 3.225 |
| 205909_at | POLE2 | chr14q21-q22 | −0.775 | 0.506 | 3.222 |
| 200601_at | ACTN4 | chr19q13 | −0.717 | 0.592 | 3.207 |
| 218245_at | TSKU | chr11q13.5 | −0.716 | 0.591 | 3.199 |
| 211494_s_at | SLC4A4 | chr4q21 | −0.718 | 0.582 | 3.178 |
| 218723_s_at | C13orf15 | chr13q14.11 | −0.654 | 0.639 | 3.116 |
| 203298_s_at | JARID2 | chr6p24-p23 | −0.527 | 0.733 | 3.081 |
| 209290_s_at | NFIB | chr9p24.1 | −0.734 | 0.511 | 3.044 |
| 209049_s_at | ZMYND8 | chr20q13.12 | −0.672 | 0.585 | 3.009 |
| 207663_x_at | GAGE3 | chrXp11.4-p11.2 | −0.705 | 0.537 | 2.993 |
| 208755_x_at | [symbol] A /// | chr17q25 /// | −0.537 | 0.699 | 2.970 |
| | H3F3B /// | chr1q41 /// | | | |
| | LOC44 [symbol] | chr2q31.1 | | | |
| 91617_at | DGCR8 | chr22q11.2 | −0.603 | 0.636 | 2.936 |
| 219584_at | PLA1A | chr3q13.13-q13.2 | −0.443 | 0.747 | 2.923 |
| 208190_s_at | LSR | chr19q13.12 | −0.644 | 0.590 | 2.922 |
| 219000_s_at | DSCC1 | chr8q24.12 | −0.689 | 0.529 | 2.907 |
| 213972_at | FOXD1 | chr5q12-q13 | −0.647 | 0.570 | 2.873 |
| 204075_s_at | KIAA0562 | chr1p36.32 | −0.663 | 0.541 | 2.845 |
| 212144_at | UNC84B | chr22q13.1 | −0.635 | 0.564 | 2.813 |
| 204457_s_at | GAS1 | chr9q21.3-q22 | −0.256 | 0.810 | 2.810 |
| 212684_at | ZNF3 | chr7q22.1 | −0.669 | 0.511 | 2.784 |
| 218880_at | FOSL2 | chr2p23.3 | −0.441 | 0.715 | 2.776 |
| 209684_at | RIN2 | chr20p11.22 | −0.545 | 0.640 | 2.773 |
| 39318_at | TCL1A | chr14q32.1 | −0.497 | 0.676 | 2.770 |
| 201502_s_at | NFKBIA | chr14q13 | −0.447 | 0.709 | 2.767 |
| 213541_s_at | ERG | chr21q22.3 | −0.591 | 0.595 | 2.766 |
| 204535_s_at | REST | chr4q12 | −0.613 | 0.571 | 2.759 |
| 200621_at | CSRP1 | chr1q32 | −0.625 | 0.556 | 2.755 |
| 207793_s_at | EPB41 | chr1p33-p32 | −0.532 | 0.637 | 2.728 |
| 216452_at | TRPM3 | chr9q21.11-q21.12 | −0.523 | 0.643 | 2.723 |
| 201876_at | PON2 | chr7q21.3 | −0.632 | 0.534 | 2.717 |
| 206026_s_at | TNFAIP6 | chr2q23.3 | −0.286 | 0.773 | 2.679 |
| 209544_at | RIPK2 | chr8q21 | −0.528 | 0.625 | 2.677 |
| 207761_s_at | METTL7A | chr12q13.13 | −0.474 | 0.667 | 2.676 |
| 207961_x_at | MYH11 | chr16p13.11 | −0.502 | 0.644 | 2.668 |
| 205112_at | PLCE1 | chr10q23 | −0.550 | 0.602 | 2.663 |
| 209710_at | GATA2 | chr3q21.3 | −0.514 | 0.629 | 2.651 |
| 204187_at | GMPR | chr6p23 | −0.604 | 0.543 | 2.651 |
| 203297_s_at | JARID2 | chr6p24-p23 | −0.537 | 0.609 | 2.648 |
| 201497_x_at | MYH11 | chr16p13.11 | −0.596 | 0.551 | 2.647 |
| 214393_at | RND2 | chr17q21 | −0.588 | 0.557 | 2.639 |
| 204373_s_at | CEP350 | chr1p36.13-q41 | −0.579 | 0.564 | 2.632 |
| 209032_s_at | CADM1 | chr11q23.2 | −0.563 | 0.577 | 2.623 |
| 218656_s_at | LHFP | chr13q12 | −0.270 | 0.765 | 2.602 |
| 216320_x_at | MST1 | chr3p21 | −0.575 | 0.550 | 2.581 |
| 208718_at | DDX17 | chr22q13.1 | −0.497 | 0.619 | 2.570 |
| 215038_s_at | SETD2 | chr3p21.31 | −0.329 | 0.726 | 2.556 |
| 200915_x_at | KTN1 | chr14q22.1 | −0.560 | 0.554 | 2.548 |
| 201845_s_at | RYBP | chr3p13 | −0.338 | 0.718 | 2.542 |
| 213638_at | PHACTR1 | chr6p24.1 | −0.562 | 0.551 | 2.541 |
| 49452_at | ACACB | chr12q24.11 | −0.533 | 0.578 | 2.538 |
| 202173_s_at | VEZF1 | chr17q22 | −0.401 | 0.679 | 2.536 |
| 201361_at | TMEM109 | chr11q12.2 | −0.517 | 0.588 | 2.526 |
| 214703_s_at | MAN2B2 | chr4p16.1 | −0.569 | 0.533 | 2.513 |
| 213891_s_at | TCF4 | chr18q21.1 | −0.585 | 0.514 | 2.511 |
| 203685_at | BCL2 | chr18q21.33\|18q21.3 | −0.499 | 0.598 | 2.509 |
| 213828_x_at | [symbol] A /// | chr17q25 /// | −0.470 | 0.619 | 2.500 |
| | H3F3B /// | chr1q41 /// | | | |
| | LOC44 [symbol] | chr2q31.1 | | | |
| 207191_s_at | ISLR | chr15q23-q24 | −0.310 | 0.721 | 2.493 |
| 212237_at | ASXL1 | chr20q11.1 | −0.074 | 0.817 | 2.478 |
| 210451_at | PKLR | chr1q21 | −0.397 | 0.663 | 2.467 |
| 201739_at | SGK1 | chr6q23 | −0.482 | 0.596 | 2.459 |
| 207390_s_at | SMTN | chr22q12.2 | −0.531 | 0.550 | 2.450 |
| 207016_s_at | ALDH1A2 | chr15q22.1 | −0.558 | 0.517 | 2.435 |
| 200665_s_at | SPARC | chr5q31.3-q32 | −0.561 | 0.513 | 2.433 |
| 204225_at | HDAC4 | chr2q37.3 | −0.185 | 0.765 | 2.419 |
| 217118_s_at | C22orf9 | chr22q13.31 | −0.495 | 0.570 | 2.410 |
| 202294_at | STAG1 | chr3q22.3 | −0.067 | 0.808 | 2.409 |
| 217856_at | RBM8A | chr1q12 | −0.517 | 0.548 | 2.408 |
| 32069_at | N4BP1 | chr16q12.1 | −0.504 | 0.559 | 2.403 |
| 219851_at | ZNF613 | chr19q13.33 | −0.541 | 0.521 | 2.399 |
| 217367_s_at | ZHX3 | chr20q12 | −0.382 | 0.653 | 2.396 |
| 207404_s_at | HTR1E | chr6q14-q15 | −0.426 | 0.621 | 2.392 |
| 221087_s_at | APOL3 | chr22q13.1 | −0.464 | 0.590 | 2.392 |
| 211940_x_at | [symbol] A /// | chr17q25 /// | −0.326 | 0.681 | 2.370 |
| | H3F3B /// | chr1q41 /// | | | |
| | LOC44 [symbol] | chr2q31.1 | | | |
| 216264_s_at | LAMB2 | chr3p21 | −0.287 | 0.703 | 2.369 |
| 202332_at | CSNK1E | chr22q13.1 | −0.484 | 0.564 | 2.366 |
| 215146_s_at | TTC28 | chr22q12.1 | −0.422 | 0.615 | 2.363 |
| 205053_at | PRIM1 | chr12q13 | −0.345 | 0.665 | 2.353 |
| 200610_s_at | NCL | chr2q12-qter | −0.075 | 0.795 | 2.349 |
| 219255_x_at | IL17RB | chr3p21.1 | −0.212 | 0.735 | 2.340 |
| 219371_s_at | KLF2 | chr19p13.13-p13.11 | −0.517 | 0.522 | 2.334 |
| 203010_at | STAT5A | chr17q11.2 | −0.480 | 0.556 | 2.328 |
| 219213_at | JAM2 | chr21q21.2 | −0.373 | 0.633 | 2.308 |
| 218089_at | C20orf4 | chr20pter-q12 | −0.248 | 0.707 | 2.298 |
| 210147_at | ART3 | [symbol] hr4p15.1- | −0.510 | 0.516 | 2.298 |
| | | p14\|4p15.1- | | | |
| | | p14\|4p15.1-p1 [symbol] | | | |
| 208763_s_at | TSC22D3 | chrXq22.3 | −0.358 | 0.640 | 2.295 |
| 213467_at | RND2 | chr17q21 | −0.330 | 0.655 | 2.284 |
| 212655_at | ZCCHC14 | chr16q24.2 | −0.249 | 0.703 | 2.284 |
| 213765_at | MFAP5 | chr12p13.1-p12.3 | −0.419 | 0.588 | 2.270 |
| 205907_s_at | OMD | chr9q22.31 | −0.482 | 0.532 | 2.267 |
| 202172_at | VEZF1 | chr17q22 | −0.403 | 0.596 | 2.257 |
| 203045_at | NINJ1 | chr9q22 | −0.405 | 0.586 | 2.231 |
| 203694_s_at | DHX16 | chr6p21.3 | 0.093 | 0.831 | 2.228 |
| 218963_s_at | KRT23 | chr17q21.2 | −0.305 | 0.654 | 2.224 |
| 212747_at | ANKS1A | chr6p21.31 | −0.208 | 0.708 | 2.218 |
| 209030_s_at | CADM1 | chr11q23.2 | −0.308 | 0.649 | 2.215 |
| 213401_s_at | TBL1X | chrXp22.3 | −0.394 | 0.589 | 2.214 |
| 209546_s_at | APOL1 | chr22q13.1 | −0.465 | 0.528 | 2.210 |
| 209497_s_at | RBM4B | chr11q13 | −0.128 | 0.744 | 2.204 |
| 202925_s_at | PLAGL2 | chr20q11.21 | −0.481 | 0.511 | 2.204 |
| 204731_at | TGFBR3 | chr1p33-p32 | −0.435 | 0.552 | 2.203 |
| 209815_at | PTCH1 | chr9q22.3 | −0.239 | 0.687 | 2.202 |
| 214721_x_at | CDC42EP4 | chr17q24-q25 | −0.333 | 0.630 | 2.202 |
| 218062_x_at | CDC42EP4 | chr17q24-q25 | −0.331 | 0.630 | 2.201 |
| 203449_s_at | TERF1 | chr8q13 | −0.458 | 0.530 | 2.200 |
| 212618_at | ZNF609 | chr15q22.31 | −0.392 | 0.586 | 2.199 |
| 203038_at | PTPRK | chr6q22.2-q22.3 | −0.426 | 0.557 | 2.197 |
| 218829_s_at | CHD7 | chr8q12.2 | −0.379 | 0.589 | 2.179 |
| 221924_at | ZMIZ2 | chr7p13 | −0.340 | 0.617 | 2.178 |
| 204872_at | TLE4 | chr9q21.31 | −0.368 | 0.595 | 2.170 |
| 212977_at | CXCR7 | chr2q37.3 | −0.337 | 0.613 | 2.157 |
| 217853_at | TNS3 | chr7p12.3 | −0.214 | 0.688 | 2.152 |
| 220032_at | C7orf58 | chr7q31.31 | −0.461 | 0.509 | 2.148 |
| 219860_at | LY6G5C | chr6p21.33 | −0.162 | 0.714 | 2.146 |
| 57532_at | DVL2 | chr17p13.2 | −0.150 | 0.719 | 2.142 |
| 212387_at | TCF4 | chr18q21.1 | −0.463 | 0.502 | 2.134 |
| 219948_x_at | UGT2A3 | chr4q13.2 | −0.443 | 0.520 | 2.131 |
| 205932_s_at | MSX1 | chr4p16.3-p16.1 | −0.448 | 0.512 | 2.123 |
| 218839_at | HEY1 | chr8q21 | −0.270 | 0.646 | 2.120 |
| 201938_at | CDK2AP1 | chr12q24.31 | 0.137 | 0.829 | 2.119 |
| 208999_at | 8-Sep | chr5q31 | 0.021 | 0.787 | 2.113 |
| 206038_s_at | NR2C2 | chr3p25 | −0.415 | 0.537 | 2.110 |
| 212816_s_at | CBS | chr21q22.3 | −0.383 | 0.557 | 2.091 |
| 206134_at | ADAMDEC1 | chr8p21.2 | −0.396 | 0.546 | 2.091 |
| 218400_at | OAS3 | chr12q24.2 | −0.312 | 0.608 | 2.083 |
| 213380_x_at | MSTP9 | chr1p36.13 | −0.408 | 0.531 | 2.078 |
| 208919_s_at | NADK | chr1p36.33-p36.21 | −0.015 | 0.765 | 2.076 |
| 204327_s_at | ZNF202 | chr11q23.3 | −0.057 | 0.747 | 2.073 |
| 206504_at | CYP24A1 | chr20q13 | −0.436 | 0.502 | 2.066 |
| 46665_at | SEMA4C | chr2q11.2 | −0.303 | 0.608 | 2.063 |
| 218413_s_at | ZNF639 | chr3q26.32 | −0.262 | 0.634 | 2.057 |
| 217905_at | C10orf119 | chr10q26.11 | −0.295 | 0.611 | 2.056 |
| 201983_s_at | EGFR | chr7p12 | −0.277 | 0.623 | 2.055 |
| 212238_at | ASXL1 | chr20q11.1 | −0.131 | 0.707 | 2.053 |
| 217184_s_at | LTK | chr15q15.1-q21.1 | −0.280 | 0.620 | 2.052 |
| 208818_s_at | COMT | chr22q11.21- | −0.326 | 0.588 | 2.052 |
| | | q11.23\|22q11.21 | | | |
| 44783_s_at | HEY1 | chr8q21 | −0.313 | 0.594 | 2.043 |
| 212848_s_at | C9orf3 | chr9q22.32 | −0.392 | 0.532 | 2.041 |
| 202866_at | DNAJB12 | chr10q22.1 | −0.236 | 0.644 | 2.038 |
| 203959_s_at | ZBTB40 | chr1pter-q31.3 | −0.231 | 0.646 | 2.035 |
| 206919_at | ELK4 | chr1q32 | −0.387 | 0.533 | 2.031 |
| 202411_at | IFI27 | chr14q32 | −0.349 | 0.563 | 2.030 |
| 212447_at | KBTBD2 | chr7p14.3 | −0.269 | 0.621 | 2.030 |
| 204638_at | ACP5 | chr19p13.3-p13.2 | −0.371 | 0.545 | 2.029 |
| 214857_at | — | — | −0.309 | 0.590 | 2.021 |
| 213058_at | TTC28 | chr22q12.1 | −0.194 | 0.664 | 2.021 |
| 212321_at | SGPL1 | chr10q21 | −0.226 | 0.645 | 2.019 |
| 221012_s_at | TRIM8 | chr10q24.3 | −0.140 | 0.692 | 2.014 |
| 212385_at | TCF4 | chr18q21.1 | −0.392 | 0.522 | 2.013 |
| 203117_s_at | PAN2 | chr12q13.2-q13.3 | −0.373 | 0.537 | 2.010 |
| 200659_s_at | PHB | chr17q21 | −0.098 | 0.713 | 2.008 |
| 221824_s_at | 8-Mar | chr10q11.21 | 0.085 | 0.791 | 2.002 |
| 221252_s_at | GSG1 | chr12p13.1 | −0.399 | 0.508 | 1.989 |
| 201783_s_at | RELA | chr11q13 | −0.390 | 0.515 | 1.989 |
| 206747_at | GPRIN2 | chr10q11.22 | −0.392 | 0.511 | 1.981 |
| 206402_s_at | NPFF | chr12q13.13 | −0.121 | 0.692 | 1.972 |
| 54051_at | PKNOX1 | chr21q22.3 | −0.083 | 0.711 | 1.972 |
| 203617_x_at | ELK1 | chrXp11.2 | −0.319 | 0.566 | 1.968 |
| 209343_at | EFHD1 | chr2q37.1 | −0.374 | 0.520 | 1.965 |
| 209427_at | SMTN | chr22q12.2 | −0.343 | 0.545 | 1.964 |

[symbol]

indicates data missing or illegible when filed

SUPPLEMENTARY TABLE 8
This file contains the lists of genes differentially correlated
with aSynL in rs356168 CCvsTT non PD-affected cortex samples. 8

| Illumina | | | Correlation wit aSynL | | |
|---|---|---|---|---|---|
| Probe | Gene Symbol | Cytoband | rs356168 CC | rs356168 TT | Diff. Score |
| GI_14589898-S | MAP2K1 | 15q22.1-q22.3 [symbol] | 0.898 | 0.961 | −2.399 |
| GI_4557816-S | OXCT | 5p13.1 | 0.863 | 0.944 | −2.267 |
| GI_4507878-S | VDAC1 | 5q31 | 0.669 | 0.944 | −4.643 |
| GI_16579891-A | WDR7 | 18q21.1-q22 | 0.861 | 0.944 | −2.282 |
| GI_37549342-S | KIAA1265 | 2q32.3 | 0.838 | 0.937 | −2.395 |
| GI_18765734-A | SNAP25 | 20p12-p11.2 | 0.852 | 0.936 | −2.135 |
| GI_41406092-I | JDP1 | 10q22.1 | 0.852 | 0.935 | −2.104 |
| GI_21361889-S | KLHL12 | 1q32.1 | 0.811 | 0.935 | −2.727 |
| GI_21536456-S | MOAP1 | 14q32 | 0.776 | 0.935 | −3.191 |
| GI_19913423-S | ATP6V1A | 3q13.2-q13.31 | 0.820 | 0.934 | −2.576 |
| GI_21735619-S | MDH1 | 2p13.3 | 0.802 | 0.930 | −2.651 |
| GI_42558244-S | GMRP-1 | 11p15.2 | 0.758 | 0.928 | −3.135 |
| GI_12597656-S | FLJ13110 | 2p11.2 | 0.782 | 0.928 | −2.845 |
| GI_34147658-S | UCHL1 | 4p14 | 0.840 | 0.926 | −1.961 |
| GI_20357538-A | ATP6V1G2 | 6p21.3 | 0.794 | 0.926 | −2.616 |
| GI_31542527-S | DKFZP566B183 | 12p13.31 | 0.833 | 0.925 | −2.054 |
| GI_6042206-S | RAM | 12q24.3 | 0.811 | 0.924 | −2.347 |
| GI_7770073-A | IARS | 9q21 | 0.745 | 0.924 | −3.140 |
| GI_19745179-S | MO25 | 2q37.1 | 0.794 | 0.922 | −2.500 |
| GI_21614509-A | FGF12 | 3q28 | 0.726 | 0.921 | −3.245 |
| GI_29744077-S | LOC340542 | Xq22.1 | 0.826 | 0.920 | −1.992 |
| GI_31341302-S | JAZF1 | 7p15.2-p15.1 | 0.715 | 0.920 | −3.321 |
| GI_21464140-I | AKAP11 | 13q14.11 | 0.785 | 0.919 | −2.528 |
| GI_24308335-S | C6orf168 | 6q16.2-q16.3 | 0.813 | 0.917 | −2.094 |
| GI_19718771-A | ASNS | 7q21.3 | 0.801 | 0.916 | −2.227 |
| GI_4758177-S | DNG1 | 7q21.3-q22.1 | 0.803 | 0.916 | −2.194 |
| GI_41352714-S | VPS35 | 16q12 | 0.787 | 0.915 | −2.363 |
| GI_31341108-S | LOC170261 | Xq24 | 0.720 | 0.914 | −3.111 |
| GI_15451903-S | MOX2 | 3q12-q13 | 0.790 | 0.914 | −2.324 |
| GI_42544237-S | DCAMKL1 | 13q13 | 0.724 | 0.910 | −2.945 |
| GI_37543748-S | KIAA1467 | 12p13.1 | 0.716 | 0.910 | −3.019 |
| GI_15011917-S | ATP6AP2 | Xp11.4 | 0.618 | 0.908 | −3.817 |
| GI_16950592-I | MRPS21 | null | 0.759 | 0.907 | −2.481 |
| GI_21361148-S | RGS7 | 1q43 | 0.763 | 0.906 | −2.411 |
| GI_38257145-S | LOC157567 | 8q22.3 | 0.787 | 0.904 | −2.060 |
| GI_27734858-S | LOC285533 | 4q31.3 | 0.783 | 0.900 | −2.019 |
| GI_38201693-S | RGS4 | 1q23.3 | 0.780 | 0.898 | −1.994 |
| GI_4502806-S | CHGB | 20pter-p12 | 0.732 | 0.896 | −2.497 |
| GI_27501445-S | DENR | 12q24.31 | 0.767 | 0.895 | −2.091 |
| GI_33946322-S | ARL1 | 12q23.2 | 0.701 | 0.895 | −2.768 |
| GI_7106298-S | E46L | 22q13.31 | 0.721 | 0.895 | −2.573 |
| GI_21536422-A | AM PH | 7p14-p13 | 0.739 | 0.894 | −2.382 |
| GI_23110933-A | PSMA1 | 11p15.1 | 0.630 | 0.894 | −3.367 |
| GI_37546739-S | KIAA1107 | 1p22.1 | 0.699 | 0.893 | −2.738 |
| GI_7657043-S | NGFRAP1 | Xq22.2 | 0.696 | 0.892 | −2.754 |
| GI_22094078-S | TRIP3 | 17q12 | 0.707 | 0.892 | −2.652 |
| GI_34222111-S | DKFZp547C176 | 11q22.3 | 0.767 | 0.892 | −2.008 |
| GI_19482173-S | CUL2 | 10p11.21 | 0.654 | 0.890 | −3.089 |
| GI_29740938-S | KIAA1136 | 10p12.1 | 0.690 | 0.890 | −2.760 |
| GI_27886536-1 | ATP2A2 | 12q23-q24.1 | 0.730 | 0.889 | −2.348 |
| GI_24462252-S | ZNF25 | 10p11.21 | 0.737 | 0.888 | −2.256 |
| GI_4755130-S | CCK | 3p22-p21.3 | 0.745 | 0.885 | −2.101 |
| GI_24475639-S | HSA272196 | 17q11.2 | 0.718 | 0.885 | −2.377 |
| GI_41350195-S | RAB1A | 2p14 | 0.716 | 0.884 | −2.383 |
| GI_13899218-S | GABARAPL1 | 12p13.2 | 0.722 | 0.884 | −2.324 |
| GI_41281397-S | SHOC2 | 10q25 | 0.737 | 0.883 | −2.146 |
| GI_15147227-S | BEX1 | Xq21-q23 | 0.505 | 0.883 | −4.014 |
| GI_24475709-S | HBLD2 | 9q21.33 | 0.748 | 0.880 | −1.971 |
| GI_17999531-S | COX6C | 8q22-q23 | 0.572 | 0.880 | −3.495 |
| GI_5730086-S | TCTE1L | Xp21 | 0.626 | 0.880 | −3.088 |
| GI_4504066-S | GOT1 | 10q24.1-q25.1 | 0.647 | 0.879 | −2.893 |
| GI_5730054-S | PLK2 | 5q12.1-q13.2 | 0.731 | 0.879 | −2.116 |
| GI_10800415-S | SCG2 | 2q35-q36 | 0.538 | 0.878 | −3.692 |
| GI_21362111-S | C7orf2 | 7q36 | 0.708 | 0.878 | −2.330 |
| GI_39930462-S | KIAA1701 | Xq23 | 0.557 | 0.878 | −3.550 |
| GI_24415993-S | SFRS7 | 2p22.1 | 0.597 | 0.877 | −3.242 |
| GI_14769619-S | KIAA1701 | Xq23 | 0.724 | 0.877 | −2.142 |
| GI_1321582-S | SUCLA2 | 13q12.2-q13.3 | 0.643 | 0.873 | −2.798 |
| GI_8922171-S | DKFZp761K1423 | 8p22 | 0.692 | 0.873 | −2.369 |
| GI_33147079-S | AMSH-LP | 10q23.31 | 0.623 | 0.873 | −2.956 |
| GI_18426903-A | WRNIP1 | 6p25.2 | 0.712 | 0.872 | −2.167 |
| GI_5031710-S | GC20 | 3p22.1 | 0.717 | 0.872 | −2.112 |
| GI_19923783-S | PC4 | 5p13.3 | 0.729 | 0.872 | −1.981 |
| GI_21464100-S | YWHAG | 7q11.23 | 0.573 | 0.869 | −3.257 |
| GI_20531764-S | C13orf1 | 13q14 | 0.721 | 0.868 | −2.006 |
| GI_22907051-S | ARPC1A | 7q22.1 | 0.717 | 0.867 | −2.023 |
| GI_29135342-S | HINT1 | 5q31.2 | 0.638 | 0.866 | −2.711 |
| GI_16554603-S | MRPS23 | 17q22-q23 | 0.692 | 0.866 | −2.236 |
| GI_32698731-S | KIAA1363 | 3q26.31 | 0.660 | 0.866 | −2.512 |
| GI_6466447-S | DSTN | 20p12.1 | 0.647 | 0.865 | −2.613 |
| GI_44771179-S | NACSIN | 2p15 | 0.711 | 0.865 | −2.032 |
| GI_4758733-S | PMPCB | 7q22.1 | 0.685 | 0.865 | −2.278 |
| GI_28461128-S | ANKMY2 | 7p21 | 0.582 | 0.864 | −3.095 |
| GI_21265077-S | MRPL15 | 8q11.2-q13 | 0.632 | 0.864 | −2.705 |
| GI_12232400-S | SMYD3 | 1q44 | 0.459 | 0.863 | −3.892 |
| GI_4502300-S | ATP5G3 | 2q31.1 | 0.693 | 0.862 | −2.157 |
| GI_6005992-I | CLTA | 9p13 | 0.559 | 0.862 | −3.216 |
| GI_4885078-S | ATP5C1 | 10p15.1 | 0.646 | 0.861 | −2.537 |
| GI_19923737-S | PRPS1 | Xq21.32-q24 | 0.553 | 0.860 | −3.232 |
| GI_38327553-S | GABRA1 | 5q34-q35 | 0.484 | 0.860 | −3.674 |
| GI_30089947-S | PPM1E | 17q22 | 0.586 | 0.858 | −2.958 |
| GI_30425545-S | C14orf11 | 14q13.1 | 0.637 | 0.857 | −2.538 |
| GI_4505812-S | DNCL1 | 12q24.23 | 0.571 | 0.856 | −3.028 |
| GI_34222347-S | LRP11 | 6q25.1 | 0.670 | 0.855 | −2.220 |
| GI_5453602-S | CCT2 | 12q15 | 0.617 | 0.854 | −2.651 |
| GI_31652246-S | STXBP5 | 6q24.3 | 0.572 | 0.853 | −2.964 |
| GI_28872807-S | GAP43 | 3q13.1-q13.2 | 0.661 | 0.849 | −2.206 |
| GI_20357567-S | AASDHPPT | 11q22 | 0.631 | 0.849 | −2.447 |
| GI_41150497-S | LOC283951 | 16p13.3 | 0.503 | 0.847 | −3.335 |
| GI_8922423-S | GSPT2 | Xp11.23-p11.2 [symbol] | 0.667 | 0.847 | −2.115 |
| GI_33359693-A | UBE2E3 | 2q32.1 | 0.675 | 0.846 | −2.034 |
| GI_6806896-I | SNCA | 4q21 | 0.668 | 0.846 | −2.089 |
| GI_21626459-S | AF1Q | 1q21 | 0.539 | 0.846 | −3.068 |
| GI_13376622-S | NIF3L1BP1 | 3p14.1 | 0.641 | 0.845 | −2.300 |
| GI_38373689-S | COPS4 | 4q21.22 | 0.662 | 0.845 | −2.124 |
| GI_41281529-S | FAM20B | 1q25 | 0.637 | 0.845 | −2.327 |
| GI_24371267-S | NAP1L5 | 4q22.1 | 0.669 | 0.843 | −2.025 |
| GI_7108354-S | LMO4 | 1p22.3 | 0.648 | 0.843 | −2.200 |
| GI_4507310-S | SUPT4H1 | 17q21-q23 | 0.627 | 0.842 | −2.361 |
| GI_24308441-S | C6orf117 | 6q14.3 | 0.620 | 0.841 | −2.403 |
| GI_37059725-S | GRPEL1 | 4p16 | 0.446 | 0.840 | −3.560 |
| GI_41349455-S | PREP | 6q22 | 0.581 | 0.839 | −2.658 |
| GI_29789280-S | KIAA1750 | 8q22.1 | 0.620 | 0.839 | −2.364 |
| GI_23065568-S | GSTA4 | 6p12.1 | 0.625 | 0.838 | −2.318 |
| GI_24475618-S | CD83 | 6p23 | 0.643 | 0.838 | −2.172 |
| GI_24308232-S | SYT13 | 11p12-p11 | 0.569 | 0.837 | −2.717 |
| GI_6912611-A | PSK-1 | 16p11.2 | 0.647 | 0.836 | −2.110 |
| GI_7706752-S | UCHL5 | 1q32 | 0.664 | 0.836 | −1.965 |
| GI_21327696-S | DDX25 | 11q24 | 0.654 | 0.836 | −2.050 |
| GI_40317631-S | NUDT4 | 12q21 | 0.621 | 0.836 | −2.309 |
| GI_13375741-S | FLJ11712 | 13q14.3 | 0.657 | 0.835 | −1.997 |
| GI_14249303-S | MGC12966 | 7p22.1 | 0.607 | 0.835 | −2.401 |
| GI_17017986-S | COX5A | 15q25 | 0.526 | 0.833 | −2.959 |
| GI_4506064-S | PRKAR2B | 7q22 | 0.641 | 0.833 | −2.115 |
| GI_38569420-S | ACLY | 17q12-q21 | 0.641 | 0.833 | −2.108 |
| GI_19923976-S | C7orf30 | 7p15.3 | 0.604 | 0.833 | −2.393 |
| GI_4507728-S | TUBB | 6p25 | 0.558 | 0.833 | −2.727 |
| GI_4503064-S | CRYM | 16p13.11-p12. [symbol] | 0.531 | 0.832 | −2.907 |
| GI_25014108-S | SELH | 11q12.1 | 0.508 | 0.832 | −3.058 |
| GI_34452698-S | ACTR3 | 2q14.1 | 0.638 | 0.832 | −2.114 |
| GI_31377757-S | AFTIPHILIN | 2p14 | 0.611 | 0.832 | −2.323 |
| GI_4506930-S | SH3GL2 | 9p22 | 0.598 | 0.827 | −2.347 |
| GI_19923361-S | THY1 | 11q22.3-q23 | 0.622 | 0.825 | −2.139 |
| GI_31542788-S | ABHD7 | 1p22.1 | 0.478 | 0.825 | −3.138 |
| GI_21264557-S | SMAP1 | 6q13 | 0.397 | 0.825 | −3.614 |
| GI_16950602-S | MRPS35 | 12p11 | 0.599 | 0.824 | −2.303 |
| GI_7657624-S | STAU2 | 8q13-q21.1 | 0.622 | 0.824 | −2.125 |
| GI_22027631-I | DGKB | 7p21.2 | 0.623 | 0.824 | −2.113 |
| GI_21361451-S | GLS | 2q32-q34 | 0.622 | 0.824 | −2.116 |
| GI_20336745-A | H2AFY | 5q31.3-q32 | 0.464 | 0.821 | −3.161 |
| GI_34335244-A | NMNAT2 | 1q25 | 0.550 | 0.821 | −2.604 |
| GI_38788154-1 | GABRG2 | 5q31.1-q33.1 | 0.529 | 0.819 | −2.725 |
| GI_41393590-S | API5 | 11p11.2 | 0.599 | 0.819 | −2.218 |
| GI_13375984-S | FLJ14007 | 8q21.13 | 0.539 | 0.819 | −2.643 |
| GI_38202242-S | YARS | 1p35.1 | 0.554 | 0.814 | −2.482 |
| GI_4505698-S | PDHX | 11p13 | 0.569 | 0.813 | −2.358 |
| GI_45359844-S | G3BP2 | 4q21.1 | 0.402 | 0.813 | −3.415 |
| GI_21536251-S | NBEA | 13q13 | 0.530 | 0.811 | −2.603 |
| GI_20127465-S | HOMER1 | 5q14.2 | 0.534 | 0.811 | −2.574 |
| GI_4557642-S | HMGCR | 5q13.3-q14 | 0.530 | 0.811 | −2.594 |
| GI_31341922-S | LOC151963 | 3q29 | 0.462 | 0.810 | −3.024 |
| GI_4503352-S | DOC2A | 16p11.2 | 0.600 | 0.810 | −2.092 |
| GI_38261964-I | ARPP-21 | 3p22.3 | 0.310 | 0.810 | −3.880 |
| GI_29244580-S | HIP14 | 12q21.2 | 0.554 | 0.810 | −2.416 |
| GI_4506566-S | RNMT | 18p11.22-p11. [symbol] | 0.551 | 0.808 | −2.414 |
| GI_10938020-S | FABP3 | 1p33-p32 | 0.483 | 0.806 | −2.835 |
| GI_42734437-S | BM-009 | 8q24.21 | 0.568 | 0.806 | −2.262 |
| GI_32130513-A | NDRG3 | 20q11.21-q11. [symbol] | 0.331 | 0.806 | −3.709 |
| GI_10092672-S | LOC57019 | 16q13-q21 | 0.523 | 0.805 | −2.563 |
| GI_40316942-S | ALAS1 | 3p21.1 | 0.482 | 0.804 | −2.809 |
| GI_4506456-S | RCN2 | 15q23 | 0.598 | 0.804 | −2.014 |
| GI_23346425-S | ATP5A1 | 18q12-q21 | 0.571 | 0.803 | −2.199 |
| GI_32189393-S | ATP5B | 12q13.13 | 0.346 | 0.799 | −3.533 |
| GI_11612654-S | FXYD6 | 11q23.3 | 0.582 | 0.799 | −2.070 |
| GI_24475975-S | TBC1D7 | 6p24.1 | 0.519 | 0.797 | −2.481 |
| GI_30260186-A | ATPIF1 | null | 0.538 | 0.796 | −2.334 |
| GI_18105038-S | COX7B | Xq21.1 | 0.590 | 0.795 | −1.960 |
| GI_8922600-S | ARL10C | 3p26.2 | 0.443 | 0.793 | −2.908 |
| GI_30025025-S | OSTM1 | 6q21 | 0.506 | 0.793 | −2.505 |
| GI_40805828-A | COPS8 | 2q37.3 | 0.433 | 0.793 | −2.956 |
| GI_40316934-S | ALS2 | 2q33.1 | 0.452 | 0.791 | −2.824 |
| GI_22095341-S | CCT6A | 7p11.2 | 0.302 | 0.791 | −3.663 |
| GI_25188178-S | VDAC3 | 8p11.2 | 0.574 | 0.790 | −2.010 |
| GI_13994272-S | C1QTNF4 | 11q11 | 0.472 | 0.790 | −2.685 |
| GI_23110938-A | PSMA3 | 14q23 | 0.498 | 0.789 | −2.516 |
| GI_5031856-S | LDHA | 11p15.4 | 0.460 | 0.789 | −2.741 |
| GI_34304321-S | MRPL45 | 17q21.2 | 0.356 | 0.788 | −3.339 |
| GI_5803110-S | EBNA1BP2 | 1p35-p33 | 0.452 | 0.788 | −2.782 |
| GI_15147332-S | TRIM37 | 17q23.2 | 0.576 | 0.788 | −1.968 |
| GI_5453857-S | PCP4 | 21q22.2 | 0.234 | 0.787 | −3.968 |
| GI_19923458-S | PAIP2 | 5q31.2 | 0.572 | 0.787 | −1.988 |
| GI_14251213-S | DDX24 | 14q32 | 0.567 | 0.787 | −2.018 |
| GI_34222125-S | RWDD2 | 6q14.2 | 0.516 | 0.786 | −2.357 |
| GI_16950656-S | CCND2 | 12p13 | 0.367 | 0.786 | −3.254 |
| GI_25914753-A | MKKS | 20p12 | 0.459 | 0.785 | −2.709 |
| GI_4502202-S | ARF3 | 12q13 | 0.419 | 0.784 | −2.926 |
| GI_42403584-A | FHL2 | 2q12-q14 | 0.532 | 0.783 | −2.215 |
| GI_24041025-S | NETO2 | 16q11 | 0.330 | 0.782 | −3.408 |
| GI_16933563-I | DNM1L | 12p11.21 | 0.556 | 0.780 | −2.011 |
| GI_34850060-S | STMN2 | 8q21.13 | 0.549 | 0.779 | −2.048 |
| GI_29744085-S | LOC340543 | Xq22.1 | 0.412 | 0.778 | −2.896 |
| GI_5032234-S | DSCR1L1 | 6p12.3 | 0.352 | 0.778 | −3.232 |
| GI_40255241-S | SOD1 | 21q22.1 | 0.558 | 0.777 | −1.963 |
| GI_27436874-S | RUNDC1 | 17q21.31 | 0.549 | 0.776 | −2.019 |
| GI_14149614-S | MGC4189 | 17p13.2 | 0.433 | 0.776 | −2.749 |
| GI_10800414-S | NDN | 15q11.2-q12 | 0.548 | 0.776 | −2.016 |
| GI_14150131-S | MGC12992 | 9q31.1 | 0.483 | 0.775 | −2.434 |
| GI_24496788-S | LARS | 5q32 | 0.378 | 0.775 | −3.054 |
| GI_23510242-S | KIAA1797 | 9p21 | 0.487 | 0.772 | −2.371 |
| GI_37549357-S | LOC375303 | 2q34 | 0.441 | 0.772 | −2.649 |
| GI_21314691-S | HRMT1L3 | 12p13.3 | 0.520 | 0.771 | −2.145 |
| GI_34147353-S | C7orf24 | 7p15-p14 | 0.517 | 0.769 | −2.143 |
| GI_42544158-S | HSPH1 | 13q12.3 | 0.483 | 0.768 | −2.342 |
| GI_19923448-S | DREV1 | 16p13-p12 | 0.351 | 0.766 | −3.097 |
| GI_32171224-S | COQ3 | 6q16.3 | 0.519 | 0.766 | −2.099 |
| GI_19923444-A | SPG3A | 14q22.1 | 0.419 | 0.766 | −2.713 |
| GI_40217821-S | SLITRK4 | Xq2.7.3 | 0.517 | 0.765 | −2.102 |
| GI_21070966-A | NRXN1 | 2p163 | 0.520 | 0.765 | −2.070 |
| GI_21361102-S | SLC25A12 | 2q24 | 0.416 | 0.764 | −2.709 |
| GI_19913444-S | HPCAL4 | 1p34.2 | 0.490 | 0.764 | −2.261 |
| GI_27734993-A | SLC22A17 | 14q11.2 | 0.387 | 0.764 | −2.867 |
| GI_42558257-S | FBXO33 | 14q21.1 | 0.417 | 0.763 | −2.685 |
| GI_21071040-S | CNTNAP2 | 7q35-q36 | 0.505 | 0.762 | −2.133 |
| GI_34916055-I | KNS2 | 14q32.3 | 0.523 | 0.761 | −2.009 |
| GI_40255108-S | GRPEL2 | 5q33.1 | 0.433 | 0.761 | −2.567 |
| GI_15100150-S | BAT5 | 6p21.3 | 0.433 | 0.760 | −2.557 |
| GI_6005726-S | CCT8 | 21q22.11 | 0.516 | 0.759 | −2.033 |
| GI_34222355-S | SIAH2 | 3q25 | 0.440 | 0.759 | −2.504 |
| GI_13325063-S | CELSR2 | 1p21 | 0.202 | 0.758 | −3.782 |
| GI_11056011-S | FLJ14084 | Xq22.1 | 0.255 | 0.756 | −3.493 |
| GI_33519464-S | NDUFA8 | 9q33.2-q34.11 | 0.462 | 0.754 | −2.319 |
| GI_38569472-S | NDUFB1 | 14q32.12 | 0.490 | 0.754 | −2.144 |
| GI_21314723-S | FLJ22490 | 8q13.2 | 0.351 | 0.753 | −2.951 |
| GI_7657479-S | GHITM | 10q23.1 | 0.464 | 0.753 | −2.297 |
| GI_33667026-S | DC50 | 14q24.3 | 0.428 | 0.752 | −2.502 |
| GI_34878876-S | NRN1 | 6p25.1 | 0.426 | 0.751 | −2.500 |
| GI_16950627-I | AP1S1 | 7q22.1 | 0.491 | 0.750 | −2.096 |
| GI_40255259-S | FLJ20701 | 2q36.3 | 0.478 | 0.748 | −2.156 |
| GI_31542934-S | HLF | 17q22 | 0.390 | 0.747 | −2.665 |
| GI_6382080-S | RASGRP1 | 15q15 | 0.132 | 0.746 | −3.999 |
| GI_7705852-S | DNCLI1 | 3p22.3 | 0.295 | 0.746 | −3.172 |
| GI_5453687-S | HSPB3 | 5q11.2 | 0.390 | 0.746 | −2.649 |
| GI_14149608-S | EXTL2 | 1p21 | 0.435 | 0.744 | −2.368 |
| GI_7662227-S | SNAP91 | 6q14.2 | 0.390 | 0.743 | −2.616 |
| GI_31543422-S | POLE3 | 9q33 | 0.388 | 0.742 | −2.624 |
| GI_21389510-S | FLJ31121 | 5q31.3 | 0.448 | 0.740 | −2.254 |
| GI_21464102-S | YWHAH | 22q12.3 | 0.361 | 0.739 | −2.735 |
| GI_6005955-S | DUSP12 | 1q21-q22 | 0.482 | 0.738 | −2.025 |
| GI_15451900-S | KCNK1 | 1q42-q43 | 0.470 | 0.738 | −2.092 |
| GI_45007001-S | LOC253782 | 2q24.3 | 0.422 | 0.738 | −2.381 |
| GI_21914880-S | LGMN | 14q32.1 | 0.356 | 0.737 | −2.751 |
| GI_21389358-S | FLJ30525 | 1p13.3 | 0.484 | 0.737 | −1.997 |
| GI_31542152-S | NPY | 7p15.1 | 0.343 | 0.736 | −2.814 |
| GI_30795230-S | BASP1 | 5p15.1-p14 | 0.379 | 0.735 | −2.597 |
| GI_19923309-S | MCF2 | Xq27 | 0.397 | 0.733 | −2.483 |
| GI_22749448-S | C6orf65 | 6p12.1 | 0.469 | 0.733 | −2.053 |
| GI_13654273-S | DKFZP566J2046 | 16p13.3 | 0.289 | 0.733 | −3.067 |
| GI_40254432-S | SST | 3q28 | 0.300 | 0.732 | −2.994 |
| GI_29568100-S | ATP5L | 11q23.3 | 0.478 | 0.731 | −1.971 |
| GI_4505684-S | PDHA1 | Xp22.2-p22.1 | 0.316 | 0.725 | −2.843 |
| GI_21536273-S | CASQ1 | 1q21 | 0.408 | 0.725 | −2.330 |
| GI_4503874-S | GAD2 | 10p11.23 | 0.078 | 0.724 | −4.033 |
| GI_44917605-S | NAPB | 20p12.3-p11.2 [symbol] | 0.465 | 0.724 | −1.985 |
| GI_13384599-S | SPATA7 | 14q31.3 | 0.311 | 0.723 | −2.850 |
| GI_32528285-A | BACH | 1p36.31-p36.1 [symbol] | 0.351 | 0.723 | −2.633 |
| GI_19924138-S | RAD23B | 9q31.2 | 0.462 | 0.723 | −1.987 |
| GI_8923943-S | NRF | Xq24 | 0.265 | 0.721 | −3.070 |
| GI_18598508-S | CDR2 | 16p12.3 | 0.368 | 0.720 | −2.509 |
| GI_20149591-S | UNRIP | 12p12.3 | 0.332 | 0.718 | −2.686 |
| GI_34222096-S | KIAA0089 | 3p22.3 | 0.326 | 0.718 | −2.715 |
| GI_37551274-S | LOC375489 | 6p25.2 | 0.104 | 0.717 | −3.831 |
| GI_5902001-S | DUSP14 | 17q12 | 0.316 | 0.716 | −2.755 |
| GI_5174744-S | UQCRH | 1p33 | 0.347 | 0.716 | −2.581 |
| GI_37545060-S | KIAA0802 | 18p11.22 | 0.424 | 0.714 | −2.129 |
| GI_5902095-S | SMT3H1 | 21q22.3 | 0.425 | 0.711 | −2.100 |
| GI_40217822-S | SLITRK5 | 13q31.2 | 0.373 | 0.711 | −2.397 |
| GI_8923764-S | CACNA2D3 | 3p21.1 | 0.385 | 0.711 | −2.323 |
| GI_21361534-S | HSPC138 | 11q14.2 | 0.435 | 0.709 | −2.017 |
| GI_24430185-S | PIGC | 1q23-q25 | 0.335 | 0.709 | −2.583 |
| GI_40353771-S | BLVRA | 7p14-cen | 0.382 | 0.709 | −2.322 |
| GI_24308076-S | C18orf10 | 18q12.2 | 0.416 | 0.709 | −2.123 |
| GI_40806213-S | VIAAT | 20q11.23 | 0.348 | 0.708 | −2.499 |
| GI_7706340-S | CGI-127 | 2p23.1 | 0.154 | 0.707 | −3.488 |
| GI_22027654-S | AP1S2 | Xp22.2 | 0.016 | 0.705 | −4.140 |
| GI_14042922-S | C9orf5 | 9q31 | 0.354 | 0.705 | −2.435 |
| GI_29728071-S | KIAA0882 | 4q31.21 | 0.172 | 0.703 | −3.359 |
| GI_38201713-S | ELAVL1 | 19p13.2 | 0.409 | 0.702 | −2.105 |
| GI_27436906-S | MRPL49 | 11q13 | 0.321 | 0.702 | −2.589 |
| GI_20149593-S | HSPCB | 6p12 | 0.425 | 0.700 | −1.990 |
| GI_13375659-S | FLJ22555 | 2q33.1 | 0.351 | 0.699 | −2.403 |
| GI_40254964-S | FLJ11753 | 2q22.2-q22.3 | 0.419 | 0.699 | −2.010 |
| GI_37546515-S | THOC2 | Xq25-q26.3 | 0.425 | 0.698 | −1.970 |
| GI_14719429-S | PNMA1 | 14q24.3 | 0.337 | 0.698 | −2.464 |
| GI_31542700-S | AHI1 | 6q23.3 | 0.185 | 0.697 | −3.249 |
| GI_7661547-S | CL25022 | 2q23.2 | 0.423 | 0.696 | −1.965 |
| GI_20336268-A | GNB5 | 15q21.2 | 0.415 | 0.695 | −1.995 |
| GI_19923886-S | DKFZp761H2121 | 10q26.13 | 0.013 | 0.694 | −4.052 |
| GI_36951161-S | D4S234E | 4p16.3 | 0.299 | 0.693 | −2.621 |
| GI_17017971-S | RPL26L1 | 5q35.2 | 0.322 | 0.693 | −2.498 |
| GI_30794499-S | AOF2 | 1p36.12 | 0.390 | 0.692 | −2.116 |
| GI_33667050-S | DP1 | 5q22-q23 | 0.098 | 0.690 | −3.609 |
| GI_34147446-S | MGC14288 | 12q13.13 | 0.331 | 0.690 | −2.424 |
| GI_7656945-S | SLC30A9 | 4p13-p12 | 0.183 | 0.689 | −3.177 |
| GI_20127458-S | CITED1 | Xq13.1 | 0.253 | 0.689 | −2.822 |
| GI_31795541-A | RFC5 | 12q24.2-q24.3 | 0.282 | 0.688 | −2.667 |
| GI_30158015-S | KIAA1580 | 11p12 | 0.319 | 0.688 | −2.468 |
| GI_16506300-S | TIGA1 | 5q21-q22 | 0.206 | 0.685 | −3.030 |
| GI_5902039-S | RABL2B | 22q13.33 | 0.078 | 0.684 | −3.647 |
| GI_31982913-S | FLJ12953 | 2p13.1 | 0.313 | 0.682 | −2.449 |
| GI_7705579-A | LCMT1 | 16p12.3-p12.1 | 0.258 | 0.682 | −2.733 |
| GI_14249553-S | FLJ14800 | 12q13.13 | 0.332 | 0.681 | −2.336 |
| GI_42655683-S | NTNG1 | 1p13.3 | 0.294 | 0.680 | −2.530 |
| GI_33188457-A | UBE2D2 | 5q31.2 | 0.192 | 0.680 | −3.046 |
| GI_24797146-S | SEPHS2 | 16p11.2 | 0.049 | 0.677 | −3.720 |
| GI_30410793-A | PSME3 | 17q21 | 0.327 | 0.675 | −2.310 |
| GI_34594658-S | FLJ39616 | 12q24.12 | 0.181 | 0.675 | −3.064 |
| GI_32401419-S | SYNPR | 3p14.2 | 0.283 | 0.674 | −2.536 |
| GI_21314689-S | NGLY1 | 3p24.2 | 0.268 | 0.673 | −2.605 |
| GI_24308166-S | DKFZp761H039 | 12q24.11 | 0.376 | 0.673 | −2.029 |
| GI_7706195-S | NEUGRIN | 15q26.1 | 0.306 | 0.670 | −2.377 |
| GI_21359821-S | RNASE3L | 5p13.3 | 0.358 | 0.669 | −2.086 |
| GI_33356141-S | C9orf91 | 9q32 | 0.310 | 0.667 | −2.334 |
| GI_21361744-S | STRBP | 9q33.3 | 0.200 | 0.666 | −2.886 |
| GI_13569955-S | ARPC5L | 9q33.3 | 0.324 | 0.666 | −2.246 |
| GI_32307135-A | NNAT | 20q11.2-q12 | 0.191 | 0.665 | −2.928 |
| GI_37556084-S | LOC375088 | 20p11.23 | 0.364 | 0.665 | −2.020 |
| GI_16306547-S | SARS | 1p13.3-p13.1 | 0.266 | 0.665 | −2.542 |
| GI_16950659-S | CDK7 | 5q12.1 | 0.277 | 0.664 | −2.483 |
| GI_13128967-S | MGC1136 | 8p12 | 0.348 | 0.663 | −2.092 |
| GI_39930484-S | MCSC | 9q34.11 | 0.201 | 0.662 | −2.850 |
| GI_29570797-S | PPAT | 4q12 | 0.257 | 0.659 | −2.539 |
| GI_8922103-S | BM045 | 16p13.3 | 0.336 | 0.659 | −2.121 |
| GI_6006024-S | MPP1 | Xq28 | 0.316 | 0.658 | −2.228 |
| GI_4506330-S | PTS | 11q22.3-q23.3 | 0.335 | 0.657 | −2.110 |
| GI_4501912-S | ADAM23 | 2q33 | 0.162 | 0.657 | −2.997 |
| GI_37545891-S | DKFZP5640092 | 14q11.2 | 0.343 | 0.656 | −2.065 |
| GI_37543775-S | KIAA1340 | 12p11.22 | 0.328 | 0.656 | −2.145 |
| GI_41222851-S | DKFZP564D166 | 17q23.3 | 0.170 | 0.656 | −2.952 |
| GI_16445392-S | CDH12 | 5p14-p13 | 0.282 | 0.655 | −2.373 |
| GI_23238232-S | HMGN4 | 6p21.3 | 0.281 | 0.653 | −2.365 |
| GI_34222360-S | ATP1A1 | 1p21 | 0.211 | 0.652 | −2.717 |
| GI_17999533-S | PRPF18 | 10p13 | 0.194 | 0.650 | −2.779 |
| GI_24308110-S | DKFZp56401863 | 12p11.23 | 0.182 | 0.649 | −2.831 |
| GI_31377794-S | CALM1 | 14q24-q31 | 0.079 | 0.648 | −3.332 |
| GI_14456712-S | HBQ1 | 16p13.3 | 0.276 | 0.647 | −2.339 |
| GI_4507128-S | SNRPE | 1q32 | 0.338 | 0.646 | −2.004 |
| GI_15451873-I | B3GALT3 | 3q25 | 0.274 | 0.644 | −2.323 |
| GI_6912393-S | GNG3 | 11p11 | 0.250 | 0.637 | −2.394 |
| GI_17999532-S | PHYH | 10pter-p11.2 | 0.305 | 0.635 | −2.088 |
| GI_45237192-S | KIAA0446 | 1q22 | 0.184 | 0.635 | −2.709 |
| GI_42734431-S | NLK | 17q11.2 | 0.236 | 0.632 | −2.429 |
| GI_31343354-S | PAQR3 | 4q21.21 | 0.321 | 0.631 | −1.975 |
| GI_27477044-A | MT | 22q13.31 | 0.267 | 0.630 | −2.250 |
| GI_32261311-S | HSPC039 | 18q12 | −0.026 | 0.630 | −3.686 |
| GI_37546978-S | LOC376872 | 2p24.1 | 0.204 | 0.626 | −2.537 |
| GI_37574716-S | TRAPPC5 | 19p13.2 | 0.148 | 0.623 | −2.789 |
| GI_4506516-S | RGS2 | 1q31 | 0.187 | 0.622 | −2.595 |
| GI_16507966-S | ENO2 | 12p13 | 0.286 | 0.621 | −2.079 |
| GI_42476192-S | MGC8721 | 8p12 | 0.220 | 0.619 | −2.403 |
| GI_4503872-I | GAD1 | 2q31 | −0.005 | 0.619 | −3.497 |
| GI_17149845-S | FKBP3 | 14q21.3 | 0.256 | 0.618 | −2.206 |
| GI_21314771-S | ESDN | 3q12.1 | 0.220 | 0.617 | −2.392 |
| GI_24308256-S | KIAA1576 | 16q23.1 | −0.012 | 0.616 | −3.514 |
| GI_12232478-S | ARV1 | 1q42.2 | 0.280 | 0.614 | −2.055 |
| GI_24308180-S | KIAA1354 | 9p22 | 0.287 | 0.613 | −2.012 |
| GI_18375679-S | WBP11 | 12p12.3 | 0.265 | 0.612 | −2.120 |
| GI_14916518-S | AP3M2 | 8p11.2 | 0.273 | 0.610 | −2.068 |
| GI_34850063-S | NDUFC1 | 4q28.2-q31.1 | 0.110 | 0.603 | −2.820 |
| GI_28933464-S | STX12 | 1p35-p34.1 | 0.241 | 0.600 | −2.152 |
| GI_19913429-S | ATP5J | 21q21.1 | 0.212 | 0.598 | −2.280 |
| GI_24430168-A | PANK2 | 20p13 | 0.194 | 0.597 | −2.364 |
| GI_20127554-S | HSPC111 | 5q35.2 | 0.091 | 0.597 | −2.866 |
| GI_42660513-S | LOC390616 | 15q25.1 | 0.089 | 0.595 | −2.867 |
| GI_16753206-S | UBQLN2 | Xp11.23-p11.1 | −0.005 | 0.593 | −3.306 |
| GI_13128995-S | CUEDC2 | 10q24.32 | 0.251 | 0.593 | −2.050 |
| GI_4503766-S | FMR2 | Xq28 | 0.022 | 0.592 | −3.164 |
| GI_16933538-A | GLMN | 1p22.1 | 0.258 | 0.587 | −1.970 |
| GI_37539736-S | LOC343990 | 2q11.2 | 0.047 | 0.585 | −2.992 |
| GI_21956644-S | MTPN | 7q33 | 0.227 | 0.585 | −2.108 |
| GI_27735126-S | SLC35F3 | 1q42.2 | 0.084 | 0.577 | −2.755 |
| GI_7549818-A | RABL2A | 2q13 | 0.088 | 0.577 | −2.735 |
| GI_6806888-S | HSF2 | 6q22.31 | 0.127 | 0.570 | −2.499 |
| GI_22035573-A | 3-Sep | 22q13.2 | 0.226 | 0.570 | −2.006 |
| GI_21389416-S | FLJ31795 | 17q21.31 | 0.197 | 0.569 | −2.148 |
| GI_4758873-S | TM9SF2 | 13q32.3 | 0.194 | 0.568 | −2.154 |
| GI_42655672-S | LOC163404 | 1p21.3 | 0.227 | 0.567 | −1.982 |
| GI_7262387-S | NARS | 18q21.2-q21.3 | 0.197 | 0.567 | −2.133 |
| GI_42734423-S | PSMD14 | 2q24.2 | 0.087 | 0.565 | −2.658 |
| GI_40255050-S | FLJ12770 | 1q23.3 | 0.156 | 0.565 | −2.321 |
| GI_24430187-S | PIGH | 14q11-q24 | 0.114 | 0.565 | −2.527 |
| GI_13259542-A | SLC25A14 | Xq24 | 0.128 | 0.564 | −2.457 |
| GI_34850073-S | CGI-150 | 17p13.3 | 0.183 | 0.564 | −2.177 |
| GI_41281989-I | TRNT1 | null | 0.121 | 0.564 | −2.485 |
| GI_37555873-S | LOC346887 | 8q23.1 | 0.184 | 0.564 | −2.171 |
| GI_28178835-S | IDH3A | 15q25.1-q25.2 | 0.209 | 0.564 | −2.049 |
| GI_6996006-A | DNM1L | 12p11.21 | 0.174 | 0.563 | −2.220 |
| GI_21361928-S | SLC38A1 | 12q13.11 | −0.015 | 0.560 | −3.115 |
| GI_4757883-S | C18orf1 | 18p11.2 | 0.159 | 0.559 | −2.266 |
| GI_33598967-S | LMO7 | 13q22.2 | 0.166 | 0.559 | −2.232 |
| GI_17921992-I | TUBA2 | 13q11 | 0.123 | 0.555 | −2.413 |
| GI_22035640-S | MGST3 | 1q23 | 0.210 | 0.554 | −1.977 |
| GI_40804463-S | C20orf103 | 20p12 | 0.144 | 0.554 | −2.305 |
| GI_34734070-S | GABRD | 1p | 0.175 | 0.554 | −2.149 |
| GI_39725933-S | SERPINF1 | 17p13.1 | 0.051 | 0.551 | −2.736 |
| GI_37538719-S | LOC377527 | 7q21.12 | 0.133 | 0.546 | −2.303 |
| GI_33519435-A | CCNB1IP1 | 14q11.2 | 0.148 | 0.544 | −2.214 |
| GI_42659948-S | LOC283400 | 12q13.13 | 0.186 | 0.541 | −2.006 |
| GI_18491027-S | C15orf15 | 15q21 | 0.114 | 0.538 | −2.343 |
| GI_8923591-S | HARC | 9p24.1 | 0.067 | 0.536 | −2.559 |
| GI_4885584-S | SAE1 | 19q13.32 | 0.141 | 0.536 | −2.196 |
| GI_24430136-S | DXS9879E | Xq28 | 0.109 | 0.535 | −2.340 |
| GI_7657300-S | KLHDC2 | 14q22.1 | 0.010 | 0.529 | −2.783 |
| GI_7549792-A | TBL2 | 7q11.23 | 0.055 | 0.528 | −2.560 |
| GI_41327765-A | ALEX2 | Xq21.33-q22.2 | 0.154 | 0.527 | −2.072 |
| GI_41327727-S | CLTC | 17q11-qter | 0.058 | 0.527 | −2.537 |
| GI_37577121-I | UBE2J1 | 6q15 | −0.094 | 0.520 | −3.227 |
| GI_31543933-S | VMP | 6p22.2 | 0.134 | 0.518 | −2.106 |
| GI_31543390-S | PEG10 | 7q21 | 0.068 | 0.512 | −2.394 |
| GI_32455261-A | PRDX5 | 11q13 | 0.145 | 0.511 | −2.009 |
| GI_22749102-S | FLJ36175 | 2q24.2 | 0.084 | 0.509 | −2.291 |
| GI_37595544-S | PCTK2 | 12q23.1 | 0.055 | 0.509 | −2.432 |
| GI_6996009-S | GARS | 7p15 | 0.064 | 0.507 | −2.381 |
| GI_37549971-S | KIAA1311 | 5q32 | 0.142 | 0.505 | −1.987 |
| GI_38327536-S | INPP5A | 10q26.3 | −0.045 | 0.501 | −2.865 |
| GI_42741681-S | ZNF265 | 1p31 | 0.036 | 0.494 | −2.431 |
| GI_42476122-S | RUSC1 | 1q21-q22 | 0.101 | 0.489 | −2.084 |
| GI_37541920-S | KIAA0789 | 12q23.3 | −0.067 | 0.487 | −2.883 |
| GI_33519472-S | NDFIP1 | 5q31.3 | 0.057 | 0.485 | −2.274 |
| GI_34147622-S | RPA2 | 1p35 | 0.095 | 0.482 | −2.069 |
| GI_27436965-A | KCNAB1 | 3q26.1 | 0.068 | 0.482 | −2.199 |
| GI_21389526-S | MGC29761 | 9q34.3 | −0.047 | 0.481 | −2.750 |
| GI_4502280-S | ATP1B3 | 3q23 | 0.060 | 0.478 | −2.212 |
| GI_37546921-S | LOC339804 | 2p15 | 0.079 | 0.475 | −2.105 |
| GI_37577147-A | NCKIPSD | 3p21 | 0.007 | 0.473 | −2.437 |
| GI_30102943-S | COAS2 | 1q21.1 | 0.098 | 0.472 | −1.992 |
| GI_24797085-S | KPNB3 | 13q32.2 | 0.062 | 0.469 | −2.145 |
| GI_37552180-S | KIAA1246 | 6p21.2-p21.1 | 0.055 | 0.467 | −2.168 |
| GI_21536352-S | ACTL6 | 7q22 | 0.071 | 0.465 | −2.078 |
| GI_22749192-S | FLJ38564 | Xq21.2 | −0.033 | 0.464 | −2.571 |
| GI_27436982-S | KCND2 | 7q31 | 0.021 | 0.460 | −2.291 |
| GI_7657674-S | VAMP2 | 17p13.1 | 0.047 | 0.459 | −2.160 |
| GI_21071055-S | SMARCA4 | 19p13.2 | −0.020 | 0.453 | −2.441 |
| GI_37059763-S | GPHN | 14q23.3 | 0.058 | 0.447 | −2.030 |
| GI_12383063-S | FNDC4 | 2p23.3 | 0.001 | 0.444 | −2.293 |
| GI_21361092-S | TPST1 | 7q11.21 | 0.040 | 0.444 | −2.098 |
| GI_18765755-A | DYRK1A | 21q22.13 | −0.061 | 0.443 | −2.580 |
| GI_37622352-S | NME5 | 5q31 | −0.054 | 0.441 | −2.537 |
| GI_31982879-S | HMGB1 | 13q12 | −0.011 | 0.441 | −2.329 |
| GI_34147695-S | C6orf93 | 6q24.2 | 0.034 | 0.438 | −2.094 |
| GI_4557712-S | LAMB3 | 1q32 | 0.036 | 0.431 | −2.045 |
| GI_4502286-S | ATP2B1 | 12q21.3 | −0.149 | 0.431 | −2.936 |
| GI_29648312-S | LOC57168 | 22.q12.1 | −0.002 | 0.423 | −2.181 |
| GI_8923321-S | FLJ20344 | Xp11.3 | −0.037 | 0.423 | −2.345 |
| GI_7661957-S | BTF | 6q22-q23 | 0.021 | 0.423 | −2.067 |
| GI_18201904-S | GPI | 19q13.1 | 0.009 | 0.420 | −2.111 |
| GI_34147515-S | UAP1 | 1q23.3 | −0.043 | 0.418 | −2.348 |
| GI_7669496-S | JWA | 3p14 | −0.040 | 0.411 | −2.291 |
| GI_21359928-S | XPNPEP1 | 10q25.3 | −0.092 | 0.411 | −2.543 |
| GI_23346417-S | MINA | 3q11.2 | −0.085 | 0.409 | −2.496 |
| GI_13899304-S | CD99L2 | Xq28 | −0.008 | 0.406 | −2.112 |
| GI_32698821-S | LOC90637 | 7p22.3 | −0.038 | 0.406 | −2.256 |
| GI_12669913-S | E2F3 | 6p22 | −0.150 | 0.403 | −2.781 |
| GI_33695108-S | RAB9P40 | 9q33.3 | −0.006 | 0.400 | −2.065 |
| GI_40556362-S | NT5C3 | 7p14.3 | 0.010 | 0.396 | −1.969 |
| GI_21071045-A | SMARCA1 | Xq25 | −0.021 | 0.395 | −2.112 |
| GI_16306542-A | FGF13 | Xq26.3 | −0.090 | 0.394 | −2.440 |
| GI_18254455-S | TSGA2 | 21q22.3 | −0.067 | 0.392 | −2.311 |
| GI_7662646-S | PTDSS1 | 8q22 | −0.014 | 0.386 | −2.023 |
| GI_13376430-S | FLJ13397 | 10p13 | −0.020 | 0.385 | −2.047 |
| GI_21362099-S | ELOVL4 | 6q14 | −0.082 | 0.385 | −2.343 |
| GI_42476300-S | TOMM70A | 3q12.2 | −0.135 | 0.383 | −2.597 |
| GI_28274685-S | ZNF545 | 19q13.12 | −0.029 | 0.380 | −2.063 |
| GI_22060272-S | LOC221424 | 6p21.1 | −0.022 | 0.379 | −2.020 |
| GI_34222114-S | DKFZp566D234 | 4q32.3 | −0.075 | 0.378 | −2.276 |
| GI_42476197-S | MGC15407 | 2p16.1 | −0.159 | 0.378 | −2.680 |
| GI_24497588-S | ARX | Xp21 | −0.174 | 0.374 | −2.735 |
| GI_32307179-S | CHCHD2 | 7p11.2 | −0.062 | 0.372 | −2.177 |
| GI_13937360-S | TRF4-2 | 16q12.1 | −0.119 | 0.371 | −2.447 |
| GI_5209326-S | AMD1 | 6q21-q22 | −0.027 | 0.369 | −1.991 |
| GI_30089957-S | MRPS36 | 5q13.2 | −0.059 | 0.368 | −2.140 |
| GI_33469953-A | RBM12 | 20q11.21 | −0.051 | 0.367 | −2.095 |
| GI_42662641-S | LOC203547 | Xq28 | −0.058 | 0.366 | −2.125 |
| GI_24308070-S | DKFZP566K1924 | 2p14 | −0.170 | 0.365 | −2.667 |
| GI_4502536-S | CACNB4 | 2q22-q23 | −0.228 | 0.364 | −2.947 |
| GI_37549396-S | LOC376965 | 2q24.1 | −0.034 | 0.363 | −1.992 |
| GI_4758403-S | FRG1 | 4q35 | −0.073 | 0.362 | −2.177 |
| GI_37546946-S | LOC375211 | 2p13.1 | −0.066 | 0.359 | −2.126 |
| GI_37546535-S | LOC377960 | Xq25 | −0.047 | 0.351 | −1.990 |
| GI_4557656-S | ICT1 | 17q25.1 | −0.087 | 0.351 | −2.183 |
| GI_4505230-S | MPDZ | 9p24-p22 | −0.073 | 0.348 | −2.095 |
| GI_7661579-I | DKFZP434J154 | 7p22.1 | −0.116 | 0.348 | −2.304 |
| GI_14042940-S | eIF2A | 3q25.1 | −0.218 | 0.330 | −2.711 |
| GI_33519463-S | NDUFA4 | 7p21.3 | −0.149 | 0.327 | −2.355 |
| GI_4506562-S | RNGTT | 6q16 | −0.121 | 0.325 | −2.208 |
| GI_14149798-S | RAB6C | 2q21.1 | −0.105 | 0.320 | −2.098 |
| GI_34222118-S | SYT4 | 18q12.3 | −0.098 | 0.306 | −1.991 |
| GI_32307149-A | OGT | Xq13 | −0.188 | 0.306 | −2.433 |
| GI_25952086-S | KCNA5 | 12p13 | −0.127 | 0.298 | −2.092 |
| GI_44680150-S | CRI1 | 15q21.1-q21.2 | −0.154 | 0.297 | −2.218 |
| GI_5729809-S | EBP | Xp11.23-p11.2 [symbol] | −0.165 | 0.296 | −2.269 |
| GI_41281590-S | MBNL1 | 3q25 | −0.166 | 0.295 | −2.270 |
| GI_33504488-S | ZD52F10 | 19q13.12 | −0.111 | 0.295 | −2.001 |
| GI_9257239-A | SDFR1 | 15q22 | −0.168 | 0.294 | −2.270 |
| GI_4758483-S | GSTO1 | 10q25.1 | −0.149 | 0.292 | −2.172 |
| GI_34147390-S | MGC4093 | 19q13.2 | −0.335 | 0.292 | −3.120 |
| GI_21265079-S | MRPL18 | 6q25.3 | −0.126 | 0.284 | −2.014 |
| GI_21735623-A | YWHAZ | 8q23.1 | −0.134 | 0.271 | −1.987 |
| GI_21314612-S | EIF2S3 | Xp22.2-p22.1 | −0.211 | 0.270 | −2.360 |
| GI_4557600-S | GABRA2 | 4p12 | −0.145 | 0.268 | −2.024 |
| GI_33859846-S | COX7A3 | 4q22.3 | −0.175 | 0.267 | −2.163 |
| GI_30061561-A | GABRB3 | 15q11.2-q12 | −0.172 | 0.266 | −2.149 |
| GI_19224662-S | my048 | Xq22.1-q22.3 | −0.149 | 0.262 | −2.012 |
| GI_7705904-S | DHRS8 | 4q22.1 | −0.139 | 0.261 | −1.960 |
| GI_4757797-S | APG5L | 6q21 | −0.222 | 0.260 | −2.363 |
| GI_4557580-S | FABP5 | 8q21.13 | −0.414 | 0.259 | −3.394 |
| GI_7669474-A | ADAR | 1q21.1-q21.2 | −0.400 | 0.256 | −3.294 |
| GI_7705962-S | RAB9B | Xq22.1-q22.3 | −0.243 | 0.254 | −2.441 |
| GI_15150808-S | LOC90701 | 18q21.32 | −0.172 | 0.247 | −2.045 |
| GI_44680134-A | BDH | 3q29 | −0.166 | 0.241 | −1.984 |
| GI_34222351-S | C1orf37 | 1q32.1 | −0.170 | 0.235 | −1.976 |
| GI_22035617-S | OSBPL8 | 12q14 | −0.188 | 0.221 | −1.995 |
| GI_34147678-S | HOOK1 | 1p32.1 | −0.421 | 0.214 | −3.203 |
| GI_42716286-S | FLJ10904 | 5q14.1 | −0.396 | 0.213 | −3.050 |
| GI_21359921-S | FLJ10581 | 17p13.3 | −0.245 | 0.210 | −2.229 |
| GI_27436972-S | KCNB1 | 20q13.2 | −0.239 | 0.195 | −2.125 |
| GI_30260191-I | ATPIF1 | null | −0.359 | 0.166 | −2.615 |
| GI_37059769-S | MGC42105 | 5p12 | −0.317 | 0.160 | −2.355 |
| GI_37541941-S | LOC376142 | 12q21.31 | −0.273 | 0.154 | −2.093 |
| GI_37537715-A | EIF5 | 14q32.32 | −0.329 | 0.153 | −2.380 |
| GI_20336240-S | PCSK1N | Xp11.23 | −0.269 | 0.135 | −1.980 |
| GI_23110945-A | PSMA7 | 20q13.33 | −0.285 | 0.133 | −2.050 |
| GI_44955928-S | KIAA1078 | 1q32.1 | −0.362 | 0.120 | −2.400 |
| GI_6598326-S | TSTA3 | 8q24.3 | −0.413 | 0.110 | −2.640 |
| GI_31377710-S | FLJ22104 | 11q14.2 | −0.330 | 0.108 | −2.170 |
| GI_39725695-S | L3MBTL2 | 22q13.31-q13. [symbol] | −0.300 | 0.107 | −2.005 |
| GI_41349440-A | SEC31L1 | 4q21.22 | −0.394 | 0.086 | −2.416 |
| GI_37577165-I | LIAS | 4p14 | −0.314 | 0.085 | −1.972 |
| GI_11968046-S | PAF53 | 9p13.2 | −0.376 | 0.051 | −2.149 |
| GI_7549807-S | DNAJA2 | 16q12.1 | −0.456 | 0.049 | −2.600 |
| GI_14195617-A | MAP2 | 2q34-q35 | −0.500 | 0.041 | −2.839 |
| GI_21314666-S | CPSF3 | 2p25.1 | −0.361 | 0.033 | −1.977 |
| GI_13375980-S | FLJ22419 | 3p24.3 | −0.461 | 0.030 | −2.538 |
| GI_27479471-S | KIAA1130 | 14q24.1 | −0.393 | 0.028 | −2.127 |
| GI_14150138-S | PYM | 12q13.2 | −0.393 | 0.028 | −2.128 |
| GI_14861835-A | ALG2 | 9q22.33 | −0.385 | 0.024 | −2.065 |
| GI_29893561-S | C6orf210 | 6q21 | −0.398 | 0.017 | −2.107 |
| GI_32698747-S | ZNF248 | null | −0.434 | 0.004 | −2.257 |
| GI_32306540-S | TRIT1 | 1p35.3-p34.1 | 0.707 | 0.375 | 2.346 |
| GI_13787188-A | CYP2C8 | 10q23.33 | 0.675 | 0.385 | 1.993 |
| GI_33354243-A | NELF | 9q34.3 | 0.569 | 0.160 | 2.331 |
| GI_14141194-S | SDF2 | 17q11.2 | 0.551 | 0.207 | 1.968 |
| GI_34147364-S | MGC4707 | 11p11.2 | 0.536 | 0.098 | 2.404 |
| GI_21536354-A | TAF6 | 7q22.1 | 0.491 | 0.058 | 2.303 |
| GI_20127520-S | C22orf5 | 22q12 | 0.474 | 0.004 | 2.460 |
| GI_4503502-S | EIF2B1 | 12q24.31 | 0.449 | 0.010 | 2.275 |
| GI_13129121-S | MGC2654 | 16p13.2 | 0.444 | −0.002 | 2.305 |
| GI_20336760-S | HEBP1 | 12p13.1 | 0.440 | −0.008 | 2.304 |
| GI_4508008-S | ZNF177 | 19p13.2 | 0.436 | 0.014 | 2.179 |
| GI_21361453-S | PYCR2 | 1q42.12 | 0.436 | 0.049 | 2.008 |
| GI_39753966-S | CSPG5 | 3p21.3 | 0.435 | 0.017 | 2.156 |
| GI_41281667-S | SP2 | 17q21.32 | 0.432 | −0.117 | 2.787 |
| GI_38569431-A | B1 | 7p14 | 0.430 | 0.044 | 2.002 |
| GI_20127496-S | PPP5C | 19q13.3 | 0.382 | −0.164 | 2.728 |
| GI_30023852-S | MTSS1 | 8p22 | 0.380 | −0.018 | 2.009 |
| GI_14971416-S | TRIM28 | 19q13.4 | 0.366 | −0.273 | 3.192 |
| GI_7706183-I | ARL61P4 | 12q24.31 | 0.359 | −0.330 | 3.452 |
| GI_27477112-S | SREBF2 | 22q13 | 0.348 | −0.054 | 2.007 |
| GI_16332359-A | CDC2L1 | 1p36.33 | 0.340 | −0.186 | 2.610 |
| GI_21450690-S | U2AF1L3 | 19q13.12 | 0.338 | −0.315 | 3.256 |
| GI_31341683-S | LOC340371 | 8q24.3 | 0.329 | −0.099 | 2.121 |
| GI_34222318-S | DULLARD | 17p13 | 0.297 | −0.315 | 3.042 |
| GI_7661599-S | DKFZP564B147 | Xq26.3 | 0.294 | −0.132 | 2.094 |
| GI_40786546-S | ANKRD11 | 16q24.3 | 0.272 | −0.136 | 2.001 |
| GI_4504724-S | IRF3 | 19q13.3-q13.4 | 0.267 | −0.153 | 2.057 |
| GI_11321616-S | DPYSL4 | 10q26 | 0.266 | −0.204 | 2.306 |
| GI_14149955-S | DKFZp564A176 | 3q21.3 | 0.265 | −0.138 | 1.972 |
| GI_13129061-S | LENG5 | 19q13.4 | 0.257 | −0.183 | 2.154 |
| GI_38683864-A | RBBP6 | 16p12.2 | 0.248 | −0.346 | 2.953 |
| GI_19718752-S | BAP1 | 3p21.31-p21.2 | 0.237 | −0.250 | 2.392 |
| GI_39811997-A | AES | 19p13.3 | 0.233 | −0.297 | 2.612 |
| GI_15431289-S | RPL11 | 1p36.1-p35 | 0.225 | −0.180 | 1.974 |
| GI_8567387-S | PER3 | 1p36.23 | 0.223 | −0.213 | 2.128 |
| GI_42660142-S | LOC387908 | 13q12.11 | 0.222 | −0.337 | 2.769 |
| GI_14150081-S | MGC4399 | 1p36.22 | 0.217 | −0.238 | 2.226 |
| GI_18379352-S | WFDC1 | 16q24.3 | 0.214 | −0.377 | 2.952 |
| GI_42734336-S | DKFZp434K0410 | 16p11.2 | 0.210 | −0.290 | 2.459 |
| GI_4502896-S | CLPTM1 | 19q13.2-q13.3 | 0.210 | −0.279 | 2.399 |
| GI_21237780-S | WASF3 | 13q12 | 0.210 | −0.222 | 2.110 |
| GI_32481212-S | MK-STYX | 7q11.23 | 0.208 | −0.254 | 2.265 |
| GI_21361675-S | FEZL | 3p14.2 | 0.204 | −0.223 | 2.086 |
| GI_18034689-S | C20orf4 | 20pter-q12 | 0.203 | −0.289 | 2.420 |
| GI_41406096-S | DVL3 | 3q27 | 0.188 | −0.256 | 2.176 |
| GI_34147334-S | FLJ20811 | Xq21.33-q22.3 | 0.181 | −0.250 | 2.107 |
| GI_32401444-S | SPRED2 | 2p14 | 0.179 | −0.384 | 2.819 |
| GI_29725623-S | COL23A1 | 5q35.3 | 0.169 | −0.332 | 2.478 |
| GI_4503664-S | FBLN2 | 3p25.1 | 0.165 | −0.446 | 3.107 |
| GI_37552472-S | LOC286088 | 8p23.3 | 0.163 | −0.253 | 2.030 |
| GI_38788371-S | AQR | 15q14 | 0.156 | −0.268 | 2.075 |
| GI_37541013-S | LOC374395 | 11q12.3 | 0.154 | −0.368 | 2.604 |
| GI_24307876-S | POR | 7q11.2 | 0.147 | −0.347 | 2.449 |
| GI_34734074-A | SLC22A18 | 11p15.5 | 0.142 | −0.395 | 2.695 |
| GI_6006015-S | LGALS1 | 22q13.1 | 0.142 | −0.329 | 2.328 |
| GI_4826959-S | QARS | 3p21.3-p21.1 | 0.140 | −0.376 | 2.581 |
| GI_13376750-S | FLJ11848 | 11q13.4 | 0.137 | −0.378 | 2.573 |
| GI_14589873-A | DOM3Z | 6p21.3 | 0.133 | −0.278 | 2.018 |
| GI_33943097-S | RAB5B | 12q13 | 0.132 | −0.329 | 2.281 |
| GI_37552345-S | LOC374876 | 19p13.3 | 0.132 | −0.315 | 2.207 |
| GI_4758315-S | ETV5 | 3q28 | 0.129 | −0.295 | 2.090 |
| GI_31317254-S | NLGN2 | 17p13.1 | 0.120 | −0.401 | 2.626 |
| GI_38202225-S | ZZEF1 | 17p13.2 | 0.117 | −0.312 | 2.116 |
| GI_16933541-I | FN1 | 2q34 | 0.116 | −0.350 | 2.316 |
| GI_15431298-S | RPL18 | 19q13 | 0.115 | −0.358 | 2.354 |
| GI_15208653-S | DGCR6 | 22q11.21 | 0.114 | −0.485 | 3.094 |
| GI_4507658-S | TPR | 1q25 | 0.108 | −0.396 | 2.536 |
| GI_4507284-S | STX10 | 19p13.13 | 0.108 | −0.398 | 2.548 |
| GI_29171741-A | PPAP2B | 1pter-p22.1 | 0.105 | −0.376 | 2.405 |
| GI_38570070-A | CLDN10 | 13q31-q34 | 0.104 | −0.389 | 2.478 |
| GI_42661292-S | LOC400586 | 17p11.2 | 0.104 | −0.374 | 2.391 |
| GI_15431299-S | RPL18A | 19p13 | 0.098 | −0.329 | 2.115 |
| GI_13569888-S | DC-TM4F2 | 10q23.1 | 0.098 | −0.326 | 2.099 |
| GI_38372922-A | BSG | 19p13.3 | 0.094 | −0.629 | 4.015 |
| GI_7661883-S | HELZ | 17q24.2 | 0.094 | −0.328 | 2.091 |
| GI_24475893-S | GNB2L1 | 5q35.3 | 0.088 | −0.359 | 2.233 |
| GI_18860906-S | USP31 | 1p36.12 | 0.087 | −0.480 | 2.932 |
| GI_21314637-S | NEUROD2 | 17q12 | 0.078 | −0.503 | 3.039 |
| GI_23097284-I | 384D8-2 | 22q13.33 | 0.078 | −0.363 | 2.206 |
| GI_14249383-S | C14orf128 | 14q12 | 0.064 | −0.394 | 2.314 |
| GI_22538458-A | NCOA1 | 2p23 | 0.054 | −0.594 | 3.548 |
| GI_38045937-S | RNF144 | 2p25.2-p25.1 | 0.054 | −0.347 | 2.000 |
| GI_31455613-S | F-LANa | 17p13.2 | 0.053 | −0.388 | 2.223 |
| GI_22749426-S | FLJ36874 | 11q12.1 | 0.053 | −0.392 | 2.244 |
| GI_21359956-S | FLJ21047 | 1q23.3 | 0.050 | −0.365 | 2.083 |
| GI_19923288-S | PIK3CD | 1p36.2 | 0.048 | −0.377 | 2.137 |
| GI_37547125-S | D2S448 | 2p25 | 0.042 | −0.387 | 2.168 |
| GI_44889474-S | RAB6IP1 | 11p15.4 | 0.039 | −0.425 | 2.365 |
| GI_34147360-S | MGC2749 | 19p13.11 | 0.034 | −0.416 | 2.292 |
| GI_4505122-S | MBP | 18q23 | 0.023 | −0.433 | 2.341 |
| GI_4758083-S | CSPG3 | 19p12 | 0.021 | −0.620 | 3.592 |
| GI_24371247-S | HCBP6 | Xq28 | 0.020 | −0.372 | 1.976 |
| GI_23397665-S | SIN3A | 15q24.2 | 0.012 | −0.408 | 2.140 |
| GI_21536450-A | PHF1 | 6p21.3 | 0.005 | −0.512 | 2.741 |
| GI_45433544-S | KIAA0460 | 1q21.2 | 0.005 | −0.391 | 2.012 |
| GI_30795195-S | LHX2 | 9q33-q34.1 | 0.003 | −0.386 | 1.973 |
| GI_38570104-S | RAIN | 19q13.33 | 0.001 | −0.449 | 2.329 |

[symbol]

indicates data missing or illegible when filed

SUPPLEMENTARY TABLE 9
This file contains the detailed results of the aSyn ratio QTL analysis, with the SNIPs found to be
associated to aSynL:total ratio in unaffected cortex with a p-value < 1.0e−3.03

| | | | Frequency | | | Genotype | | | Mean log-ratio | | |
|---|---|---|---|---|---|---|---|---|---|---|---|
| CHR | SNP | p-value | G11 | G12 | G22 | G11 | G12 | G22 | G11 | G12 | G22 |
| 4 | rs356168 | 2.70E−07 | 0.2409 | 0.4678 | 0.2913 | C/C | C/T | T/T | 0.219 | −0.0311 | −0.1256 |
| 16 | rs1115023 [symbol] | 2.55E−06 | 0.259 | 0.4601 | 0.281 | T/T | T/G | G/G | 0.1442 | 0.02985 | −0.1591 |
| 23 | rs5970014 | 5.78E−06 | 0.3177 | 0.2265 | 0.4558 | A/A | A/G | G/G | 0.1697 | −0.03346 | −0.0857 |
| 8 | rs1095497 [symbol] | 7.37E−06 | 0.2396 | 0.5014 | 0.2591 | A/A | A/G | G/G | 0.1533 | 0.01286 | −0.1502 |
| 6 | rs2842846 | 1.35E−05 | 0.06887 | 0.4325 | 0.4986 | C/C | C/A | A/A | 0.3844 | 0.03439 | −0.07017 |
| 18 | rs754789 | 1.52E−05 | 0.01729 | 0.2363 | 0.7464 | C/C | C/T | T/T | −0.1298 | −0.1868 | 0.07097 |
| 3 | rs7629689 | 1.56E−05 | 0.1378 | 0.5044 | 0.3578 | A/A | A/T | T/T | 0.1398 | 0.06685 | −0.1419 |
| 12 | rs1104672 [symbol] | 2.10E−05 | 0.002755 | 0.1377 | 0.8595 | C/C | C/A | A/A | 0.161 | 0.264 | −0.03542 |
| 6 | rs6907063 | 2.91E−05 | 0.1437 | 0.4655 | 0.3908 | A/A | A/G | G/G | 0.3147 | −0.04062 | −0.06416 |
| 2 | rs1257178 | 2.96E−05 | 0.1243 | 0.4641 | 0.4116 | C/C | C/T | T/T | −0.2197 | −0.01866 | 0.1009 |
| 17 | rs1476462 | 3.54E−05 | 0.03824 | 0.3059 | 0.6559 | T/T | T/C | C/C | −0.2575 | −0.1116 | 0.08288 |
| 6 | rs9384860 | 3.78E−05 | 0.06685 | 0.429 | 0.5042 | C/C | C/A | A/A | 0.3594 | 0.03505 | −0.06963 |
| 2 | rs4667454 | 3.81E−05 | 0.1191 | 0.4515 | 0.4294 | C/C | C/T | T/T | −0.1869 | −0.03337 | 0.1089 |
| 15 | rs4778757 | 4.40E−05 | 0.03581 | 0.27 | 0.6942 | C/C | C/G | G/G | −0.1789 | −0.1412 | 0.07331 |
| 6 | rs7451240 | 4.93E−05 | 0.03047 | 0.2992 | 0.6704 | A/A | A/G | G/G | −0.3104 | −0.1065 | 0.06814 |
| 21 | rs1444358 | 5.63E−05 | 0.04665 | 0.3265 | 0.6268 | C/C | C/T | T/T | −0.3686 | −0.06907 | 0.06838 |
| 6 | rs9296193 | 6.00E−05 | 0.04959 | 0.3554 | 0.595 | C/C | C/G | G/G | −0.2418 | −0.08327 | 0.08057 |
| 4 | rs7686587 | 6.64E−05 | 0.09706 | 0.3588 | 0.5441 | G/G | G/A | A/A | 0.1722 | 0.09876 | −0.08532 |
| 6 | rs9377153 | 6.96E−05 | 0.0303 | 0.3003 | 0.6694 | A/A | A/G | G/G | −0.3104 | −0.1005 | 0.06862 |
| 10 | rs4394764 | 6.98E−05 | 0.03581 | 0.3388 | 0.6253 | T/T | T/C | C/C | 0.2054 | 0.1213 | −0.06732 |
| 6 | rs6925433 | 7.14E−05 | 0.06977 | 0.4419 | 0.4884 | A/A | A/G | G/G | −0.1704 | −0.0691 | 0.1079 |
| 11 | rs1104244 [symbol] | 7.21E−05 | 0.05556 | 0.3306 | 0.6139 | A/A | A/G | G/G | 0.363 | 0.06239 | −0.05111 |
| 8 | rs7013706 | 7.27E−05 | 0.169 | 0.4626 | 0.3684 | A/A | A/C | C/C | −0.1484 | −0.02445 | 0.1182 |
| 6 | rs1547334 | 7.59E−05 | 0.005935 | 0.1.662 | 0.8279 | A/A | A/G | G/G | −0.7411 | −0.1738 | 0.04595 |
| 1 | rs1203323 [symbol] | 7.79E−05 | 0 | 0.1709 | 0.8291 | C/C | C/T | T/T | NA | −0.2098 | 0.04206 |
| 21 | rs9980326 | 7.93E−05 | 0.04696 | 0.3287 | 0.6243 | A/A | A/G | G/G | −0.3369 | −0.0629 | 0.06848 |
| 13 | rs2764015 | 8.01E−05 | 0 | 0 1191 | 0.8809 | C/C | C/A | A/A | NA | −0.2529 | 0.03883 |
| 11 | rs1104244 [symbol] | 8.07E−05 | 0.05234 | 0.3333 | 0.6143 | A/A | A/G | G/G | 0.3743 | 0.05818 | −0.05311 |
| 21 | rs9984859 | 8.19E−05 | 0.04482 | 0.3305 | 0.6246 | A/A | A/T | T/T | −0.3686 | −0.06057 | 0.06632 |
| 3 | rs1093501 [symbol] | 8.62E−05 | 0.008264 | 0.2397 | 0.7521 | T/T | T/C | C/C | 0.2497 | 0.1677 | −0.04774 |
| 6 | rs1320588 [symbol] | 8.85E−05 | 0.04638 | 0.3333 | 0.6203 | C/C | C/G | G/G | 0.3959 | 0.07865 | −0.04231 |
| 1 | rs1203678 [symbol] | 9.44E−05 | 0 | 0.1737 | 0.8263 | C/C | C/T | T/T | NA | −0.2014 | 0.04723 |
| 6 | rs6905873 | 9.50E−05 | 0.2051 | 0.486 | 0.309 | A/A | A/T | T/T | −0.05891 | −0.07648 | 0.178 |
| 6 | rs2065147 | 9.56E−05 | 0.01412 | 0.3136 | 0.6723 | C/C | C/A | A/A | −0.2691 | −0.1151 | 0.07394 |
| 6 | rs812479 | 0.000102 | 0.2171 | 0.4543 | 0.3286 | C/C | C/A | A/A | −0.1062 | −0.04047 | 0.143 |
| 11 | rs1089490 [symbol] | 0.000107 | 0.07438 | 0.4904 | 0.4353 | C/C | C/G | G/G | 0.2659 | 0.04396 | −0.08035 |
| 14 | rs1162145 [symbol] | 0.000108 | 0.0112 | 0.2353 | 0.7535 | T/T | T/G | G/G | 0.438 | 0.1466 | −0.04678 |
| 6 | rs6912415 | 0.00011 | 0.234 | 0.4791 | 0.2869 | T/T | T/C | C/C | −0.1021 | −0.02471 | 0.1505 |
| 11 | rs1229015 [symbol] | 0.00011 | 0.2219 | 0.5216 | 0.2565 | A/A | A/C | C/C | 0.1041 | 0.05804 | −0.1634 |
| 11 | rs1122170 [symbol] | 0.000111 | 0.008772 | 0.1257 | 0.8655 | A/A | A/G | G/G | −0.7268 | −0.1698 | 0.04287 |
| 4 | snp_a-189 [symbol] | 0.000112 | 0.03274 | 0.2589 | 0.7083 | G/G | G/A | A/A | −0.3652 | −0.09996 | 0.05433 |
| 17 | rs9905834 | 0.000114 | 0.03641 | 0.3501 | 0.6134 | G/G | G/A | A/A | 0.3837 | 0.0811 | −0.0528 |
| 6 | rs9366911 | 0.000122 | 0.0854 | 0.4325 | 0.4821 | C/C | C/G | G/G | −0.2337 | −0.03488 | 0.08587 |
| 8 | rs1947299 | 0.000129 | 0.1523 | 0.454 | 0.3937 | G/G | G/A | A/A | −0.1644 | −0.02246 | 0.1066 |
| 6 | rs1115341 [symbol] | 0.000131 | 0.1602 | 0.4724 | 0.3674 | G/G | G/A | A/A | −0.0651 | −0.08328 | 0.1472 |
| 18 | rs1108243 [symbol] | 0.000134 | 0.03324 | 0.3435 | 0.6233 | G/G | G/A | A/A | −0.1636 | −0.112 | 0.07553 |
| 14 | rs4902348 | 0.00014 | 0.01111 | 0.2389 | 0.75 | T/T | T/C | C/C | 0.4574 | 0.1409 | −0.04414 |
| 6 | rs9400760 | 0.000144 | 0.06354 | 0.4254 | 0.511 | G/G | G/C | C/C | 0.233 | 0.07083 | −0.07372 |
| 12 | rs1106206 [symbol] | 0.000147 | 0.04167 | 0.3361 | 0.6222 | A/A | A/G | G/G | −0.1549 | −0.1097 | 0.07935 |
| 6 | rs4523125 | 0.000147 | 0.1278 | 0.4801 | 0.392 | A/A | A/G | G/G | 0.1625 | 0.05209 | −0.1013 |
| 6 | rs1246940 | 0.0001.52 | 0.108 | 0.4602 | 0.4318 | G/G | G/A | A/A | −0.2095 | −0.0189 | 0.09587 |
| 2 | rs1703328 [symbol] | 0.000156 | 0.01497 | 0.1617 | 0.8234 | A/A | A/G | G/G | 0.3843 | 0.171 | −0.05067 |
| 23 | rs1731946 [symbol] | 0.000157 | 0.174 | 0.2072 | 0.6188 | A/A | A/G | G/G | 0.2218 | −0.02051 | −0.04656 |
| 21 | rs992039 | 0.000162 | 0.0423 | 0.3746 | 0.5831 | C/C | C/T | T/T | 0.2686 | 0.1035 | −0.06498 |
| 12 | rs1084860 [symbol] | 0.000162 | 0.04132 | 0.3361 | 0.6226 | G/G | G/A | A/A | −0.1549 | −0.1072 | 0.07838 |
| 7 | rs4948033 | 0.000164 | 0.1989 | 0.4779 | 0.3232 | G/G | G/C | C/C | 0.1478 | 0.02155 | −0.1058 |
| 6 | rs2357128 | 0.000171 | 0.1302 | 0.5042 | 0.3657 | T/T | T/C | C/C | 0.1585 | 0.04918 | −0.1016 |
| 6 | rs1707850 [symbol] | 0.000173 | 0.01705 | 0.2926 | 0.6903 | T/T | T/C | C/C | −0.2011 | −0.1352 | 0.0594 |
| 7 | rs4947522 | 0.000176 | 0.211 | 0.4624 | 0.3266 | A/A | A/G | G/G | 0.1368 | 0.04659 | −0.106 |
| 6 | rs2220790 | 0.000177 | 0.2319 | 0.4232 | 0.3449 | C/C | C/T | T/T | −0.129 | 0.001802 | 0.1179 |
| 16 | rs4454988 | 0.00018 | 0.1657 | 0.4061 | 0.4282 | G/G | G/C | C/C | −0.1339 | −0.0366 | 0.1.047 |
| 12 | snp_a-206 [symbol] | 0.000191 | 0.005525 | 0.1575 | 0.837 | G/G | G/A | A/A | −0.6251 | −0.1706 | 0.04345 |
| 22 | rs1003846 | 0.000192 | 0.002841 | 0.1023 | 0.8949 | A/A | A/G | G/G | −0.1864 | −0.2494 | 0.0466 |
| 4 | rs1343493 [symbol] | 0.000195 | 0.08033 | 0.4377 | 0.482 | A/A | A/G | G/G | 0.1605 | 0.07867 | −0.08609 |
| 6 | rs9355389 | 0.000199 | 0.005634 | 0.1606 | 0.8338 | T/T | T/C | C/C | 0.4423 | 0.1906 | −0.03709 |
| 6 | rs829813 | 0.000203 | 0.1424 | 0.5029 | 0.3547 | G/G | G/A | A/A | 0.166 | 0.03407 | −0.1014 |
| 6 | rs2452955 | 0.000209 | 0.2127 | 0.4586 | 0.3287 | C/C | C/T | T/T | −0.125 | −0.00795 | 0.1179 |
| 16 | rs8059713 | 0.00021 | 0.1983 | 0.4626 | 0.3391 | T/T | T/A | A/A | 0.1385 | 0.02927 | −0.1076 |
| 8 | rs4874138 | 0.000212 | 0.1337 | 0.4875 | 0.3788 | A/A | A/G | G/G | −0.1323 | −0.03917 | 0.1164 |
| 16 | rs9940998 | 0.000213 | 0.1333 | 0.4111 | 0.4556 | T/T | T/G | G/G | −0.1935 | −0.02114 | 0.08012 |
| 16 | rs1731222 [symbol] | 0.000215 | 0.01681 | 0.1541 | 0.8291 | GIG | G/A | A/A | 0.5988 | 0.1263 | −0.02999 |
| 20 | rs1467414 | 0.000215 | 0.02521 | 0.2633 | 0.7115 | T/T | T/C | C/C | −0.2498 | −0.1201 | 0.06196 |
| 16 | rs8043932 | 0.000216 | 0.1364 | 0.3864 | 0.4773 | A/A | A/T | T/T | −0.1833 | −0.02606 | 0.08384 |
| 6 | rs7742701 | 0.000216 | 0.1961 | 0.4945 | 0.3094 | T/T | T/C | C/C | −0.05967 | −0.06896 | 0.1653 |
| 2 | rs6737952 | 0.00022 | 0 1188 | 0.5028 | 0.3785 | A/A | A/G | G/G | 0.256 | 0.002549 | −0.06936 |
| 6 | rs6568860 | 0.000223 | 0.06534 | 0.4119 | 0.5227 | T/T | T/C | C/C | 0.233 | 0.06564 | −0.07297 |
| 18 | rs8089950 | 0.000226 | 0.1025 | 0.4183 | 0.4792 | T/T | T/G | G/G | −0.08719 | −0.09137 | 0.1069 |
| 6 | rs1687799 [symbol] | 0.000229 | 0.01934 | 0.1768 | 0.8039 | C/C | C/A | A/A | −0.3282 | −0.1485 | 0.04833 |
| 10 | rs1118781 [symbol] | 0.000232 | 0.1924 | 0.481 | 0.3265 | G/G | G/A | A/A | −0.1287 | −0.01116 | 0.1258 |
| 6 | rs2811686 | 0.000232 | 0.06509 | 0.3639 | 0.571 | G/G | G/T | T/T | −0.1616 | −0.09668 | 0.0821 |
| 16 | rs8045969 | 0.000235 | 0.1529 | 0.3971 | 0.45 | T/T | T/C | C/C | −0.1624 | −0.01524 | 0.09801 |
| 15 | rs1243819 [symbol] | 0.000237 | 0.002941 | 0.2618 | 0.7353 | G/G | G/A | A/A | −0.3194 | 0.1675 | −0.05314 |
| 4 | rs1002620 [symbol] | 0.000241 | 0.1625 | 0.4876 | 0.3499 | T/T | T/G | G/G | −0.09707 | −0.04915 | 0.1318 |
| 3 | rs1051362 [symbol] | 0.000244 | 0.01404 | 0.1826 | 0.8034 | A/A | A/G | G/G | 0.392 | 0.1589 | −0.03679 |
| 10 | rs1119799 [symbol] | 0.000246 | 0.01201 | 0.08408 | 0.9039 | C/C | C/A | A/A | 0.5139 | 0.2338 | −0.02604 |
| 6 | rs7745469 | 0.000247 | 0.1183 | 0.4958 | 0.3859 | G/G | G/A | A/A | 0.1788 | 0.04608 | −0.08751 |
| 13 | rs2764020 | 0.000251 | 0 | 0.1163 | 0.8837 | A/A | A/G | G/G | NA | −0.2375 | 0.03671 |
| 6 | rs2637534 | 0.000252 | 0.1933 | 0.4986 | 0.3081 | G/G | G/A | A/A | −0.04636 | −0.06691 | 0.1751 |
| 18 | rs1783649 [symbol] | 0.000252 | 0.01108 | 0.1662 | 0.8227 | C/C | C/T | T/T | −0.5124 | −0.1449 | 0.04504 |
| 18 | rs1187523 [symbol] | 0.000255 | 0.1185 | 0.5069 | 0.3747 | A/A | A/G | G/G | −0.1356 | −0.04017 | 0.1142 |
| 5 | rs620224 | 0.00026 | 0.005797 | 0.1217 | 0.8725 | T/T | T/C | C/C | 0.06811 | 0.2686 | −0.025 |
| 8 | rs966740 | 0.000271 | 0 | 0.1188 | 0.8812 | A/A | A/C | C/C | NA | −0.2315 | 0.03855 |
| 5 | rs4921336 | 0.000275 | 0.2039 | 0.4573 | 0.3388 | G/G | G/A | A/A | 0.1138 | 0.04843 | −0.1151 |
| 12 | rs1223079 [symbol] | 0.000278 | 0.04237 | 0.3418 | 0.6158 | T/T | T/C | C/C | −0.1549 | −0.1087 | 0.07165 |
| 4 | rs1250236 [symbol] | 0.000284 | 0.1653 | 0.427 | 0.4077 | A/A | A/G | G/G | −0.1312 | −0.03303 | 0.1034 |
| 10 | rs7897082 | 0.000287 | 0.002793 | 0.1369 | 0.8603 | A/A | A/G | G/G | −0.9303 | −0.1859 | 0.03722 |
| 15 | rs1051921 [symbol] | 0.000297 | 0.0117 | 0.2398 | 0.7485 | T/T | T/A | A/A | 0.1464 | 0.1637 | −0.05119 |
| 16 | rs1045986 [symbol] | 0.000307 | 0.1298 | 0.4116 | 0.4586 | G/G | G/C | C/C | −0.1646 | −0.03143 | 0.08898 |
| 11 | rs1229431 [symbol] | 0.000308 | 0.00277 | 0.1302 | 0.867 | C/C | C/A | A/A | 0.7897 | 0.2052 | −0.02755 |
| 10 | rs363309 | 0.00031 | 0.008264 | 0.2011 | 0.7906 | A/A | A/G | G/G | 0.3791 | 0.1613 | −0.03694 |
| 7 | rs7811683 | 0.000314 | 0.2201 | 0.4791 | 0.3008 | T/T | T/A | A/A | 0.1134 | 0.03613 | −0.1233 |
| 8 | rs7003443 | 0.000316 | 0.1547 | 0.4834 | 0.3619 | T/T | T/C | C/C | −0.1595 | −0.01191 | 0.09927 |
| 1 | rs292004 | 0.000321 | 0.005525 | 0.105 | 0.8895 | G/G | G/A | A/A | −1.04 | −0.1604 | 0.03294 |
| 4 | rs2306597 | 0.000322 | 0.0338 | 0.338 | 0.6282 | A/A | A/G | G/G | 0.1982 | 0.1072 | −0.06389 |
| 12 | rs1084385 [symbol] | 0.000327 | 0.05785 | 0.3802 | 0.562 | A/A | A/T | T/T | 0.287 | 0.05775 | −0.0573 |
| 15 | rs1695443 [symbol] | 0.000336 | 0.005935 | 0.1484 | 0.8457 | G/G | G/T | T/T | −0.511 | −0.1769 | 0.04922 |
| 1 | rs1091728 [symbol] | 0.000351 | 0.03064 | 0.2869 | 0.6825 | G/G | G/C | C/C | −0.4282 | −0.06178 | 0.05525 |
| 13 | rs831208 | 0.000356 | 0.2271 | 0.5103 | 0.2625 | T/T | T/C | C/C | −0.06575 | −0.0429 | 0.1805 |
| 13 | rs7336145 | 0.000361 | 0.002762 | 0.1077 | 0.8895 | T/T | T/C | C/C | 1.221 | 0.2011 | −0.02248 |
| 12 | rs4103862 | 0.000362 | 0.1412 | 0.4859 | 0.3729 | A/A | A/G | G/G | 0.1903 | 0.02494 | −0.07796 |
| 8 | rs1827153 | 0.000372 | 0.05249 | 0.2928 | 0.6547 | G/G | G/A | A/A | −0.2116 | −0.08413 | 0.06684 |
| 20 | rs1766582 [symbol] | 0.000373 | 0.00578 | 0.1676 | 0.8266 | T/T | T/C | C/C | −0.4202 | −0.1514 | 0.05861 |
| 7 | rs2214654 | 0.000374 | 0.1922 | 0.4791 | 0.3287 | A/A | A/C | C/C | 0.1381 | 0.02617 | −0.1022 |
| 6 | rs9320441 | 0.000376 | 0.2051 | 0.5169 | 0.2781 | A/A | A/G | G/G | −0.05849 | −0.04612 | 0.1735 |
| 11 | rs4572098 | 0.000376 | 0.03039 | 0.2238 | 0.7459 | C/C | C/T | T/T | 0.2609 | 0.1351 | −0.0404 |
| 12 | rs4559767 | 0.000382 | 0.2176 | 0.4882 | 0.2941 | G/G | G/C | C/C | −0.1547 | 0.006372 | 0.09822 |
| 7 | rs6465471 | 0.000385 | 0.2216 | 0.4737 | 0.3047 | A/A | A/G | G/G | 0.1104 | 0.04165 | −0.1184 |
| 12 | rs1111109 [symbol] | 0.000387 | 0.02632 | 0.2222 | 0.7515 | C/C | C/T | T/T | 0.4676 | 0.1033 | −0.03032 |
| 4 | rs6838244 | 0.000388 | 0.149 | 0.4269 | 0.4241 | A/A | A/G | G/G | 0.1788 | 0.03158 | −0.07683 |
| 6 | rs6925886 | 0.00039 | 0.08564 | 0.4641 | 0.4503 | T/T | T/C | C/C | 0.1776 | 0.06221 | −0.07878 |
| 20 | rs2179604 | 0.000397 | 0.00554 | 0.1745 | 0.8199 | C/C | C/G | G/G | −0.7831 | −0.1383 | 0.04422 |
| 10 | rs4244260 | 0.000398 | 0.1478 | 0.458 | 0.3942 | A/A | A/G | G/G | −0.1374 | −0.03003 | 0.1047 |
| 12 | rs2717446 | 0.000398 | 0.01117 | 0.2737 | 0.7151 | C/C | C/T | T/T | −0.667 | −0.08584 | 0.05585 |
| 4 | rs4241838 | 0.000401 | 0.09366 | 0.449 | 0.4573 | G/G | G/T | T/T | −0.05585 | −0.08981 | 0.1135 |
| 3 | snp_a-189 [symbol] | 0.000407 | 0.00554 | 0.09972 | 0.8947 | G/G | G/C | C/C | 0.3893 | 0.2456 | −0.02336 |
| 5 | rs462498 | 0.000407 | 0.1662 | 0.5163 | 0.3175 | C/C | C/T | T/T | −0.1661 | −0.00633 | 0.1009 |
| 7 | rs2598044 | 0.000409 | 0.04444 | 0.3556 | 0.6 | T/T | T/C | C/C | 0.3881 | 0.0497 | −0.04737 |
| 11 | rs2658785 | 0.000411 | 0.01377 | 0.1433 | 0.843 | C/C | C/T | T/T | −0.7832 | −0.08467 | 0.03473 |
| 6 | rs457492 | 0.000419 | 0.1606 | 0.5183 | 0.3211 | C/C | C/T | T/T | −0.1616 | −0.00083 | 0.1008 |
| 15 | rs8034910 | 0.00042 | 0.1773 | 0.4622 | 0.3605 | C/C | C/G | G/G | 0.2014 | −0.02409 | −0.0713 |
| 6 | rs942923 | 0.000423 | 0.1285 | 0.4804 | 0.3911 | T/T | T/C | C/C | 0.1414 | 0.05715 | −0.09106 |
| 2 | rs1167758 [symbol] | 0.000429 | 0.05833 | 0.3778 | 0.5639 | T/T | T/C | C/C | 0.135 | 0.09752 | −0.07427 |
| 11 | rs1222245 [symbol] | 0.00043 | 0.01681 | 0.3361 | 0.6471 | T/T | T/A | A/A | 0.3537 | 0.1073 | −0.04728 |
| 6 | rs7763648 | 0.000437 | 0.06 | 0.4286 | 0.5114 | G/G | G/C | C/C | 0.2424 | 0.06584 | −0.06348 |
| 19 | rs9989732 | 0.000438 | 0.08571 | 0.4343 | 0.48 | T/T | T/C | C/C | 0.2629 | 0.03547 | −0.05898 |
| 16 | rs1770505 [symbol] | 0.000438 | 0 | 0.1474 | 0.8526 | A/A | A/G | G/G | NA | 0.223 | −0.02179 |
| 6 | rs774407 | 0.000441 | 0.2312 | 0.468 | 0.3008 | A/A | A/G | G/G | 0.1213 | 0.01363 | −0.1101 |
| 9 | rs9308278 | 0.000442 | 0.07778 | 0.4028 | 0.5194 | G/G | G/A | A/A | −0.1653 | −0.06164 | 0.08352 |
| 20 | rs1232602 | 0.00045 | 0.1278 | 0.4389 | 0.4333 | T/T | T/C | C/C | −0.1464 | −0.0368 | 0.09473 |
| 20 | rs2296236 | 0.000451 | 0.02778 | 0.2417 | 0.7306 | T/T | T/C | C/C | −0.08059 | −0.1505 | 0.06416 |
| 4 | rs6534723 | 0.000452 | 0.05638 | 0.3294 | 0.6142 | T/T | T/A | A/A | −0.09423 | −0.122 | 0.08067 |
| 8 | rs966738 | 0.000452 | 0 | 0.1219 | 0.8781 | G/G | G/C | C/C | NA | −0.2222 | 0.03536 |
| 5 | rs4580760 | 0.000457 | 0.005714 | 0.1229 | 0.8714 | T/T | T/G | G/G | −0.5833 | −0.1828 | 0.04296 |
| 12 | rs2292503 | 0.000459 | 0.1598 | 0.4793 | 0.3609 | C/C | C/T | T/T | 0.1781 | 0.01315 | −0.07871 |
| 18 | rs2112058 | 0.000462 | 0.002833 | 0.1133 | 0.8839 | C/C | C/A | A/A | −0.7783 | −0.2101 | 0.03105 |
| 6 | rs2145144 | 0.000463 | 0.1208 | 0.4944 | 0.3848 | T/T | T/G | G/G | 0.1599 | 0.04135 | −0.09306 |
| 10 | rs7075577 | 0.000467 | 0.1298 | 0.4586 | 0.4116 | A/A | A/G | G/G | −0.07667 | −0.07472 | 0.1161 |
| 13 | rs9317632 | 0.000469 | 0.03693 | 0.2699 | 0.6932 | G/G | G/A | A/A | 0.3721 | 0.0778 | −0.04073 |
| 11 | rs1938736 | 0.000469 | 0.04213 | 0.2949 | 0.6629 | T/T | T/G | G/G | −0.1699 | −0.1067 | 0.06563 |
| 10 | rs3816785 | 0.00047 | 0.03591 | 0.3149 | 0.6492 | C/C | C/T | T/T | 0.1253 | 0.1269 | −0.05939 |
| 11 | rs1735575 [symbol] | 0.000479 | 0.03478 | 0.3275 | 0.6377 | G/G | G/A | A/A | 0.3936 | 0.07446 | −0.04477 |
| 5 | rs2910029 | 0.000488 | 0.07345 | 0.3616 | 0.565 | G/G | G/C | C/C | −0.2436 | −0.04554 | 0.06536 |
| 8 | rs1688558 [symbol] | 0.000489 | 0.008523 | 0.1392 | 0.8523 | A/A | A/G | G/G | −0.1583 | −0.2151 | 0.04 |
| 12 | rs6580890 | 0.000489 | 0.1611 | 0.475 | 0.3639 | T/T | T/C | C/C | 0.1723 | 0.02061 | −0.07871 |
| 4 | rs1712493 | 0.000494 | 0.1803 | 0.5437 | 0.2761 | T/T | T/C | C/C | −0.1342 | −0.00971 | 0.1194 |
| 7 | rs6465472 | 0.000499 | 0.2377 | 0.4493 | 0.313 | T/T | T/A | A/A | 0.1041 | 0.02725 | −0.1215 |
| 13 | rs2875248 | 0.000504 | 0.01404 | 0.1489 | 0.8371 | T/T | T/C | C/C | −0.06123 | −0.2165 | 0.04768 |
| 17 | rs572850 | 0.000506 | 0.08989 | 0.4551 | 0.4551 | T/T | T/C | C/C | −0.2073 | −0.03265 | 0.0815 |
| 2 | rs6751992 | 0.000511 | 0.2417 | 0.5 | 0.2583 | A/A | A/G | G/G | −0.1128 | −0.00435 | 0.1231 |
| 1 | rs234115 | 0.000515 | 0.2201 | 0.5348 | 0.2451 | T/T | T/A | A/A | −0.09565 | −0.02005 | 0.1478 |
| 1 | rs2748937 | 0.000516 | 0.2222 | 0.5278 | 0.25 | G/G | G/C | C/C | −0.08939 | −0.02374 | 0.1511 |
| 8 | rs6991453 | 0.000528 | 0.1111 | 0.5333 | 0.3556 | T/T | T/C | C/C | 0.1753 | 0.03667 | −0.09067 |
| 1 | rs179853 | 0.000531 | 0.2238 | 0.5276 | 0.2486 | T/T | T/C | C/C | −0.08775 | −0.02425 | 0.1511 |
| 13 | rs9567402 | 0.000537 | 0.08333 | 0.4306 | 0.4861 | C/C | C/T | T/T | 0.1697 | 0.0609 | −0.07761 |
| 6 | rs1252603 [symbol] | 0.000539 | 0.01934 | 0.2597 | 0.721 | T/T | T/C | C/C | −0.2348 | −0.1179 | 0.0566 |
| 14 | rs7400989 | 0.000543 | 0.1657 | 0.5 | 0.3343 | G/G | G/C | C/C | −0.111 | −0.03202 | 0.1179 |
| 11 | rs481843 | 0.000544 | 0.01676 | 0.1536 | 0.8296 | T/T | T/C | C/C | −0.253 | −0.1704 | 0.04511 |
| 12 | rs1105017 [symbol] | 0.000544 | 0 | 0.09917 | 0.9008 | T/T | T/C | C/C | NA | −0.2433 | 0.03384 |
| 10 | rs3781264 | 0.000547 | 0.08621 | 0.454 | 0.4598 | G/G | G/A | A/A | 0.1733 | 0.06823 | −0.07425 |
| 4 | rs2672477 | 0.000548 | 0.225 | 0.5083 | 0.2667 | C/C | C/T | T/T | 0.08047 | 0.05938 | −0.1488 |
| 8 | rs6990940 | 0.000552 | 0.1343 | 0.5045 | 0.3612 | A/A | A/T | T/T | 0.163 | 0.03853 | −0.09452 |
| 4 | rs3922809 | 0.000553 | 0.1763 | 0.4855 | 0.3382 | A/A | A/T | T/T | −0.1966 | 0.03877 | 0.07661 |
| 12 | rs2024077 | 0.000557 | 0.09749 | 0.4373 | 0.4652 | G/G | G/A | A/A | −0.08045 | −0.07761 | 0.1053 |
| 4 | rs4865142 | 0.000557 | 0.169 | 0.462 | 0.369 | G/G | G/A | A/A | −0.1297 | −0.02431 | 0.103 |
| 16 | rs4547336 | 0.000558 | 0.2044 | 0.4779 | 0.3177 | G/G | G/T | T/T | 0.1244 | 0.02902 | −0.1039 |
| 6 | rs7749910 | 0.00056 | 0.1243 | 0.4972 | 0.3785 | A/A | A/G | G/G | 0.1625 | 0.03813 | −0.08751 |
| 7 | rs2108016 | 0.000577 | 0.02793 | 0.3659 | 0.6061 | T/T | T/C | C/C | 0.03543 | −0.1174 | 0.08486 |
| 6 | rs4945528 | 0.000581 | 0.03989 | 0.4359 | 0.5242 | C/C | C/G | G/G | 0.2495 | 0.07965 | −0.06299 |
| 4 | rs1687626 [symbol] | 0.000583 | 0.02528 | 0.2837 | 0.691 | A/A | A/G | G/G | −0.236 | −0.09633 | 0.0654 |
| 7 | rs1754720 [symbol] | 0.000589 | 0.1605 | 0.4585 | 0.3811 | A/A | A/G | G/G | 0.09187 | 0.08251 | −0.1062 |
| 4 | rs6850107 | 0.000591 | 0.03955 | 0.3418 | 0.6186 | A/A | A/G | G/G | −0.1798 | −0.08566 | 0.0731 |
| 3 | rs1078001 [symbol] | 0.000594 | 0.08287 | 0.3812 | 0.5359 | G/G | G/A | A/A | −0.144 | −0.06465 | 0.08329 |
| 10 | rs1118783 [symbol] | 0.000609 | 0.08146 | 0.4972 | 0.4213 | A/A | A/G | G/G | 0.1253 | 0.06594 | −0.09393 |
| 18 | rs4987853 | 0.00061 | 0.02755 | 0.3251 | 0.6474 | G/G | G/A | A/A | −0.2699 | −0.083 | 0.06299 |
| 3 | rs2727952 | 0.000616 | 0.06886 | 0.3263 | 0.6048 | T/T | T/G | G/G | 0.2309 | 0.08723 | −0.05021 |
| 1 | rs599839 | 0.00062 | 0.08215 | 0.3569 | 0.5609 | G/G | G/A | A/A | 0.217 | 0.05458 | −0.05976 |
| 14 | rs1782413 [symbol] | 0.000621 | 0.006098 | 0.2226 | 0.7713 | T/T | T/C | C/C | 0.424 | 0.1589 | −0.034 |
| 8 | rs1747044 [symbol] | 0.000623 | 0.1078 | 0.4581 | 0.4341 | G/G | G/A | A/A | −0.2657 | 0.01291 | 0.07141 |
| 10 | rs1074937 [symbol] | 0.000628 | 0.03039 | 0.3315 | 0.6381 | T/T | T/C | C/C | 0.1298 | 0.1195 | −0.05797 |
| 6 | rs9487172 | 0.000631 | 0.1271 | 0.4972 | 0.3757 | A/A | A/C | C/C | 0.137 | 0.04321 | −0.09885 |
| 17 | rs1470034 | 0.000632 | 0.04735 | 0.3565 | 0.5961 | C/C | C/G | G/G | −0.3551 | −0.03408 | 0.05793 |
| 13 | rs1050769 [symbol] | 0.000636 | 0.04986 | 0.3352 | 0.615 | G/G | G/A | A/A | −0.2057 | −0.07115 | 0.06954 |
| 19 | rs1297558 [symbol] | 0.000639 | 0.1691 | 0.49 | 0.341 | C/C | C/A | A/A | −0.113 | −0.03274 | 0.1165 |
| 6 | rs9368942 | 0.00064 | 0.08056 | 0.4333 | 0.4861 | G/G | G/C | C/C | −0.2161 | −0.02747 | 0.07867 |
| 20 | rs998934 | 0.000642 | 0.03003 | 0.2763 | 0.6937 | T/T | T/C | C/C | 0.1818 | 0.1317 | −0.04602 |
| 2 | rs6547844 | 0.000646 | 0.01114 | 0.1783 | 0.8106 | T/T | T/A | A/A | −0.02266 | −0.1858 | 0.05107 |
| 6 | rs9285409 | 0.000647 | 0.2069 | 0.4914 | 0.3017 | A/A | A/C | C/C | 0.1646 | −0.00446 | −0.07945 |
| 12 | rs33223 | 0.000649 | 0.06077 | 0.3785 | 0.5608 | G/G | G/A | A/A | −0.2134 | −0.05555 | 0.0712 |
| 11 | rs1222538 [symbol] | 0.000652 | 0.02035 | 0.2994 | 0.6802 | A/A | A/G | G/G | 0.3103 | 0.1149 | −0.04484 |
| 12 | rs2300245 | 0.000655 | 0.03683 | 0.3456 | 0.6176 | T/T | T/G | G/G | 0.1245 | 0.1134 | −0.06489 |
| 6 | rs6568591 | 0.000655 | 0.08746 | 0.4723 | 0.4402 | G/G | G/A | A/A | 0.1786 | 0.05377 | −0.08087 |
| 10 | rs1118789 [symbol] | 0.000661 | 0.05 | 0.3972 | 0.5528 | C/C | C/G | G/G | 0.168 | 0.08801 | −0.06695 |
| 6 | rs1291389 | 0.000662 | 0.04545 | 0.2614 | 0.6932 | T/T | T/C | C/C | −0.2088 | −0.09985 | 0.05898 |
| 12 | rs1282436 [symbol] | 0.000669 | 0.1643 | 0.4791 | 0.3565 | G/G | G/A | A/A | 0.1684 | 0.01357 | −0.07884 |
| 10 | rs1124440 [symbol] | 0.000669 | 0.002755 | 0.1185 | 0.8788 | G/G | G/A | A/A | −0.8605 | −0.1857 | 0.03497 |
| 11 | rs1126359 [symbol] | 0.00067 | 0.1994 | 0.4848 | 0.3158 | G/G | G/C | C/C | 0.1314 | 0.02355 | −0.09843 |
| 1 | rs7547134 | 0.000671 | 0.116 | 0.4807 | 0.4033 | C/C | C/G | G/G | 0.1175 | 0.06845 | −0.09272 |
| 1 | rs234106 | 0.000673 | 0.2295 | 0.5269 | 0.2436 | C/C | C/T | T/T | −0.09602 | −0.01813 | 0.1423 |
| 15 | rs1464150 | 0.000677 | 0.003021 | 0.142 | 0.855 | G/G | G/C | C/C | −0.61 | −0.1877 | 0.04106 |
| 7 | rs1024479 [symbol] | 0.000677 | 0.2183 | 0.5044 | 0.2773 | C/C | C/G | G/G | 0.1153 | 0.01939 | −0.1247 |
| 4 | rs1912806 | 0.000679 | 0.1385 | 0.3989 | 0.4626 | A/A | A/G | G/G | −0.1924 | 0.004378 | 0.07278 |
| 13 | rs2219499 | 0.000681 | 0.1569 | 0.479 | 0.3641 | G/G | G/A | A/A | 0.2256 | −0.00859 | −0.0508 |
| 4 | rs1702175 [symbol] | 0.000694 | 0.04444 | 0.3333 | 0.6222 | A/A | A/G | G/G | −0.06333 | −0.1115 | 0.07985 |
| 11 | rs6485604 | 0.000696 | 0.005525 | 0.1934 | 0.8011 | T/T | T/C | C/C | −0.1836 | −0.159 | 0.04672 |
| 23 | rs6627369 | 0.000699 | 0.3295 | 0.2436 | 0.4269 | C/C | C/T | T/T | 0.1329 | −0.06575 | −0.066 |
| 8 | rs3104327 | 0.000703 | 0.07536 | 0.3304 | 0.5942 | T/T | T/C | C/C | −0.2523 | −0.04404 | 0.05676 |
| 6 | rs2029555 | 0.000705 | 0.2681 | 0.4337 | 0.2982 | C/C | C/T | T/T | −0.04752 | −0.07574 | 0.172 |
| 6 | rs9398341 | 0.000714 | 0.2074 | 0.4972 | 0.2955 | A/A | A/C | C/C | −0.04422 | −0.06976 | 0.1685 |
| 7 | rs2189817 | 0.000715 | 0.1798 | 0.4663 | 0.3539 | A/A | A/G | G/G | 0.1805 | −0.00236 | −0.07037 |
| 1 | rs3766465 | 0.00072 | 0.03621 | 0.2535 | 0.7103 | G/G | G/A | A/A | −0.3885 | −0.05485 | 0.04733 |
| 4 | rs7664565 | 0.000722 | 0.07736 | 0.361 | 0.5616 | A/A | A/G | G/G | 0.1463 | 0.09328 | −0.06652 |
| 13 | rs2706411 | 0.000724 | 0.05817 | 0.3684 | 0.5734 | T/T | T/C | C/C | 0.1852 | 0.08378 | −0.06134 |
| 9 | rs7868056 | 0.000726 | 0.1601 | 0.427 | 0.4129 | T/T | T/C | C/C | 0.1632 | 0.02922 | −0.07369 |
| 12 | rs1230127 [symbol] | 0.000726 | 0 | 0.1131 | 0.8869 | T/T | T/C | C/C | NA | 0.2394 | −0.0314 |
| 13 | rs2329285 | 0.000728 | 0.02479 | 0.2782 | 0.697 | A/A | A/C | C/C | 0.4289 | 0.08223 | −0.03896 |
| 4 | rs1310978 [symbol] | 0.000731 | 0.02241 | 0.2241 | 0.7535 | G/G | G/C | C/C | −0.2206 | −0.1317 | 0.05025 |
| 12 | rs1780657 [symbol] | 0.000731 | 0.005731 | 0.1203 | 0.8739 | G/G | G/T | T/T | 0.5085 | 0.1977 | −0.02333 |
| 6 | rs9488153 | 0.000735 | 0.2099 | 0.449 | 0.3411 | T/T | T/C | C/C | −0.07309 | −0.05302 | 0.1367 |
| 11 | rs1079339 [symbol] | 0.000735 | 0.2327 | 0.482 | 0.2853 | A/A | A/T | T/T | 0.1597 | −0.0212 | −0.07189 |
| 6 | rs7761223 | 0.000737 | 0.157 | 0.4821 | 0.3609 | C/C | C/T | T/T | −0.04138 | −0.07833 | 0.1403 |
| 22 | rs1305362 [symbol] | 0.000742 | 0.0554 | 0.2825 | 0.662 | A/A | A/T | T/T | 0.314 | 0.05655 | −0.03938 |
| 16 | rs4780416 | 0.000745 | 0.1629 | 0.4829 | 0.3543 | A/A | A/G | G/G | −0.1596 | −0.00713 | 0.09141 |
| 8 | rs776394 | 0.000746 | 0.01381 | 0.1823 | 0.8039 | G/G | G/A | A/A | −0.4552 | −0.1192 | 0.04272 |
| 16 | rs4888055 | 0.000746 | 0.1268 | 0.3746 | 0.4985 | T/T | T/C | C/C | −0.1918 | −0.0259 | 0.07173 |
| 5 | rs6893633 | 0.000746 | 0.0442 | 0.2928 | 0.663 | A/A | A/G | G/G | −0.22 | −0.08584 | 0.0565 |
| 8 | rs1373896 | 0.000747 | 0.002778 | 0.1806 | 0.8167 | C/C | C/T | T/T | −0.5627 | −0.1526 | 0.04561 |
| 8 | rs1760418 [symbol] | 0.000749 | 0.005618 | 0.09551 | 0.8989 | A/A | A/G | G/G | −0.1315 | −0.2555 | 0.03594 |
| 18 | rs1050285 [symbol] | 0.000752 | 0.1153 | 0.5043 | 0.3804 | T/T | T/G | G/G | −0.1402 | −0.04014 | 0.101 |
| 5 | rs6451758 | 0.000761 | 0.04132 | 0.3388 | 0.6198 | A/A | A/T | T/T | 0.2549 | 0.08177 | −0.05144 |
| 4 | rs1705028 [symbol] | 0.000767 | 0.01928 | 0.2176 | 0.7631 | A/A | A/G | G/G | 0.09917 | 0.1672 | −0.04187 |
| 6 | rs199024 | 0.000769 | 0.102 | 0.3768 | 0.5212 | G/G | G/A | A/A | 0.1932 | 0.06011 | −0.05507 |
| 2 | rs1704289 [symbol] | 0.00077 | 0.01108 | 0.1801 | 0.8089 | A/A | A/G | G/G | −0.02266 | −0.1834 | 0.04723 |
| 11 | rs948133 | 0.000775 | 0.2485 | 0.5 | 0.2515 | A/A | A/G | G/G | 0.1139 | 0.0245 | −0.1217 |
| 10 | rs7906986 | 0.000776 | 0.01408 | 0.1803 | 0.8056 | T/T | T/C | C/C | −0.33 | −0.148 | 0.03712 |
| 10 | rs8178980 | 0.000777 | 0 | 0.133 | 0.867 | T/T | T/C | C/C | #VALUE! | −0.2003 | 0.03814 |
| 20 | rs1773846 [symbol] | 0.000781 | 0.005731 | 0.1347 | 0.8596 | G/G | G/C | C/C | −0.4689 | −0.1739 | 0.0438 |
| 8 | rs1841019 | 0.000782 | 0.1117 | 0.5391 | 0.3492 | C/C | C/A | A/A | 0.1753 | 0.03637 | −0.08368 |
| 8 | rs1050330 [symbol] | 0.000782 | 0.04959 | 0.3196 | 0.6309 | T/T | T/C | C/C | −0.1877 | −0.08085 | 0.06579 |
| 14 | rs4981259 | 0.000782 | 0.2 | 0.5099 | 0.2901 | G/G | G/A | A/A | 0.1493 | 0.002226 | −0.08736 |
| 12 | rs1074353 [symbol] | 0.000782 | 0.1568 | 0.426 | 0.4172 | G/G | G/A | A/A | −0.1597 | −0.01702 | 0.08365 |
| 10 | rs2601749 | 0.000784 | 0.1528 | 0.4639 | 0.3833 | C/C | C/T | T/T | −0.06595 | −0.06051 | 0.1258 |
| 13 | rs1878410 | 0.000785 | 0.1385 | 0.4488 | 0.4127 | T/T | T/C | C/C | −0.1834 | −0.00264 | 0.07712 |
| 4 | rs2119787 | 0.000786 | 0.175 | 0.425 | 0.4 | G/G | G/A | A/A | −0.1043 | −0.03613 | 0.104 |
| 2 | rs2380609 | 0.000787 | 0.1185 | 0.383 | 0.4985 | T/T | T/G | G/G | −0.239 | 0.008973 | 0.06636 |
| 6 | rs2452965 | 0.000794 | 0.1637 | 0.4167 | 0.4196 | A/A | A/G | G/G | −0.1266 | −0.03083 | 0.1006 |
| 23 | rs732572 | 0.000794 | 0.04249 | 0.08215 | 0.8754 | G/G | G/A | A/A | −0.3466 | −0.1023 | 0.03273 |
| 10 | rs7099178 | 0.000796 | 0.01994 | 0.2934 | 0.6866 | G/G | G/C | C/C | 0.1735 | 0.1227 | −0.05402 |
| 3 | rs3816529 | 0.000809 | 0.005634 | 0.2225 | 0.7718 | G/G | G/C | C/C | 0.2453 | 0.1601 | −0.02994 |
| 15 | rs7169200 | 0.00081 | 0.2194 | 0.4986 | 0.2821 | T/T | T/C | C/C | −0.127 | 0.01505 | 0.1096 |
| 11 | rs1089710 [symbol] | 0.000812 | 0.207 | 0.5015 | 0.2915 | C/C | C/T | T/T | −0.1406 | 0.01057 | 0.1014 |
| 4 | rs1540052 | 0.000813 | 0.07182 | 0.3508 | 0.5773 | C/C | C/T | T/T | −0.2118 | −0.05003 | 0.06519 |
| 19 | rs2974211 | 0.000817 | 0.1047 | 0.3953 | 0.5 | G/G | G/A | A/A | 0.1391 | 0.06821 | −0.08017 |
| 4 | rs2726686 | 0.000817 | 0.231 | 0.507 | 0.262 | G/G | G/A | A/A | 0.07072 | 0.06316 | −0.1516 |
| 8 | rs1709201 [symbol] | 0.000818 | 0.008621 | 0.2241 | 0.7672 | A/A | A/G | G/G | −0.1745 | 0.1808 | −0.04582 |
| 4 | rs1399404 | 0.000819 | 0.2284 | 0.507 | 0.2646 | G/G | G/C | C/C | 0.07839 | 0.05154 | −0.1447 |
| 1 | rs6692930 | 0.00082 | 0.03047 | 0.2936 | 0.6759 | C/C | C/T | T/T | 0.2622 | 0.1007 | −0.04659 |
| 7 | rs1153163 [symbol] | 0.000829 | 0.1737 | 0.465 | 0.3613 | A/A | A/C | C/C | 0.1674 | 0.009214 | −0.07542 |
| 5 | rs1051267 [symbol] | 0.000832 | 0.02493 | 0.2909 | 0.6842 | C/C | C/A | A/A | 0.343 | 0.0948 | −0.04114 |
| 10 | rs1254531 | 0.000832 | 0.01393 | 0.234 | 0.7521 | A/A | A/G | G/G | 0.5048 | 0.1092 | −0.03538 |
| 17 | rs8064630 | 0.000833 | 0.0554 | 0.3435 | 0.6011 | A/A | A/G | G/G | −0.1943 | −0.06592 | 0.06965 |
| 21 | rs4591420 | 0.000834 | 0.1091 | 0.528 | 0.3628 | T/T | T/C | C/C | −0.1945 | −0.00368 | 0.09313 |
| 6 | rs1291402 | 0.000836 | 0.05292 | 0.2702 | 0.6769 | C/C | C/A | A/A | −0.1507 | −0.1069 | 0.06325 |
| 8 | rs1325582 [symbol] | 0.000837 | 0.1127 | 0.5268 | 0.3606 | G/G | G/C | C/C | 0.1753 | 0.04075 | −0.08086 |
| 5 | rs1368378 | 0.000846 | 0.1425 | 0.4644 | 0.3932 | C/C | C/T | T/T | 0.1762 | 0.02572 | −0.07179 |
| 8 | rs884530 | 0.00085 | 0.01111 | 0.175 | 0.8139 | T/T | T/C | C/C | −0.4837 | −0.1258 | 0.04277 |
| 17 | rs4890120 | 0.000854 | 0.01412 | 0.1949 | 0.791 | A/A | A/G | G/G | −0.4886 | −0.1111 | 0.03947 |
| 21 | rs9306015 | 0.000855 | 0.213 | 0.5 | 0.287 | T/T | T/A | A/A | −0.1439 | −0.00939 | 0.09452 |
| 8 | rs1199174 [symbol] | 0.000857 | 0.01111 | 0.2306 | 0.7583 | C/C | C/A | A/A | −0.4911 | −0.1008 | 0.05121 |
| 11 | rs4073610 | 0.000858 | 0.1954 | 0.4828 | 0.3218 | A/A | A/T | T/T | 0.1798 | −0.01599 | −0.06832 |
| 13 | rs1050736 [symbol] | 0.000862 | 0.08832 | 0.4387 | 0.4729 | G/G | G/A | A/A | −0.1919 | −0.03362 | 0.08084 |
| 13 | rs9571907 | 0.000872 | 0.1524 | 0.482 | 0.3657 | G/G | G/A | A/A | 0.2328 | −0.01815 | −0.04794 |
| 9 | rs1076069 [symbol] | 0.000872 | 0.1264 | 0.4425 | 0.431 | C/C | C/T | T/T | −0.158 | −0.03299 | 0.08297 |
| 6 | rs1115328 [symbol] | 0.000874 | 0.1597 | 0.5266 | 0.3137 | A/A | A/C | C/C | −0.1472 | −0.00152 | 0.1011 |
| 13 | rs1329682 | 0.000881 | 0.1552 | 0.4684 | 0.3764 | C/C | C/A | A/A | 0.2166 | −0.00503 | −0.05142 |
| 13 | rs9526671 | 0.000882 | 0.02228 | 0.2368 | 0.7409 | G/G | G/A | A/A | −0.2693 | −0.1051 | 0.05565 |
| 14 | rs1048404 [symbol] | 0.000891 | 0.04749 | 0.2626 | 0.6899 | G/G | G/C | C/C | 0.3505 | 0.0576 | −0.03562 |
| 2 | rs1169527 | 0.000891 | 0.04709 | 0.2964 | 0.6565 | A/A | A/G | G/G | −0.2039 | −0.08766 | 0.05547 |
| 13 | rs4884976 | 0.000892 | 0.05248 | 0.3499 | 0.5977 | C/C | C/T | T/T | 0.2316 | 0.06443 | −0.06439 |
| 16 | rs1164566 [symbol] | 0.000901 | 0.06887 | 0.3774 | 0.5537 | T/T | T/C | C/C | 0.2201 | 0.05959 | −0.05651 |
| 5 | rs1051266 [symbol] | 0.000902 | 0.005714 | 0.1171 | 0.8771 | C/C | C/T | T/T | −0.6711 | −0.1728 | 0.03419 |
| 1 | rs1252579 | 0.000905 | 0.2095 | 0.5391 | 0.2514 | G/G | G/A | A/A | −0.1249 | 0.02001 | 0.1122 |
| 4 | rs795985 | 0.000907 | 0.03878 | 0.3324 | 0.6288 | T/T | T/G | G/G | 0.385 | 0.04943 | −0.04303 |
| 13 | rs7319124 | 0.000913 | 0.06069 | 0.3468 | 0.5925 | C/C | C/T | T/T | 0.3256 | 0.02981 | −0.05108 |
| 2 | rs4408769 | 0.000917 | 0.163 | 0.3978 | 0.4392 | A/A | A/G | G/G | −0.1733 | 0.006445 | 0.07041 |
| 9 | rs4842173 | 0.000917 | 0.08939 | 0.3855 | 0.5251 | C/C | C/T | T/T | −0.1539 | −0.0486 | 0.08194 |
| 13 | rs342673 | 0.000919 | 0.07182 | 0.3204 | 0.6077 | A/A | A/G | G/G | −0.2137 | −0.0541 | 0.05856 |
| 4 | rs1172373 [symbol] | 0.00092 | 0.09366 | 0.449 | 0.4573 | G/G | G/A | A/A | −0.04395 | −0.08712 | 0.1085 |
| 4 | rs4861163 | 0.00092 | 0.03922 | 0.381 | 0.5798 | A/A | A/G | G/G | 0.1576 | 0.08954 | −0.06706 |
| 10 | rs1235895 [symbol] | 0.000922 | 0.005587 | 0.1229 | 0.8715 | C/C | C/G | G/G | −0.3843 | −0.1883 | 0.03832 |
| 4 | rs1003258 [symbol] | 0.000936 | 0.1946 | 0.515 | 0.2904 | T/T | T/C | C/C | −0.08545 | −0.02474 | 0.1464 |
| 7 | rs2722269 | 0.000937 | 0.04942 | 0.3634 | 0.5872 | G/G | G/A | A/A | 0.3199 | 0.04282 | −0.0553 |
| 6 | rs4895759 | 0.000939 | 0.01462 | 0.307 | 0.6784 | T/T | T/A | A/A | −0.1892 | −0.1063 | 0.06716 |
| 12 | rs33229 | 0.00094 | 0.06128 | 0.3844 | 0.5543 | T/T | T/C | C/C | −0.2134 | −0.05398 | 0.06677 |
| 16 | rs8059982 | 0.000953 | 0.1519 | 0.49 | 0.3582 | G/G | G/C | C/C | 0.1374 | 0.03751 | −0.09402 |
| 8 | rs2935295 | 0.000961 | 0.03047 | 0.3019 | 0.6676 | C/C | C/T | T/T | 0.1944 | 0.1118 | −0.04856 |
| 6 | rs1291401 | 0.000963 | 0.05234 | 0.2617 | 0.686 | T/T | T/C | C/C | −0.1507 | −0.1063 | 0.06132 |
| 7 | rs4947934 | 0.000963 | 0.259 | 0.4711 | 0.27 | T/T | T/A | A/A | 0.1682 | −0.04941 | −0.05155 |
| 10 | rs1050967 [symbol] | 0.000963 | 0.09915 | 0.4448 | 0.4561 | C/C | C/T | T/T | 0.1218 | 0.06403 | −0.08596 |
| 6 | rs4946854 | 0.000966 | 0.1326 | 0.4475 | 0.4199 | C/C | C/T | T/T | 0.138 | 0.05223 | −0.08014 |
| 3 | rs4973856 | 0.000966 | 0.0884 | 0.3646 | 0.547 | T/T | T/A | A/A | 0.2109 | 0.04895 | −0.05517 |
| 10 | rs7904517 | 0.000968 | 0.131 | 0.4226 | 0.4464 | C/C | C/T | T/T | −0.1225 | −0.04399 | 0.09724 |
| 6 | rs7765175 | 0.000976 | 0.1547 | 0.489 | 0.3564 | T/T | T/C | C/C | −0.05086 | −0.0666 | 0.1345 |
| 23 | rs1109436 [symbol] | 0.000982 | 0.1232 | 0.1541 | 0.7227 | A/A | A/G | G/G | −0.181 | −0.05225 | 0.05134 |
| 2 | rs1092868 [symbol] | 0.000986 | 0.1891 | 0.4957 | 0.3152 | C/C | C/G | G/G | 0.1318 | 0.007078 | −0.1019 |
| 8 | rs1113608 [symbol] | 0.000986 | 0.006061 | 0.1758 | 0.8182 | T/T | T/G | G/G | 0.431 | 0.1596 | −0.03907 |
| 4 | rs1760079 [symbol] | 0.000991 | 0.03933 | 0.3287 | 0.632 | A/A | A/C | C/C | −0.1798 | −0.08865 | 0.06467 |
| 20 | rs2143618 | 0.000999 | 0.01183 | 0.3077 | 0.6805 | G/G | G/A | A/A | 0.3223 | 0.1157 | −0.04602 |

[symbol]

indicates data missing or illegible when filed

1. A method to determine predisposition or risk to develop Parkinson's Disease (PD) in a subject in need thereof comprising: (a) providing a biological sample from a subject in need thereof, (b) determining a ratio of SNCA long transcript to SNCA total transcript in the subject's biological sample and (c) comparing the ratio of SNCA long transcript to SNCA total transcript from the subject sample to a reference ratio of SNCA long transcript to SNCA total transcript, wherein the reference ratio is associated with a non-PD status, and wherein an increased ratio of SNCA long transcript to SNCA total transcript in the subject's sample compared to the reference ratio of SNCA long transcript to SNCA total transcript is indicative of a risk for developing Parkinson's Disease.

2. A method to diagnose PD in a subject in need thereof, the method comprising: (a) providing a biological sample from a subject in need thereof, (b) determining a ratio of SNCA long transcript to SNCA total transcript in the subject's sample and (c) comparing the ratio of SNCA long transcript to SNCA total transcript from the subject's sample to a ratio of SNCA long transcript to SNCA total transcript in a reference sample from healthy individuals/non-PD status, wherein an increased ratio of SNCA long transcript to SNCA total transcript in the subject's sample compared to the ratio of SNCA long transcript to SNCA total transcript in the reference non-PD status sample indicates that the subject is suffering from Parkinson's Disease.

3. The method of claim 2, further comprising comparing the ratio of SNCA long transcript to SNCA total transcript from the subject to a reference ratio of SNCA long transcript to SNCA total transcript for a PD disease status; wherein a ratio of SNCA long transcript to SNCA total transcript in the subject's sample which is similar or comparable to the reference ratio of SNCA long transcript to SNCA total transcript for a PD status indicates that the subject is suffering from PD.

4. A method to diagnose PD in a subject in need thereof, comprising: (a) providing a biological sample from a subject, (b) determining a ratio of SNCA long transcript to SNCA total transcript in the sample obtained from the subject; (c) comparing the ratio of SNCA long transcript to SNCA total transcript from the subject to a reference ratio of SNCA long transcript to SNCA total transcript for a PD disease status; wherein a ratio of SNCA long transcript to SNCA total transcript in the subject's sample which is similar or comparable to the reference ratio of SNCA long transcript to SNCA total transcript for a PD status indicates that the subject is suffering from PD.

5. The method of claim 4, further comprising comparing the ratio of SNCA long transcript to SNCA total transcript from the subject's sample to a ratio of SNCA long transcript to SNCA total transcript in a reference sample from healthy individuals/non-PD status, wherein an increased ratio of SNCA long transcript to SNCA total transcript in the subject's sample compared to the ratio of SNCA long transcript to SNCA total transcript in the reference non-PD status sample indicates that the subject is suffering from Parkinson's Disease.

6. The method of claim 3, 4 or 5, wherein the PD disease status is determined by any suitable method, including but not limited to a physical examination of the subject, a neurological examination of the subject, a brain scan, or a combination thereof.

7. The method of claim 1 to 4, wherein the subject is not diagnosed with PD.

8. The method of claim 1 to 4, further comprising a physical examination of the subject, a neurological examination of the subject, a brain scan, or a combination thereof.

9. The method of any one of claims 1 to 4 further comprising a step of sequencing nucleic acids isolated from the subject's sample to determine the presence or absence of a PD-risk associated SNP, wherein the presence of a PD-risk associated SNP is further indicative that the subject is at risk of developing PD or is suffering from PD.

10. The method of claim 9, wherein the SNP is rs356168C/C risk-associated variant, rs356165 risk-associated variant, rs2736990 risk-associated variant, any other risk associated SNP, or any combination thereof.

11. The method of claim 1-4, wherein the subject is suspected of having PD or is at risk of developing PD based on the presence of any one of parkinsonism symptoms.

12. The method of any one of claims 1 to 4, wherein the method is carried out in the absence or presence of dopamine affecting agent administered to the subject, wherein an increased ratio of SNCA long transcript to SNCA total transcript in the presence of dopamine compared to the ratio of SNCA long transcript to SNCA total transcript in the absence of dopamine is indicative of a subject having an increased risk to develop PD.

13. A method to identify a candidate agent which has a therapeutic effect on PD, the method comprising: (a) providing a sample from a cortical neuron cell culture, (b) determining a ratio of SNCA long transcript to SNCA total transcript in a sample from the cortical neuron cell culture, wherein the sample is obtained in the presence and absence of a candidate agent, wherein a lowered ratio of SNCA long transcript to SNCA total transcript in the sample in the presence of the candidate agent is indicative of an agent which is a therapeutic agent for treatment of PD.

14. A method to identify a candidate agent which has a therapeutic effect on PD, the method comprising: (a) providing a sample from an animal model of PD; (b) determining a ratio of SNCA long transcript to SNCA total transcript in the sample from an animal model of PD, wherein the sample is obtained in the presence and absence of a candidate agent, administered to the animal model of PD, wherein a lowered ratio of SNCA long transcript to SNCA total transcript in the sample in the presence of the candidate agent is indicative of an agent which is a therapeutic agent for treatment of PD.

15. A method to determine a therapeutic effect of a candidate agent in a subject suffering from PD, the method comprising: (a) determining a ratio of SNCA long transcript to SNCA total transcript in a sample from a subject suffering from PD, wherein the sample is obtained in the presence and absence of a candidate agent, wherein a lowered ratio of SNCA long transcript to SNCA total transcript in the sample in the presence of the candidate agent is indicative of an agent which is a therapeutic agent for treatment of PD.

16. The method of claim 13, 14 or 15, wherein the lowered ratio of SNCA long transcript to SNCA total transcript in the sample in the presence of the candidate agent is due to a reduced level of SNCA long transcript in the presence of the candidate agent compared to level of SNCA long transcript the absence of the candidate agent.

17. The method of claim 13, 14 or 15, wherein the subject is diagnosed with PD and is not administered dopamine affecting agents.

18. The method of claim 15, wherein the subject is diagnosed by clinical symptoms, imaging of dopamine uptake, or combination thereof.

19. The method of any one of claims 13 to 15, wherein a ratio of SNCA long transcript to SNCA total transcript is determined by quantifying SNCA long transcript and SNCA total transcript.

20. The method of any one of claim 1, 2, 4, 13, 14, or 15, further comprising isolating nucleic acids from the subject's biological sample.

21. The method of any one of claim 1, 2, 4, 13, 14, or 15, further comprising quantifying the levels of SNCA long transcript and SNCA total transcript, wherein the levels of SNCA long transcript and SNCA total transcript are quantified.

22. The method of claim 1, 2, 4, 13, 14, or 15, wherein the ratio of SNCA long transcript to SNCA total transcript is determined in a CSF sample, blood sample, plasma, or serum.

23. A kit comprising PCR primers to carry out step (b) of the method of any one of claim 1, 2, or 4 and instructions to carry out steps (a), (b) and (c) of the method of any of claim 1, 2, or 4.

24. A kit comprising at least one PCR primer to selectively quantify the SNCA long transcript and SNCA total transcript in a sample from a subject according to any one of claim 1, 2, or 4, so as to determine the ratio of SNCA long transcript and SNCA total transcript, and instructions to carry out steps (a) and (b) of the method of any of claim 1, 2, or 4.

25. A method of treating PD in a subject in need thereof, the method comprising: (a) providing a biological sample from a subject in need thereof, (b) determining a ratio of SNCA long transcript to SNCA total transcript in the subject's sample, (c) comparing the ratio of SNCA long transcript to SNCA total transcript from the subject's sample to a reference ratio of SNCA long transcript to SNCA total transcript, wherein the reference ratio is associated with a non-PD status, and (d) administering a dopamine affecting agent, wherein the dopamine affecting agent is administered if there is an increased ratio of SNCA long transcript to SNCA total transcript in the subject's sample compared to the ratio of SNCA long transcript to SNCA total transcript in the reference non-PD status sample.

26. The method of claim 25, further comprising comparing the ratio of SNCA long transcript to SNCA total transcript from the subject to a reference ratio of SNCA long transcript to SNCA total transcript, wherein the reference ratio is associated with a PD disease status; wherein the dopamine affecting agent is administered if the ratio of SNCA long transcript to SNCA total transcript in the subject's sample is similar or comparable to the reference ratio of SNCA long transcript to SNCA total transcript for a PD status.

27. The method of claim 25, wherein the subject is not administered a dopamine affecting agent.

28. The method of claim 25, further comprising isolating nucleic acids from the subject's biological sample.

29. The method of claim 25, further comprising quantifying the levels of SNCA long transcript and SNCA total transcript, wherein the levels of SNCA long transcript and SNCA total transcript are quantified.

30. The method of claim 25 or 26, wherein the dopamine affecting agent is levodopa, a dopamine agonist, a MAO-B inhibitor, a dopa decarboxylase inhibitor, a COMT inhibitor, or any combination thereof.

31. The method of claim 30, wherein the MAO-B inhibitor is selegiline or rasagiline.
